(19) United States
(12) Patent Application Publication
(10) Pub. No.: US 2007/0078085 A1
(43) Pub. Date: Apr. 5, 2007

(54) Methods and compositions for the utilization and targeting of osteomimicry

(76) Inventors: Leland W.K. Chung, Atlanta, GA (US); Wen-Chin Huang, Atlanta, GA (US); Valerie Odero-Marah, Tucker, GA (US); Daqing Wu, Atlanta, GA (US); Chia-Ling Hsieh, Decatur, GA (US); Haiyen Zhau, Atlanta, GA (US)

Correspondence Address: PATENT ADMINISTRATOR;KATTEN MUCHIN ROSENMAN LLP, 1025 THOMAS JEFFERSON STREET, N.W., EAST LOBBY: SUITE 700, WASHINGTON, DC, 20007-5201, US

(21) Appl. No.: 11/248,337

(22) Filed: Oct. 13, 2005

Related U.S. Application Data
(60) Provisional application No. 60/618,452, filed on Oct. 13, 2004.

(51) Int. Cl. A61K 48/00; A61K 38/18; C12Q 1/68; G01N 33/574

(52) U.S. Cl. 514/12; 514/44; 435/6; 435/7.23

(57) ABSTRACT

The present invention relates to compositions and methods for modulating osteomimicry within tumor and tissue cells with calcification potential. The invention further relates to screening compounds that modulate osteomimicry within tumor and tissue cells with calcification potential. Methods for using compounds identified by the screening assays for therapeutic treatments also are provided. The invention further relates to methods of treating those tumors and other diseases and disorders with calcification potential with compounds that modulate their osteomimetic potential.

### CROSS REFERENCE TO RELATED APPLICATIONS

This application claims priority under 35 USC Section 119(e) of U.S. Provisional Application No. 60/618,452, filed Oct. 13, 2004, the contents of which are hereby incorporated by reference in their entirety.

### FIELD OF THE INVENTION

The present invention relates to compositions and methods for modulating expression of osteomimicry within tumor and tissue cells with calcification potential. The invention further relates to screening compounds that modulate expression of osteomimicry within tumor and tissue cells with calcification potential. Methods for using molecules and compounds identified by the screening assays for therapeutic treatments also are provided. The invention further relates to methods of treating tumors and other diseases and disorders involving tumor and tissue cells with calcification potential with compounds that modulate their osteomimetic potential.

### BACKGROUND OF THE INVENTION

Cancer Bone Metastases

In 2004, bone metastases accounted for two thirds of an estimated 560,000 cancer deaths in the United States (1). Over 80% of cancer bone metastases come from prostate, breast, lung and renal cancers. Bone metastases often develop after patients fail hormonal therapy, are lethal, and have no effective therapy. Previous work targeting prostate cancer cells using conventional hormone therapy, chemotherapy or radiation therapy in men with hormonal refractory disease did not improve patient survival (2). New approaches targeting bone with zoledronic acid (Zometa) for breast and prostate cancers to slow down skeletal events in patients treated with hormonal therapy, bone-directed chemotherapy and radiation therapy using strontium-89 or samarium-153 for prostate and breast cancers have been approved by the Food and Drug Administration for the clinical treatment of osteoblastic/osteolytic bone metastases (3, 4). New chemotherapy modalities have shown promise for reducing the overall incidence of skeletal complications and improving survival in selected hormone-refractory prostate and breast cancer patients (5). These promising approaches are supported by laboratory results using gene therapy approaches to co-target tumor and stroma (6) and drug therapy targeting osteoblasts (7), osteoclasts (8); (9), marrow stromal cells (10-12), bone derived endothelium (13), cell adhesion to extracellular matrices (14) or selected growth factor pathways (15, 16), all of which have shown promise in a large number of bone metastasis models (8).

Bone is the second most common site of human cancer metastasis, harboring over 80% of the metastases from prostate, lung, breast and renal cancers (31). Bone metastasis contributes directly to cancer mortality and morbidity, with an estimated total of 560,000 deaths per year in the US and more than 85% of patients presenting with evidence of skeletal metastasis at autopsy (32). The extent of osseous involvement has been claimed to correlate directly with patient survival and the quality of life of cancer patients with bone pain, cancer-associated bone fractures and spinal compression, bone-metastasis-evoked cranial neuropathy from base of skull syndromes, anemia and infection (31). Despite some success transiently controlling the clinical symptoms with radiation, hormones, surgery and chemotherapy, advanced cancer inevitably becomes resistant to treatment. Recently, directly targeting the bone with bisphophonate to slow bone turnover in prostate and breast cancer patients treated with hormone withdrawal therapy has been credited with reducing bone pain and skeletal complications (33). The use of strontium 89 in combination with chemotherapy for the treatment of hormone refractory prostate cancer has resulted in statistically significant increases in patient survival (34). The development of the endothelin-1 receptor antagonist, atrasentan, to control osteoblastic lesions associated with prostate cancer bone metastasis (35, 36), and the inclusion of thalidomide to control angiogenesis associated with tumor progression (37), have been used clinically to improve the control of cancer bone metastases.

Tumor-Stroma Interaction

Tumor progression involves changes in genetic constituents as well as the gene expression of cancer cells. Changes are acquired through germ line transmission, somatic mutation and via epigenetic mechanisms through tumor-host interactions (38, 39). Laboratory and clinical data show that tumor-stroma interaction contributes to the development and progression of solid tumors (40). Cell culture studies, transgenic mouse models and carcinogenesis studies show that the progression of breast (41), prostate (42, 43), lung (44), skin (45) and gastric cancers (46) is promoted by stromal cells in the microenvironment surrounding the tumor. Using the human LNCaP prostate cancer cell line as a model, we demonstrated that prostate cancer is not a single-cell disease but involves intimate interaction between prostate cancer cells and prostate or bone stromal cells (47). Multiple early carcinogenetic steps defined at the genetic level can also be rationalized at the cellular level. For example, prostate cancer cells co-evolve with host stroma and both are needed for rapid tumor progression. Prostate cancer cells can evoke a “reactive stroma” response that drives further genetic and gene expression changes in prostate cancer (48). Through a series of complex, intimate bi-directional communications between prostate cancer and host stroma, which includes host fibromuscular stroma, endothelium, neural and infiltrating inflammatory cells, cancer cells gain additional growth and survival advantages and ultimately expand locally and disseminate to distant organs with lethal effect (49-51).

The inventors' previous studies (17, 23, 52) and others (18, 24, 53-57) show that human prostate cancer cells mimic osteoblasts by expressing proteins normally restricted to osteoblasts, such as osteocalcin (OC), bone sialoprotein (BSP), SPARC/osteonectin (ON), osteopontin (OPN) and the receptor activator of NF-κB ligand (RANKL). The metastatic human prostate cancer cells, C4-2 and C4-2B, also form bone when placed under mineralizing conditions (18). These results illustrate that human prostate cancer cells can functionally participate in the normal process of bone formation, bone turnover and mineralization like osteoblasts. Increasing osteoclastogenesis (creating bone pitting through increased bone turnover) could enhance the implantation and subsequent growth of prostate cancer cells in bone (58, 59).

Tissue Specific Expression within Tumor and Tissue Cells with Calcification Potential

The treatment of osteotropic tumors such as breast, osteosarcoma and prostate which have metastacised is a major challenge. These seemingly unrelated diseases, however, unite through a molecular analysis of the gene(s) that may be overexpressed in these forms of cancer during disease progression.

BSP is a noncollagenous bone protein in which tissue expression is limited to fully-differentiated osteoblasts in bone or other rare mineralized tissues, including tumors (Sodek J. et al., 1995, Conn. Tissue Research, 32:209-217). When evaluating human prostate cancer cells that have a propensity to metastasize to the skeleton, a surprising finding was that these cells have the ability to synthesize and secrete large amounts of non-collagenous bone matrix proteins, such as osteopontin (OPN) (Thalman G N, et al., 1997, Principles of Practice of Genitourinary Oncology, 409-416), osteocalcin (OC) (Curatolo C, et al., 1992, European Urology, 1:105-107), and BSP (Withold W., et al., 1997, Clinical Chemistry, 85-91). BSP is a 34 kilodalton protein rich in aspartic acid, glutamic acid, and glycine, with 50% of its carbohydrate rich in sialic acid. BSP is sulfated and phosphorylated (30% of serine residues), and contains a cell binding motiff sequence which is homologous to vitronectin (Oldbrg et al., 1988, J. Biol. Chem., 263:19433). BSP is involved in the nucleation front of mineralization during new bone formation, and binds hydroxyappatite tightly (Hunter et. al., 1993, PNAS, 90:8562; Chen et. al., 1992, JBMR, 7:987; Kobayashi et. al., 1996, J. Biochem., 119:475). Human BSP exists as a single copy gene on chromosome 4 (Fisher L. W. et. al., 1990, J. B. C., 265:4:2347-2351) and has 7 exons and 6 introns. BSP is distinct from other sialoproteins, such as, for example, dentin sialoprotein, osteopontin, IL-1, IL-6, TNF and other bone associated sialoproteins.

BSP is found in mature, bone-forming cells, but not in immature precursors (Bianco, et. al., 1991, Conn. Tiss. Int., 49:421; Chen et. al., 1991, Matrix, 11:133). BSP is found in trophoblastic cells of the placenta, and in cementum and dentin of teeth, but is absent in most other tissues, including unlineralized cartilage, intestine, kidney, liver, heart, and skeletal muscle (Macneil et. al., 1994, JBMR, 9:1597). In a transgenic mouse system, the activity of the BSP promoter was present at high levels in bone, but absent in kidney, liver, stomach, intestine, and spleen (Chen et. al., 1996, JBMR, 11:5:654-64), and the administration of exogenous glucocorticoid stimulated the expression of reporter gene 1.6 to 11 fold (Chen, et. al., 1996, Conn. Tiss. Res., 35:33-39). The DNA sequence of the BSP promoter is over 2000 base pairs long and contains numerous regulatory elements which include vitamin D, AP-1, glucocorticoid (GRE), hox, NF.kappa.b, TGF-.beta., CRE, etc. (Kim R. H., et. al., 1994, Matrix Biology. 14:31-40; Sodek J., et. al., 1996, Connective Tissue Research, 35:23-31; Kim R. H., et. al., 1996, Biochem. J., 318:219-226; Yamauchi M., et. al., 1996, Matrix Biology, 15:119-130; Kerr J. M., et. al., 1997, Calcif. Tiss. Int., 60:276282; Ogata Y., et. al., 1995, European J. Biochem., 230:183-192).

Osteocalcin

Osteocalcin (OC), a noncollagenous Gla protein produced specifically in osteoblasts, is synthesized, secreted, and deposited at the time of bone mineralization (Price, P. A. Vitamin-K dependent formation of bone GLA protein (onteocalcin) and its function. Vitam. Horm., 42:65-108, 1985). A recent study showed that immunohistochemical staining of OC as positive in primary osteoblastic osteosarcoma and chondroblastic osteosarcoma specimens as well as in five of seven fibroblastic osteosarcomas (Park, Y. K., Yung, M. H., Kim, Y. W., and Park, H. R. Osteocalcin expression in primary bone tumors: in situ hybridization and immunohistochemical study. J. Korean Med. Sci., 10:268-273, 1995). In addition, OC activity was detected in a wide spectrum of human tumors. This is consistent with the clinical observations that many human tumors exhibited calcification characteristics both in the primary and at distant metastases.

β2M Biology and Cancer

β2M forms a small invariable light chain subunit of the class I major histocompatibility complex (MHC, or HLA in humans) on the cell membrane of all nucleated cells. During the continuous turnover of the MHC molecules, β2M is shed from the cell membrane into blood. Lymphocytes are the main source of serum free β2M (60). Serum or urine β2M concentration is increased in several malignant diseases including prostate cancer (25), myeloma (61), lung cancer (62), renal cancer (63), lymphocytic malignancies (64, 65) and some inflammatory and autoimmune disorders (66-68). In these malignancies, serum β2M has significant prognostic value. Interferons (IFNs) have the ability to enhance the expression of class I and II MHC molecules. Accordingly, IFNs cause a rise in the formation β2M, which helps to present MHC molecules onto cell membranes, decrease tumor evasiveness and thus enhance host defense mechanisms against tumor growth. IFN alpha is used in diseases like multiple myeloma, where serum β2M measurements can assess tumor burden. Since MHC presentation is associated with host acquired immunity (69), decreased β2M or lost MHC expression could contribute to tumor cells' evasiveness (70-72) as with enhanced engraftment in patients who received bone marrow transplantation (73). It should be stressed that increased β2M levels have direct growth-promoting effects on prostate cancer, myeloma and bone and dendritic cell growth (74-77). The mechanisms could involve the increased expression of IL 6, 8 and 10 by a number of cancer cell types (78, 79) bone-like proteins in prostate cancer cells (52), and critical growth factor receptors, notably type 1 and 2 IGF receptors and EGF receptor, that enhance tumor growth (80). Previous work on β2M in myeloma revealed that the concentrations of this protein in serum and bone marrow aspirate correlated inversely with patient prognosis (61).

In view of the above, it remains important to pursue new molecular pathways that can be used to improve prognosis and treatment of cancer patients with lethal cancer phenotypes, bone metastases and associated complications. The present invention focusses on how osteomimicry contributes to intracellular cell signaling through augmentation of soluble factors and extracellular matrix-mediated interaction between cancer and stroma in the tumor microenvironment. This invention addresses a long-felt need for safe and effective methods of treatment of cancers and disorders with calcification potential by providing compositions and methods for directly interfering with the process of osteomimicry itself.

### SUMMARY OF THE INVENTION

The invention disclosed herein is based upon the concept that the specific targeting of the process of osteomimicry, either alone or in combination with chemotherapy and/or radiation therapy can prevent prostate, renal, breast, lung cancer cell growth and survival in bone through both direct and indirect actions on cancer cells and cells in the cancer microenvironment, and prolong the survival of cancer patients with bony metastases. The invention also has utility in predicting cancer dissemination to bone and visceral organs before radiographic or biochemical evidence of cancer metastases.

The invention disclosed herein is thus based in part upon the identification of novel targeted therapeutic agents for preventing, treating, curing and/or ameliorating tumors with calcification potential, including, but not limited to, localized or disseminated osteosarcoma, lung cancer, renal cancer, colon cancer, melanoma, thyroid cancer, brain cancer, multiple myeloma, and especially including, without limitation, breast and prostate cancers. The invention specifically targets sites of metastases of the above-mentioned osteotropic tumors, and where applicable, their supporting osseous stroma in the metastatic environment. In addition, the present invention also relates to therapeutic agents which may also be applicable to benign conditions, such as benign prostatic hyperplasia (BPH) or arterial sclerotic conditions where calcification and/or mineralization occurs.

The invention also provides relevant biomarkers based on the understanding of molecular pathways leading to cancer bone and visceral organ metastasis. The assessment of combinations of biomarkers relating to osteomimicry is predictive of cancer dissemination prior to radiographic and biochemical evidence of such events.

In one aspect of the present invention, a method is provided for treating and/or ameliorating an osteotropic-related disease or proliferative disorder in a mammal comprising administering to a subject in need thereof a therapeutically effective amount of a composition comprising a compound or substance exhibiting anti-osteomimetic activity or osteomimicry interfering activity or a functional derivative thereof; and a pharmaceutically acceptible excipient.

The anti-osteomimetic or osteomimicry interfering compound or a functional derivative thereof that exhibits anti-osteomimetic or osteomimicry interfering activity can include, but is not limited to, small organic molecules including naturally-occurring, synthetic, and biosynthetic molecules, small inorganic molecules including naturally-occurring and synthetic molecules, natural products including those produced by plants and fungi, nucleic acids, chemically modified nucleic acids, peptides, chemically modified peptides, and proteins. The anti-osteomimetic or osteomimicry interfering agent has the capability of inhibiting the osteomimetic potential and/or the osteomimetic properties of any known disease or disorder with osteomimetic or calcification and/or minerlization potential.

In another aspect of the present invention, a method is provided for interfering with the osteomimetic properties of a cell comprising introducing into a cell of a subject in need thereof an osteomimecry interfering compound, wherein said compound prevents or ameliorates the expression of the osteomimetic properties of said cell.

In one embodiment of the present invention, a method is provided for interfering with the osteomimetic properties of a cancer cell comprising introducing into a cancer cell of a subject in need thereof an osteomimecry interfering compound, wherein said compound prevents or ameliorates the expression of the osteomimetic properties of said cancer cell.

In yet another embodiment of the present invention, a method is provided for interfering with the osteomimetic properties of a prostate cancer cell comprising introducing into a prostate cancer cell of a subject in need thereof of an -osteomimecry interfering compound, wherein said compound prevents or ameliorates the expression of the osteomimetic properties of said prostate cancer cell.

In yet another aspect of the present invention, a method is provided for interfering with the osteomimetic potential of a cancer cell comprising introducing into a cancer cell of a subject in need thereof of an osteomimecry interfering compound, wherein said compound interferes with the osteomimetic potential of said cancer cell, prevents its growth, abrogates its supportive blood vessels, eliminates the survival androgen receptor signaling and causes massive cell death in pre-existing cancer or any combination thereof.

In one embodiment of the latter aspect of the present invention, the osteomimicry interfering compound inhibits one or more determinants governing prostate cancer bone colonization wherein said determinants comprise prostate cancer cell adhesion, extravasation, migration, invasion and interaction with bone cells or any combination thereof.

In yet another aspect of the present invention, a method is provided for identifying osteomimetic related and/or downstream genes and assessing the levels of expression and the variant forms of osteomimetic related and/or downstream genes as effective diagnostics for use in methods for predicting cancer bone and visceral organ metastases prior to radiographic and biochemical evidence of such events.

In each of the aforementioned aspects and embodiments of the present invention, the osteomimicry interfering compound interferes with the ability of the cell or cancer cell to express highly restricted bone-like proteins comprising one or more of osteocalcin (OC), bone sialoprotein (BSP), SPARC/osteonectin (ON), osteopontin (OPN) and the receptor activator of NF-κB ligand (RANKL), and/or to increase calcification, mineralization and bone turnover through the expression of genes normally restricted to osteoblasts and through epithelial to mesenchymal transition (EMT), a basic biologic process associated with increases of the motility, migration, invasion and survival of the cell or the cancer cell.

In each of the aforementioned aspects and embodiments of the present invention, the osteomimicry interfering compound comprises a compound whose osteomimicry-specific target is a gene or gene product from the vascular endothelial growth factor (VEGF) axis, a gene or gene product from the androgen receptor (AR) axis, a gene or gene product from the 7 transmembrane G protein-coupled receptor (GPCR) axis, a gene or gene product from the Protein Kinase A (PKA)/cyclic AMP responsive element binding protein (CREB) axis, and those current genes or gene products identified by microarray analyses as shown in shown in Appendix A, or any combination thereof.

In each of the aforementioned aspects and embodiments of the present invention, the osteomimicry interfering compound comprises a beta 2 microglobulin (β2M) siRNA, a β2M antibody, a Runx2 (i.e., cbfa1) transcription factor-specific siRNA, antibody, or antagonist, a GPCR antagonist, an AR or signaling antagonist, a VEGF or signaling antagonist, a PKA/CREB signal activation interrupter, a selective agent that interferes with β2M /PKA/CREB signaling, a selective agent that interferes with CREB transcription, phosphorylation and complex formation, a selective agent that interferes with β2M complex formation with either an intracellular protein or a membrane receptor or any combination thereof.

In one embodiment of the present invention, β2M siRNA encapsulated in liposomes, either with or without a targeting ligand (such as for example, and not by way of limitation, PSMA Ab, or aptamer), β2M siRNA or ribozyme viral vectors may be used to inhibit prostate cancer growth by inhibiting cancer cell proliferation and also the growth of neighboring stromal cells that support cancer growth, including osteoblasts, marrow stromal cells, endothelial cells and inflammatory cells.

In yet another embodiment of the present invention, viral vectors may used for the construction and delivery of therapeutic genes under the control of tissue-specific and tumor-restrictive promoters to co-target tumor and stroma. Representative examples of viral vectors and tissue-specific and tumor-restrictive promoters that may be used for the construction and delivery of therapeutic genes under the control of tissue-specific and tumor-restrictive promoters may be found in published U.S. Patent Application Nos. 20020025307, 20030078224, and 20040101840, and in U.S. Pat. No. 6,596,534, the contents of each which are incorporated herein by reference in their entirety. Such viral vectors may additionally used to study the targeting of β2M-mediated signaling pathways and their regulation of cancer growth and bone metastasis.

In each of the aforementioned aspects and embodiments of the present invention, the osteomimicry interfering compound is administered either alone or in combination with agents that target in parallel with osteomimicry interfering compounds, such as the use of compounds that block VEGF axis, AR axis, GPCR axis, PKA/CREB axis, genes depicted in Appendix A and any combination thereof. Representative, non-limiting examples of compounds that block VEGF axis, AR axis, GPCR axis, PKA/CREB axis may be found in Appendix B infra. In addition, this concept of targeting can be extended to target cells in the microenvironment (such as, for example, and not by way of limitation, fibromuscular stromal cells, bone marrow stromal cells, endothelial cells and inflammatory cells) and the combination with cytotoxic (for example, and not by way of limitation, paclitaxel, doxorubicin, cisplatin, mitoxantrone, estramustine, etoposide, ketoconazole vinblastine) and ionizing radiation agents (for example, and not by way of limitation, strontium-89, Yituim-90 or Lu-177 tagged Abs).

In each of the aforementioned aspects and embodiments of the present invention, the administration of the osteomimicry interfering compound to a subject in need thereof has several beneficial effects including, inter alia, to block or retard cancer and benign growth, to reduce bone turnover for the treatment of osteoporosis, to prevent tumor progression through inhibition of epithelial to mesenchymal transition (EMT) and development of hormone independent disease, to induce massive apoptosis in cancer but not in normal cells, to facilitate bone marrow engraftment and transplantation, or any combination thereof, and to eliminate vascular plaque and calcification for the treatment of cardiovascular diseases.

In yet another aspect of the present invention, a method is provided for treating and/or ameliorating an osteotropic-related cancer or proliferative disorder comprising introducing into osteotropic cells of a subject in need thereof a vector comprising an interfering regulatory region sequence, or transcriptionally active fragment thereof of one or more osteomimecry target genes including, but not limited to, genes that are related to or downstream from the VEGF axis, AR axis, GPCR axis, PKA/CREB axis, or any of the genes depicted in Appendix A, or any combination thereof, wherein said osteomimecry interfering regulatory region sequence can regulate the activity or activities of one or more of said genes by interfering with the osteomimetic potential of said osteotropic cells.

In one embodiment of the aforementioned method, the cancer or other proliferative disorder is selected from the group consisting of osteosarcoma, prostate cancer, breast cancer, colon cancer, lung cancer, renal cancer, brain cancer, multiple myeloma, thyroid cancer, melanoma or any other disease consisting of benign prostate hyperplasia (BPH), vascular plaque formation in cardiovascular conditions or disorders with excessive calcification and mineralization potential, or any combination thereof.

In another embodiment of the aforementioned method, the osteotropic-related disease or proliferative disorder comprises osteoporosis, increased bone turnover through enhanced interaction between RANK and RANKL, and increased cancer bone colonization through enhanced osteomimicry and recruitment of host cells that promote osteoclastogenesis and osteoblastogenesis.

In yet another aspect of the present invention, a screening method is provided for identifying a compound which modulates the osteomimetic potential or properties of a cell comprising: (a) contacting a test compound to a cell that exhibits osteomimetic potential or properties; (b) measuring expression of one or more osteomimetic gene products in the cell; and (c) comparing the level of expression of one or more osteomimetic gene products in the cell in the presence of the test compound to a level of expression of one or more osteomimetic gene products in the cell in the absence of the test compound; wherein, if the level of the expression of one or more osteomimetic gene products in the cell in the presence of the test compound differs from the level of expression of the one or more osteomimetic gene products in the cell in the absence of the test compound, a compound that modulates the osteomimetic potential or properties of a cell is identified.

In one embodiment of the aforementioned screening method, the cell that exhibits osteomimetic potential comprises a cancer cell from osteosarcoma, prostate, breast, colon, lung, brain, renal, multiple myeloma, thyroid, melanoma or any other known disease or disorder with osteomimetic or calcification potential, or any combination thereof.

In another embodiment of the aforementioned screening method, the test compound comprises an osteomimicry interfering compound that interferes with the ability of the cell or cancer cell to express highly restricted bone-like proteins comprising one or more of osteocalcin (OC), bone sialoprotein (BSP), SPARC/osteonectin (ON), osteopontin (OPN) and the receptor activator of NF-κB ligand (RANKL), and/or to increase bone turnover through epithelial to mesenchymal transition (EMT), or any combination thereof.

In another embodiment of the aforementioned screening method, the test compound comprises a β2M siRNA, a β2M antibody, a Runx2 (i.e., cbfa1) transcription factor-specific siRNA, antibody, or antagonist, a GPCR antagonist, an AR or signaling antagonist, a VEGF or signaling antagonist, a PKA/CREB signal activation interrupter, a selective agent that interferes with β2M/PKA/CREB signaling, a selective agent that interferes with CREB transcription, phosphorylation and/or complex formation, a selective agent that interferes with β2M complex formation with other intracellular proteins, or any combination thereof.

In another embodiment of the aforementioned screening method, the osteomimicry interfering compound is administered in combination with one or more more antagonists interfering with AR axis (see Appendix B), interfering with GPCR axis (see Appendix B), one or more anti-angiogenic agents (See Appendix B), one or more cytotoxic drugs (for example, and not by way of limitation, paclitaxel, doxorubicin, cisplatin, mitoxantrone, estramustine, etoposide, ketoconazole vinblastine), or any combination thereof.

For each of the above-recited methods of the present invention, the therapeutically effective amount of one or more substances exhibiting anti-osteomimetic or osteomimetic interfering activity or a functional derivative thereof may be administered to a subject in need thereof in conjunction with a therapeutically effective amount of one or more anti-microbacterial drugs and/or inflammatory compounds and/or a therapeutically effective amount of one or more immunomodulatory agents.

In certain embodiments of the method of the present invention, the anti-inflammatory compound or immunomodulatory drug comprises interferon; interferon derivatives comprising betaseron, .beta.-interferon; prostane derivatives comprising iloprost, cicaprost; glucocorticoids comprising cortisol, prednisolone, methyl-prednisolone, dexamethasone; immunsuppressives comprising cyclosporine A, FK-506, methoxsalene, thalidomide, sulfasalazine, azathioprine, methotrexate; lipoxygenase inhibitors comprising zileutone, MK-886, WY-50295, SC-45662, SC-41661A, BI-L-357; leukotriene antagonists; peptide derivatives comprising ACTH and analogs thereof; soluble TNF-receptors; TNF-antibodies; soluble receptors of interleukines, other cytokines, T-cell-proteins; antibodies against receptors of interleukines, other cytokines, T-cell-proteins; and calcipotriols and analogues thereof taken either alone or in combination.

In one embodiment, the reduction or inhibition of pain and/or symptoms associated with one or more of each of the above-recited cancers or proliferative disorders is on the order of about 10-20% reduction or inhibition. In another embodiment, the reduction or inhibition of pain is on the order of 30-40%. In another embodiment, the reduction or inhibition of pain is on the order of 50-60%. In yet another embodiment, the reduction or inhibition of the pain associated with each of the recited cancers or proliferative disorders is on the order of 75-100%. It is intended herein that the ranges recited also include all those specific percentage amounts between the recited range. For example, the range of about 75 to 100% also encompasses 76 to 99%, 77 to 98%, etc, without actually reciting each specific range therewith.

In yet another aspect, the present invention is directed to a method of relieving or ameliorating the pain or symptoms associated with any one or more of the above-identified cancers or proliferative disorders in a mammal suffering from any one or more of the above-identified cancers or proliferative disorders which comprises administering to the mammal in need thereof a therapeutically effective pain or symptom-reducing amount of a pharmaceutical composition comprising effective amounts of a substance exhibiting anti-osteomimetic or osteomimetic interfering activity, either alone or in combination with one or more anti-inflammatory compounds or immunomodulatory agents; and a pharmaceutically acceptable carrier or excipient, wherein said anti-osteomimetic or osteomimetic interfering substance or compound is sufficient to inhibit the osteomimetic property and/or potential of said cancer or proliferative disorder.

The present invention also relates to the combined use of the pharmaceutical composition exhibiting anti-osteomimetic or osteomimetic interfering activity in combination with one or more antibacterial or antiviral compositions or any combination thereof for treating any one of the aforementioned cancers or proliferative disorders, or any combination thereof.

The present invention provides methods for therapeutically or prophylactically treating cancers or proliferative disorders in a subject.

The method for therapeutically treating cancers or proliferative disorders comprises the step of administering pharmaceutically effective amounts of a compound or substance exhibiting anti-osteomimetic or osteomimetic interfering actvity or derivatrive thereof to the subject after occurrence of the cancers or proliferative disorders.

The method for prophylactically treating cancers or proliferative disorders comprises the step of administering pharmaceutically effective amounts of a compound or substance exhibiting anti-osteomimetic or osteomimetic interfering activity or derivatrive thereof to the subject prior to the occurrence of the cancers or proliferative disorders.

Either methodology inhibits the cancers or proliferative disorders.

The present invention also provides compositions and methods for screening compounds that modulate expression within osteotropic cells and tissues. In particular, it provides compositions comprising polynucleotide sequences from osteomimecry regulatory region polynucleotide sequences or transcriptionally active fragments thereof, as well as nucleic acids that hybridize under highly stringent conditions to such polynucleotide sequences, such as for example, and not by way of limitation, osteocalcin promoter sequence (SEQ ID NO. 1), bone sialoprotein (SEQ ID NO. 2), SPARC/osteonectin promoter sequence (SEQ ID NO. 3), osteopontin promoter sequence (SEQ ID NO. 4), the receptor activator of NF-κB ligand promoter sequence (SEQ ID NO. 5), and the androgen receptor promoter sequence (SEQ ID NO. 6), and use of those polynucleotide sequences to screen compounds that modulate expression within osteotropic cells and tissues and/or that interfere with the ability of cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL).

Specifically also provided are expression vectors comprising one or more of the aforementioend osteomimicry regulatory region sequence, and/or transcriptionally active fragments thereof, operably associated to a heterologous reporter gene, e.g., luciferase, and host cells and transgenic animals containing such vectors. The invention also provides methods for using such vectors, cells and animals for screening candidate molecules for agonists and antagonists of osteotropic-related disorders. Methods for using molecules and compounds identified by the screening assays for therapeutic treatments also are provided.

In another embodiment, the transgenic animal models of the invention can be used for in vivo screening to test the mechanism of action of candidate drugs for their effect on osteotropic-related disorders. Specifically, the effects of drugs on osteotropic-related cancers or disorders can be assayed including, for example, but not limited to, localized or disseminated osteosarcoma, lung cancer, colon cancer, thyroid cancer, brain cancer, melanoma, multiple myeloma, and especially including, without limitation, breast, lung, renal, and prostate cancers, in benign conditions such as BPH or arterial sclerotic conditions, where calcification and mineralization occurs, and in bone marrow or stem cell transplantation, where increased osteomimicry signaling via cAMP/PKA/CREB could prevent engraftment. Therapeutic drugs that interfere with this signaling will increase the efficiency and success of bone marrow or stem cell transplantation.

For example, and not by way of limitation, a composition comprising a reporter gene is operatively linked to an osteomimecry regulatory region sequence, or transcriptionally active fragment thereof such as for example, and not by way of limitation, osteocalcin (OC), bone sialoprotein (BSP), SPARC/osteonectin (ON), osteopontin (OPN) and the receptor activator of NF-κB ligand (RANKL). The osteomimicry regulatory region sequence, or transcriptionally active fragment thereof driven reporter gene is expressed as a transgene in animals. The transgenic animal, and cells derived from osteotropic cells of such a transgenic animal, can be used to screen for candidate compounds that interfere with the ability of cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL).

In addition, the serum and/or circulating cells from such transgenic animals can be used for the assay and serve as the end points to define the effects of osteomimicry interfering drugs in experimental animals. Moreover, serum and/or circulating cells from such experimental animals and/or from human patients could also be used as diagnostic indicators since they will reflect the status of osteomimicry and hence predict the ability of cancer cells to home to the skeleton and visceral organs. The analyses of osteomimicry related target genes and gene products in serum and other biologic fluids and tissue samples can also be used to predict the therapeutic response of patients to therapy.

Without being bound by any particular theory, such anti-osteomimetic or osteomimicry interfering compounds or functional derivatives thereof are likely to interfere with the function of trans-acting factors, such as transcription factors, cis-acting elements, such as promoters and enhancers, as well as any class of post-transcriptional, translational or post-translational compounds involved in osteotropic-related disorders. As such, they are powerful candidates for treatment of such disorders, including, but not limited to, localized or disseminated osteosarcoma, lung cancer, renal cancer, colon cancer, melanoma cancer, thyroid cancer, brain cancer, multiple myeloma, and especially including, without limitation, breast and prostate cancers, and benign conditions, such as BPH or arterial sclerotic conditions where calcification occurs, and bone marrow and stem cell transplantation where increased osteomimicry may prevent the engraftment of foreign cells to the immune intact host. The compounds of the invention additionally can be used to interfere with the expression of crucial growth and differentiation-associated genes such as growth factors, growth factor receptors, non-collagenous bone matrix proteins, bone morphogenic proteins, host immune regulatory molecules, etc, for repairing the damages acquired during aging and/or degenerative conditions.

In one embodiment, the invention provides methods for high throughput screening of compounds that modulate specific expression of genes within osteotropic cells and tissues. In this aspect of the invention, cells from osteotropic-cells or tissues, are removed from a transgenic animal or directly established from human cancer and non-cancer tissues, and cultured in vitro. The expression of a reporter gene is used to monitor osteotropic-specific gene activity. In a specific embodiment, luciferase is the reporter gene. Compounds identified by this method can be further tested for their effect on osteotropic-related disorders in experimental animal models with defined conditions that mimic human diseases as transgenes or as transplanted xenografts.

In each of the aforementioned aspects and embodiments of the present invention, due to the tissue specificity of the anti-osteomimecry regulatory region sequence, or transcriptionally active fragment thereof, the anti-osteomimicry regulatory region sequence, or transcriptionally active fragment thereof therapeutically active agents of the present invention are effective not only when administered via direct application, such as by injection, but also when administered systemically to the body via intravenous administration, intra-arterial administration, intra-tumoral administration, perfusion, oral administration or the like, because gene expression will be limited and localized to specific, cell and tissue types, including, but not limited to, osteoblasts and osteoblast-mimicking cancer and benign cells, osteotropic benign and cancer cells. Furthermore, since many of the therapeutic agents of the invention exhibit pleiotropic effects and targeting selectively cells dependent upon osteomimicry to grow and survive, expression of the therapeutic agents in only specifically targeted cells is essential in order to prevent numerous, harmful side effects to normal cells. A representative example of a harmful sife effect includes the development of autoimmune diseases in the host.

As described in more detail herein, an anti-osteomimecry regulatory region sequence, or transcriptionally active fragment thereof can comprise any number of configurations based upon the promoter sequences and/or enhancer sequences for osteocalcin (OC), bone sialoprotein (BSP), SPARC/osteonectin (ON), osteopontin (OPN) and the receptor activator of NF-κB ligand (RANKL), including, but not limited to, a promoter or fragment thereof; an enhancer or fragment thereof or enhancer-like sequence or fragment thereof; a silencer or fragment thereof; a promoter or fragment thereof and a enhancer or fragment thereof or enhancer-like sequence or fragment thereof; a promoter and a heterologous enhancer or fragment thereof; a heterologous promoter or fragment thereof and a enhancer or fragment thereof or enhancer-like sequence or fragment thereof; and multiple copies of promoters, enhancers or fragments thereof; and multimers of the foregoing.

Methods are also provided herein for measuring the activity of an anti-osteomimecry regulatory region sequence, or transcriptionally active fragment thereof based upon available promoter sequences and enhancer sequences for osteocalcin (OC), bone sialoprotein (BSP), SPARC/osteonectin (ON), osteopontin (OPN) and the receptor activator of NF-κB ligand (RANKL), and thus for determining whether a candidate anti-osteomimetic or osteomimicry interfering compound or a functional derivative thereof has the ability to modulate an anti-osteomimicry regulatory region sequence, or transcriptionally active fragment thereof and/or has the ability to modulate the osteomimetic properties and/or potential of a cancer or proliferative disorder.

In one embodiment, the anti-osteomimecry regulatory region sequence, or transcriptionally active fragment thereof based upon the promoter and enhancer or enhancer-like sequence of one or more of available promoter sequences and enhancer sequences for osteocalcin (OC), bone sialoprotein (BSP), SPARC/osteonectin (ON), osteopontin (OPN) and the receptor activator of NF-κB ligand (RANKL) may be in any orientation and/or distance from the coding sequence of interest, and may comprise multimers of the foregoing, as long as the desired inhibition or interruption of cell-specific transcriptional activity is obtained. Transcription activation or inhibition of transcriptional activation can be measured in a number of ways known in the art (and as described in more detail below), but is generally measured by detection and/or quantitation of mRNA or the protein product of the coding sequence under control of (i.e., operatively linked to) a transcriptional regulatory sequence. As discussed herein, an anti-osteomimecry regulatory region sequence, or transcriptionally active fragment thereof can be of varying lengths, and of varying sequence composition.

In one aspect of the invention, the pharmaceutical compositions of the present invention are administered orally, systemically, via an implant, intravenously, topically, intrathecally, intracranially, intraventricularly, by inhalation or nasally.

In yet another embodiment of the method of the present invention, the osteomimicry related and downstream target genes are expected to be expressed in the blood circulation, in other biologic fluid and/or biopsy specimens. Assessment of the level of expression of these gene products has prognostic value in predicting the expression of one or more lethal phenotypes by cancer cells. These non-invasive methods are expected to be more sensitive than the existing radiographic or biochemical procedures which fail to distinguish cancer cells with different malignant and metastatic potential.

In certain embodiments of the methods of the present invention, the subject or mammal is a human.

In other embodiments of the methods of the present invention, the subject or mammal is a veterinary and/or a domesticated mammal.

There has been thus outlined, rather broadly, the important features of the invention in order that a detailed description thereof that follows can be better understood, and in order that the present contribution can be better appreciated. There are additional features of the invention that will be described hereinafter.

In this respect, before explaining at least one embodiment of the invention in detail, it is to be understood that the invention is not limited in its application to the details as set forth in the following description and figures. The present invention is capable of other embodiments and of being practiced and carried out in various ways. Additionally, it is to be understood that the terminology and phraseology employed herein are for the purpose of description and should not be regarded as limiting.

As such, those skilled in the art will appreciate that the conception, upon which this disclosure is based, can readily be used as a basis for designing other methods for carrying out the several features and advantages of the present invention. It is important, therefore, that the claims be regarded as including such equivalent constructions insofar as they do not depart from the spirit and scope of the present invention.

### BRIEF DESCRIPTION OF THE DRAWINGS

FIG. 1. Immunohistochemical staining of OC and BSP in human primary and bone metastatic prostate cancer tissue specimens, and conditioned media stimulate hOC and hBSP promoter activities and the steady-state levels of OC and BSP mRNA expression in human prostate cancer cell lines. A, Positive and strong OC and BSP stains were detected in both primary and bone metastatic clinical prostate cancer specimens (bold arrows). Some areas of the prostate cancer cells were found only lightly or not stained at all (arrow heads). Osteomimicry was found to exist in prostate cancer cells when present in primary. Magnification X 75. B, CM were collected from human prostate cancer cell lines (LNCaP, C4-2B, DU145, PC3 and ARCaP), a normal human osteoblastic cell line (Kees II) and a human osteosarcoma cell line (MG63). The hOC promoter-reporter construct was co-transfected with CMV promoter-driven β-galactosidase plasmid (for the correction of transfection efficiency as an internal control) into an androgen-independent and metastatic LNCaP cell subline, C4-2B. CM induced hOC promoter activity in a dose-dependent manner (total protein concentration ranged from 0 to 15 μg/ml). C, ARCaP CM also stimulated hBSP promoter activity in a dose-dependent manner (total protein concentration ranged from 0 to 15 μg/ml). D, hOC and hBSP promoter-reporter activities were determined in LNCaP, C4-2B, DU145, PC3, ARCaP and MG63 cell lines in the presence or absence of ARCaP CM (15 μg/ml). hOC and hBSP promoter activities were dramatically elevated by ARCaP CM in LNCaP and C4-2B cells. Fold induction was calculated from the promoter activities assayed in the presence or absence of CM. Data are expressed as the mean±S.D. of three independent experiments with duplicate assays in each experiment. Significant differences of the fold inductions of hOC or hBSP reporter activity were observed by the addition of ARCaP CM: **, p<0.005. E, RT-PCR was performed using total RNAs isolated from LNCaP, C4-2B, PC3 and MG63 cells in the absence (−) or presence (+) of ARCaP CM (15 μg/ml of total protein) for a 12 h incubation period. Expression of the housekeeping gene GAPDH was used as a loading control. The relative expression values of OC and BSP mRNA, normalized by the amounts of GAPDH mRNA expression, were measured by Gel Doc gel documentation software (Bio-Rad). Fold induction represents the ratios of ARCaP CM-treated versus vehicle-treated control of each cell line.

FIG. 2. The cAMP-responsive element (CRE) is responsible for the hOC and hBSP promoter activation induced by ARCaP CM. A, Deletion analysis of hOC promoter. Three cis-elements, AV, OSE2 and OSE1 (9) are not critical for the hOC promoter activation regulated by ARCaP CM (The basal luciferase activities, expressed as RLA, in control hOC/Luc and ΔAV/OSE2/OSE1, were 1440±58 and 1160±200, respectively). ARCaP CM-mediated hOC promoter reporter activity was not affected by the elimination of these three cis-elements. B, Deletion of CRE element abrogated the ARCaP CM-mediated activation of hOC promoter reporter activity. Region A (374 bp), upstream from AV element, contains three cis-acting elements, Tst-1 (-848 to -834), CRE (-643 to -636) and IRF-1 (-609 to -597). Deletion of region A (ΔA) mutant in hOC promoter dramatically decreased the CM-mediated activation of the promoter activity. Subsequently, ΔTst-1, ΔCRE, and ΔIRF-1 mutant constructs were generated from the hOC promoter using the recombinant PCR method. Only the ΔCRE construct abolished ARCaP CM-induced hOC promoter activity. The relative activities of various hOC mutation reporter constructs were determined in the presence or absence of ARCaP CM (minus ARCaP CM of the hOC/Luc promoter activity was designed as 1.0). Significant differences of the relative luciferase activity were indicated: *, p<0.05; **, p<0.005. C, Two putative CRE sites were cooperatively regulated in the hBSP promoter activity by ARCaP CM. Single deletion of CRE1 (ΔCRE1, -79 to -72) or CRE2 (ΔCRE2, -674 to -667) in hBSP promoter reduced partially the promoter activation; the double deletion ΔCRE2/CRE1 construct markedly decreased the ARCaP CM-induced hBSP promoter activity (the hBSP/Luc promoter activity was assigned as 1.0 in the absence of ARCaP CM). Significant differences were calculated: *, p<0.05; **, p<0.005. Data are expressed as the mean±S.D. from three independent studies with duplicate assays in each experiment. D, Point-mutation constructs of the CRE site within hOC promoter were constructed using the QuikChange Site-Directed Mutagenesis Kit (Stratagene, see Materials and Methods). Relative activities of hOC mutation reporter constructs were determined and compared to the hOC/Luc construct (assigned as 1.0 without adding ARCaP CM). Data are expressed as the mean±S.D. from three independent studies with duplicate assays in each experiment. Significant differences from the relative luciferase activity of hOC/Luc: *, p<0.05; **, p<0.005.

FIG. 3. hOC and hBSP promoter activities were stimulated by dibutyryl cAMP (db cAMP) and forskolin (FSK) in a dose-dependent manner. C4-2B cells were co-transfected with A, hOC or B, hBSP promoter plus CMV/β-galactosidase plasmid (an internal control plasmid for transfection efficiency). The transient transfected cells were treated with different concentrations of db cAMP (from 10⁻⁶ M to 10⁻³ M), FSK (from 10⁻⁸ M to 10⁻⁵ M) or ARCaP CM (15 μg/ml) for 16 h. hOC and hBSP promoter activities were induced by these pharmacologic reagents in a concentration-dependent manner through the activation of cAMP-dependent PKA pathway. Fold induction represents the mean±S.D. of three separate studies with duplicate assays in each experiment. Significant differences from control: *, p<0.05; **, p<0.005. C, RT-PCR was performed using 5 μg of total RNAs isolated from LNCaP, C4-2B, PC3 and MG63 cell lines in the absence (−) or presence (+) of FSK (10⁻⁶ M) exposure for 12 h. The relative expression values of OC and BSP mRNA, normalized by the amounts of GAPDH mRNA, were measured by Gel Doc gel documentation software (Bio-Rad). Fold induction represents the ratios of FSK-treated versus untreated specimens from each cell line.

FIG. 4. Effects of a selective inhibitor of PKA pathway H-89 on ARCaP CM-, db cAMP- or FSK-induced elevation of hOC and hBSP promoter activities. A, C4-2B cells, transfected with hOC or hBSP promoter-reporter constructs, were treated with various concentrations of H-89 (from 10⁻⁸ M to 10⁻⁶ M) for 2 h, and subsequently exposed to FSK (10⁻⁶ M) for an additional 16 h. H-89 exerted a concentration-dependent inhibition of hOC and hBSP promoter-reporter activities induced by FSK. Fold induction represents the folds of FSK treated reporter activities assayed in the presence or absence of H-89. Levels of significance were calculated: *, p<0.05; **, p<0.005. B, H-89 inhibited ARCaP CM-, db cAMP- or FSK-induced hOC promoter activity, but did not inhibit the promoter-reporter activity assayed under stimulation by PMA. H-89 (10⁻⁶ M) was added to hOC promoter-reporter transiently transfected C4-2B cells for 2 h, then exposed to ARCaP CM (15 μg/ml), db cAMP (10⁻³ M), FSK (10⁻⁶ M), or the PKC pathway activator PMA (10⁻⁶ M) for 16 h. C, H-89 also abolished the hOC promoter activation induced by C4-2B, DU145, PC3 or MG63 CM. Data are expressed as the mean±S.D. of three independent experiments with duplicate assays. Significant differences were calculated as: *, p<0.05; **, p<0.005.

FIG. 5. ARCaP CM and FSK enhanced CREB and CRE binding through cAMP-dependent PKA signaling pathway in selective human prostate cancer but not bone cells. A, C4-2B cells were exposed to ARCaP CM (CM, 15 μg/ml) or FSK (F, 10⁻⁵ M) for 16 h; control cells were exposed to vehicles. Cells were harvested and nuclear extracts (NE) prepared. EMSA was performed by incubating nuclear extracts and the ³²P-labeled CRE probe. Lanes 3 and 5 show ARCaP CM and FSK enhanced the formation of CRE-nuclear protein complexes. The complexes were competed off by unlabeled specific CRE-oligo probe (lanes 4 and 6). Lane 9 presents that the Mut6-oligo (the CRE mutant of two-point substitution, see FIG. 2D) did not compete with the nuclear proteins and ³²P-CRE-oligo complexes. H-89 (10⁻⁶ M) blocked both ARCaP CM- and FSK-induced CRE binding to the nuclear factors extracted from C4-2B cells (lanes 7 and 8). B, C4-2B (lanes 1-3) and MG63 (lanes 4-8) cells treated with ARCaP CM (CM, 15 μg/ml) or vehicle for 16 h and nuclear extracts were prepared. Lanes 4 and 5 show no or minimum changes of the binding complex formation when experiments were conducted using nuclear extracts from MG63 cells either exposed to ARCaP CM or not. The arrow indicates the CRE and CREB complexes which were supershifted by adding anti-CREB antibody to the nuclear extracts from C4-2B (lane 2), but not from MG63 cells (lane 7). The specificity of the supershift complex was confirmed by the lack of a supershift band when naive Runx2 antibody was used as a reagent (lanes 3 and 8).

FIG. 6. A proposed the cAMP-dependent PKA signaling mechanism describing the regulation of hOC and hBSP promoter activities in human prostate cancer cells. An unknown soluble factor with a molecular mass of less than 30 kDa was proposed to be secreted by human prostate cancer and bone stromal cells. This putative factor is believed to interact with a cell surface receptor in prostate cancer cells and subsequently to activate adenylate cyclase (AC), resulting in a signal cascade through the PKA signaling pathway to enhance hOC and hBSP promoter activities. The molecular basis for osteomimicry is proposed as follows: cAMP generated by ligand-receptor interaction promoted PKA activation, the activated PKA translocated into the cell nucleus and induced CREB phosphorylation which interacted with CRE cis-elements in hOC (CRE) and hBSP (CRE1 and CRE2) promoter regions and triggered marked downstream promoter activation and endogenous mRNA expression in human prostate cancer cells.

FIGS. 7A-E A. The endogenous b2M mRNA expression (RT-PCR) and the b2M protein expression in human prostate cancer cell lines, LNCaP, C4-2B, DU145, PC3 and ARCaP and a human osteosarcoma cell line, MG63. Note despite similar levels of b2M mRNA expression in prostate cancer and bone cells, the secreted form of b2M protein correlated positively with the malignant status of prostate cancer cells. B. b2M stimulated the growth of all human prostate cancer (ARCaP, C4-2B, DU145 and LNCaP cells) but not bone (MG-63) cell lines in culture. * p<0.05. C. b2M over-expression in C4-2B cells markedly increased the endogenous OC and BSP mRNA expression (upper panel). Recombinant b2M protein (0-0.6 mg/ml of b2M) stimulated hOC and hBSP promoter activities and these increased promoter activities can be blocked specifically by anti-b2M antibody (10 mg/ml) but not the isotype control IgG. **, p<0.005 (right panel). D. b2M- but not scramble-siRNA inhibited both cell proliferation and b2M expression of Neo and b2M-overexpressed C4-2B clones. However, scramble b2M-siRNA did not affect these parameters in Neo and b2M clones. **, p<0.005. E. b2M-overexpressed C4-2B cells (C4-2B b2M) grew expolsively in nude mice bones with rapid rise of serum PSA (compared to Neo transfected C4-2B cells), but only small differences were observed with tumor grown in subcutaneous space (N=8-12). Both osteolytic (TRAP+) and osteoblastic lesions were observed in mouse bone (bottom panels). Serum PSA, x-ray and histopathology were routinely assessed in our studies.

FIG. 8. Establishment of overexpression b2M in breast cancer (MCF7), lung cancer (H358) and renal cancer (RCC) cell lines. Different b2M expression levels of MCF7, H358 and RCC clones were assayed by semi-quantitative RT-PCR (top panels). b2M promoted cancer cell proliferation on plastic (middle panels) and in soft agars as revealed by increased number and size of the colonies (bottom panels). Increased cancer cell proliferation by b2M is b2M concentration-dependent in various human cancer cell lines. An asterisk indicates P<0.05 compared with parental and Neo transfected cells.

FIGS. 9A-C. (A) Phosphorylation of CREB and its highly homologous transcription factor ATF-1 in C4-2Bneo and C4-2BB2M cells as determined by Western blotting; (B) Confirmation of phospho-CREB expression in human prostate cancer specimens by immunohistigochemistry staining; (C) Expression of VEGF isoforms and the coreceptor neuropilin-1 in C4-2Bneo and C4-2BB2M cells by RT-PCR analysis.

FIGS. 10A-C. Non-invasive bioluminescence imaging assesses real-time visualization of prostate cancer metastasis transgenic mouse models. (A) A representative bioluminescence profile in TRAMP-Luc models with an exception of the #7 mouse (column) showed an increase signal at mouse jaw, and hind limbs at week 22. (B) The prostate tumor and lymph node metastases were confirmed by IHC of SV-40 T antigen. (C) Abnormal cellular component (see arrow) was observed on the section of jaw bone derived from #7 mouse by histomorpathological (H&E) analysis.

FIG. 11. In vivo detection of experimental metastasis after intracadiac injection of luciferase gene transduced PC3M (PC3M-Luc) human prostate cancer cells into athymic nude mice. Selected in vivo imaging of a representative mouse with metastasis are shown over time. Micro metastases (arrows) to liver (day 21), adrenal gland and tibia (Day 28) were detected by CCD camera.

FIGS. 12A-D. Liposome encapsulated b2M-siRNA but not scramble-siRNA inhibits the growth of pre-established PC3-Luc and C4-2-Luc tumor in athymic nude mice. The anti-tumor effect of siRNA in subcutaneous bone powder tumors (A, B) and intratibial bone tumors (C) was demonstrated by BLI (A, N=5) and serum PSA (B, N=5 and C, N=7-9) over a period of 28 days. ** p<0.005. (D) histomorpathological (H&E) analysis (Magnification: 200×) showed massive prostate cancer cell death in b2M-siRNA treated specimens.

FIG. 13. Adhesion assay of b2M-siRNA and scramble-siRNA infected C4-2B cells using 96-well plate pre-coated with Col I, LM, FN and Col IV. BSA was used as control (Con). *, p<0.05, **, p<0.005.

FIG. 14. Western blot analysis of parental C4-2B (P), b2M-siRNA (siRNA) and scramble-siRNA (Scramble) retrovirally infected cells. Note AR and PSA expression were abolished by b2M-siRNA in C4-2B cells. EFIa was used as loading control.

FIG. 15. Dotplot for b2M and VEGF between two groups (1 is for bone metatsasis group, 2 is tumor confined group).

FIG. 16. This schematic depicts the involvement of osteomimicry in driving epithelial to mesenchymal transition (EMT) and EMT-associated gene expression during malignant progression of cancer cells.

FIG. 17. An X-Ray photograph depicting the development of osteoblastic/osteolytic mixed tumors in control versus b2m knockout SCID mice.

FIG. 18. β2M regulation of VEGF expression and signaling in prostate cancer cells. β2M-induced activation of cAMP-PKA-CREB pathway facilitate the formation of a dynamic transcriptional complex, recruiting several important transcriptional factors, i.e., CBP/p300, HIF-1, STAT3, AR and SRC-1 to bind the VEGF promoter and activate transcription. Elevated VEGF expression and secretion in turn activates certain downstream signaling in an NP-1-dependent manner. This autocrine loop antagonizes the pro-apoptotic effects of Sema3A/3B, therefore promoting cancer cell proliferation and metastasis.

FIG. 19. This schematic depicts the signaling of the GPCR axis.

FIG. 20. This schematic depicts the signaling of the androgen receptor (AR) pathway.

### DETAILED DESCRIPTION OF THE INVENTION

The inventors identified a novel molecular target, osteomimicry, which confers the ability of prostate cancer cells to mimic the gene expression and behaviors of osteoblasts, thus allowing prostate cancer cells to adhere to bone cells and grow and survive in bone. Osteomimetic prostate cancer cells express not only highly restricted bone-like proteins such as osteocalcin (OC), bone sialoprotein (BSP) osteopontin (OPN) and receptor activator of NF-κB ligand (RANKL), they are also capable of forming mineralized bone under certain cell culture conditions. Bone matrix proteins are highly expressed in both localized and metastatic prostate cancers but not in normal prostate.

Rationale for Osteomimicry as a Novel Cancer Target

Osteomimicry, defined as the ability of non-malignant benign cells [benign prostate hyperplasia and fibromuscluar stromal cells around the blood vessels to grow and proliferate] or cancer cells to mimetic the gene expression and behaviors of bone cells thereby allowing the cancer cells to grow, survive and invade in the bone microenvironment Osteomimicry may also regulate host immunity and other immune status.

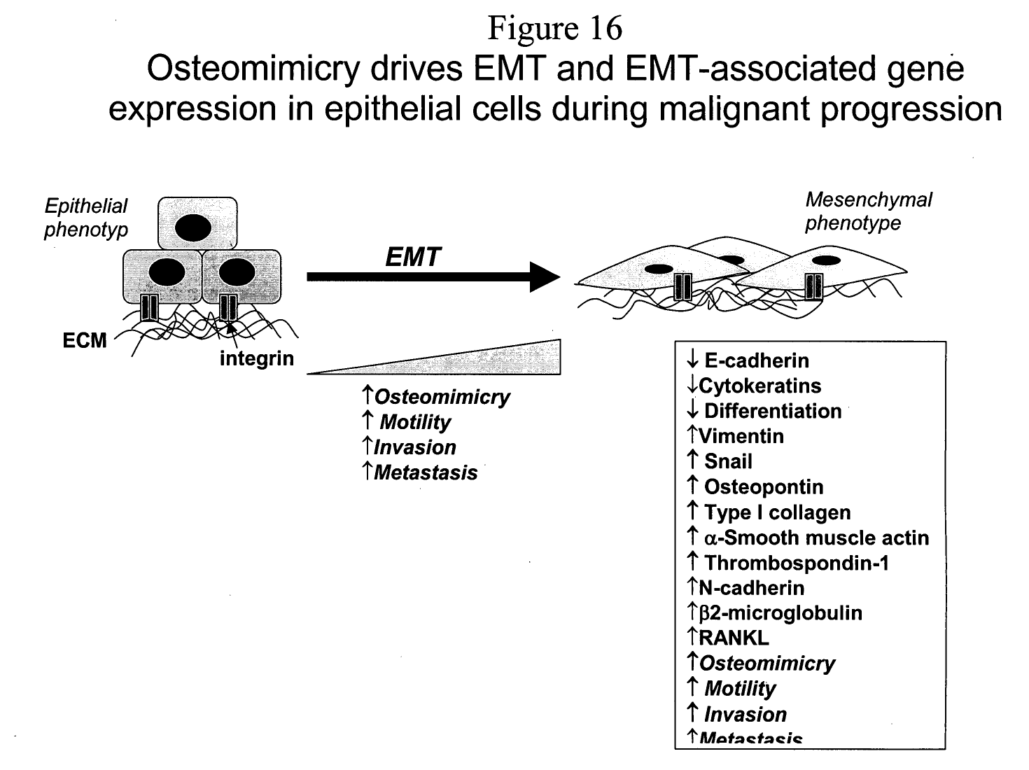

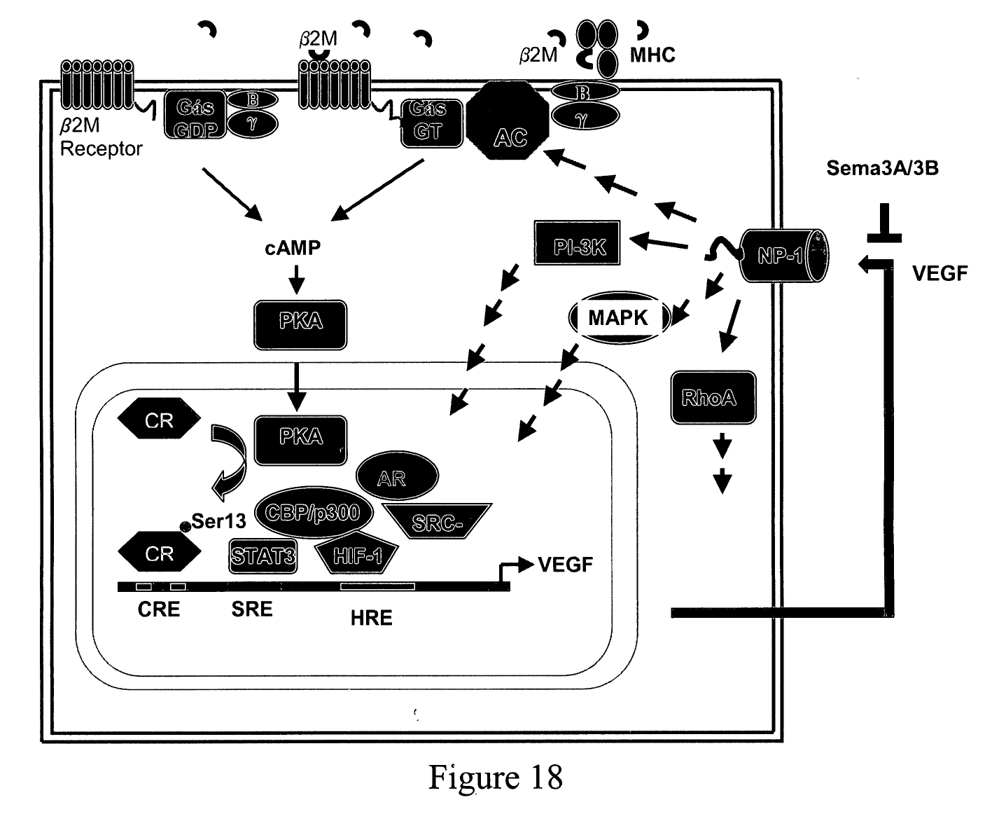

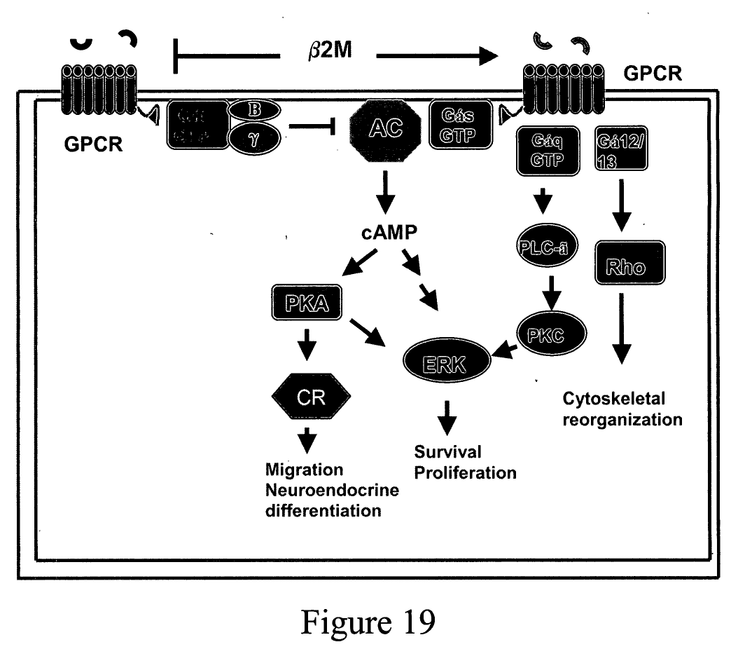

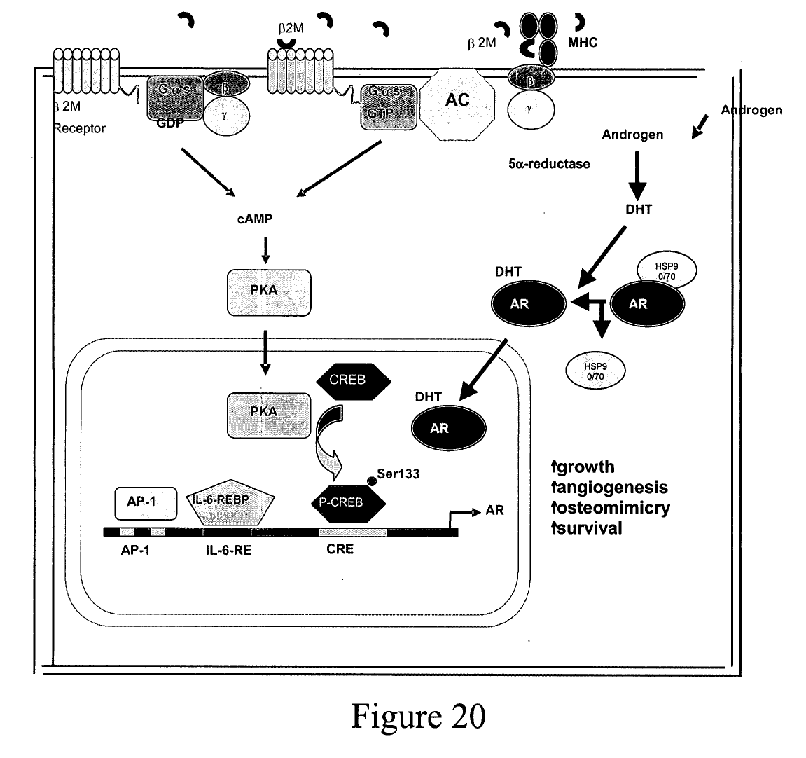

Osteomimicry is controlled by: 1) the cAMR/PKA/CREB pathway which is intimately tied to GPCR-mediated downstream signaling (See FIG. 19), AR axis (See FIG. 20), VEGF axis (See FIG. 18), EMT, integrin-ECM signaling (See FIG. 16); 2) the Runx2/cbfa1 signaling pathway, and controls the ability of cancer cells and cells in cancer microenvironment to grow, to undergo apoptosis, to gain survival advantages, to invade, to migrate or to metastize and differentiate. Osteomimicry is responsible for the synthesis, secretion and deposition of the bone like proteins: OC, OPN, ON, BSP and RANKL by benign and cancer cells.

Osteomimicry is responsible for the up and down regulation of a series of genes related to the control of cell growth, cell death, oxidative stress, cell differentiation and cell cycle progression. These genes can be regulated to affect the fate of benign and cancer cells (see Appendix A).

Osteomimicry occurs in normal cells which allows them to calcify and mineralize, providing a foundation for the development of BPH and athleroschlerotic plaques. Osteomimicry affects the presentation of MHC class-1 antigen in normal cells and effects the immunity and immune status of the host.

Osteomimicry has dual functions: 1) when overexpressed expressed in benign or cancer cells, increased growth survival and decreased apoptosis of cells are expected. By antagonizing osteomimicry through the use of osteomimetic interfering drugs, we expect an inhibition of the growth and increased apoptosis of benign or cancer1 cells in the host, 2) when overexpressed in normal host cells there is enhanced host immunity, thus decreased efficiency of bone marrow and stem cell engraphments. This condition can be reversed through the admistration of osteomimetic interfering drugs.

Drugs that interfere with osteomimicry in cancer and benign cells can block cancer progression by causing massive cancer cell death, abrogating neovascular endothelial sprouting and ingrowth of endothelium into the invasive tumor, preventing EMT, inhibiting attachment of cancer cell to selected ECM and attenuating cancer cell survival. These drugs are also expected to decrease calcification and mineralization of normal benign cells and cause apoptotic death of BPH and fibromuscular smooth muscle cells.

Drugs interefere with osteomimicry include, but are not limited to those compounds or drugs identified in Appendix B, as wel as those compounds known to interefere with the Runx2 signaling pathway. These drugs are expected to inhibit osteomimicry, resulting in decreased benign and cancer cell growth, and improved efficiency of bone marrow and stem cell transplantation.

Drugs that interfere with osteomimicry can be used in combination with other cytotoxic drugs and/or radiation therapies that could work either additively or synergistically to enhance the pharmacologic affect of osteomimitic interfering drugs.

Drugs interring with osteomimicry can be used either alone or in combination (as described above) for the inhibition of use in the control of the growth and metastasis of cancer cell which include but not limit to prostate, breast, multiple myeloma, renal, lung, brain, thyroid, colon, and osteosarcoma, the abnormal growth of the benign cell which include but not limit to smooth muscle and fibroblast related to mesenchymal lineage in the benign conditions such as BPH and atherosclerosis, and the host immunity during bone marrow and stem cell transfer.

Biomarkers (see Appendix A, bone matrix proteins and signal componenets involved in AR axis, VEGF axis, GPCR axis, cAMP/PKA/CREB axis, and Runx2 signalling pathways as described above) in the biologic fluids or tissues related to osteomimicry are predictors for cancer, bone and visceral organ metastases, the lethal phenotypes of cancers.

Osteomimicry is determined by a soluble factor, b2m or b2m like protein or peptide. B2m is secreted by cancerous or normal cells with the ability to activate downstream target genes (see Appendix A bone matrix proteins and signal componenets involved in AR axis, VEGF axis, GPCR axis, cAMP/PKA/CREB axis, and Runx2 signalling pathways as described above through activation of transcription of, but not limited to CREB.

Osteomimicry can be assayed by transfecting a target cell with an osteomimicry target gene promoter reporter construct, either alone or in combination with a host of other osteomimicry target gene promoter reporter constructs, and the extent of osteomimicry in a normal condition is expected to be in proportion with the activation of these promoter reporter constructs in a target cell. However, it is also expected that variations can occur due to heterogeneity of transcription factors, modifiers and interactive proteins in cells so that basal osteomimicry status and its responsiveness to regulators are expected to be varied in a cell context dependent manner.

Drugs that interfere with osteomimicry in a typical assay include but are not limited to the assessment of osteomimicry related genes, such as human OC promoter luciferase activity, either alone or in combination with a series of other osteomimicry related gene promoter reporter constructs, in a target cell are effective agents for the clinical use in the control of the growth and metastasis of cancer cell which include but not limit to prostate, breast, multiple myeloma, renal, lung, brain, thyroid, colon, and osteosarcoma, the abnormal growth of the benign cell which include but not limit to smooth muscle and fibroblast related to mesenchymal lineage in the benign conditions such as BPH and atherosclerosis, and the host immunity during bone marrow and stem cell transfer.

Drugs interfering with osteomimicry may include, but not limited to small molecules, antibodies, nucleic acids and naturally occurring pharmaceuticals which can be assayed to interfere with osteomimicry by interfering promoter reporter activity, cell growth, cell survival, apoptosis, cell invasion, cell migration and cell spreading.

Drugs interefering with osteomimicry may include, but not limited to nucleotide sequences or their fragments that recognize the critical promoter regions that regulate target downstream from osteomimicry which include, but not limited to AR axis, VEGF axis, GPCR axis, cAMP/PKA/CREB axis, and Runx2 signalling pathways and genes described in Appendix A.

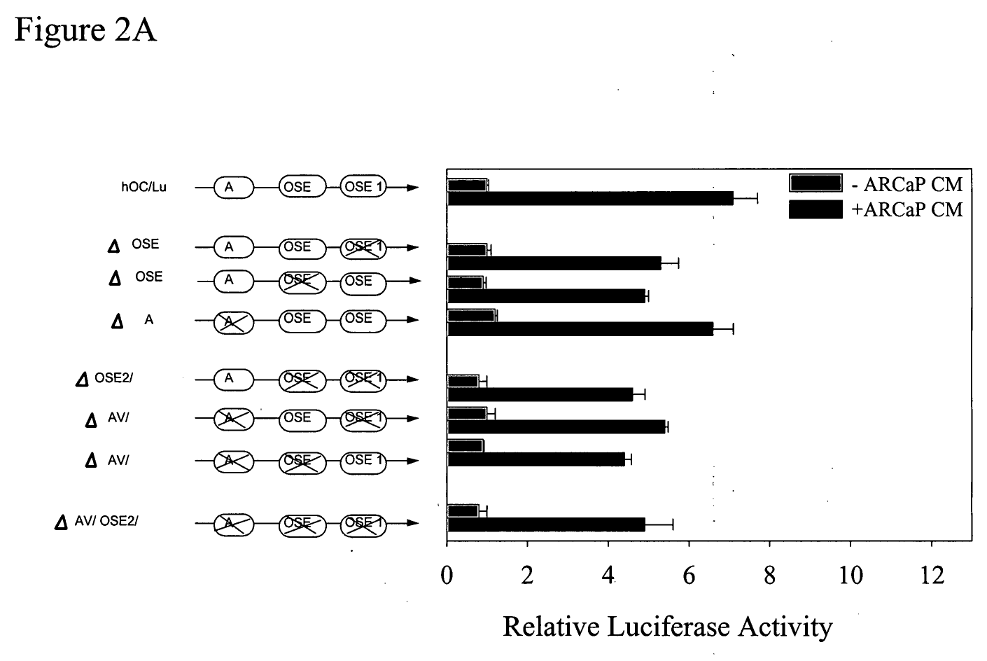

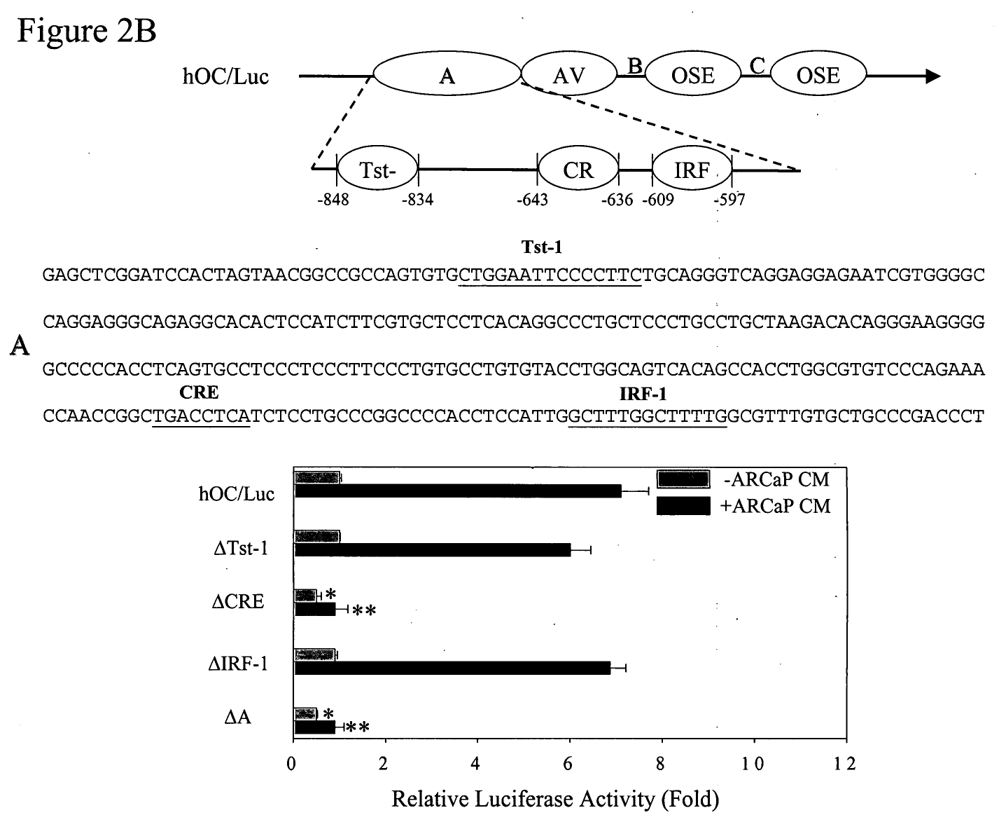

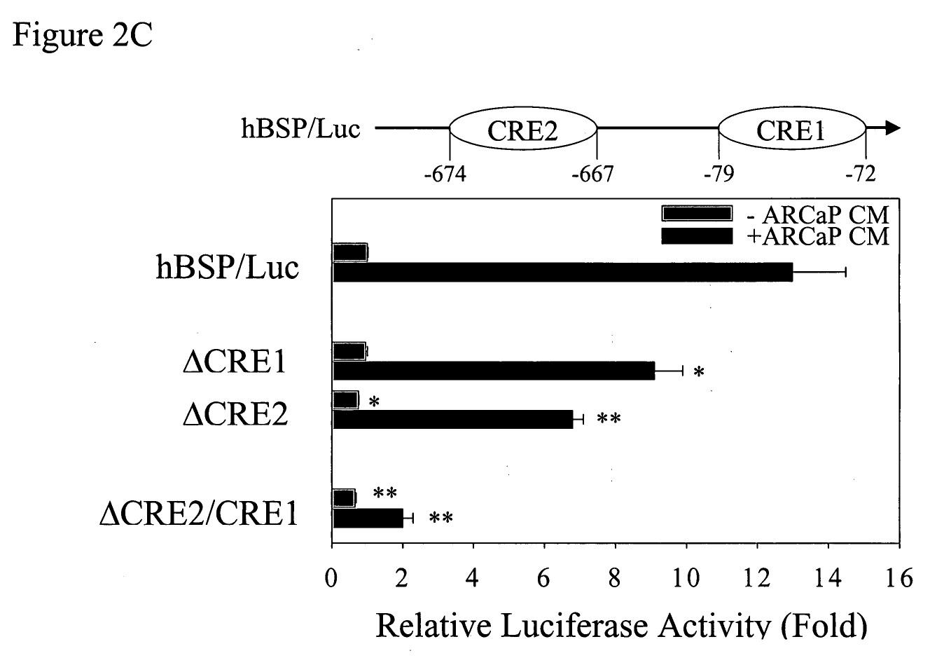

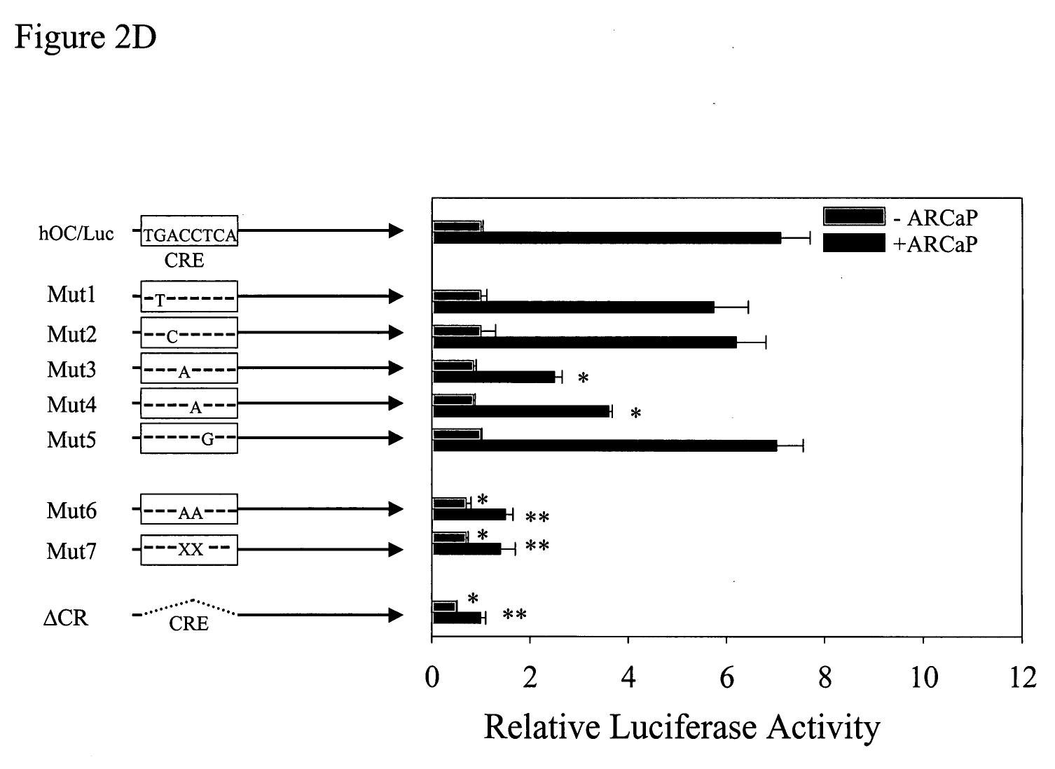

Drugs or compunds interfering with osteomimicry may include, but not limited to analogs of small molecules that interfere with the AR axis, GPCR axis, VEGF axis, and PKA/CREB axis as exhibited in the accompannying figures. Representative, non-limiting examples of selective agents that interfere with PKA/CREB signal activation include those selective agents that target the specific region of the cis-element in hOC promoter, located between -643 to -636 (CRE) (FIG. 2), which the inventors have shown is responsible for conferring cAMP regulation of hOC promoter activity in human prostate cancer cells. Additional representative, non-limiting examples of selective agents that interfere with PKA/CREB signal activation include those selective agents that target other regions of CRE within hBSP promoter, -79 to -72 (CRE1) and -674 to -667 (CRE2) (FIG. 2), that must also be activated upon the exposure of human prostate cancer cells to cAMP mimetics and yet unidentified growth factor(s) in the CM of prostate cancer and bone stromal cells.

Antibodies interfering with osteomimicry may include, but not limited to specific binders or interference molecules as a protein, peptide, nucleic acid, radioactive/cytotoxic derivatives that inter with osteomimicry related downstream signaling.

As described herein in Example 1, the inventors found that osteomimicry in prostate cancer cells is maintained by the activation of G-protein coupled Protein Kinase A (PKA) signaling mediated by a downstream cyclic AMP responsive element binding protein (CREB). By specifically targeting osteomimetic processes, the inventors observed the retardation of prostate cancer cell growth, induction of apoptosis in vitro and massive tumor cell death in prostate cancer bone xenografts in vivo. Thus, specifically targeting osteomimicry, either alone or in combination with chemotherapy, can prevent prostate, breast, lung and renal cancer cell growth and survival in bone and prolong the survival of cancer patients with bony metastases.

The inventors identified β2 microglobulin (β2M) as a key soluble factor secreted by cancer cells as well as cells in the cancer microenvironment. β2M is a critical autocrine and paracrine growth factor that maintains cancer cells' ability to synthesize and deposit bone-like proteins such as OC and BSP, and stimulates the growth and survival of cancer cells by activating vascular endothelial growth factor (VEGF) and androgen receptor (AR) signaling that eventually enables cancer cells to resist hormone withdrawal, exposure to chemotherapy and radiation therapy. β2M overexpressed prostate, breast, lung and renal cancer cells showed an increased growth rate in vitro on plastic dishes (anchorage-dependent) and in soft agar and matrigel (anchorage-independent). As shown herein in Example 2, β2M overexpressed prostate cancer cells introduced into mouse femur provoked explosive tumor growth in bone with rapidly elevated serum PSA, suggesting direct growth-promoting effects by this factor in prostate cancer bone metastasis. Immunohistochemical (IHC) studies of human prostate cancer primary and/or bone metastatic specimens demonstrated overexpression of β2M, and its target genes, OC (26-28), BSP (22, 29) and OPN (30) were associated with increased malignant status of prostate and breast cancers.

The results indicating that β2M plays a previously heretofore unrecognized role in promoting both anchorage-dependent and independent growth of human prostate, breast, lung and renal cancer cells and explosive growth of prostate cancer in mouse bone. These results collectively support β2M's identity as a novel factor responsible for maintaining osteomimicry by cancer cells. Further defining the molecular mechanisms of the β2M-mediated signaling pathway (See, FIGS. 16, 18. 19, and 20) and target genes in cancer cells will result in new prognostic factors for human prostate cancer bone metastasis and new targeting possibilities for prostate cancer bone metastasis. as well as other cancer types such as breast, lung and renal cancers, which have also been shown to depend on β2M mediated signaling for cancer cell growth in vitro.

What follows is a detailed description of the osteomimicry-specific polynucleotides and nucleic acids of the invention (for example, and not by way of limitation, osteomimicry regulatory region sequences, and transcriptionally active fragments thereof), in conjunction with reporter constructs utilizing such osteomimicry-specific polynucleotides and nucleic acids can then be used to screen for candidate compounds or substances that interfere with the expression of the heterologous coding sequence. Such identified compounds or substances that interfere with osteomimicry regulatory region sequence, and transcriptionally active fragments thereof will be likely candidate compounds that interfere with the ability of cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL).

Osteomimecry Polynucleotides and Nucleic Acids of the Invention

The present invention encompasses polynucleotide sequences comprising the 5′ regulatory region, and transcriptionally active fragments thereof, of an osteomimicry gene, including, for example, and not by way of limitation, osteocalcin (OC), bone sialoprotein (BSP), SPARC/osteonectin (ON), osteopontin (OPN), and the receptor activator of NF-κB ligand (RANKL). The nuceotide sequences of the promoter regions of each of osteocalcin (OC) (SEQ ID NO. 1), bone sialoprotein (BSP) (SEQ ID NO. 2), SPARC/osteonectin (ON) (SEQ ID NO. 3), osteopontin (OPN) (SEQ ID NO. 4), the receptor activator of NF-κB ligand (RANKL) (SEQ ID NO. 5), and the androgen receptor (AR)(SEQ ID NO. 6) are depicted below. The promoter sequences of VEGF, NP-1 and Runx2 are available in the public domain and one of ordinary skill in the art may obtain the promoter sequences of VEGF, NP-1 and Runx2 and use such promoter sequences in the methods of the present invention without undue experimentation.

```
hOC promoter (0.9 kb)
(SEQ ID NO.1)
GAGCTCGGATCCACTAGTAACGGCCGCCAGTGTGCTGGAATTCCCCTTCTGCAG
GGTCAGGAGGAGAATCGTGGGGCCAGGAGGGCAGAGGCACACTCCATCTTCGTG
CTCCTCACAGGCCCTGCTCCCTGCCTGCTAAGACACAGGGAAGGGGGCCCCCAC
CTCAGTGCCTCCCTCCCTTCCCTGTGCCTGTGTACCTGGCAGTCACAGCCACCT
GGCGTGTCCCAGAAACCAACCGGCTGACCTCATCTCCTGCCCGGCCCCACCTCC
ATTGGCTTTGGCTTTTGGCGTTTGTGCTGCCCGACCCTTTCTCCTGTCCGGATG
CGCAGGGCAGGGCTGAGCCGTCGAGCTGCACCCACAGCAGGCTGCCTTTGGTGA
CTCACCGGGTGAACGGGGGCATTGCGAGGCATCCCCTCCCTGGGTTTGGCTCCT
GCCCACGGGGCTGACAGTAGAAATCACAGGCTGTGAGACAGCTGGAGCCCAGCT
CTGCTTGAACCTATTTTAGGTCTCTGATCCCCGCTTCCTCTTTAGACTCCCCTA
GAGCTCAGCCAGTGCTCAACCTGAGGCTGGGGGTCTCTGAGGAAGAGTGAGTTG
GAGCTGAGGGGTCTGGGGCTGTCCCCTGAGAGAGGGGCCAGAGGCAGTGTCAAG
AGCCGGGCAGTCTGATTGTGGCTCACCCTCCATCACTCCCAGGGCCCCTGGCCC
AGCAGCCGCAGCTCCCAACCACAATATCCTTTGGGGTTTGGCCTACGGAGCTGG
GGCGGATGACCCCAAATAGCCCTGGCAGATTCCCCCTAGACCCGCCCGCACCAT
GGTCAGGCATGCCCCTCCTCATCGCTGGGCACAGCCCAGAGGGTATAAACAGTG
CTGGAGGCTGGCGGGGCAGGCCAGCTGAGTCCTGAGCAGCAAAGGGCGAATTCT
GCAGATATCCATCACACTGGCGGCCGCT
hBSP promoter (1.5 kb)
(SEQ ID NO.2)
GTGGCACATATACACCATGGAATACTATGCAGCCATAAAAAATGATGAGTTCAT
GTCCTTTGTGGGGACATGTATGAAATTGGAAACCATCATTCTCAGTAAACTATC
ACAAGAACAAAAAACCAAACACCACATATTCTCACTCATAGGTGGGAATTGAAC
AATGAGATCACATGGACACAGGAAGGGGAACATCACACTCTGGGGACTGTTGTG
GGGTGGGGGGAGTAAGGGGAGGGATAGCATTGGGAGATATACCTAATGCTAGAT
GATGAGTTAGTGGGTGCAGCACACCAGCATGGCACATGTGTACGTATGTAACTA
ACCTGCACACAATGTGCACATGTACCCTAAAACTTAAAGTATAATAATAAAAAA
AATTAAGAGAAAAAAAGAAAAAAAATGATATTCATTAATTTTTGATTTCTCAAG
CAGACTTCGCAACTGGAGGAAGAATAAAATGACTAGACTAGGAGAATATGCAAA
CTATTAAGCTAGATTTCCCTTTATAAATTAAAAAATTAGTACTTTAGTTTATCA
ATCCATTCTTTGTGGTGTTGGTTTCATGAATCATTTCAAAAACAATGGATCACT
CCTGCTAGCTCTAGTCATTTTGTTATTCTCATAGGAAAAAAATTAAATATGAAA
ATGAATAGAAAAGATATATATAGAAGCCCAAGAAAAATCAGCTGACCTCACATG
CACGACAGGAAGGCCACATAAATGGACAATATACAGAGATTTAATTTACAAAAC
AAAATATAAAATCTGCCTCTCAGTGGTATGATTCTCAAAAGTTCTAACTTTTAT
ACTCAGCATCATGTTTTAGCAACTATATGTTACAAAGTCTGACCGACTTAATCA
TATCAACTTTAATTTATGAGTCAATGAAGTATATTTCAGGAGGAAACATCAAAT
GATATTAAAATATTGATGGTTCATCTGCTGGTTTCCCTTATTATTTAGTTTTTC
TTTCTTTTTTTAGCTAAACTAATGTAAAAGTTATATCTAATGACAGCAAGCTTT
CCTTTCTTTCGACATAGTGAAAACTTGTGTAATTATGAAATTTTTAAAAGGTTA
AAGCCTTTGTTATTTATTTTAATTCAAATCCAGTATATTATTATACATATTCGG
AGCCCAAACTATTCATCTTCATCTAAACCTTCAATTAAATTCCACAATGCAAAC
CTCTTGGCTCTAGAATCACGTTTCTTGTTTATTCAACTGAGCCTGTGTCTTGAA
AAAGTGTTGAAGTTTGGGGGTTTTCTGGTGAGAATCCACGTTCTGACATCACCT
TGGTCGTGACAGTGATTGGCTGTTGGAAGGCAAAGAAGAGTTTATAGCCAGCAA
GAGCAAGTGAATGAGTGAGTGAGAGGGCAGAGGAAATACTCAATCTGTGCCACT
CACTGCCTTGAGCCTGCTTCCTCACTCCAGGACTGCCAGAGGGTAAGATTTAAT
AGAACAACGG
hOPN promoter (2.2 kb)
(SEQ ID NO.3)
GGGAAGGAGA CAATAGTGTC AACTTGGGAT TGCCTAAGGC AACAACAGAG
CAAAACAAGA ACGCTTTGGT TCTCTGGGTC TCTGTCCCTG ATTGCATAGC
GGGTCATTGT TGGGAAATAT TTCCTCACCT GGCATTCCAA GAAATGGTGA
GCTCCACAGC TGTATATAGT CCTGTCATTA AATACAGGAG TGTTCTATCC
CGCTGGAATT AAGAAAATTG GTAGAACCAG ATTGTGGTCT GAAATCTTTT
TTCAGAAATG CTGCCATCGT GTGGCACTGC GGAGCTATGA CCAGAAGAGT
CCTGTAAAGG GTCGTATGGT TCATCTCAAG ATGGCTGGGC TCCAGCATAA
TCTATTCCTA TAATTAATTC TAGCTTCATA TTGAATCATT CCCGTGGGCA
CAGAGTAAAC TACAGTAAAT CCTGTGGAAA TTTTGTTGTT TTTAGAATTT
TCGGACTTCC CTCCACTAAA TTGACAACAT GACACGCTTA TGCGNGTATG
TTTAAAGGAA AAAAATAGTT TTTAGAAGCA GAAAAAAGAA GTCTATTTTG
CAACTTTATA ATCTGTGTGC TTNCTATTTT ATAGAGATAG TCGTCATCTT
ACTTATTAAA ATGGGTGCTT ATTACCTACA AACCAATCAT ATCAATTCAT
CTGGAATACA TCCAATTTAA GGGAGACATA TTTCCCCCTA CCAAATGTTC
ATGAAACCTA TGAATTAGCT ATACACTATC ACTGCAAGAC ATTATTTAAT
CTATATTTAT ATTAAAAGTA ATATTTGGCA AAAGGAAGCT GACACTTTAG
GACTAATAAA AACCACAATT ACTTTTGCAG CAACCTAATA ATAAATAGGA
CCATTTATTT TTCATCTCAA TTACACACAA GTCTTAACAA TAAAGGTGTA
AGGTAAATAA ATAGTGCAAT CTGCATTTCA CAACTGAGAA GCAAATGAAG
ATAAGTAATC TCAAGGCAAT ATTAAATATT TTAAAAGGAC CCAGAGCTCT
GCTATCCCTG AATTCTGCTC TAATATTCGG ACTTTCCCTG TAATTTTCTT
TCATTCAGAC ACCTTTTAAA TACCTAGTAA AGTGTTTTTT AATACAGAAA
TTTTTAAAAA TGTTTTTCTT TTTAAGTGGC CTACTTTACA TACCTTGGGA
GAAAAACTAG AAAAAAAGAT GATTCCAAAA TCGAATCTGT TCCTTTAGAA
ATGTGCAAAA TTTCCTTATT GATGCATACA ATTTAAAGAT CTTACGTCTA
CTCTCATTTT AATAACCTGT TCTTTTAAAG GACATTACAA TTCGTGACTG
CCTGCCCCTC TTAAAAATTT CATAATAGTT AACACACATA TAGTCCTTAA
GATACGCAGA GCATTTGCAT CTAATATGTG CTAAGCATTG CTAGTTTAAC
ATACTAATTC ATTTAAACCC CTCAAAAACC CCATGACCTA GGTAATAGTA
TTGCATTTCA TGGATGAGGG AACAAGGATA GGTAGGCTGG GCGATTTGCC
CAAGGTTGCA CAGGTCAGCA GTGACACAGC GGAATTCAGA ACCACGGTCT
GGCTCCTGAA GCAGCCCTCT CAAGCAGTCA TCCTTCTCTC AGTCAGAAAC
TGCTTTACTT CTGCAACATC TAGAATAAAT TACCATTCTT CTATTTCATA
TAGAATTTTA TATTTTAATG TCACTAGTGC CATTTGTCTA AGTAACAAGC
TACTGCATAC TCGAAATCAC AAAGCTAAGC TTGAGTAGTA AAGGACAGAG
GCAAGTTTTC TGAACTCCTT GCAGGCTTGA ACAATAGCCT TCTGGCTCTT
CAATAAGTAC AATCATACAG GCAAGAGTGG TTGCAGATAT TACCTTTATG
TTACTTAAAC CGAAAGAAAC AAAAATCCAT TGTATTTAAT TTTACATTAA
TGTTTTTCCC TACTTTCTCC CTTTTTCATG GGATCCCTAA GTGCTCTTCC
TGGATGCTGA ATGCCCATCC CGTAAATGAA AAAGCTAGTT AATGATATTG
TACATAAGTA ATGTTTTAAC TGTAGATTGT GTGTGTGCGT TTTTGGTTTT
TTTTTGTTTT AACCACAAAA CCAGAGGGGG AAGTGTGGGA GCAGGTGGGC
TGGGCAGTGG CAGAAAACCT CATGACACAA TCTCTCCGCC TCCCTGTGTT
GGTGGAGGAT GTCTGCAGCA GCATTTAAAT TCTGGGAGGG CTTGGTTGTC
hON promoter (2.3 kb)
(SEQ ID NO.4)
GAATTCCTTGTACTTTTTTTCCCTTCTCAGTTCTGCACTTAACTCGTCTAAAAA
AATTAAAAAAGAATTTAAGAAACCACAAAGCTAAGCTGGGTGCGGTGGCTCACG
CCTGTAATCCTAGCACTTTGGGAAGCCAAGGCATTCGGATTGCCCAAGCTCAGG
AGTTCGAGACCAGCCTGGGCAACATGTTGAAACCCCATTTCTACTAAAAATACA
ATAAATTAGCTGGGTGTTGTGGCATGTGCGCCTGTAATCCCAGCTACTCTGGAG
GCTGAGGCGCGATAATTGCTTGAACCCGGGAGGCAGAGGTTGCAGTGAGCCGAA
ATCATACCACTGCACTCCAGCCTGGGCGACAGAGTGAGTGAGACTCTGTCTCAA
AACAAAACAAAACAAACAAACAAAAAAACCGGAAACCAACAAAACTTTTTGAGG
AACAAAGGGAACCAGGTATTTTATTAATTCTCATACCTCCAGAGTGTTAGGCAC
AAAATAAACATTCAACCAAGACCTGTTGCACTGAGCAGTTCATATATAACAGGA
GTGACCCAAGTTGAAACGTAGAATCAGCCCTCTCATACCACTTTTTGCCAGGTG
ATCATAGGCAAGTTACTTAGCATCTATGTTTCCTTATTATTAAAATGGTCATAA
TTACAATGCCTAAGATAAGGGGGTTGCTGTGAAGATTATTAAATCCTCAGTAAA
CTTTGGCTATTGTTACTCCTATGATTATCATCAATATCATCAATTACCTTATCT
GTTCAATACTGGTGGCACAGGTCCACCAGCTAGATGTCTAATCCCTTATGTGTC
TATTAGTGGTACAAGTGGAGTTTGAGTGGGATTTTTTTTTTTTTTTTTAAGACC
AGTTCCAAATCATCAAGGATGATACCACTAGTAGCAGCTTGTCTTGTCTGTACA
GTGGTAAGTCCTGGCCTTGCCTTTGTGGCAAATACAACCCCCTTGAATTGCTTG
GCCCTTCTCAGCATTGCCTAATATTAGGGAGGACTCCTGTAAAGCTCACTGGTT
AGAAGATCAAGACACTTGGGCCTGGTTCTGCCCCTGGGGGCCATTGGGTAATTC
CTTGGAGTCTCCAGGCCTCACTTGCCCTCTGAACAAGAAAGAGGCCTGTTCTGG
TCATCCCTCCAGCCTGTCCAGCCCTGGCACTCTGTGAGTCGGTTTAGGCAGCAG
CCCCGGAACAGATGAGGCAGGCAGGGTTGGGACGTTTGGTCAGGACAGCCCACC
GCAAAAAGAGGAGGAAAGAAATGAAAGACAGAGACAGCTTTGGCTATGGGAGAA
GGAGGAGGCCGGGGGAAGGAGGAGACAGGAGGAGGAGGGACCACGGGGTGGAGG
GGAGATAGACCCAGCCCAGAGCTCTGAGTGGTTTCCTGTTGCCTGTCTCTAAAC
CCCTCCACATTCCCGCGGTCCTTCAGACTGCCCGGAGAGCGCGCTCTGCCTGCC
GCCTGCCTGCCTGCCACTGAGGTATGTGTGACCCCCGCCCAGCCTTTCCCTTCT
ATAGTTGCACCAACCCCGACACCCCCGTTCACGCCGTCAGCTCGTGTGCAAGGG
AGGGAAGCTCTGCTGAGGATGCGCCTCTCCTCCCGGCTCCATCACGGCTCCCCT
TAAGAGCATGGCCCTCGGTCCTGTCTGCCTGTTGCTTTTCAGAAGGTGGACTCA
CTGTGTAACTTTGTCTTCCCTTACAGGTTTACAGGAAAATAATCTCACTATGTT
CTTCGGGGGAGCATTTTCTCACTCTCTGTTTTTCTCTGTGTCTGTCTCTGGTTT
CAGAGGCTGCCTGCCTGTCCTCTTTGCTCCCTTTGCAAATGTGGCAGCCTCCTC
CTTTCCTGGGAATCTGATCCCATCACAGCTGCCACAGGGACCTGGCCAGCAACC
GGAGTCTGTCCTCCAGATCTCGGTCAGGGGTTCTGTTTTCCAAAAAGGGACTTT
GCAGAACAATCAGTTGATCTCTGAAAGGGAAAGGGGGAGGCTTCACCATTAATC
CACACCTCTGGGAAGCTTCTGTTTTCCTCTAATTCTCCTCACTCCCAAACACCA
CCTTCCGTCCCCCCAATACACAAATTTCAGCACCATTCTGCCTGAAATGGCACC
ATCACAACCTCAGTCTTGGGTTAGGTGTTGTTCCTGTCCTGAGTTCCTTGGGAT
GGTAAACACAGGCAGTAGCCCTTAGTTTATCTAGATCTGAAAACCCAGACATCA
GATATCGTCAACCAAGACATGGGTGTAATGGGAGGTGGAGTGTGCTGGGGGAGA
TATTCTCAGAAGGGGGAAAGGGGGAAGGGAAGAGGGAGAGAATTC
Human RANKL Promoter Sequence
(SEQ ID NO.5)
      1 acaccaaata tttataaata taactcacac aaataaaacc tctttggtgt tctcaaaatt
    61 ttgaagaatg taaaaggttt gaaaattgct gatctagcaa atgactgaac atgaacagct
  121 atagtatttg tacctgccca gcagtgcagc aattccttat ccttctcata tctgcacttt
  181 aattttcctt tgacaaatat ctctccctcc tctcagccca tgacatgagg ttcacatggg
  241 gttaacttaa ttccctggct caaaggaaag gtattaaatt cagacttgta tccaaccatt
  301 cctgaagcta gacttagccc tatttttcaa taacatgaac caatcaattt tcacatgagt
  361 ccaaaataat tctatgttaa tacactaagg tactaggaaa tatagtttga gaaatgttga
  421 tccaaacatt gtgttattta cagtggagta ttgacataaa ctttgaatct tcaaatatgt
  481 tctggtgtct tggcatctct taatacctat tagcttacaa ggctttcact caactatttt
  541 ataattttga taatgactta attgattagt tgatatattg ttaaaataaa tatattaatg
  601 aatttatgat aaataaggca gataaataag acatgcaatt aggaagacat gttaaacaaa
  661 ttgttataat aatacaatca ctctcagctt aggatagctc ctggccactt tctctctggg
  721 tggtttttac tctgggagta gtttaaatca ttatctagta gtagtttaaa gcattatctt
  781 tgcctaagag ctttcgctga ctccccacat ttgcattgta ctaagagttt tctctgactc
  841 cccacatagg tctagaccct agtattataa gattctcatt gtacttgcac tttgccttca
  901 aagtactaat cacggttttg ttagtgattt gtgtgatgat ttgttgaatc tttttttttt
  961 tcccactagg gtgtaagccc catgttccat cttgatcacc atgtttctag cccagtgctg
1021 gcatatagtg ggttctcact aatatatctg tagagtaaat gaagaaatgc atgagtgaca
1081 tgacaggaga atttaaggat gccatgggag cataaaacag agggagccac ctgggtgagg
1141 agagctgaga aagacttctg gagaggcgac atttgagctg agaaaggaaa gacaagtggg
1201 agagtcctcc aggtgtagaa gttggagaga tgagcgctcc agttaggtag tatttgaagc
1261 tgatgtagaa aaggagtctt gagccagctt gtgaaggact attggagagt tttattttta
1321 tttttatctt ttttttaatt tttgagacag aatcttgctt tgtctcccag gctggagtgc
1381 agtggcatga ttgtagctta ctgcagcttc gacctcctgg gctcaaacaa tccacctatc
1441 tcagccttct gagtaactgg gaccagagat gtgcaccaaa atgcctggct aatttgttca
1501 ttttttgtaa agatagggtc tccctatgtt ccccaggcta ttctccatct cctgggctcc
1561 agtgatcctc acgcctcggc cacccaaagt gctgggatta tagaagtgaa ccactgcgcc
1621 tggcctattg aaggttttta atcttcagag tttcgacttt atcaacaaca cttagaagcc
1681 accaaagaat tgcaggtatg gaaatgacat atacttttgc ttttagaaga aaatcctgat
1741 cagtgtgcac agaattcttc agggggcaag tgtgattcat tctgataaga tatagcatgg
1801 cttagactgg gagactggca gaggctttga agatttcttt gctcaaattt tattcagcaa
1861 gtatttacca tgcacctact atagcaggca acatttttag gaaatggtga atgttacaga
1921 ggtgaataat acagcaagag tcgttgaaca tatggagttt atctattagt tggggagtga
1981 atgttgacaa aggaataagt aaatacatag gcaagaaaga tacattacct gtgaaacagc
2041 agcaggtaga ctgacagtgg agtatctaat acagcctatg gaagccagaa gatagtggga
2101 tgacattttt ggagtactag tagaaatgtc atatgaagaa ctctgtagga atgtaacata
2161 cggtcccata tatgaagctc ctgggtcaag tatacctgaa cataattcag ggatttgagg
2221 gactttcttg taacctgagg atcaagatgt caaggaatta aaaacatgta taaaacattg
2281 ttgtataaaa acccattaaa aagaatggaa gacactatag taaaatcatt gtgggtttag
2341 ttgttataac acattttaaa aatctttgat cccaatcaat atttataaga aagaagaaat
2401 atggaattat ttcctgagtc aaggagcagg gagagaatga ggaagaagag gaggaggagg
2461 agggggagga ggagacaata aacctacttc ccaaagttaa caaacaaaaa gtgggaagag
2521 gtcaaagact acaaggagta gaattaacgt caattgtttc tatgtttgag tctgaaaatt
2581 ttttgtccct tctccaccaa cctatatatt gatacacata taaatgctaa aggcattttt
2641 gaatttgaac agatcatttt ctttgtatgg ctgcctttaa aaaaaattca acctggtcac
2701 tcttcctcaa catttactga ggtctaagtg ttcaatttag aacacatgct ttaataactc
2761 agagacctgt catttgtcac aaatcttgcc tagagaaata ctcattagcg aattaggcag
2821 aaagaggatg caaaataaaa aggcacagta gtcccctgat atccatggaa gactggttcc
2881 aggacaccac caaacccctc cccgcaaata ccaaaatcca tggatgttca agtttcttaa
2941 catatcatgg catagtattt gcatttaacc tacacacatc ctcttgtaca cttgaaatta
3001 tctttagatt atttataata cttaatagaa tgtaaatgct atgtaactag ttgtgtatca
3061 tttaggaaat gatcacaaga aaaaaagtct acagatgtta gtccagacac agccatcctt
3121 tttttttttt tcaaatattt ttgatctgtg gttcattgca tccacagatg tggaacccat
3181 ggatactgtg ggctaactgt attaataaaa aagtggaaac atcctaagtt tcatgggtgt
3241 ttaaattggt cagcaacttc cttctgaaga agtatcagaa tttgtgagca atgttaatat
3301 ttttgttttc tcactaagag ccacagttct gaatagaggt ttttaaaaag ccctagcaag
3361 gtttctttag caatgaaact aacatttaac tgtatcatca gcttcgtgtt acatctcttt
3421 cctgactgtt gggtgagccc tcctcggatg cttgcttctg gctacacgcc cctttaccct
3481 tttctctgca ctgttttcat ctttataaag tcagagttgg tgtctatagg ctctctactg
3541 ccacattcaa gacctgcctc gctcaatgtc accttcaaga tgcagaaata gggatttggg
3601 aaggggattg tgaaattttc gaagtcttcc aaaatacttt gagaaactat atttggaagc
3661 actttggggg gagaggttgg acaggaaggg tcttcagaga tcatcaaatt taactttcta
3721 aatcctaagg aggaaaccga gactccagga tgtgaagtcc cttctctacc aaactagaat
3781 ggatgcagga ggaatgtctg aggtgcaatc cttatccttt agcaaaggtg tcctctgcgt
3841 cttctttaac ccatctcttg gacctccaga aagacagctg aggatggcaa ggggagtctg
3901 gaaccactgg agtagccccc agcctcctcc ttggagggcc cccatgaagg aggcccttca
3961 gtgacagaga ttgagagaga gggagggcga aaggaaggaa ggggagccag aggtgggagt
4021 ggaagaggca gcctcgcctg gggctgattg gctcccgagg ccagggctct ccaagcggtt
4081 tataagagtt ggggctgccg ggcgccctgc ccgctcgccc gcgcgcccca ggacccaaag
4141 ccgggctcca agtcggcgcc ccacgtcgag gctccgccgc agcctccgga gttggccgca
4201 gacaagaagg ggagggagcg ggagagggag gagagctccg aagcgagagg gccga
Human Androgen Receptor Promoter Sequence
(SEQ ID NO.6)
      1 tctagaaaat aattcccaat attgaatccc aaagaattca acatttgggc tgtcgtttga
    61 aagataagtt gaatttggtc atgaaggaag agagggggga tacaatttca gtaaaaggta
  121 acagcaaggt ccaaagacag tcaggtcttc agtagtatgg agtatattca gagggagcca
  181 agatgtctga tgtgaactaa aaagattggt ggttggtagg aggaagaggt gtgagaagag
  241 gctgtaaaga aaaattgaaa cttgattgtg atggacttta aaggctaggc tatgggactt
  301 ggacatgaat ctgcaggcca gtgtttgcag actggcgccc ataactgtct atcacagcaa
  361 cacagacatg tgttgtttgg cctgcagagg tttggcctgc atgatgattt taaaccatct
  421 gaattagtag ccatcatttt caaaaatcaa gagatgccac attaaaatat ggaatgctgc
  481 tgttcttgaa aataatgaaa catctggaac attgaggcca cattcctgac tgacagcaat
  541 cagttggagc tgcgtagtga ctgcccactt tacatggggc atctgatccc tagtcgatta
  601 cagctgccac cacttccctt tatctctcta ataccaagct cttttcactc atttttgtta
  661 cttaagagat atttgggttt gaaacctctg atgcaggtaa ttgagggtta tagagcagag
  721 gacagatgct atcagagttg tcttttaaga aagaaccctc tgttcttcat tttgttgaag
  781 atagcctgga agagggcagc caggggagaa gttagggctg gagctatgag aaagcataag
  841 atgagatgat ggcttcaaca ttgaggacag aaagaatatt gagatgagaa agtagtccat
  901 ataagcatct atgcaaagga aatagcagat gtcctcaaat cagcagaggc aacaactctg
  961 aaagtttatt cataagcccc tcttttcatc tccaatccag ttcaaatgta attatttaaa
1021 ttgttcttca ctctccttcc tggatcatga atgagctcct taaatgcagg gtccacagtg
1081 tcctattcat cagtgaattc caagtgccta gcacagagcc tggcaaatag taaatgctta
1141 acaaatattc gttcagtgca tgaattggag tgattctcta ctttgcctca taagttgaaa
1201 aaaggtttat tacataccta aatatgctga aatcacaggg catttggcaa ccccccaaaa
1261 ccaaaactcc cagtttggaa acagaatttt aattctgtga aaataaaatc cattcattta
1321 ttcaaaaaat atttattaaa caatgaccat gtccacacca ggctgagtcc taaggattca
1381 atgatgaaca aaaaccaaca tgattcctgc tcttaggaaa catacagttc agtgaggaaa
1441 acagattgtg agaagtcctc caacaaatac tgggtgctat taaaatatat taaaaggtga
1501 gtgggtgagg gacttgagct agcctaggtg gttcaggaag tcttcctgga tgtgctgata
1561 tgcataggca ttaactagat aaatagagag aaggatgaac caacattgca ggtagaggga
1621 acagaatatg caaaggcagg aaggattatg gagtcgttgg aggacctgaa taaaggccca
1681 gtgtaagtgg atctcagaaa acaggaggaa aggtgtatga gatgagatca gagaggcaga
1741 tcatgtgggg tatggttaat gttttggact tttctattaa gagcaatggg gagacagtga
1801 caggacttaa acggggaaat aatatgacca gattaaactt tctaaaaaac cctctatgca
1861 aatatatatt gagagttaat tattgacaaa gattcaaagg caacaaagtg gagagagaat
1921 agtattttca aaaaatggtg ccaaaacaat aggacatcta tattaaaagt tgggtatctg
1981 tctacaaaac ttaattcaaa atggatcaca gacctaaatg taaaactgaa agctatacaa
2041 cttctggaag gaaaacacag atgggaatct gtgtgatctt gagtttgaaa atgatttatt
2101 atatctgaca ccataatccg taagttaaca taattcataa gtgaacaaag tgatgaactg
2161 gacttcatca gaatttaaaa tgtttgtgct tcaaaagaca ctggtatgat aatgaagaca
2221 aactacagat aagatattgt tgaatcatat ttctgataaa ggaattgtgg ctcagaatac
2281 ataactctaa acccccataa taaattacaa gtagcccaat taaaaaaaaa aaaagagaaa
2341 aaatttacag tcttcatcaa agaaagtatc aattgtaaaa taagcacatg aaaaatgctc
2401 tgcatcttta ttcatggggg gatgaaataa aaattaaatg ggaaagacac ctctaattag
2461 aatactaaaa ttaaaaagac tgaccatacc aagtattggt gaagtggaaa tgtaaaatga
2521 tacaatcaac ttaggtagat gatttggaag tttcttacaa aagtaggtgt atacctaccc
2581 tgtgactcac ccattccatg gctaagtatt tacctgagag aaatgaaaga atacatccat
2641 acaaagatgt ttatacaaat atttatagca gttttatttg tagtagcccc aaactgaaaa
2701 gaacccaaat gtccatcaaa agtgaatgga taaacaaagc gtggtacagc aatgcaatag
2761 aatactactt agcaataaag aagaatgagc tagtgatata cataacagct taaatgtaca
2821 tcaaaggcat tgtgctcagt gaaagatgca agtaaaaaaa aaaaagagta catgctgtat
2881 agttccattg acataaaact ctggaaagtg aaaaacagtc tatactgaca gaaagcagat
2941 cattggttgc ctgaggagga ggagtatagg agaggtggag ggaaaatgta caaagtggca
3001 caataaaaac ttttggaatc atagatatat tcactatctt gattgagtga tgatttcatg
3061 agtgcacgtg cgtgtgtcaa aaatgatcaa tttatgcaac tttaaatatg tgcagtttat
3121 tgtatatatc aattatacct cagtacggct attaaaaaga aaccctctgg ctgcacaatg
3181 cagaactgat tctaggaaag agtggaggga ggatgaccat ttacagtgct ccaggtggaa
3241 gagaacggtg ccttctggaa gtgaactagg ttggcaacaa cagagatgaa ataaatgggc
3301 agatgtgtga gatacttagg aaataaaacc cgatggtcac cattttccaa aggtcagctc
3361 atcctggctt tccagagcaa agagctaggg aagactttat taataaatcc ctcttgaagt
3421 tgcagaggaa gcttatagca gaaacttact ctcaacctga ctaatctgag agaacacctc
3481 tggttccatt tgattactaa aaaactgcaa agaacaggag gagaaagaag aagaaagctg
3541 gtacaaacag tgaacttata taatattaat caataattgt ctcttgttct taaaagcaat
3601 gggaagaaaa tgagatttga gctggaagat cagagttcaa aatccaaata aagtatatgg
3661 ccctaatatg cttatagtag ttaacctttc ctgataatga tataattgtt gacagcacca
3721 tctttaaaat aaaataacat agtaatcctt cagatttgta gaagatcttt cctgtttaca
3781 agtttgttct atacacatta tgtcttttaa atgacacact agccttctga gggtaactta
3841 tattggcaac agttttcaga tgtggaaact gtgaagacaa tgttggtgat gtggaagcaa
3901 cataaacttt ggagtctttc agacccaggt ttgaatgtca gactgctttt tattcagagt
3961 aacttcagag cattatttct caccttaatt ttttttcagg cctctttgtg tctatgtgtc
4021 ctcttcactc ctgtccattg tttcttcagt gatttttgcc accttccttc actgttagtg
4081 tgtagacaca tagttctcct ggctctgaga gcctatgtta attccattct accatcctgc
4141 cacggcccac tcaattccta ttgagcaatg ctagttgaaa gttgtggtgg gattaaatgt
4201 tgcaatgagt attcaaatga ggttgaagta tctacgcatt ctacttacat atggtgaggt
4261 atattcaagg aagctgtagc cattaaaatc tcaggaaata atttttcacc tcctcaggtg
4321 aaagggtctt caggcctttg tgttctggaa ggttcattta tagccatttc ccaaatgaca
4381 atgcgattga tgagtctaga gtctagctca aatagcaatg gactggaaga ctagtttagg
4441 ttttactaat gtggaacata gaacaaatta tgtccttgtt tcagcctgtt catctgtgaa
4501 atagagccta tcatatccag tcttccttgc ctttaggttt gagttacctt ctttggtcaa
4561 ggtaagtaaa tgcctatgat gtttggctgt gcacaagata aagctacaac aaagctacaa
4621 cccatctttt ctctgtagaa gactcaaaaa gcaaaagaga cccaggaaaa tctcggaatg
4681 acttttggaa cagagagcct ccccagaatc agaagtcaag gaatttaaac atagggaagg
4741 cccaggtctc tactgacata aaggaaagat gttttcttat aggtttcacg tttacatttt
4801 ctctctcttg atcccattcc cacttgcatc tgccaccttt acacagggct tatgggacct
4861 cctccacaaa agagcagttg cagtaaccca catcatcctc tacgccctgg ctgtccatca
4921 agaggcgaaa agcagcccta tataggttct atccttggat agttccagtt gtaaagttta
4981 aaatatgcga aggcaacttg gaaaagcaag cggctgcata caaagcaaac gtttacagag
5041 ctctggacaa aattgagcgc ctatgtgtac atggcaagtg tttttagtgt ttgtgtgttt
5101 acctgcttgt ctgggtgatt ttgcctttga gagtctggag agtagaagta ctggttaaag
5161 gaacttccag acaggaagaa ggcagagaag agggtagaaa tgactctgat tcttggggct
5221 gagggttcct agagcaaatg gcacaatgcc acgaggcccg atctatccct atgacggaat
5281 ctaaggtttc agcaagtatc tgctggcttg gtcatggctt gctcctcagt ttgtaggaga
5341 ctctcccact ctcccatctg cgcgctctta tcagtcctga aaagaacccc tggcagccag
5401 gagcaggtat tcctatcgtc cttttcctcc ctccctcgcc ccaccctgtt ggttttttag
5461 attgggcttt ggaaccaaat ttcctgagtg ctggcctcca ggaaatctgg agccctggcg
5521 cctaaacctt ggtttaggaa accaggagct attcaggaag caggggtcct ccagggctag
5581 agctagcctc tcctgccctc gcccacgctg cgccagcact tgtttctcca aagccactag
5641 gcaggcgtta gcgcgcggtg aggggagggg agaaaaggaa aggggagggg agggaaaagg
5701 aggtgggaag gcaaggaggc cggcccggtg ggggcgggac ccgactcgca aactgttgca
5761 tttgctctcc acctcccagc gccccctccg agatcccggg gagccagctt gctgggagag
5821 cgggacggtc cggagcaagc ccacaggcag aggaggcgac agagggaaaa agggccgagc
5881 tagccgctcc agtgctgtac aggagccgaa gggacgcacc acgccagccc cagcccggct
5941 ccagcgacag ccaacgcctc ttgcagcgcg gcggcttcga agccgccgcc cggagctgcc
6001 ctttcctctt cggtgaagtt tttaaaagct gctaaagact cggaggaagc aaggaaagtg
6061 cctggtagga ctgacggctg cctttgtcct cctcctctcc accccgcctc cccccaccct
6121 gccttccccc cctcccccgt cttctctccc gcagctgcct cagtcggcta ctctcagcca
6181 acccccctca ccacccttct ccccacccgc ccccccgccc ccgtcgccca gcgctgccag
6241 cccgagtttg cagagaggta actccctttg gctgcgagcg ggcgagctag ctgcacattg
6301 caaagaaggc tcttaggagc caggcgactg gggagcggct tcagcactgc agccacgacc
6361 cgcctggtta ggctgcacgc ggagagaacc ctctgttttc ccccactctc tctccacctc
6421 ctcctgcctt ccccaccccg agtgcggagc cagagatcaa aagatgaaaa ggcagtcagg
6481 tcttcagtag ccaaaaaaca aaacaaacaa aaacaaaaaa caagaaataa aagaaaaaga
6541 taataactca gttcttattt gcacctactt cagtggacac tgaatttgga aggtggagga
6601 ttttgttttt ttcttttaag atctgggcat cttttgaatc tacccttcaa gtattaagag
6661 acagactgtg agcctagcag ggcagatctt gtccaccgtg tgtcttcttc tgcacgagac
6721 tttgaggctg tcagagcgct ttttgcgtgg ttgctcccgc aagtttcctt ctctggagct
6781 tcccgcaggt gggcagctag ctgcagcgac taccgcatca tcacagcctg ttgaactctt
6841 ctgagcaaga gaaggggagg cggggtaagg gaagtaggtg gaagattcag ccaagctcaa
6901 ggatg
```

The invention further provides probes, primers and fragments of the osteomimicry regulatory region, and transcriptionally active fragments thereof. In one embodiment, purified nucleic acids consisting of at least 8 nucleotides (i.e., a hybridizable portion) of a regulatory region, and transcriptionally active fragments thereof gene sequence are provided; in other embodiments, the nucleic acids consist of at least 20 (contiguous) nucleotides, 25 nucleotides, 50 nucleotides, 100 nucleotides, 200 nucleotides, 500, 1000, 2000, 3000, 4000 or 5000 nucleotides of an osteomimicry regulatory region sequence, or transcriptionally active fragment thereof sequence. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al., 1989, supra; also see the techniques described in “Oligonucleotide Synthesis”, 1984, Gait M. J. ed., IRL Press, Oxford, which is incorporated herein by reference in its entirety.

In another embodiment, the nucleic acids are smaller than 20, 25, 35, 200 or 500 nucleotides in length. Nucleic acids can be single or double stranded. The invention also encompasses nucleic acids hybridizable to or complementary to the foregoing sequences. In specific aspects, nucleic acids are provided which comprise a sequence complementary to at least 10, 20, 25, 50, 100, 200, 500 nucleotides or the entire osteomimicry regulatory region and transcriptionally active fragments gene.

The nucleotide sequences of the invention also include nucleotide sequences that have at least 65%, 70%, 75%, 80%, 85%, 90%, 95%, 98%, 99% or more nucleotide sequence identity to the nucleotide sequence depicted in SEQ ID NOs. 1, 2, 3, 4, 5, and 6, and/or transcriptionally active fragments thereof, which are capable of driving expression specifically within tumor and tissue cells with calcification potential.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions .times.100). In one embodiment, the two sequences are the same length.

The determination of percent identity between two sequences also can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res.25:3389-3402. Alternatively, PSI-Blast can be used to perform an iterated search which detects distant relationships between molecules (Id.). When utilizing BLAST, Gapped BLAST and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12 and a gap penalty of 4 can be used. In an alternate embodiment, alignments can be obtained using the NA_MULTIPLE_ALIGNMENT 1.0 program, using a GapWeight of 5 and a GapLengthWeight of 1.

The percent identity between two sequences can be determined using techniques similar to those described above, with or without allowing gaps. In calculating percent identity, typically only exact matches are counted.

The invention also encompasses:

(a) DNA vectors that contain any of the foregoing osteomimicry regulatory sequences and/or their complements (i.e., antisense);

(b) DNA expression vectors that contain any of the foregoing osteomimicry regulatory element sequences operatively associated with a heterologous gene, such as a reporter gene; and

(c) genetically engineered host cells that contain any of the foregoing osteomimicry regulatory element sequences operatively associated with a heterologous gene such that the osteomimicry regulatory element directs the expression of the heterologous gene in the host cell.

Also encompassed within the scope of the invention are various transcriptionally active fragments of this regulatory region. A “transcriptionally active” or “transcriptionally functional” fragment of the osteomimicry regulatory region according to the present invention refers to a polynucleotide comprising a fragment of said polynucleotide which is functional as a regulatory region for expressing a recombinant polypeptide or a recombinant polynucleotide in a recombinant cell host. For the purpose of the invention, a nucleic acid or polynucleotide is “transcriptionally active” as a regulatory region for expressing a recombinant polypeptide or a recombinant polynucleotide if said regulatory polynucleotide contains nucleotide sequences which contain transcriptional information, and such sequences are operably associated to nucleotide sequences which encode the desired polypeptide or the desired polynucleotide.

In particular, the transcriptionally active fragments of the osteomimicry regulatory region of the present invention encompass those fragments that are of sufficient length to promote transcription of a heterologous gene, such as a reporter gene, when operatively linked to the osteomimicry regulatory sequence and transfected into tumor and tissue cells with calcification potential. Typically, the regulatory region is placed immediately 5′ to, and is operatively associated with the coding sequence. As used herein, the term “operatively associated” refers to the placement of the regulatory sequence immediately 5′ (upstream) of the reporter gene, such that transacting factors required for initiation of transcription, such as transcription factors, polymerase subunits and accessory proteins, can assemble at this region to allow RNA polymerase dependent transcription initiation of the reporter gene.

In one embodiment, the polynucleotide sequence chosen may further comprise other nucleotide sequences, either from the osteomimicry regulatory region, and transcriptionally active fragments thereof gene, or from a heterologous gene. In another embodiment, multiple copies of a promoter sequence, or a fragment thereof, may be linked to each other. For example, the promoter sequence, or a fragment thereof, may be linked to another copy of the promoter sequence, or another fragment thereof, in a head to tail, head to head, or tail to tail orientation. In another embodiment, an osteotropic-specific enhancer may be operatively linked to the osteomimicry regulatory sequence, or fragment thereof, and used to enhance transcription from the construct containing the osteomimicry regulatory sequence.

Also encompassed within the scope of the invention are modifications of the osteomimicry nucleotide sequences depicted in SEQ ID Nos. 1-6, respectively, without substantially affecting its transcriptional activities. Such modifications include additions, deletions and substitutions. In addition, any nucleotide sequence that selectively hybridizes to the complement of the sequence depicted in SEQ ID Nos. 1-6, respectively, under stringent conditions, and is capable of activating the expression of a coding sequence specifically within tumor and tissue cells with calcification potential is encompassed by the invention. Exemplary moderately stringent and high stringency hybridization conditions Ausubel F. M. et al., eds., 1989, Current Protocols in Molecular Biology, Vol. I, Green Publishing Associates, Inc., and John Wiley & sons, Inc., New York, at p. 2.10.3. Other conditions of high stringency which may be used are well known in the art.

The osteomimicry regulatory region, or transcriptionally functional fragments thereof, is preferably derived from a mammalian organism. Screening procedures which rely on nucleic acid hybridization make it possible to isolate gene sequences from various organisms. The isolated polynucleotide sequence disclosed herein, or fragments thereof, may be labeled and used to screen a cDNA library constructed from mRNA obtained from appropriate cells or tissues (e.g., calcified tissue) derived from the organism of interest. The hybridization conditions used should be of a lower stringency when the cDNA library is derived from an organism different from the type of organism from which the labeled sequence was derived. Further, mammalian osteomimicry regulatory region homologues may be isolated from, for example, bovine or other non-human nucleic acid, by performing polymerase chain reaction (PCR) amplification using two primer pools designed on the basis of the nucleotide sequence of the osteomimicry regulatory region disclosed herein. The template for the reaction may be cDNA obtained by reverse transcription of the mRNA prepared from, for example, bovine or other non-human cell lines, or tissue known to express the osteomimicry gene. For guidance regarding such conditions, see, e.g., Innis et al. (Eds.) 1995, PCR Strategies, Academic Press Inc., San Diego; and Erlich (ed) 1992, PCR Technology, Oxford University Press, New York, each of which is incorporated herein by reference in its entirety.

Promoter sequences within the 5′ non-coding regions of the osteomimicry gene may be further defined by constructing nested 5′ and/or 3′ deletions using conventional techniques such as exonuclease III or appropriate restriction endonuclease digestion. The resulting deletion fragments can be inserted into the promoter reporter vector to determine whether the deletion has reduced or obliterated promoter activity, such as described, for example, by Coles et al. (Hum. Mol. Genet., 7:791-800, 1998). In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. These types of assays are well known to those skilled in the art (WO 97/17359, U.S. Pat. No. 5,374,544, EP 582 796, U.S. Pat. No. 5,698,389, U.S. Pat. No. 5,643,746, U.S. Pat. No. 5,502,176, and U.S. Pat. No. 5,266,488).

The osteomimicry regulatory regions and transcriptionally functional fragments thereof, and the fragments and probes described herein which serve to identify osteomimicry regulatory regions and fragments thereof, may be produced by recombinant DNA technology using techniques well known in the art. Methods which are well known to those skilled in the art can be used to construct these sequences, either in isolated form or contained in expression vectors. These methods include, for example, in vitro recombinant DNA techniques, synthetic techniques and in vivo genetic recombination. See, e.g., the techniques described in Sambrook et al., 1989, supra, and Ausabel et al, 1989, supra; also see the techniques described in “Oligonucleotide Synthesis”, 1984, Gait M. J. ed., IRL Press, Oxford, which is incorporated herein by reference in its entirety.

Alterations in the regulatory sequences can be generated using a variety of chemical and enzymatic methods which are well known to those skilled in the art. For example, regions of the sequences defined by restriction sites can be deleted. Oligonucleotide-directed mutagenesis can be employed to alter the sequence in a defined way and/or to introduce restriction sites in specific regions within the sequence. Additionally, deletion mutants can be generated using DNA nucleases such as Bal31, ExoIII, or S1 nuclease. Progressively larger deletions in the regulatory sequences are generated by incubating the DNA with nucleases for increased periods of time (see, e.g., Ausubel et al., 1989, supra).

The altered sequences are evaluated for their ability to direct expression of heterologous coding sequences in appropriate host cells. It is within the scope of the present invention that any altered regulatory sequences which retain their ability to direct expression of a coding sequence be incorporated into recombinant expression vectors for further use.

Analysis of Osteomimecry Regulatory Region Activity

The osteomimicry regulatory region sequence, or transcriptionally active fragment thereof such as for example, and not by way of limitation, nucleotide sequences encoding the osteocalcin (OC), bone sialoprotein (BSP), SPARC/osteonectin (ON), osteopontin (OPN) and the receptor activator of NF-κB ligand (RANKL) regulatory region shows selective tissue and cell-type specificity; i.e., induces gene expression in osteotropic cells. Thus, the osteomimicry regulatory region sequence, and transcriptionally active fragments thereof, of the present invention may be used to induce expression of a heterologous coding sequence specifically in osteotropic cells. The activity and the specificity of the osteomimicry regulatory region sequence, and transcriptionally active fragments thereof can further be assessed by monitoring the expression level of a detectable polynucleotide operably associated with the osteomimicry regulatory region sequence, and transcriptionally active fragments thereof in different types of cells, tissues and cell lines engineered to contain the osteomimicry regulatory region sequence, and transcriptionally active fragments thereof. As discussed hereinbelow, the detectable polynucleotide may be either a polynucleotide that specifically hybridizes with a predefined oligonucleotide probe, or a polynucleotide encoding a detectable protein. The osteomimicry regulatory region sequence, and transcriptionally active fragments thereof can then be used to screen for candidate compounds or substances that interfere with the expression of the heterologous coding sequence. Such identified compounds or substances that interfere with osteomimicry regulatory region sequence, and transcriptionally active fragments thereof will be likely candidate compounds that interfere with the ability of cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL).

Osteomimecry Regulatory Region Driven Reporter Constructs

The regulatory polynucleotides according to the invention may be advantageously part of a recombinant expression vector that may be used to express a coding sequence, or reporter gene, in a desired host cell or host organism. The osteomimicry regulatory region sequence, and transcriptionally active fragments thereof of the present invention, and transcriptionally active fragments thereof, may be used to direct the expression of a heterologous coding sequence. In particular, the present invention encompasses mammalian osteomimicry regulatory region sequence, and transcriptionally active fragments thereof. In accordance with the present invention, transcriptionally active fragments of the osteomimicry regulatory region sequence, and transcriptionally active fragments thereof encompass those fragments of the region which are of sufficient length to promote transcription of a reporter coding sequence to which the fragment is operatively linked.

A variety of reporter gene sequences well known to those of skill in the art can be utilized, including, but not limited to, genes encoding fluorescent proteins such as green fluorescent protein (GFP), enzymes (e.g. CAT, beta-galactosidase, luciferase) or antigenic markers. For convenience, enzymatic reporters and light-emitting reporters analyzed by colorometric or fluorometric assays are preferred for the screening assays of the invention.

In one embodiment, for example, a bioluminescent, chemiluminescent or fluorescent protein can be used as a light-emitting reporter in the invention. Types of light-emitting reporters, which do not require substrates or cofactors, include, but are not limited to the wild-type green fluorescent protein (GFP) of Victoria aequoria (Chalfie et al., 1994, Science 263:802-805), and modified GFPs (Heim et al., 1995, Nature 373:663-4; PCT publication WO 96/23810). Transcription and translation of this type of reporter gene leads to the accumulation of the fluorescent protein in test cells, which can be measured by a fluorimeter, or a flow cytometer, for example, by methods that are well known in the art (see, e.g., Lackowicz, 1983, Principles of Fluorescence Spectroscopy, Plenum Press, New York).

Another type of reporter gene that may be used are enzymes that require cofactor(s) to emit light, including but not limited to, Renilla luciferase. Other sources of luciferase also are well known in the art, including, but not limited to, the bacterial luciferase (luxAB gene product) of Vibrio harveyi (Karp, 1989, Biochim. Biophys. Acta 1007:84-90; Stewart et al. 1992, J. Gen. Microbiol, 138:1289-1300), and the luciferase from firefly, Photinus pyralis (De Wet et al. 1987, Mol. Cell. Biol. 7:725-737), which can be assayed by light production (Miyamoto et al., 1987, J. Bacteriol. 169:247-253; Loessner et al 1996, Environ. Microbiol. 62:1133-1140; and Schultz & Yarus, 1990, J. Bacteriol. 172:595-602).

Reporter genes that can be analyzed using colorimetric analysis include, but are not limited to, .beta.-galactosidase (Nolan et al. 1988, Proc. Natl. Acad. Sci. USA 85:260307), .beta.-glucuronidase (Roberts et al. 1989, Curr. Genet. 15:177-180), luciferase (Miyamoto et al., 1987, J. Bacteriol. 169:247-253), or .beta.-lactamase. In one embodiment, the reporter gene sequence comprises a nucleotide sequence which encodes a LacZ gene product, Pgalactosidase. The enzyme is very stable and has a broad specificity so as to allow the use of different histochemical, chromogenic or fluorogenic substrates, such as, but not limited to, 5-bromo-4-chloro-3-indoyl-.beta.-D-galactoside (X-gal), lactose 2,3,5-triphenyl-2H-tetrazolium (lactose-tetrazolium) and fluorescein galactopyranoside (see Nolan et al., 1988, supra).

In another embodiment, the product of the E. coli .beta.-glucuronidase gene (GUS) can be used as a reporter gene (Roberts et al. 1989, Curr. Genet. 15:177-180). GUS activity can be detected by various histochemical and fluorogenic substrates, such as Xglucuronide (Xgluc) and 4-methylumbelliferyl glucuronide.

In addition to reporter gene sequences such as those described above, which provide convenient colorimetric responses, other reporter gene sequences, such as, for example, selectable reporter gene sequences, can routinely be employed. For example, the coding sequence for chloramphenicol acetyl transferase (CAT) can be utilized, leading to osteomimicry regulatory region sequence, and transcriptionally active fragments thereof-dependent expression of chloramphenicol resistant cell growth. The use of CAT and the advantages of a selectable reporter gene are well known to those skilled in the art (Eikmanns et al. 1991, Gene 102:93-98). Other selectable reporter gene sequences also can be utilized and include, but are not limited to, gene sequences encoding polypeptides which confer zeocin (Hegedus et al. 1998, Gene 207:241-249) or kanamycin resistance (Friedrich & Soriano, 1991, Genes. Dev. 5:1513-1523).

Other coding sequences, such as toxic gene products, potentially toxic gene products, and antiproliferation or cytostatic gene products, also can be used. In another embodiment, the detectable reporter polynucleotide may be either a polynucleotide that specifically hybridizes with a predefined oligonucleotide probe, or a polynucleotide encoding a detectable protein, including a BSP polypeptide or a fragment or a variant thereof. This type of assay is well known to those skilled in the art (U.S. Pat. No. 5,502,176 and U.S. Pat. No. 5,266,488).

Osteomimecry regulatory region sequence, and transcriptionally active fragments thereof driven reporter constructs can be constructed according to standard recombinant DNA techniques (see, e.g., Methods in Enzymology, 1987, volume 154, Academic Press; Sambrook et al. 1989, Molecular Cloning—A Laboratory Manual, 2nd Edition, Cold Spring Harbor Press, New York; and Ausubel et al. Current Protocols in Molecular Biology, Greene Publishing Associates and Wiley Interscience, New York, each of which is incorporated herein by reference in its entirety).

Methods for assaying promoter activity are well-known to those skilled in the art (see, e.g., Sambrook et al., Molecular Cloning A Laboratory Manual, Cold Spring Harbor Laboratory, Cold Spring Harbor, N.Y., 1989). An example of a typical method that can be used involves a recombinant vector carrying a reporter gene and genomic sequences from the osteomimicry regulatory region sequence depicted in SEQ ID NOs. 1-6, respectively. Briefly, the expression of the reporter gene (for example, green fluorescent protein, luciferase, .beta.-galactosidase or chloramphenicol acetyl transferase) is detected when placed under the control of a biologically active polynucleotide fragment. Genomic sequences located upstream of the first exon of the gene may be cloned into any suitable promoter reporter vector. For example, a number of commercially available vectors can be engineered to insert the osteomimicry regulatory region sequence, and transcriptionally active fragments thereof of the invention for expression in mammalian host cells. Non-limiting examples of such vectors are pSEAPBasic, pSEAP-Enhancer, ppgal-Basic, p.beta.gal-Enhancer, or pEGFP-1 Promoter Reporter vectors (Clontech, Palo Alto, Calif.) or pGL2-basic or pGL3-basic promoterless luciferase reporter gene vector (Promega, Madison, Wis.). Each of these promoter reporter vectors include multiple cloning sites positioned upstream of a reporter gene encoding a readily assayable protein such as secreted alkaline phosphatase, green fluorescent protein, luciferase or .beta.-galactosidase. The osteomimicry regulatory region sequence, and transcriptionally active fragments thereof are inserted into the cloning sites upstream of the reporter gene in both orientations and introduced into an appropriate host cell. The level of reporter protein is assayed and compared to the level obtained with a vector lacking an insert in the cloning site. The presence of an elevated expression level in the vector containing the insert with respect the control vector indicates the presence of a promoter in the insert.

Expression vectors that comprise a osteomimicry regulatory region sequence, and transcriptionally active fragments thereof may further contain a gene encoding a selectable marker. A number of selection systems may be used, including but not limited to, the herpes simplex virus thymidine kinase (Wigler et al., 1977, Cell 11:223), hypoxanthine-guanine phosphoribosyltransferase (Szybalska & Szybalski, 1962, Proc. Natl. Acad. Sci. USA 48:2026) and adenine phosphoribosyltransferase (Lowy et al., 1980, Cell 22:817) genes, which can be employed in tk.sup.-, hgprt.sup.- or aprt.sup.-cells, respectively. Also, antimetabolite resistance can be used as the basis of selection for dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147) genes. Additional selectable genes include trpB, which allows cells to utilize indole in place of tryptophan; hisD, which allows cells to utilize histinol in place of histidine (Hartman & Mulligan, 1988, Proc. Natl. Acad. Sci. USA 85:8047); ODC (ornithine decarboxylase) which confers resistance to the ornithine decarboxylase inhibitor, 2-(difluoromethyl)-DL-ornithine, DFMO (McConlogue L., 1987, In: Current Communications in Molecular Biology, Cold Spring Harbor Laboratory ed.) and glutamine synthetase (Bebbington et al., 1992, Biotech 10:169).

Characterization of Transcriptionally Active Osteomimecry Regulatory Region Sequences, and Transcriptionally Active Fragments Thereof

A fusion construct comprising an osteomimicry regulatory region sequence, and transcriptionally active fragments thereof, or a fragment thereof, can be assayed for transcriptional activity. As a first step in promoter analysis, the transcriptional start point (+1 site) of the osteotropic-specific gene under study has to be determined using primer extension assay and/or RNAase protection assay, following standard methods (Sambrook et al.,1989, Molecular Cloning: A Laboratory Manual, Cold Spring Harbor, Cold Spring Harbor Press). The DNA sequence upstream of the +1 site is generally considered as the promoter region responsible for gene regulation. However, downstream sequences, including sequences within introns, also may be involved in gene regulation. To begin testing for promoter activity, a −3 kb to +3 kb region (where +1 is the transcriptional start point) may be cloned upstream of the reporter gene coding region. Two or more additional reporter gene constructs also may be made which contain 5′ and/or 3′ truncated versions of the regulatory region to aid in identification of the region responsible for osteotropic-specific expression. The choice of the type of reporter gene is made based on the application.

In a preferred embodiment, a GFP reporter gene construct is used. The application of green fluorescent protein (GFP) as a reporter is particularly useful in the study of osteotropic-specific gene promoters. A major advantage of using GFP as a reporter lies in the fact that GFP can be detected in freshly isolated tumor and tissue cells with calcification potential without the need for substrates.

In another embodiment of the invention, a Lac Z reporter construct is used. The Lac Z gene product, .beta.-galactosidase, is extremely stable and has a broad specificity so as to allow the use of different histochemical, chromogenic or fluorogenic substrates, such as, but not limited to, 5-bromo-4-chloro-3-indoyl-.beta.-D-galactoside (X-gal), lactose 2,3,5-triphenyl-2H-tetrazolium (lactose-tetrazolium) and fluorescein galactopyranoside (see Nolan et al., 1988, supra).

For promoter analysis in transgenic mice, GFP that has been optimized for expression in mammalian cells is preferred. The promoterless cloning vector pEGFP1 (Clontech, Palo Alto, Calif.) encodes a red shifted variant of the wild-type GFP which has been optimized for brighter fluorescence and higher expression in mammalian cells (Cormack et al., 1996, Gene 173:33; Haas et al., 1996, Curr. Biol. 6:315). Moreover, since the maximal excitation peak of this enhanced GFP (EGFP) is at 488 nm, commonly used filter sets such as fluorescein isothiocyanate (FITC) optics which illuminate at 450-500 nm can be used to visualize GFP fluorescence. pEGFP1 proved to be useful as a reporter vector for promoter analysis in transgenic mice (Okabe et al, 1997, FEBS Lett. 407:313). In an alternate embodiment, transgenic mice containing transgenes with an osteomimicry regulatory region sequence, and transcriptionally active fragments thereof upstream of a luciferase reporter gene are utilized.

Putative osteomimicry regulatory region sequences, and transcriptionally active fragments thereof can be prepared (usually from a parent phage clone containing 8-10 kb genomic DNA including the promoter region) for cloning using methods known in the art. In one embodiment, for example, promoter fragments are cloned into the multiple cloning site of a luciferase reporter vector. In one embodiment, restriction endonucleases are used to excise the osteomimicry regulatory region sequence, and transcriptionally active fragments thereof to be inserted into the reporter vector. However, the feasibility of this method depends on the availability of proper restriction endonuclease sites in the regulatory fragment. In a preferred embodiment, the required promoter fragment is amplified by polymerase chain reaction (PCR; Saiki et al., 1988, Science 239:487) using oligonucleotide primers bearing the appropriate sites for restriction endonuclease cleavage. The sequence necessary for restriction cleavage is included at the 5′ end of the forward and reverse primers which flank the regulatory fragment to be amplified. After PCR amplification, the appropriate ends are generated by restriction digestion of the PCR product. The osteomimicry regulatory region sequence, and transcriptionally active fragments thereof, generated by either method, are then ligated into the multiple cloning site of the reporter vector following standard cloning procedures (Sambrook et al., 1989, supra). It is recommended that the DNA sequence of the PCR generated promoter fragments in the constructs be verified prior to generation of transgenic animals. The resulting reporter gene construct will contain the putative osteomimicry regulatory region sequence, and transcriptionally active fragments thereof located upstream of the reporter gene open reading frame, e.g., GFP or luciferase cDNA. The osteomimicry regulatory region sequence, and transcriptionally active fragments thereof with the reporter gene can then be used to screen for candidate compounds or substances that interfere with the expression of the heterologous coding sequence. Such identified compounds or substances that interfere with osteomimicry regulatory region sequence, and transcriptionally active fragments thereof will be likely candidate compounds that interfere with the ability of cancer cells to express highly restricted bone-like proteins comprising, inter alia, one or more of osteocalcin (OC), bone sialoprotein (BSP), SPARC/osteonectin (ON), osteopontin (OPN) and the receptor activator of NF-κB ligand (RANKL).

Osteomimecry Regulatory Region Sequence Analysis Using Transgenic Mice

The mammalian osteomimicry regulatory region sequences, and transcriptionally active fragments thereof can be used to direct expression of, inter alia, a reporter coding sequence, a homologous gene or a heterologous gene in transgenic animals specifically within tumor and tissue cells with calcification potential. Animals of any species, including, but not limited to, mice, rats, rabbits, guinea pigs, pigs, micro-pigs, goats, sheep, and non-human primates, e.g., baboons, monkeys and chimpanzees may be used to generate transgenic animals. The term “transgenic,” as used herein, refers to non-human animals expressing osteomimicry regulatory region and transcriptionally active fragments thereof sequences from a different species (e.g., mice expressing human osteomimicry regulatory region and transcriptionally active fragments thereof sequences), as well as animals that have been genetically engineered to over-express endogenous (i.e., same species) osteomimicry regulatory region and transcriptionally active fragments thereof sequences or animals that have been genetically engineered to knock-out specific sequences.

In one embodiment, the present invention provides for transgenic animals that carry a transgene such as a reporter gene, therapeutic and/or toxic coding sequence under the control of the osteomimicry regulatory region and transcriptionally active fragments thereof, in all their cells, as well as animals that carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (1992, Proc. Natl. Acad. Sci. USA 89:6232-6236). When it is desired that the transgene be integrated into the chromosomal site of the endogenous corresponding gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene.

Any technique known in the art may be used to introduce a transgene under the control of the osteomimicry regulatory region and transcriptionally active fragments thereof into animals to produce the founder lines of transgenic animals. Such techniques include, but are not limited to, pronuclear microinjection (Hoppe & Wagner, 1989, U.S. Pat. No. 4,873,191); nuclear transfer into enucleated oocytes of nuclei from cultured embryonic, fetal or adult cells induced to quiescence (Campbell et al., 1996, Nature 380:64-66; Wilmut et al., Nature 385:810-813); retrovirus gene transfer into germ lines (Van der Putten et al., 1985, Proc. Natl. Acad. Sci., USA 82:6148-6152); gene targeting in embryonic stem cells (Thompson et al., 1989, Cell 65:313-321); electroporation of embryos (Lo, 1983, Mol. Cell. Biol. 31:1803-1814); and sperm-mediated gene transfer (Lavitrano et al., 1989, Cell 57:717-723; see, Gordon, 1989, Transgenic Animals, Intl. Rev. Cytol. 115:171-229).

For example, for microinjection of fertilized eggs, a linear DNA fragment (the transgene) containing the regulatory region, the reporter gene and the polyadenylation signals, is excised from the reporter gene construct. The transgene may be gel purified by methods known in the art, for example, by the electroelution method. Following electroelution of gel fragments, any traces of impurities are further removed by passing through Elutip D column (Schleicher & Schuell, Dassel, Germany).

In a preferred embodiment, the purified transgene fragment is microinjected into the male pronuclei of fertilized eggs obtained from B6 CBA females by standard methods (Hogan, 1986, Manipulating the Mouse Embryo, A Laboratory Manual. Cold Spring Harbor Laboratory Press, Cold Spring Harbor, N.Y.). Mice are analyzed transiently at several embryonic stages or by establishing founder lines that allow more detailed analysis of transgene expression throughout development and in adult animals. Transgene presence is analyzed by PCR using genomic DNA purified from placentas (transients) or tail clips (founders) according to the method of Vemet et al., Methods Enzymol. 1993;225:434-451. Preferably, the PCR reaction is carried out in a volume of 100 .mu.l containing 1 .mu.g of genomic DNA, in 1.times. reaction buffer supplemented with 0.2 mM dNTPs, 2 mM MgCl.sub.2, 600 .mu.M each of primer, and 2.5 units of Taq polymerase (Promega, Madison, Wis.). Each of the 30 PCR cycles consists of denaturation at 94.degree. C. for 1 min, annealing at 54.degree. C. for 1 min, and extension at 72.degree. C. for 1 min. The founder mice are then mated with C57B1 partners to generate transgenic F.sub.1 lines of mice.

Screening Assays for Compounds or Substances that Modulate Osteomimicry

Compounds or substances that interfere with the abnormal function and/or growth of tumor and tissue cells with calcification potential can provide therapies targeting defects in osteotropic-related disorders including, but not limited to, localized or disseminated osteosarcoma, lung, renal, colon, melanoma, thyroid, brain, multiple myeloma, breast and prostate cancers, and benign conditions, such as benign prostatic hyperplasia (BPH) or arterial sclerotic conditions where calcification occurs. Such compounds may be used to interfere with the onset or the progression of osteotropic-related disorders. Compounds or substances that stimulate or inhibit promoter activity also may be used to ameliorate symptoms of osteotropic-related disorders.

Genetically engineered cells, cell lines and/or transgenic animals containing a osteomimicry regulatory region and transcriptionally active fragments thereof, operably linked to a reporter gene, can be used as systems for the screening of agents that modulate osteomimicry regulatory region and transcriptionally active fragments thereof activity. Such transgenic mice provide an experimental model in vivo (or can be used as a source of primary cells or cell lines for use in vitro) which can be used to develop new methods of treating osteotropic-related disorders by targeting therapeutic agents to cause arrest in the progression of such disorders.

The present invention encompasses screening assays designed to identify compounds or substances that modulate activity of the osteomimicry regulatory region and transcriptionally active fragments thereof. The present invention encompasses in vitro and cell-based assays, as well as in vivo assays in transgenic animals. As described hereinbelow, compounds to be tested may include, but are not limited to, oligonucleotides, peptides, proteins, small organic or inorganic compounds, antibodies, etc.

Examples of compounds may include, but are not limited to, peptides, such as, for example, soluble peptides, including, but not limited to, Ig-tailed fusion peptides, and members of random peptide libraries; (see, e.g., Lam, et al, 1991, Nature 354:82-84; Houghten, et al., 1991, Nature 354:84-86), and combinatorial chemistry-derived molecular library made of D- and/or L-configuration amino acids, phosphopeptides (including, but not limited to members of random or partially degenerate, directed phosphopeptide libraries; see, e.g., Songyang, et al., 1993, Cell 72:767-778), antibodies (including, but not limited to, polyclonal, monoclonal, humanized, anti-idiotypic, chimeric or single chain antibodies, and FAb, F(ab′).sub.2 and FAb expression library fragments, and epitope-binding fragments thereof), and small organic or inorganic molecules.

Such compounds may further comprise compounds, in particular drugs or members of classes or families of drugs, known to ameliorate the symptoms of an osteotropic-related disorder.

Such compounds include, but are not limited to, families of antidepressants such as lithium salts, carbamazepine, valproic acid, lysergic acid diethylamide (LSD), pchlorophenylalanine, p-propyldopacetamide dithiocarbamate derivatives e.g., FLA 63; antianxiety drugs, e.g., diazepam; monoamine oxidase (MAO) inhibitors, e.g., iproniazid, clorgyline, phenelzine and isocarboxazid; biogenic amine uptake blockers, e.g., tricyclic antidepressants such as desipramine, imipramine and amitriptyline; serotonin reuptake inhibitors e.g., fluoxetine; antipsychotic drugs such as phenothiazine derivatives (e.g., chlorpromazine (thorazine) and trifluopromazine)), butyrophenones (e.g., haloperidol (Haldol)), thioxanthene derivatives (e.g., chlorprothixene), and dibenzodiazepines (e.g., clozapine); benzodiazepines; dopaminergic agonists and antagonists e.g., L-DOPA, cocaine, amphetamine, .alpha.-methyl-tyrosine, reserpine, tetrabenazine, benzotropine, pargyline; noradrenergic agonists and antagonists e.g., clonidine, phenoxybenzamine, phentolamine, tropolone; nitrovasodilators (e.g., nitroglycerine, nitroprusside as well as NO synthase enzymes); and antagosists of growth factors (e.g., VEGF, FGF, angiopoetins and endostatin), androgen receptor antagonists, GPCR antagonists, PKA/CREB signal activation interrupters, b2m/PKA/CREB signaling interupters, CREB transcription factor and complex formation signal activation interrupters, or any combination thereof.

In one preferred embodiment, genetically engineered cells, cell lines or primary cultures of germ and/or somatic cells containing a mammalian osteomimicry regulatory region and transcriptionally active fragments thereof operatively linked to a heterologous gene are used to develop assay systems to screen for compounds which can inhibit sequence-specific DNA-protein interactions. Such methods comprise contacting a compound or substance to a cell that expresses a gene under the control of a osteomimicry regulatory region and transcriptionally active fragments thereof, measuring the level of the gene expression or gene product activity and comparing this level to the level of gene expression or gene product activity produced by the cell in the absence of the compound or substance, such that if the level obtained in the presence of the compound or substance differs from that obtained in its absence, a compound capable of modulating the expression of the mammalian osteomimicry regulatory region and transcriptionally active fragments thereof has been identified. Alterations in gene expression levels may be by any number of methods known to those of skill in the art e.g., by assaying for reporter gene activity, assaying cell lysates for mRNA transcripts, e.g. by Northern analysis or using other methods known in the art for assaying for gene products expressed by the cell.

In another embodiment, microdissection and transillumination can be used. These techniques offer a rapid assay for monitoring effects of putative drugs on osteotropic cells in transgenic animals containing a osteomimicry regulatory region and transcriptionally active fragments thereof-driven reporter gene. In this embodiment, a test agent is delivered to the transgenic animal by any of a variety of methods. Methods of introducing a test agent may include oral, intradermal, intramuscular, intraperitoneal, intravenous, subcutaneous, intranasal and via scarification (scratching through the top layers of skin, e.g., using a bifurcated needle) or any other standard routes of drug delivery. The effect of such test compounds on the osteotropic cells can be analyzed by the microdissection and transillumination of the osteoblastic cells. If the level of reporter gene expression observed or measured in the presence of the compound differs from that obtained in its absence, a compound capable of modulating the expression of the mammalian osteomimicry regulatory region and transcriptionally active fragments thereof has been identified.

In various embodiments of the invention, compounds that may be used in screens for modulators of osteotropic-related disorders include peptides, small molecules, both naturally occurring and/or synthetic (e.g., libraries of small molecules or peptides), cell-bound or soluble molecules, organic, non-protein molecules and recombinant molecules that may have osteomimicry regulatory region and transcriptionally active fragments thereof binding and/or interfering capacity and, therefore, may be candidates for pharmaceutical agents.

Alternatively, the proteins and compounds include endogenous cellular components which interact with osteomimicry regulatory region and transcriptionally active fragments thereof sequences in vivo. Cell lysates or tissue homogenates may be screened for proteins or other compounds which bind to the osteomimicry regulatory region and transcriptionally active fragments thereof. Such endogenous components may provide new targets for pharmaceutical and therapeutic interventions.

In one embodiment, libraries can be screened. Many libraries are known in the art that can be used, e.g., peptide libraries, chemically synthesized libraries, recombinant (e.g., phage display libraries), and in vitro translation-based libraries. In one embodiment of the present invention, peptide libraries may be used to screen for agonists or antagonists of osteomimicry regulatory region and transcriptionally active fragments thereof-linked reporter expression. Diversity libraries, such as random or combinatorial peptide or non-peptide libraries can be screened for molecules that specifically modulate osteomimicry regulatory region and transcriptionally active fragments thereof activity. Random peptide libraries consisting of all possible combinations of amino acids attached to a solid phase support may be used to identify peptides that are able to activate or inhibit osteomimicry regulatory region and transcriptionally active fragments thereof activities (Lam, K. S. et al., 1991, Nature 354:82-84). The screening of peptide libraries may have therapeutic value in the discovery of pharmaceutical agents that stimulate or inhibit the expression of osteomimicry regulatory region and transcriptionally active fragments thereof.

Examples of chemically synthesized libraries are described in Fodor et al., 1991, Science 251:767-773; Houghten et al., 1991, Nature 354:84-86; Lam et al., 1991, Nature 354:82-84; Medynski, 1994, BioTechnology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al, 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian, et al., 1992, J. Mol. Biol. 227:711-718; Lenstra, 1992, J. hnmunol. Meth. 152:149-157; Kay et al., 1993, Gene 128:59-65; and PCT Publication No. WO 94/18318 dated Aug. 18, 1994.

By way of example of non-peptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) also can be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

A specific embodiment of such an in vitro screening assay is described below. The osteomimicry regulatory region and transcriptionally active fragments thereof-reporter vector is used to generate transgenic mice from which primary cultures of osteomimicry regulatory region and transcriptionally active fragments thereof-reporter vector germ cells are established. About 10,000 cells per well are plated in 96-well plates in total volume of 100 .mu.l, using medium appropriate for the cell line. Candidate inhibitors of the osteomimicry regulatory region and transcriptionally active fragments thereof are added to the cells. The effect of the inhibitors of the osteomimicry regulatory region and transcriptionally active fragments thereof can be determined by measuring the response of the reporter gene driven by the osteomimicry regulatory region and transcriptionally active fragments thereof. This assay could easily be set up in a high-throughput screening mode for evaluation of compound libraries in a 96-well format that reduce (or increase) reporter gene activity, but which are not cytotoxic. After 6 hours of incubation, 100 .mu.l DMEM medium +2.5% fetal bovine serum (FBS) to 1.25% final serum concentration is added to the cells, which are incubated for a total of 24 hours (18 hours more). At 24 hours, the plates are washed with PBS, blot dried, and frozen at −80.degree. C. The plates are thawed the next day and analyzed for the presence of reporter activity.

In a preferred example of an in vivo screening assay, tumor or tissue cells with calcification potential derived from transgenic mice can be transplanted into mice with a normal or other desired phenotype (Brinster et al., 1994, Proc. Natl. Acad. Sci. USA 91:11298-302; Ogawa et al., 1997, Int. J. Dev. Biol. 41:111-12). Such mice can then be used to test the effect of compounds and other various factors on osteotropic-related disorders. In addition to the compounds and agents listed above, such mice can be used to assay factors or conditions that can be difficult to test using other methods, such as dietary effects, internal pH, temperature, etc.

Once a compound has been identified that inhibits or enhances osteomimicry regulatory region and transcriptionally active fragments thereof activity, it may then be tested in an animal-based assay to determine if the compound exhibits the ability to act as a drug to ameliorate and/or prevent symptoms of an osteotropic-related disorder, including, but not limited to, localized or disseminated osteosarcoma, lung, renal, colon, melanoma, thyroid, brain, multiple myeloma, breast and prostate cancers, and benign conditions, such as benign prostatic hyperplasia (BPH) or arterial sclerotic conditions where calcification occurs.

The assays of the present invention may be first optimized on a small scale (i.e., in test tubes), and then scaled up for high-throughput assays. The screening assays of the present invention may be performed in vitro, i.e., in test tubes, using purified components or cell lysates. The screening assays of the present invention may also be carried out in intact cells in culture and in animal models. In accordance with the present invention, test compounds which are shown to modulate the activity of the osteomimicry regulatory region and transcriptionally active fragments thereof in vitro, as described herein, will further be assayed in vivo in cultured cells and animal models to determine if the test compound has the similar effects in vivo and to determine the effects of the test compound on osteotropic-related disorders.

Osteomimicry Modulatory Antisense, Ribozyme and Triple Helix Approaches

In another embodiment, the types of conditions, disorders, or diseases involving tumor and tissue cells with calcification potential which may be prevented, delayed, or rescued by modulating osteotropic-specifc gene expression by using a osteomimicry regulatory region and/or transcriptionally active fragments thereof in conjunction with well-known antisense, gene “knock-out,” ribozyme and/or triple helix methods, are described. Such molecules may be designed to modulate, reduce or inhibit either unimpaired, or if appropriate, mutant osteotropic gene activity. Techniques for the production and use of such molecules are well known to those of skill in the art.

Antisense RNA and DNA molecules act to directly block the translation of mRNA by hybridizing to targeted mRNA and preventing protein translation. Antisense approaches involve the design of oligonucleotides which are complementary to an mRNA sequence. The antisense oligonucleotides will bind to the complementary mRNA sequence transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of the sequence of interest could be used in an antisense approach to inhibit translation of endogenous mRNA. Antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides or at least 50 nucleotides.

Regardless of the choice of target sequence, it is preferred that in vitro studies are first performed to quantitate the ability of the antisense oligonucleotide to inhibit sequence expression. It is preferred that these studies utilize controls that distinguish between antisense gene inhibition and nonspecific biological effects of oligonucleotides. Additionally, it is envisioned that results obtained using the antisense oligonucleotide are compared with those obtained using a control oligonucleotide. It is preferred that the control oligonucleotide is of approximately the same length as the test oligonucleotide and that the nucleic acid of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger, et al., 1989, Proc. Natl. Acad. Sci. U.S.A. 86:6553-6556; Lemaitre, et al., 1987, Proc. Natl. Acad. Sci. U.S.A. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6:958-976) or intercalating agents (see, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosin-e, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an .alpha.-anomeric oligonucleotide. An .alpha.-anomeric oligonucleotide forms specific double-stranded hybrids with complementary RNA in which, contrary to the usual .beta.-units, the strands run parallel to each other (Gautier, et al., 1987, Nucl. Acids Res. 15:6625-6641). The oligonucleotide is a 2′-0-methylribonucleotide (Inoue, et al., 1987, Nucl. Acids Res. 15:6131-6148), or a chimeric RNA-DNA analogue (Inoue, et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein, et al. (1988, NucL Acids Res. 16:3209), methylphosphonate oligonucleotides can be prepared by use of controlled pore glass polymer supports (Sarin, et al., 1988, Proc. Natl. Acad. Sci. U.S.A. 85:7448-7451), etc.

While antisense nucleotides complementary to an osteotropic-specific coding region sequence could be used, those complementary to the transcribed, untranslated region (for example, osteomimicry regulatory region and/or transcriptionally active fragments thereof) are most preferred.

Antisense molecules should be delivered to cells that express the osteotropic sequence in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies which specifically bind receptors or antigens expressed on the target cell surface) can be administered systemically.

A preferred approach to achieve intracellular concentrations of the antisense sufficient to suppress translation of endogenous mRNAs utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs which will form complementary base pairs with the endogenous sequence transcripts and thereby prevent translation of the mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290:304-310), the promoter contained in the 3′-long terminal repeat of Rous sarcoma virus (Yamamoto, et al., 1980, Cell 22:787-797), the herpes thymidine kinase promoter (Wagner, et al., 1981, Proc. Natl. Acad. Sci. U.S.A. 78:1441-1445), the regulatory sequences of the metallothionein gene (Brinster, et al., 1982, Nature 296:39-42), etc. Any type of plasmid, cosmid, YAC or viral vector can be used to prepare the recombinant DNA construct that can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver, et al, 1990, Science 247, 1222-1225).

Ribozymes are enzymatic RNA molecules capable of catalyzing the specific cleavage of RNA. (For a review, see Rossi, 1994, Current Biology 4:469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

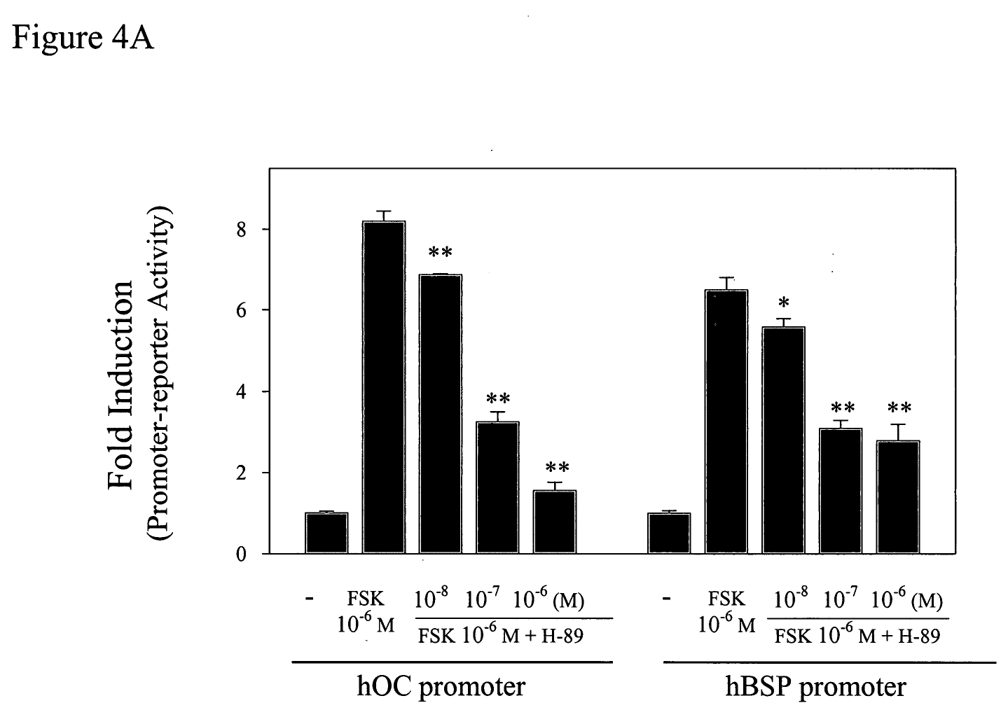

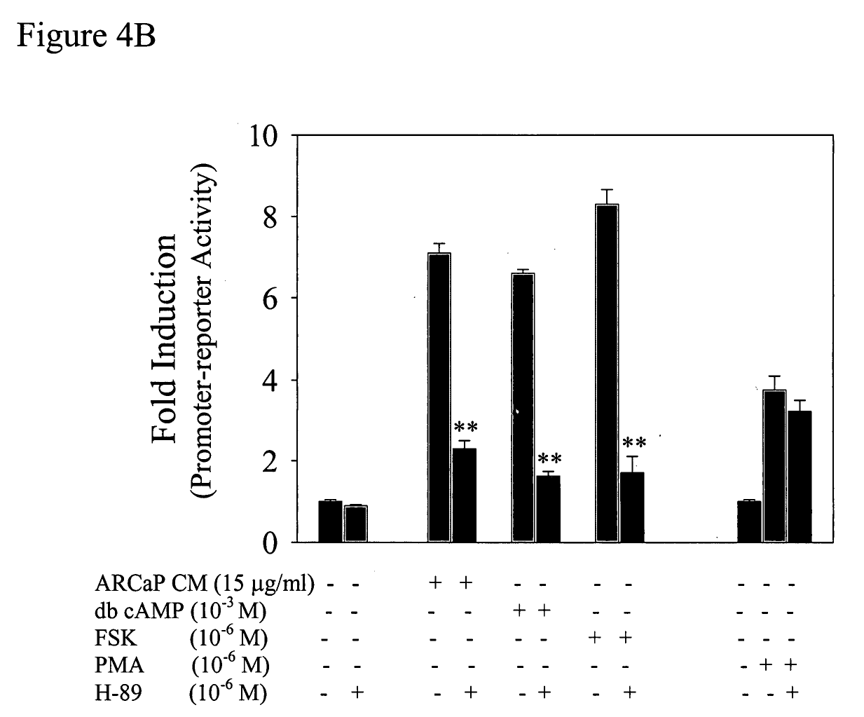

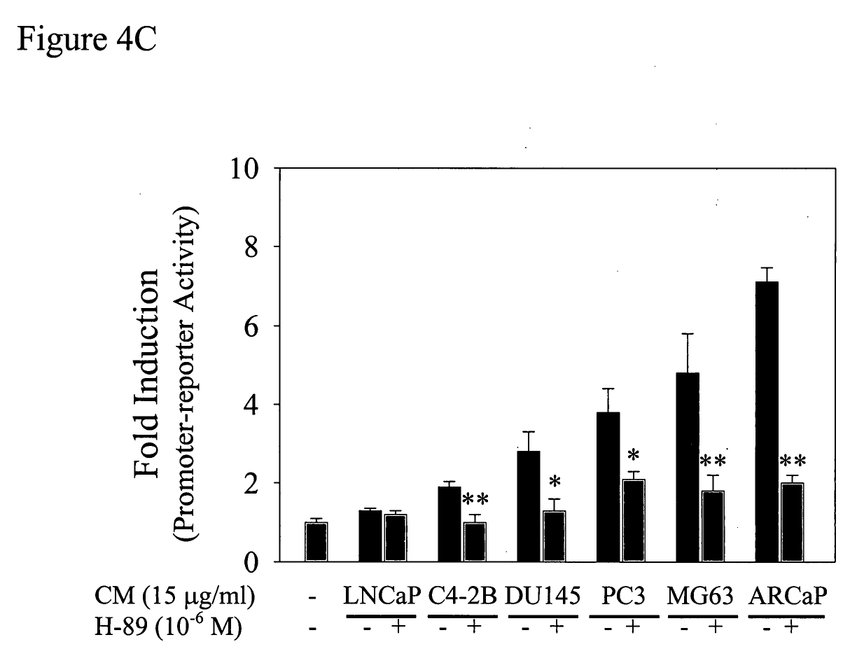

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions which form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, page 833) and in Haseloff and Gerlach, 1988, Nature, 334:585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470-475; Zaug, et al., 1986, Nature, 324:429-433; published International patent application No. WO 88/04300 by University Patents Inc.; Been and Cech, 1986, Cell, 47:207-216). The Cech-type ribozymes have an eight base pair active site that hybridizes to a target RNA sequence whereafter cleavage of the target RNA takes place. The invention encompasses those Cech-type ribozymes that target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies, et al, 1985, Nature 317:230-234; Thomas and Capecchi, 1987, Cell 51:503-512; Thompson, et al., 1989, Cell 5:313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells which express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures which prevent transcription of the target gene in target cells in the body. (See generally, Helene, 1991, Anticancer Drug Des., 6(6):569-584; Helene, et al., 1992, Ann. N.Y. Acad. Sci., 660:27-36; and Maher, 1992, Bioassays 14(12):807-815).

Nucleic acid molecules to be used in triple helix formation for the inhibition of transcription should be single stranded and composed of deoxynucleotides. The base composition of these oligonucleotides must be designed to promote triple helix formation via Hoogsteen base pairing rules, which generally require sizable stretches of either purines or pyrimidines to be present on one strand of a duplex. Nucleic acids may be pyrimidine-based, which will result in TAT and CGC+triplets across the three associated strands of the resulting triple helix. The pyrimidine-rich molecules provide base complementarity to a purine-rich region of a single strand of the duplex in a parallel orientation to that strand. In addition, nucleic acid molecules may be chosen which are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles which the possibility may arise wherein the concentration of normal target gene product present may be lower than is necessary for a normal phenotype. In such cases, to ensure that substantially normal levels of target gene activity are maintained, therefore, nucleic acid molecules which encode and express target gene polypeptides exhibiting normal target gene activity may be introduced into cells via gene therapy methods such as those described, below, which do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid-phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Gene Replacement Therapy

The nucleic acid sequences of the invention, described above, can be utilized for transferring recombinant nucleic acid sequences to cells and expressing said sequences in recipient cells. Such techniques can be used, for example, in marking cells or for the treatment of a disorder involving tumor or tissue cells with calcification potential. Such treatment can be in the form of gene replacement therapy. Specifically, one or more copies of a normal gene or a portion of the gene that directs the production of a gene product exhibiting normal gene function, may be inserted into the appropriate cells within a patient, using vectors that include, but are not limited to adenovirus, adeno-associated virus and retrovirus vectors, in addition to other particles that introduce DNA into cells, such as liposomes.

Methods for introducing genes for expression in mammalian cells are well known in the field. Generally, for such gene therapy methods, the nucleic acid is directly administered in vivo into a target cell or a transgenic mouse that expresses a osteomimetic-cancer specific regulatory region operably linked to a heterologous coding sequence. This can be accomplished by any method known in the art, e.g., by constructing it as part of an appropriate nucleic acid expression vector and administering it so that it becomes intracellular, e.g., by infection using a defective or attenuated retroviral or other viral vector (see U.S. Pat. No. 4,980,286), by direct injection of naked DNA, by use of microparticle bombardment (e.g., a gene gun; Biolistic, Dupont), by coating with lipids or cell-surface receptors or transfecting agents, by encapsulation in liposomes, microparticles, or microcapsules, by administering it in linkage to a peptide which is known to enter the nucleus or by administering it in linkage to a ligand subject to receptor-mediated endocytosis (see e.g., Wu and Wu, 1987, J. Biol. Chem. 262:4429-4432), which can be used to target cell types specifically expressing the receptors. In another embodiment, a nucleic acid-ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180 dated Apr. 16, 1992; WO 92/22635 dated Dec. 23, 1992; WO92/20316 dated Nov. 26, 1992; WO93/14188 dated Jul. 22, 1993; WO 93/20221 dated Oct. 14, 1993). Alternatively, the nucleic acid can be introduced intracellularly and incorporated within host cell DNA for expression, by homologous recombination (Koller and Smithies, 1989, Proc. Natl. Acad. Sci. USA 86:8932-8935; Zijlstra et al., 1989, Nature 342:435-438).

In one embodiment, techniques for delivery involve direct administration, e.g., by stereotactic delivery of such gene sequences to the site of the cells in which the gene sequences are to be expressed.

Additional methods that may be utilized to increase the overall level of gene expression and/or gene product activity include using targeted homologous recombination methods, as discussed above, to modify the expression characteristics of an endogenous gene in a cell or microorganism by inserting a heterologous DNA regulatory element such that the inserted regulatory element is operatively linked with the endogenous gene in question. Targeted homologous recombination can thus be used to activate transcription of an endogenous gene that is “transcriptionally silent”, i.e., is not normally expressed or is normally expressed at very low levels, or to enhance the expression of an endogenous gene that is normally expressed.

Further, the overall level of target gene expression and/or gene product activity may be increased by the introduction of appropriate target gene-expressing cells, preferably autologous cells, into a patient at positions and in numbers that are sufficient to ameliorate the symptoms of an osteotropic-related disorder. Such cells may be either recombinant or non-recombinant.

When the cells to be administered are non-autologous cells, they can be administered using well known techniques that prevent a host immune response against the introduced cells from developing. For example, the cells may be introduced in an encapsulated form which, while allowing for an exchange of components with the immediate extracellular environment, does not allow the introduced cells to be recognized by the host immune system.

Additionally, compounds or substances, such as those identified via techniques such as those described above that are capable of modulating activity of an osteomimicry regulatory region and transcriptionally active fragments thereof can be administered using standard techniques that are well known to those of skill in the art.

Combination Therapies for Utilization and Targeting of Osteomimicry using the Methods of the Invention

In each of the aforementioned aspects and embodiments of the invention, combination therapies other than those enumerated above are also specifically contemplated herein. In particular, the compositions of the present invention may be admininistered with one or more macrolide or non-macrolide antibiotics, anti-bacterial agents, anti-fuingicides, anti-viral agents, and anti-parasitic agents, anti-inflammatory or immunomodulatory drugs or agents.

Examples of macrolide antibiotics that may be used in combination with the composition of the present invention include, inter alia, the following synthetic, semi-synthetic or naturally occurring microlidic antibiotic compounds: methymycin, neomethymycin, YC-17, litorin, erythromycin A to F, oleandomycin, roxithromycin, dirithromycin, flurithromycin, clarithromycin, davercin, azithromycin, josamycin, kitasamycin, spiramycin, midecamycin, rokitamycin, miokamycin, lankacidin, and the derivatives of these compounds. Thus, erythromycin and compounds derived from erythromycin belong to the general class of antibiotics known as “macrolides.” Examples of preferred erythromycin and erythromycin-like compounds include: erythromycin, clarithromycin, azithromycin, and troleandomycin.

Additional antibiotics, other than the macrolidic antibiotics described above, which are suitable for use in the methods of the present invention include, for example, any molecule that tends to prevent, inhibit or destroy life and as such, and as used herein, includes anti-bacterial agents, anti-fuingicides, anti-viral agents, and anti-parasitic agents. These agents may be isolated from an organism that produces the agent or procured from a commercial source (e.g., pharmaceutical company, such as Eli Lilly, Indianapolis, Ind.; Sigma, St. Louis, Mo.). For example, the anti-TB antibiotic isoniazid (isonicotinic acid hydrazide), rifampin, ethambutol, ethionamide, streptomycin, amikacin, clofazimine, ofloxacin, levofloxacin, troveofloxacin, Pefloxacin, gatifloxacin, and moxifloxacin. Other examples of anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, oxazalidinones, and fluoroquinolones; and their various salts, acids, bases, and other derivatives.

Anti-fungal agents include, but are not limited to, caspofungin, terbinafine hydrochloride, nystatin, amphotericin B, griseofulvin, ketoconazole, miconazole nitrate, flucytosine, fluconazole, itraconazole, clotrimazole, benzoic acid, salicylic acid, and selenium sulfide.

Anti-viral agents include, but are not limited to, valgancyclovir, amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha, and edoxudine.

Anti-parasitic agents include, but are not limited to, pirethrins/piperonyl butoxide, permethrin, iodoquinol, metronidazole, diethylcarbamazine citrate, piperazine, pyrantel pamoate, mebendazole, thiabendazole, praziquantel, albendazole, proguanil, quinidine gluconate injection, quinine sulfate, chloroquine phosphate, mefloquine hydrochloride, primaquine phosphate, atovaquone, co-trimoxazole(sulfamethoxazole/trimethoprim), and pentamidine isethionate.

In another aspect, in each of the aforementioned methods of the present invention, one may, for example, supplement the composition by administration of a therapeutically effective amount of one or more an anti-inflammatory or immunomodulatory drugs or agents. By “immunomodulatory drugs or agents”, it is meant, e.g., agents which act on the immune system, directly or indirectly, e.g., by stimulating or suppressing a cellular activity of a cell in the immune system, e.g., T-cells, B-cells, macrophages, or antigen presenting cells (APC), or by acting upon components outside the immune system which, in turn, stimulate, suppress, or modulate the immune system, e.g., hormones, receptor agonists or antagonists, and neurotransmitters; immunomodulators can be, e.g., immunosuppressants or immunostimulants. By “anti-inflammatory drugs”, it is meant, e.g., agents which treat inflammatory responses, i.e., a tissue reaction to injury, e.g., agents which treat the immune, vascular, or lymphatic systems.

Anti-inflammatory or immunomodulatory drugs or agents suitable for use in this invention include, but are not limited to, interferon derivatives, e.g., betaseron, .beta.-interferon; prostane derivatives, e.g., compounds disclosed in PCT/DE93/0013, e.g., iloprost, cicaprost; glucocorticoid, e.g., cortisol, prednisolone, methylprednisolone, dexamethasone; immunsuppressives, e.g., cyclosporine A, FK-506, methoxsalene, thalidomide, sulfasalazine, azathioprine, methotrexate; lipoxygenase inhibitors, e.g., zileutone, MK-886, WY-50295, SC-45662, SC-41661A, BI-L-357; leukotriene antagonists, e.g., compounds disclosed in DE 40091171 German patent application P 42 42 390.2; WO 9201675; SC-41930; SC-50605; SC-51146; LY 255283 (D. K. Herron et al., FASEB J. 2: Abstr. 4729, 1988); LY 223982 (D. M. Gapinski et al. J. Med. Chem. 33: 2798-2813, 1990); U-75302 and analogs, e.g., described by J. Morris et al., Tetrahedron Lett. 29: 143-146, 1988, C. E. Burgos et al., Tetrahedron Lett. 30: 5081-5084, 1989; B. M. Taylor et al., Prostaglandins 42: 211-224, 1991; compounds disclosed in U.S. Pat. No. 5,019,573; ONO-LB-457 and analogs, e.g., described by K. Kishikawa et al., Adv. Prostagl. Thombox. Leukotriene Res. 21: 407-410, 1990; M. Konno et al., Adv. Prostagl. Thrombox. Leukotriene Res. 21: 411-414, 1990; WF-11605 and analogs, e.g., disclosed in U.S. Pat. No. 4,963,583; compounds disclosed in WO 9118601, WO 9118879; WO 9118880, WO 9118883, antiinflammatory substances, e.g., NPC 16570, NPC 17923 described by L. Noronha-Blab. et al., Gastroenterology 102 (Suppl.): A 672, 1992; NPC 15669 and analogs described by R. M. Burch et al., Proc. Nat. Acad. Sci. USA 88: 355-359, 1991; S. Pou et al., Biochem. Pharmacol. 45: 2123-2127, 1993; peptide derivatives, e.g., ACTH and analogs; soluble TNF-receptors; TNF-antibodies; soluble receptors of interleukines, other cytokines, T-cell-proteins; antibodies against receptors of interleukins, other cytokines, and T-cell-proteins.

The therapeutic agents of the instant invention may be used for the treatment of animal subjects or patients, and more preferably, mammals, including humans, as well as mammals such as non-human primates, dogs, cats, horses, cows, pigs, guinea pigs, and rodent

Pharmaceutical Preparations and Methods of Administration

The compounds or substances that are determined to modulate osteomimicry regulatory region and transcriptionally active fragments thereof activity or osteomimicry gene product activity can be administered to a patient at therapeutically effective doses to treat or ameliorate a disorder involving tumor or tissue cells with calcification potential. A therapeutically effective dose refers to that amount of the compound sufficient to result in amelioration of symptoms of such a disorder.

Effective Dose

Toxicity and therapeutic efficacy of such compounds can be determined by standard pharmaceutical procedures in cell cultures or experimental animals, e.g., for determining the LD.sub.50 (the dose lethal to 50% of the population) and the ED.sub.50 (the dose therapeutically effective in 50% of the population). The dose ratio between toxic and therapeutic effects is the therapeutic index and it can be expressed as the ratio LD.sub.50/ED.sub.50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED.sub.50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC.sub.50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In certain embodiments, it may be desirable to administer the pharmaceutical compositions of the invention locally to the area in need of treatment. This may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of a malignant tumor or neoplastic or pre-neoplastic tissue.

For topical application, the compounds may be combined with a carrier so that an effective dosage is delivered, based on the desired activity.

In addition to the formulations described previously, the compounds also may be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### EXAMPLES

The following specific examples are provided to better assist the reader in the various aspects of practicing the present invention. As these specific examples are merely illustrative, nothing in the following descriptions should be construed as limiting the invention in any way. Such limitations are, or course, defined solely by the accompanying claims.

### EXAMPLE 1

### Identification and Characterization of CRE Cis-Elements within Human Osteocalcin and Bone Sialoprotein Promoters Mediating Osteomimicry of Prostate Cancer Cells: Role of cAMP-Dependent PKA Signaling Pathway

Abstract

Osteocalcin (OC) and bone sialoprotein (BSP) are the most abundant noncollagenous bone matrix proteins expressed by osteoblasts. Surprisingly, OC and BSP are also expressed by malignant but not normal prostate epithelial cells. The purpose of this study is to investigate how OC and BSP expression are regulated in prostate cancer cells. Our investigation revealed: 1) Human OC (hOC) and human BSP (hBSP) promoter activities in an androgen-independent prostate cancer cell line of LNCaP lineage, C4-2B, were markedly enhanced 7 to 12-fold in a concentration-dependent manner by conditioned media (CM) collected from prostate cancer and bone stromal cells. 2) Deletion analysis of hOC and hBSP promoter regions identified cAMP-responsive elements (CREs) as the critical determinants for CM-mediated OC and BSP gene expression in prostate cancer cells. Consistent with these results, the PKA pathway activators forskolin (FSK) and dibutyryl cAMP (db cAMP), and the PKA pathway inhibitor H-89, respectively increased or repressed hOC and hBSP promoter activities. 3) Electrophoretic mobility shift assay (EMSA) showed that CM-mediated stimulation of hOC and hBSP promoter activities occurs through increased interaction between CRE and CRE-binding protein (CREB). 4) CM was found to induce hOC and hBSP promoter activities via increased CRE-CREB interaction in a cell-background-dependent manner, with marked stimulation in selected prostate cancer but not bone stromal cells. Collectively, these results suggest that OC and BSP expression are coordinated and regulated through cAMP-dependent PKA signaling, which may define the molecular basis of the osteomimicry exhibited by prostate cancer cells.

Introduction

The progression of prostate cancer to androgen independence and bone metastasis is generally lethal. Death often results from bone and visceral organ metastasis (1). Despite the prevalence of prostate cancer metastasis to the skeleton, the molecular mechanisms of bone tropism are poorly understood. Previous studies suggest that prostate cancer cell adhesion, extravasation, migration and interaction with bone cells are critical determinants that govern prostate cancer bone colonization (2-4). Reports using clinical prostate cancer metastasis specimens (5-7) and experimental cell and animal models (8-10) found the bone-specific proteins osteocalcin (OC) and bone sialoprotein (BSP) to be expressed in a heterogeneous manner by human prostate cancer specimens. We proposed that prostate cancer cells acquire osteomimetic or bone-like properties to improve their adhesion, proliferation and survival in bone (11). This communication delineates the molecular mechanisms underlying the induction of human OC (hOC) and human BSP (hBSP) promoter activities and their endogenous gene expression in the bone microenvironment by factors secreted from prostate cancer and bone stromal cells.

OC (5-6 kDa) and BSP (72-80 kDa) are synthesized and secreted by normal maturing osteoblasts. They are major noncollagenous bone matrix proteins, with OC comprising 1-2% of the total proteins in the skeleton (12). OC binds with high affinity to hydroxyapatite crystals, the key mineral component of bone, and regulates bone crystal growth (13). OC can also act as a chemoattractant in the recruitment of osteoblasts and osteoclasts, contributing to the dynamics of new bone formation and bone resorption (14). Ducy and colleagues (15) reported that OC-null mice exhibit increased bone formation without impaired bone resorption, suggesting a more complex interaction between recruited osteoblasts and osteoclasts and the participation of BSP in this process. BSP is a highly sulfated, phosphorylated and glycosylated protein that mediates cell attachment through a RGD motif to extracellular matrices (16). Due to its highly negatively charged characteristics, BSP can sequester calcium ions while conserving polyglutamate regions which have hydroxyapatite crystal nucleation potential (17). Through the RGD motif, BSP mediates the attachment and activation of osteoclasts (18) and can facilitate attachment of normal bone or cancer cells to mineralized tissue surfaces (19, 20). Through its binding to Factor H, BSP can protect cells from complement-mediated cell lysis, which may be important for cancer cell survival (21). In the absence of OC, BSP could contribute to an overall metabolic shift toward new bone formation (22-24). The published data suggest OC and BSP could complement each other by regulating the homeostasis of bone formation and bone resorption via controlled osteosclerotic and osteoclastogenic reactions.

The coupling of G-proteins with a cAMP-dependent PKA signaling pathway is known to be triggered by a large number of ligand and receptor systems (25). Activation of this signaling pathway is associated with the control of cell growth and differentiation (26, 27), ion channel conductivity (28) and gene transcription (29-31). The cAMP-responsive element (CRE), a cis-acting element, interacts with a basic domain/leucine zipper motif contained within a transcription factor termed CRE-binding protein (CREB), through the cAMP-dependent PKA pathway (32). Studies in which both OC (30) and BSP (33, 34) promoter activities were stimulated by the cAMP-dependent PKA pathway suggest the presence of the putative CRE site within these promoter regions that may interact with CREB to initiate OC and BSP expression.

In this communication, we demonstrate that conditioned media (CM) collected from various human prostate cancer and bone stromal cell lines induce hOC and hBSP promoter activities and increase their steady-state levels of endogenous mRNA through a cAMP-dependent PKA pathway in human prostate cancer cells that targets CRE cis-elements within the hOC and hBSP promoter regions.

Materials and Methods

Reagents. Tissue culture medium and fetal bovine serum were obtained from Life Technologies Inc. (Rockville, Md.) and Sigma (St. Louis, Mo.), respectively. Reagents used for the study of cAMP-dependent signaling pathway, forskolin (FSK), dibutyryl 3′,5′-cyclic AMP (db cAMP), phorbol 12-myristate 13-acetate (PMA), and N-[2-(p-bromocinnamylamino)ethyl]-5-isoquinolinesulfonamide (H-89) were purchased from Alexis Biochemicals (San Diego, Calif.). Synthetic oligonucleotides were ordered from Invitrogen (Carlsbad, Calif.). Restriction enzymes, T4 DNA ligase and T4 polynucleotide kinase were obtained from New England BioLabs (Beverly, Mass.). Radioactive nucleotides were purchased from Amersham Biosciences Corp. (Piscataway, N.J.). Taq DNA polymerase was obtained from Roche Molecular Biochemicals (Indianapolis, Ind.). A site-directed mutagenesis kit was obtained from Stratagene (La Jolla, Calif.).

Cells, Cell Culture and Conditioned Media Collection. Human prostate cancer cell lines LNCaP, C4-2B, DU145, PC3 and ARCaP, and human bone stromal cell lines MG63 (a human osteosarcoma cell line) and KeesII (a human normal osteoblast cell line) were cultured in T-medium (Life Technologies Inc.) supplemented with 5% fetal bovine serum and 1% penicillin/streptomycin as previously described (35). The cells were maintained at 37° C. in 5% CO₂. For CM collection, cells were cultured in T-medium with serum until 80% confluent. The cells were washed subsequently twice in PBS and incubated in T-medium without serum. After two days additional incubation, CM were collected and centrifuged, and stored at −20° C. until use. The concentration of total proteins in CM was determined by the Bradford method using Coomassie plus protein reagent (Pierce, Rockford, Ill.).

Plasmid Construction. Genomic DNA was used in the PCR of the 0.8-kb hOC promoter inserting KpnI and XhoI sites, subsequently cloned into pGL3-Basic-luciferase reporter vector (Promega, Madison, Wis.) as described before (9). The deletion constructs, ΔA [374 bp upstream of AP1/VDRE (AV) element], ΔTst-1 (POU-factor Tst-1/Oct-6 binding site), ΔCRE (cAMP-responsive element) and ΔIRF-1 (interferon regulatory factor-i binding site) in hOC promoter/pGL3 were generated by recombinant PCR technique. The point mutation plasmids in CRE (from -643 to -636 in hOC promoter, 5′-TGACCTCA-3′) were constructed with a QuikChange Site-Directed Mutagenesis Kit (Stratagene). The mutation constructs (see below) are one-point substitution mutants, Mut1 (-642 G→T), Mut2 (-641 A→C), Mut3 (-640 C→A), Mut4 (-639 C→A) and Mut5 (-638 T→G), a two-point substitution mutant, Mut6 (-640 and -639 CC→AA), and a two-point deletion construct, Mut7 (-640 and -639 CC→XX). The 1.5-kb hBSP promoter construct prepared in the pGL3-luciferase reporter vector has been described previously (36). The single deletion constructs ΔCRE1 (-79 to -72) and ΔCRE2 (-674 to -667), and a double deletion construct ΔCRE2/CRE1 in the hBSP promoter/pGL3-luciferase plasmid were generated by recombinant PCR technique. All plasmid constructs were confirmed by DNA sequencing.

Transfection and Luciferase Activity Assay. Cells were trypsinized and seeded at a density of 1.5×10⁵ cells/well (LNCaP, C4-2B, DU145 and ARCaP) and 1.0×10⁵ cells/well (PC3 and MG63) in 12-well plates 24 h before transfection. Plasmid DNAs were introduced into cells by complexing with a commercial reagent DOTAP (Roche Molecular Biochemicals) according to the manufacturer's protocol. Each transfection reaction contained 1.25 μg of tested DNA constructs and 0.25 μg of the transfection efficiency control CMV promoter-driven β-galactosidase plasmid DNA. After 6 h transfection, DNA-liposome mixtures were replaced by fresh T-medium or CM. Tansfected cells were harvested and lysed in 1× Reporter Lysis buffer (Promega) after 36 h additional incubation. Cell lysates were vortexed for 15 s and spun for 10 min. For luciferase activity assay, 20 μl of the lysate supernatant was mixed with 100 μl of the luciferase substrate (Promega) and detected by a luminometer (Monolight 3010, luminometer PharMingen, San Diego, Calif.). For β-galactosidase activity assay, 100 μl of the supernatant was mixed with 100 μl of 2×β-galactosidase substrate [200 mM sodium phosphate buffer, pH 7.3, 2 mM MgCl₂, 100 mM β-mercaptoethanol and 1.33 mg/ml o-nitrophenyl-β-D-galactopyranoside (ONPG)], and incubated at 37° C. for 30 min. β-galactosidase activity was detected by Microplate spectrophotometer (Molecular Devices Corp., Sunnyvale, Calif.) at 405 nm wavelength. Data were presented as normalized luciferase activity (means±standard deviations), defined as luciferase activity normalized to internal control β-galactosidase activity for transfection efficiency. All studies were performed in three independent experiments with duplicate assays.

Reverse Transcription-Polymerase Chain Reaction (RT-PCR) Analysis. Total RNA was isolated from confluent monolayers of cells using RNAZol B (Teltest Inc., Friendswood, Tex.). Five μg of total RNA was used as template and 0.15 μg of random hexanucleotide primers were added for reverse transcription and amplification in a reaction volume of 20 μl, according to the manufacturer's instruction (Invitrogen, Carlsbad, Calif.). After RT reaction, 3-5 μl of the 1st-strand cDNA was used for PCR with a PTC-100 programmable Thermal Controller (MJ Research Inc.). The oligonucleotide primer sets used for PCR analysis of cDNA are: hOC, 5′-ACACTCCTCGCCCTATTG-3′ (forward) and 5′-GATGTGGTCAGCCAACTC-3′ (reverse); hBSP, 5′-GCATCGAAGAGTCAAAATAG-3′ (forward) and 5′-TTCTTCTCCATTGTCTTCTC-3′ (reverse); and glyceraldehyde phosphate dehydrogenase (GAPDH), 5′-ACCACAGTCCATGCCATCA-3′ (forward) and 5′-TCCACCACCCTGTTGCTGT-3′ (reverse), respectively. The thermal profile for hOC amplification is 30 cycles starting with denaturation 1 min at 94° C., followed by 1 min of annealing at 55° C. and 1 min of extension at 72° C. For hBSP amplification the thermal profile is 35 cycles starting with denaturation 1 min at 94° C., followed by 30 s of annealing at 48° C. and 40 s of extension at 72° C. The program for GAPDH amplification is 25 cycles starting with denaturation 30 s at 94° C., followed by 30 s of annealing at 60° C. and 1 min of extension at 72° C. The RT-PCR products were analyzed by 1.2% agarose gel electrophoresis. Quantity one-4.1.1 Gel Doc gel documentation software (Bio-Rad) or NIH image were used for quantification of hOC and hBSP mRNA expression normalized by GAPDH mRNA expression.

Immunohistochemical Staining. Human primary and bone metastatic prostate cancer tissue specimens were deparaffinized, treated with 3% H₂O₂, blocked with SuperBlock (Scytek Laboratories, Logan, Utah), and reacted with antibodies against OC (OC 4-30, PanVera Corp. Madison, Wis.) or BSP (kindly provided by Dr. J. Sodek, University of Toronto, Toronto, Canada). The staining signals were amplified by biotinylated peroxidase-conjugated streptavidin system (Bio-Genex Laboratories, San Ramen, Calif.). OC and BSP were visualized in selective areas of the clinical tissue specimens where the immunohistochemical staining was revealed by the conjugated peroxidase reacted with 3-amino-9-ethylcarbazole (AEC) or diaminobenzidine (DAB) as the substrate. Positive OC and BSP are defined as higher than 15% of the cell populations reacted positively with either anti-OC or BSP antibody.

Electrophoretic Mobility Shift Assay (EMSA). A LNCaP-lineaged metastatic human prostate cancer cell line, C4-2B, and the human osteosarcoma cell line MG63 were plated in 15-cm diameter tissue culture dishes in T-medium (with 5% serum) until 80% confluence. Cells were then switched to 1-day complete serum-free condition and then treated with or without ARCaP CM (15 μg/ml) or FSK (10⁻⁵ M) for an additional 16 h. Nuclear extracts were prepared from C4-2B and MG63 for EMSA as described by Ausubel et al (37). Briefly, cells were washed with cold PBS twice, harvested and homogenized in hypotonic buffer (10 mM HEPES, pH 7.9, 1.5 mM MgCl₂, 10 mM KCl, 0.5 mM DTT and 0.2 mM PMSF). After centrifugation, the nuclear pellets were stirred and incubated for 30 min in high-salt buffer (20 mM HEPES, pH 7.9, 25% glycerol, 1.5 mM MgCl₂, 1.2 M KCl, 0.2 mM EDTA, 0.5 mM DTT and 0.2 mM PMSF) on ice. The nuclear pellets were centrifuged, and the nuclear extract supernatants were dialyzed twice against dialysis buffer (20 mM HEPES, pH 7.9, 20% glycerol, 100 mM KCl, 0.2 mM EDTA, 0.5 mM DTT and 0.2 mM PMSF) overnight. The concentration of nuclear proteins was determined by the Bradford method using Coomassie plus protein reagent (Pierce). Synthetic oligonucleotides were purified by PAGE. Appropriate pairs were annealed by heating up to 95° C. for 10 min and naturally cooled down to room temperature. The oligo sequences used as probes or competitors were as follows: wild type CRE, 5′-ACCAACCGGCTGACCTCATCTCCTGCC-3′; Mut6, 5′-ACCAACCGGCTGAAATCATCTCCTGCC-3′. The double-stranded probes were end-labeled with [γ-³²P]ATP (3,000 Ci/mmol at 10 mCi/ml) using T4 polynucleotide kinase. Forty thousand cpm of the labeled probe and 10 μg of nuclear extracts were incubated with binding buffer containing 10 mM Tris-HCl (pH 7.5), 50 mM NaCl, 0.5 mM EDTA, 0.5 mM DTT, 4% glycerol, 1 mM KCl, and 1 μg of poly(dI-dC) (Amersham Pharmacia Biotech.) at 30° C. for 30 min. The samples were subjected to 6% non-denaturing polyacrylamide gel electrophoresis in 1×TGE buffer (25 mM Tris-HCl, pH 8.5, 188 mM glycine and 1 mM EDTA) at 35 mA and 200 V for 2 h at room temperature. In competition experiments, unlabeled competitor-oligos were preincubated with nuclear extracts for 30 min at room temperature before the addition of the probe. For the supershift experiment, 2 μg of anti-CREB antibody (Santa Cruz Biotechnology, Inc., Santa Cruz, Calif.) or anti-AML3/Runx2 antibody (Active Motif, Inc., Carlsbad, Calif.) were added to the nuclear extract reaction mixtures at room temperature for 30 min before the incubation of the probe. After electrophoresis, gels were dried by Gel Dryer (Model 583, Bio-Rad) and exposed to BioMax film (Kodak).

Statistical Analysis. All statistical analyses were performed using Microsoft Excel software. Significant differences were analyzed using Student's t test and two-tailed distribution.

Results

Expression of OC and BSP Proteins by Clinical Prostate Cancer Tissue Specimens, and Conditioned Media (CM) Collected from Human Prostate Cancer and Bone Stromal Cells Stimulated Human OC (hOC) and Human BSP (hBSP) Promoter Activities and The Steady-State Levels of Endogenous OC and BSP mRNA Expression in Human Prostate Cancer Cell Lines.

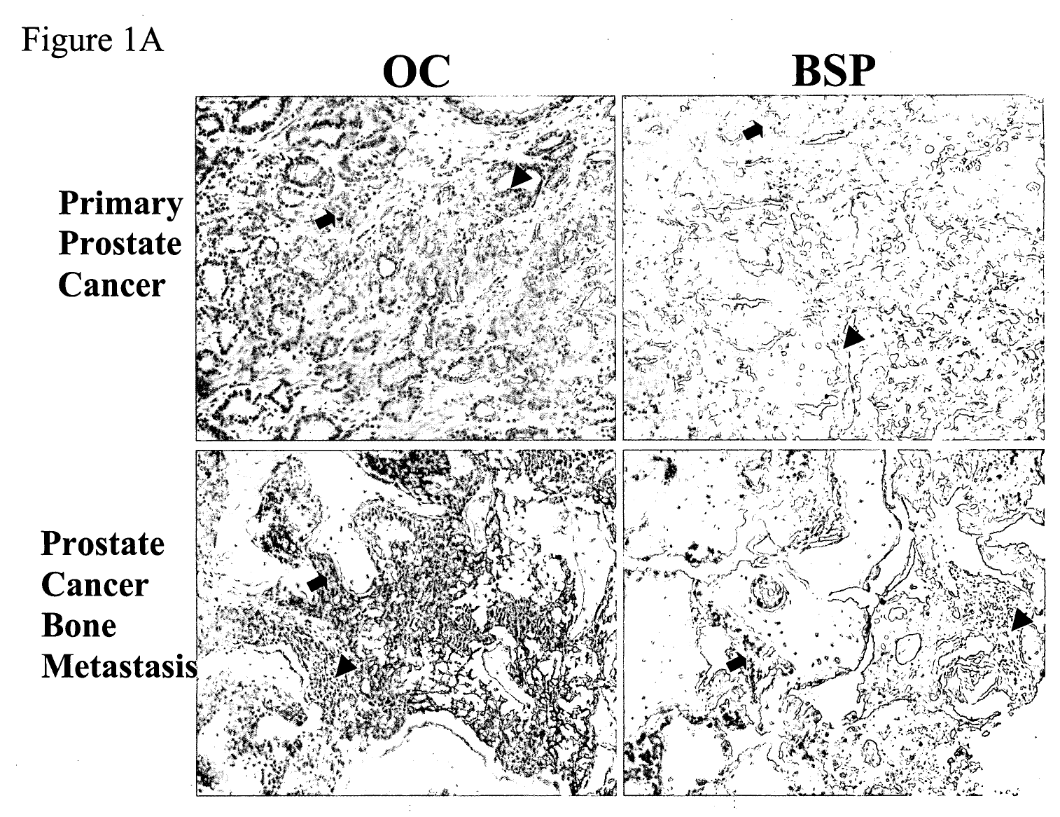

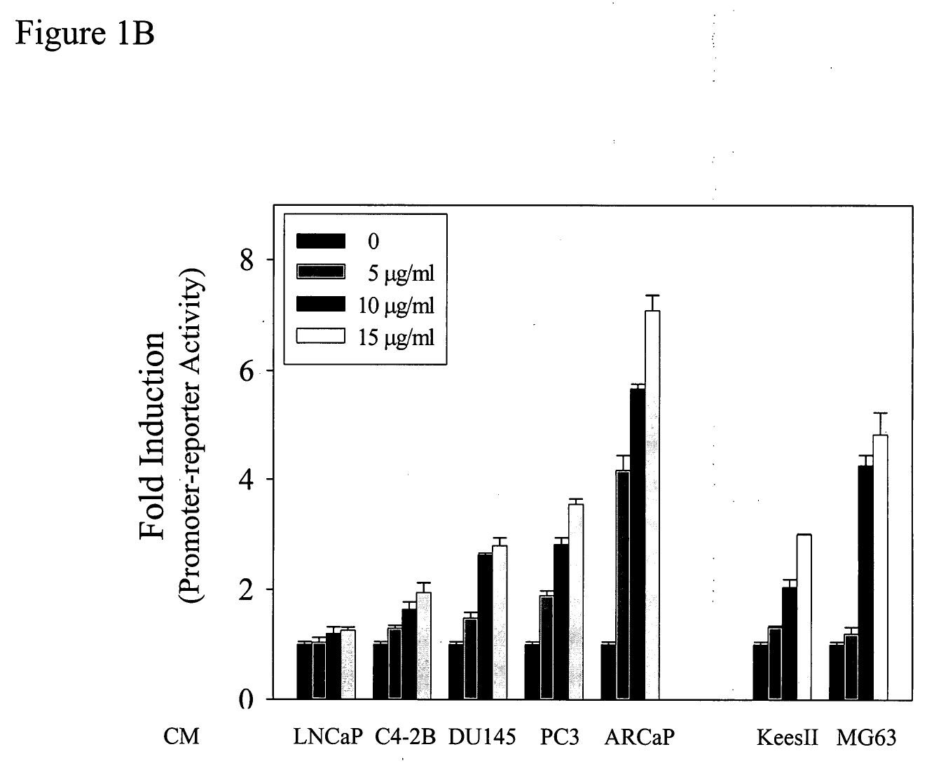

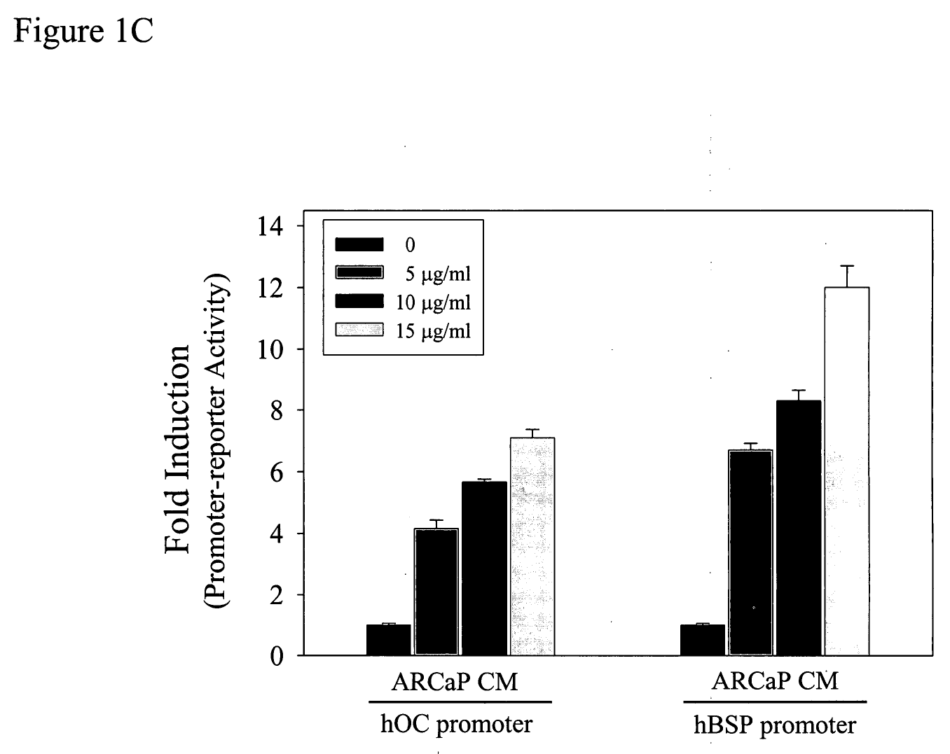

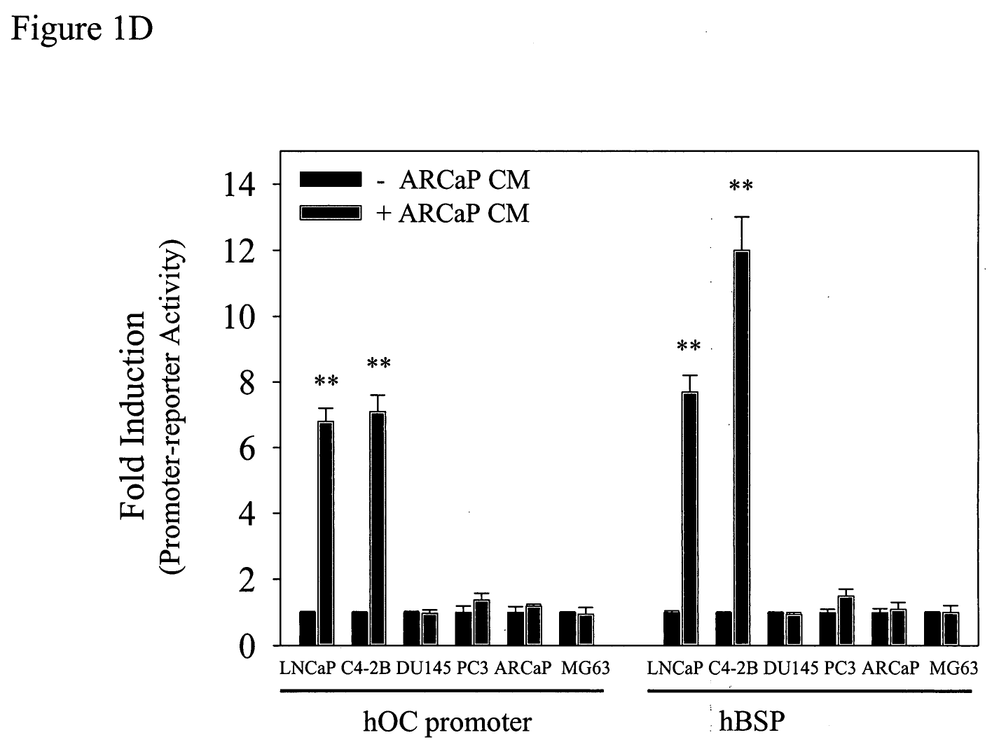

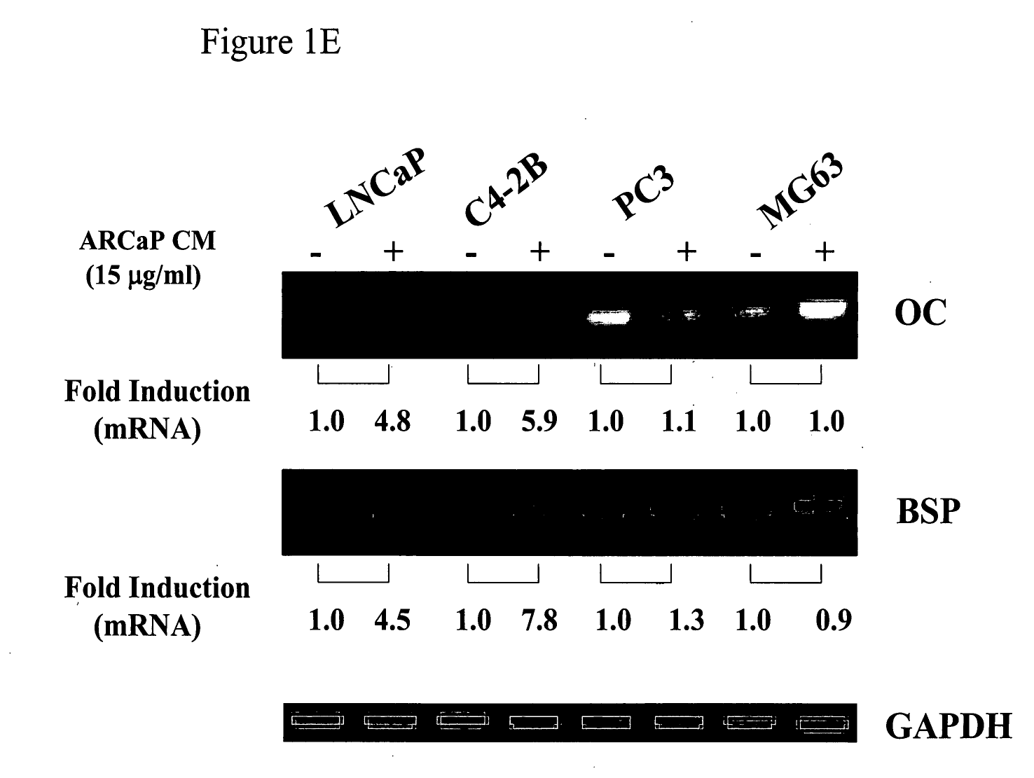

OC protein is prevalently expressed by both primary (85% positively stained) and metastatic (both lymph node and bone were 100% positively stained) human prostate cancer specimens (8). Likewise, BSP protein is also expressed preferentially by malignant (89-100%) primary prostate cancer tissues (7). One common feature of OC and BSP immunostaining in human prostate cancer tissues is the marked heterogeneities among prostate cancer cells in primary and bone metastatic specimens. Some cells stain strongly for OC and BSP proteins (FIG. 1A, bold arrows) and others seem to be lightly stained or not stained at all (FIG. 1A, arrow heads). Differential staining could reflect either intrinsic genetic variations among the prostate cancer cells or be an epiphenomenon of prostate cancer cell interaction with the microenvironment. We sought to evaluate the regulation of OC and BSP expression in human prostate cancer and bone cells to define whether extrinsic factor(s) secreted by prostate cancer or bone cells could mediate OC and BSP promoter activities and their respective steady-state levels of endogenous mRNA expression.

To test the hypothesis that bone-specific protein expression by prostate cancer cells may be induced by factor(s) secreted from prostate cancer and bone stromal cells, we compared the effects of CM harvested from either prostate cancer or bone stromal cells on the expression of OC and BSP in an androgen-independent C4-2B prostate cancer cell line of LNCaP lineage. A luciferase reporter construct with a 0.8-kb hOC promoter or a 1.5-kb hBSP promoter was transfected into C4-2B cells, and the transfected cells were exposed to serum-free CM collected from various human prostate cancer cell lines with a gradient of malignant potentials [from LNCaP (the least malignant), C4-2B, DU145, and PC3 (cells with intermediate levels of aggressiveness) to ARCaP (the most malignant)], a normal non-malignant osteoblast KeesII cell line or a malignant osteosarcoma MG63 cell line. As shown in FIG. 1B, CM stimulated hOC promoter activity in a concentration-dependent manner (from 0 to 15 μg/ml of total proteins in CM). CM from LNCaP cells maximally stimulated hOC promoter activity by only 1.2±0.1-fold, whereas CM collected from the most aggressive ARCaP prostate cancer cell line maximally enhanced the highest hOC promoter activity at 7.1±0.3-fold. CM from other cell lines, C4-2B, DU145, PC3, KeesII and MG63, induced hOC promoter activity at intermediate levels (from 2.0±0.2 to 5.1±0.4-fold). These data suggest that the extent of stimulation of hOC promoter activity by CM correlated positively with the aggressiveness of the prostate cancer. In parallel with the induction of hOC promoter activity, ARCaP CM also up-regulated hBSP promoter activity as much as 12-fold in a concentration-dependent manner in C4-2B cells (FIG. 1C).

To determine further whether ARCaP CM is capable of stimulating hOC and hBSP promoter activities in a series of other human prostate cancer and bone stromal cell lines, we tested these promoter activities in LNCaP, DU145, PC3, ARCaP and MG63 cells. As shown in FIG. 1D, both hOC and hBSP promoter activities were elevated by ARCaP CM in LNCaP and C4-2B, but not in DU145, PC3, ARCaP and MG63 cell lines.

We compared the effect of ARCaP CM in inducing hOC and hBSP promoter-reporter activities in LNCaP and C4-2B cells, and its effects on the steady-state levels of OC and BSP mRNA, in several human prostate cancer cell lines by semi-quantitative RT-PCR. FIG. 1E shows that adding ARCaP CM (15 μg/ml) to LNCaP and C4-2B cells for a 12 h period enhanced the steady-state levels of endogenous OC and BSP mRNA expression by 4.8 and 5.9-fold, and 4.5 and 7.8-fold (GAPDH as an internal control), respectively. In cells that already had high basal levels of OC and BSP mRNA, such as PC3 and MG63 (38) cells, ARCaP CM did not enhance further OC and BSP mRNA expression (0.9 to 1.3-fold induction, respectively). As with the promoter activity assay, the steady-state levels of endogenous OC and BSP mRNA also did not show an increase in DU145 and ARCaP cell lines upon exposure to ARCaP CM (data not shown).

The cAMP-Responsive Element (CRE) is Responsible for Regulation of CM-Mediated hOC and hBSP Promoter Activities.

Our previous data (9) demonstrated that three cis-acting elements are critical for the regulation of hOC promoter activity: OSE1, OSE2, and AP-1/VDRE (AV). To determine whether these elements are important for ARCaP CM-activated hOC promoter activity, we assessed the activities of several hOC promoter deletion constructs, including single, double or triple deletion constructs of OSE1, OSE2 and AV generated by the recombinant PCR method as described previously (9). FIG. 2A shows that among the single deletion constructs, ΔAV did not appear to affect ARCaP CM-induced hOC promoter luciferase activity. In comparison, a slight drop of hOC promoter activity was observed upon the deletion of OSE1 or OSE2. No further decrease in hOC promoter-luciferase activity induced by ARCaP CM was noted by deleting additional cis-elements including the complete deletion of all three critical hOC regulatory elements, ΔAV, ΔOSE2 and ΔOSE1. These data suggest that regions outside of OSE1, OSE2 and AV must be responsible for hOC promoter activation by ARCaP CM.

To address this question, we generated three new deletion constructs with regions outside of the OSE1, OSE2 or AV element systematically deleted. These are designated ΔA (upstream of the AV element, 374 bp, FIG. 2B), ΔB (between AV and OSE2 site, 327 bp) and ΔC (between OSE2 and OSE1 site, 99 bp). As shown in FIG. 2B, a dramatic decrease in hOC promoter activity was observed only when region A was deleted. Minimal loss of ARCaP CM-induced hOC promoter luciferase activity was detected with deletion of region B or C (data not shown).

To find the specific cis-DNA element within region A that may be responsible for ARCaP CM-induced hOC promoter activity, we site-specifically deleted selected regions of A, ΔTst-1 (POU-factor Tst-1/Oct-6, -848 to -834), ΔCRE (cAMP-responsive element, -643 to -636) and ΔIRF-1 (interferon regulatory factor-1, -609 to -597), based on a computer database search, and tested the activity of these constructs in C4-2B cells either exposed to ARCaP CM or control media. FIG. 2B shows that only the ACRE construct exhibited a marked decreased in ARCaP CM-induced hOC promoter luciferase activity, suggesting cAMP may mediate downstream signaling through CRE, regulating ARCaP CM-induced hOC promoter activity.

Then we generated CRE deletion constructs of hBSP promoter. On the basis of computer search, there are two putative CRE sites, CRE1 (-79 to -72) and CRE2 (-674 to -667), within hBSP promoter. As shown in FIG. 2C, hBSP promoter luciferase activity decreased partially in the two single deletion constructs of either CRE1 or CRE2 (designated as ΔCRE1 and ΔCRE2). However, hBSP promoter activation was markedly reduced in the double-deleted ΔCRE2/CRE1 construct when exposed to ARCaP CM. This study demonstrates that CREs are also important for the regulation of hBSP promoter reporter activity enhanced by ARCaP CM.

To delineate the specific nucleotide(s) within the CRE of hOC promoter that may be responsible for ARCaP CM-regulated promoter activity, we introduced either one or two point mutations in CRE, and examined ARCaP CM-induced hOC promoter luciferase activity in C4-2B cells. Only Mut3 (-640 C→A) and Mut4 (-639 C→A) greatly diminished the ARCaP CM-activated hOC promoter activity (FIG. 2D). Other single base mutations, -642 G→T (Mut1), -641 A→C (Mut2) and -638 T→G (Mut5), failed to exert much influence on CM-mediated hOC promoter activity when assayed under the same conditions. Consistent with these results, double-base mutations at -640 and -639 CC→AA (Mut6) and deletion at this same region, CC→XX (Mut7), dramatically reduced ARCaP CM-induced hOC promoter activity. Although we did not perform similar mutational analysis of hBSP promoter, because of the structural identity between CREs among hOC and hBSP promoters (with the exception of a single base difference found in CRE1), it is highly likely that similar mutations in CRE1 and CRE2 will result in disruption of hBSP promoter activity when assayed in prostate cancer cells. Taken together, the results show that two nucleotides, -640 (C) and -639 (C) within the CRE cis-element of hOC promoter, are cooperatively responsible for the ARCaP CM-mediated hOC promoter activation.

The cAMP-Dependent PKA Signaling Pathway is Essential for Mediating ARCaP CM-Activated OC and BSP Gene Expression in Human Prostate Cancer Cells.

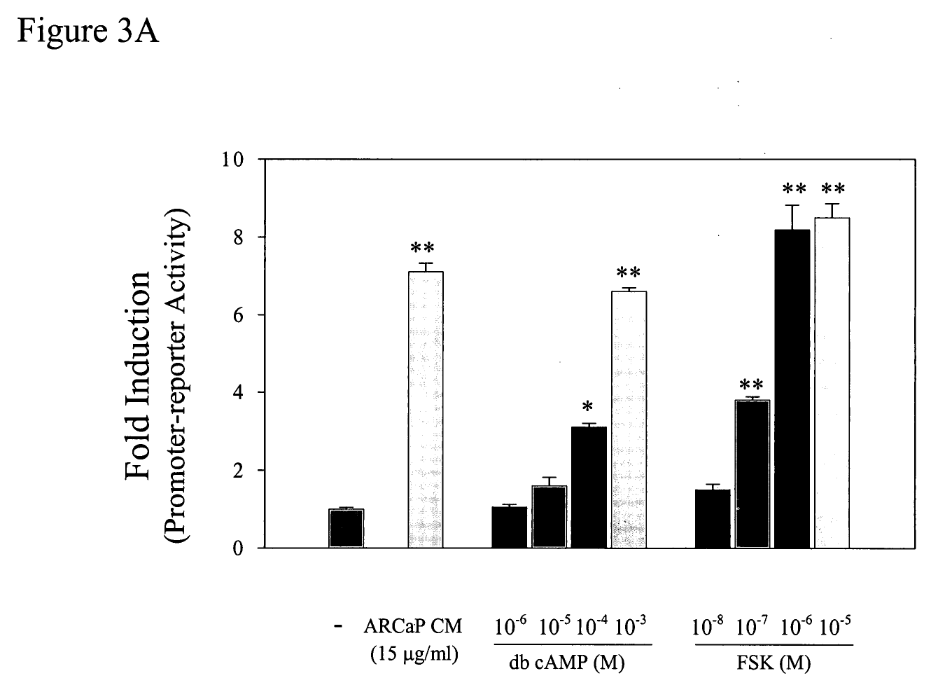

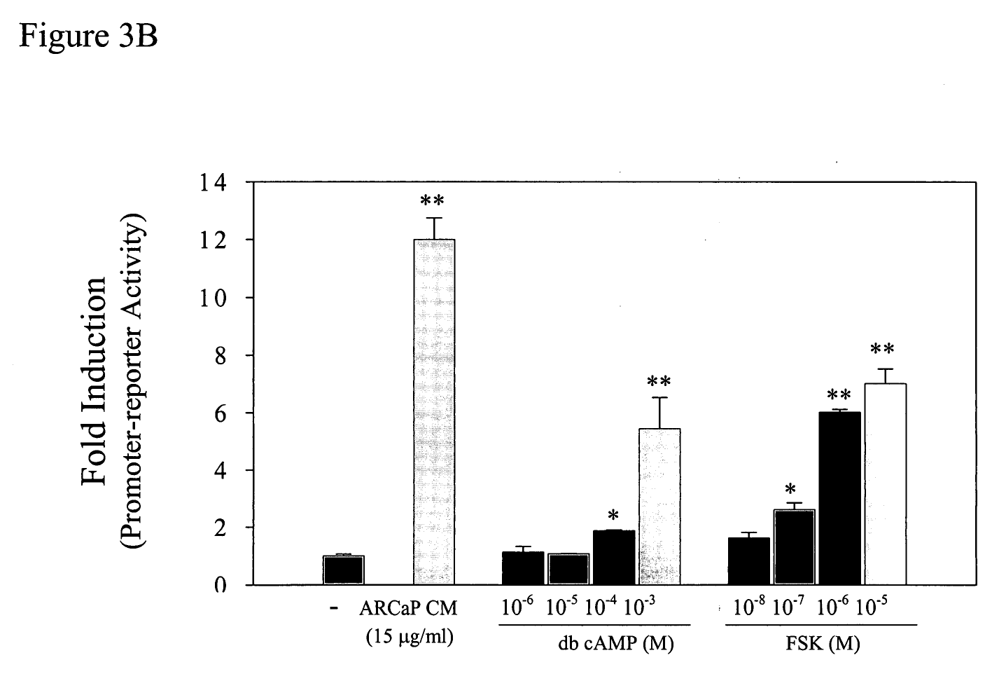

The cAMP-dependent PKA pathway has long been shown to mediate specific intracellular signaling events, including the transcription of specific genes via the CRE cis-element (25, 30, 31). To determine whether ARCaP CM stimulation of hOC and hBSP promoter activities may be mediated through an activation of the PKA signaling pathway, we assessed the effects of PKA pathway activators, dibutyryl cAMP (db cAMP) and forskolin (FSK), on hOC and hBSP promoter activities in C4-2B cells and correlated this with endogenous mRNA expression in various human prostate cancer cell lines. The PKA pathway activators, db cAMP (10⁻⁶ to 10⁻³ M) and FSK (10⁻⁸ to 10⁻⁵ M) stimulated hOC (FIG. 3A) and hBSP (FIG. 3B) promoter activities in a ligand concentration-dependent manner in C4-2B cells. These results were confirmed by an assessment of endogenous OC and BSP mRNA expression subsequent to induction by a PKA activator, FSK. Following FSK treatment (10⁻⁶ M), OC and BSP mRNA expression in LNCaP and C4-2B but not in PC3 and MG63 were elevated (FIG. 3C). In this study, the steady-state levels of OC mRNA were elevated by 5.2 and 7.8-fold whereas the levels of BSP mRNA were increased by 3.2 and 5.4-fold after FSK stimulation in LNCaP and C4-2B cells, respectively. This result was in general agreement with the effect of ARCaP CM on endogenous OC and BSP mRNA expression (FIG. 1E), further supporting the involvement of PKA as the key downstream signaling pathway regulating soluble factor-mediated OC and BSP gene expression in LNCaP and C4-2B human prostate cancer cells.

The involvement of the PKA pathway in mediating ARCaP CM activation of OC and BSP expression was further confirmed using a selective inhibitor of PKA, H-89 (39). FSK-stimulated hOC and hBSP promoter activities were both inhibited by H-89 (10⁻⁸ to 10⁻⁶ M) in a concentration-dependent manner (FIG. 4A). Consistent with this observation, H-89 also inhibited ARCaP CM- and db cAMP-mediated activation of hOC promoter activity in prostate cancer cells (FIG. 4B). Interestingly, PMA, an activator of the PKC pathway, also induced hOC promoter activity to a lesser extent and such activation, as expected, was not blocked by H-89 (FIG. 4B). In agreement with the above results from the hOC and hBSP promoter study, we confirmed that H-89 also inhibited the induction of endogenous OC and BSP mRNA expression by ARCaP CM or FSK in LNCaP and C4-2B cells (data not shown). We evaluated further whether the cAMP/PKA signaling pathway may be operative under induction by CM harvested from C4-2B, DU145, PC3 and MG63 cell lines. FIG. 4C shows that H-89 (10⁻⁶ M) nearly abolished CM-induced hOC promoter activity in all of CM harvested from prostate cancer and bone stromal cell lines. These data suggest that stimulation of bone-specific OC and BSP gene expression in human prostate cancer cell lines by soluble factor(s) in prostate cancer or bone CM occurs primarily through the PKA signaling pathway involving cAMP as a mediator.

Evidence in Support of Nuclear CRE-Binding Protein (CREB) and Cis-Acting Element, CRE, in the Regulation of Bone-Specific Gene Expression in Human Prostate Cancer Cells: Electrophoretic Mobility Shift Assay (EMSA).

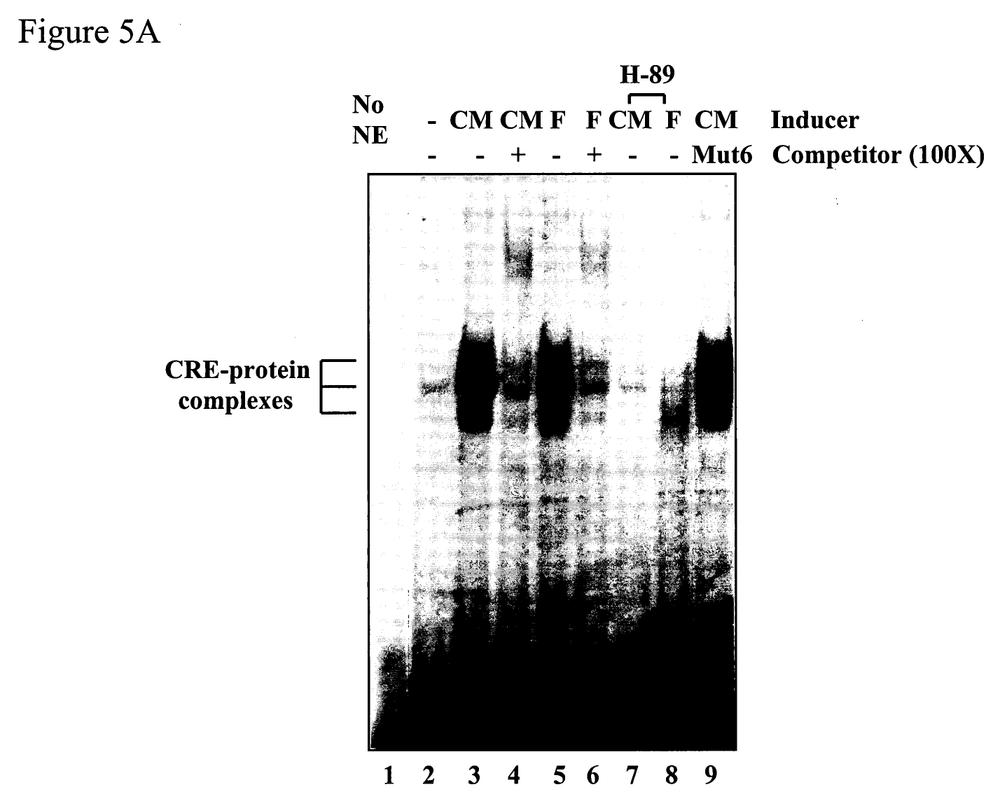

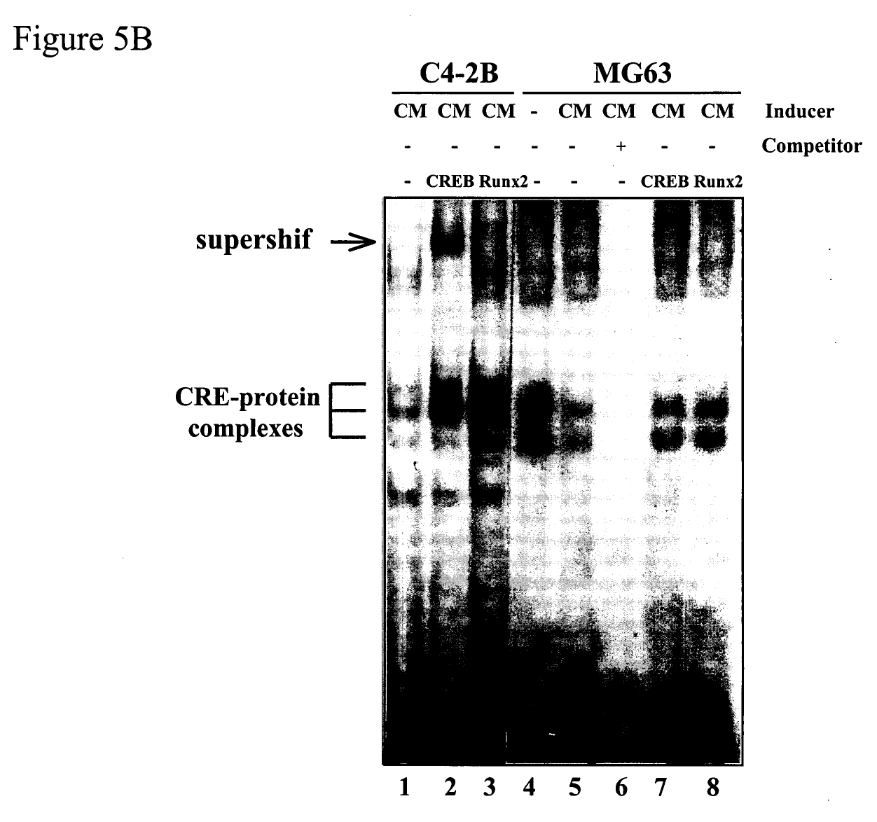

To further establish a downstream link between the cAMP-dependent PKA signaling pathway and hOC and hBSP promoter activation in prostate cancer cells, we conducted EMSA to compare the binding of a ³²P-labeled oligonucleotide CRE probe and nuclear factors extracted from C4-2B cells (an ARCaP CM-positive responder) and MG63 cells (an ARCaP CM-negative responder) that had previously been exposed to ARCaP CM (15 μg/ml) or FSK (10⁻⁵ M) for 16 h in culture. Cells exposed to vehicle were used as controls. Nuclear factors extracted from either ARCaP CM (CM) or FSK (F) treated C4-2B cells strongly enhanced the specific CRE-nuclear protein complex formation (FIG. 5A, lanes 3 and 5) in comparison to cells exposed to control media (FIG. 5A, lane 2). These DNA-protein complexes could be competed off by unlabeled specific CRE-oligo probe (lanes 4 and 6). However, no competition was observed with a mutant form of CRE-oligo probe, the Mut6-oligo (two-point substitution, see FIG. 2D) (lane 9). Consistent with the biochemical data, H-89 was shown to abolish both ARCaP CM- and FSK-induced CRE binding to the nuclear proteins extracted from C4-2B cells (lanes 7 and 8). Nuclear extracts from MG63 cells, in contrast, formed a low but detectable basal level of complexes with ³²P-labeled-CRE probe before and after treatment with ARCaP CM (FIG. 5B, lanes 4 and 5). These complexes could be competed off by unlabeled-CRE probe (lane 6), but failed to be supershifted by anti-CREB-specific antibody (lane 7). As expected, nuclear extracts from C4-2B cells exposed to ARCaP CM did bind to CRE and these CRE-nuclear protein complexes can be supershifted by anti-CREB antibody (lane 2) but not by anti-Runx2 antibody in both cases (lanes 3 and 8). These data demonstrate that the trans-acting factor CREB may play a critical role in regulating bone-specific gene transcription through the cAMP/PKA pathway by ARCaP CM in human prostate cancer but not in bone stromal cells.

Discussion

Prostate cancer expresses proteins normally restricted to bone, such as OC, BSP, osteopontin, and osteonectin (6, 7, 11, 40-43). We hypothesized that the osteomimetic property of prostate cancer cells results from transcription factor switching (9, 44). Because of the potential importance of osteomimicry in enhancing cancer cell adhesion, invasion and metastasis (6, 7, 11), we designed studies to define how the expression of bone-specific proteins in prostate cancer cells is regulated. We chose to study OC and BSP because of the prevalence of expression of these genes by prostate cancer cell lines (8, 45) and in clinical prostate cancer specimens (7, 8, 46). Our data showed that OC and BSP expression are stimulated in a selective manner in human prostate cancer but not in bone stromal cell lines. We observed that the induction of OC and BSP expression in prostate cancer cell lines is mediated by paracrine/autocrine factors harvested from CM of prostate cancer and bone stromal cells (47). The selective nature of soluble factors serving as paracrine mediators in stimulating OC and BSP expression in prostate cancer cell lines agrees with their immunohistochemical staining patterns which are generally heterogeneous (see FIGS. 1A and 7, 8, 46). Using the stimulatory response of hOC/hBSP promoter reporter activities and the endogenous steady-state levels of OC/BSP mRNA in C4-2B as the assay end points, we showed that the extent of hOC promoter activation by CM collected from prostate cancer and bone stromal cell lines correlated directly with the malignant potential of these cells in laboratory immune-compromised mice. We also observed that CM stimulated hOC and hBSP promoter activities in a dose-dependent manner and that these increased promoter reporter activities corresponded with the enhancement of the steady-state levels of endogenous OC and BSP mRNA expressed in LNCaP and C4-2B cells. These results support the idea that OC and BSP, once induced in prostate cancer cells, could facilitate the formation of hydroxyapatite complexes, leading to altered biologic functions of prostate cancer cells, i.e. increased cell adhesion, migration and invasion and recruitment of bone cells. Enhanced bone turnover could contribute to prostate cancer “seeding” in the skeleton (4, 11).

A CRE cis-element, located upstream of the AV region, is responsible for mediating ARCaP CM activated hOC promoter activity and OC mRNA expression in human prostate cell lines (FIG. 2B). This cis-element appears to be functional in LNCaP and C4-2B cells but remains silent in a number of other human prostate cancer (DU145, PC3 and ARCaP) and osteosarcoma (MG63) cell lines. This is supported by the lack of increased OC expression in these prostate cancer and osteosarcoma cell lines after exposure to ARCaP CM (FIGS. 1D and 1E). Further, nuclear extracts from prostate cancer and bone cells that failed to respond to PKA pathway activators showed a lack of CRE binding and supershift activity. In sharp contrast, however, an increased complex formation between cis-element CRE and trans-acting factor CREB, and an expected supershift of this complex, were observed in the responsive C4-2B cells upon the addition of anti-CREB antibody (FIGS. 5A and 5B). The complex formation is likely to account for increased hOC promoter activity and enhanced endogenous OC mRNA expression in human prostate cancer cells, in response to factor(s) from the tumor cell microenvironment. A similar mechanism regulating hBSP promoter activity and mRNA expression was also observed in LNCAP and C4-2B cells after exposure to ARCaP CM, a result supported by additional studies using interfering pharmacologic agents that either stimulated (db cAMP and FSK) or repressed (H-89) cAMP accumulation in target cells (FIGS. 1D, 1E, 2C, 3B, 3C and 4A). Our results demonstrate for the first time that CM harvested from prostate cancer and bone stromal cells stimulated OC and BSP expression primarily through a cAMP-dependent PKA signaling pathway in LNCaP and C4-2B human prostate cancer cells. This stimulation by CM lends further support to the observation that OC and BSP are prevalently expressed by clinically localized and bone and lymph node metastatic human prostate cancer tissue specimens and can be the molecular basis of osteomimicry during disease progression.

A number of growth factors (FGF-2 or bFGF, IGF-1 and TGF-β) and hormones (glucocorticoids, estrogens, PTH and PTHrP) have been shown to regulate OC and BSP expression in rodent, chick or human cell lines (48-54). Boudreaux and Towler (55) demonstrated the induction of OC promoter activity by a growth factor, FGF-2, in MC3T3-E1 cells. Boguslawski and colleagues (30) observed that rat and human osteoblast-like cell lines stably transfected with OC promoter reporter construct responded to PTH and growth factors (FGF-2 and IGF-1) via enhanced OC transcription mediated by a PKA-dependent pathway. A similar activation of BSP promoter activity was documented for FGF-2 (54, 56), PTH (33, 53) and PGE₂ (34) in which BSP transcription was stimulated through a PKA-dependent pathway, although a PKC-mediated pathway may play a minor role (33). The present study differs from previous reports in two important aspects. First, none of the earlier observations demonstrated conclusively at the molecular level that the involvement of CRE elements within OC and BSP promoters, as defined in the present study, conferred soluble factor-induced OC and BSP proteins expression by cancer or normal cells. Our results emphasized the roles of CRE cis-elements in osteomimicry by prostate cancer cells. Second, none of the previously reported factors tested in our assay system activated hOC and hBSP promoter activities in C4-2B cells (data not shown). These important distinctions suggest the following possibilities. 1) There are fundamental differences, in terms of the soluble factors, downstream signaling network and cis-elements within hOC and hBSP promoters that are responsible for mediating the osteomimetic properties of prostate cancer and bone cells. 2) At the molecular level, prostate cancer and bone cells may differ in their cell surface receptors and downstream cell signaling pathways responding to soluble factor(s) secreted by prostate cancer and bone stromal cells and their elicited activation of OC and BSP expression. 3) With respect to the osteomimetic response of prostate cancer cells to soluble factors, prostate cancer cell lines clearly show heterogeneity. Since OC and BSP expression in LNCaP, C4-2 and C4-2B cells are responsive to soluble factors and OC and BSP proteins are prevalently expressed in both primary and metastatic human prostate cancer tissues, we suggest that LNCAP and its derivative cell lines are superior models for the study of human prostate cancer progression. This conclusion is also supported by a large body of literature devoted to the study of the molecular mechanisms of androgen receptor and androgen-independent metastatic progression using this LNCaP prostate cancer progression model (57-59). To further understand differences in responsiveness among prostate cancer and bone stromal cells to soluble factors in CM, we must isolate and characterize the responsible factors and evaluate cell surface or intracellular receptors coupling to cell signaling systems in prostate cancer and bone cells.

In summary, our data using human prostate cancer cell lines demonstrate dramatic cell background-dependent differences in responsiveness to ARCaP CM and the involvement of the cAMP-PKA signaling cascade in the induction of OC and BSP gene expression in LNCaP and C4-2B cells. We established at the molecular level that a specific region of cis-element in hOC promoter, located between -643 to -636 (CRE), must be responsible for conferring cAMP regulation of hOC promoter activity in human prostate cancer cells. Likewise, other regions of CRE within hBSP promoter, -79 to -72 (CRE1) and -674 to -667 (CRE2), must also be activated upon the exposure of human prostate cancer cells to cAMP mimetics and yet unidentified growth factor(s) in the CM of prostate cancer and bone stromal cells.

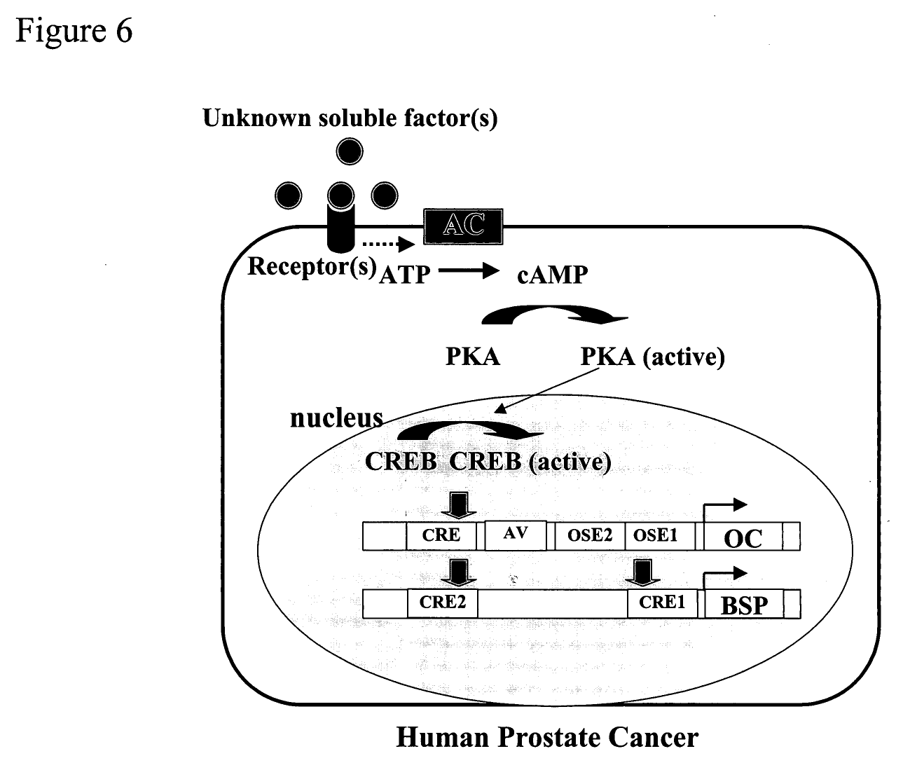

We propose that unknown soluble factors secreted by human prostate cancer or bone stromal cells could assume the key regulatory role in OC and BSP expression in human prostate cancer (FIG. 6). An ongoing study to identify and characterize the unknown soluble factors in our laboratory has revealed a small polypeptide (less than 30 kDa) with thermal sensitivity and ammonium sulfate-precipitable characteristics in CM, which appears exclusively responsible for the activation of hOC and hBSP promoter activities in prostate cancer cells. Characterization of this soluble factor is presented in Example 2, infra. These results could have significant implications for understanding osteomimicry and targeting it therapeutically in human prostate cancer.

Acknowledgments

The inventors thank their colleagues at the Molecular Urology and Therapeutics Program for helpful suggestions and discussion and Gary Mawyer for editing the manuscript.

### REFERENCES

1. Greenlee R T, Murray T, Bolden S, Wingo P A. Cancer statistics, 2000. CA Cancer J Clin 2000;50:7-33.
2. Martinez J, Silva S, Santibanez J F. Prostate-derived soluble factors block osteoblast differentiation in culture. J Cell Biochem 1996;61:18-25.
3. Lang S H, Clarke N W, George N J, Allen T D, Testa N G. Interaction of prostate epithelial cells from benign and malignant tumor tissue with bone-marrow stroma. Prostate 1998;34:203-13.
4. Chung L W. Prostate carcinoma bone-stroma interaction and its biologic and therapeutic implications. Cancer 2003;97:772-8.
5. Curatolo C, Ludovico G M, Correale M, et al. Advanced prostate cancer follow-up with prostate-specific antigen, prostatic acid phosphatase, osteocalcin and bone isoenzyme of alkaline phosphatase. Eur Urol 1992;21 Suppl 1:105-7.
6. Waltregny D, Bellahcene A, Van Riet I, et al. Prognostic value of bone sialoprotein expression in clinically localized human prostate cancer. J Natl Cancer Inst 1998;90:1000-8.
7. Carlinfante G, Vassilioul D, Svensson O, Wendel M, Heinegard D, Andersson G. Differential expression of osteopontin and bone sialoprotein in bone metastasis of breast and prostate carcinoma. Clin Exp Metastasis 2003;20:437-44.
8. Matsubara S, Wada Y, Gardner T A, et al. A conditional replication-competent adenoviral vector, Ad-OC-Ela, to cotarget prostate cancer and bone stroma in an experimental model of androgen-independent prostate cancer bone metastasis. Cancer Res 2001;61:6012-9.
9. Yeung F, Law W K, Yeh C H, et al. Regulation of human osteocalcin promoter in hormone-independent human prostate cancer cells. J Biol Chem 2002;277:2468-76.
10. Brown L F, Papadopoulos-Sergiou A, Berse B, et al. Osteopontin expression and distribution in human carcinomas. Am J Pathol 1994;145:610-23.
11. Koeneman K S, Yeung F, Chung L W. Osteomimetic properties of prostate cancer cells: a hypothesis supporting the predilection of prostate cancer metastasis and growth in the bone environment. Prostate 1999;39:246-61.
12. Price P A, Otsuka A A, Poser J W, Kristaponis J, Raman N. Characterization of a gamma-carboxyglutamic acid-containing protein from bone. Proc Natl Acad Sci USA 1976;73:1447-51.
13. Boskey A L, Gadaleta S, Gundberg C, Doty S B, Ducy P, Karsenty G. Fourier transform infrared microspectroscopic analysis of bones of osteocalcin-deficient mice provides insight into the function of osteocalcin. Bone 1998;23:187-96.
14. Hauschka P V. Osteocalcin: the vitamin K-dependent Ca2+-binding protein of bone matrix. Haemostasis 1986;16:258-72.
15. Ducy P, Desbois C, Boyce B, et al. Increased bone formation in osteocalcin-deficient mice. Nature 1996;382:448-52.
16. Ganss B, Kim R H, Sodek J. Bone sialoprotein. Crit Rev Oral Biol Med 1999;10:79-98.
17. Hunter G K, Goldberg H A. Nucleation of hydroxyapatite by bone sialoprotein. Proc Natl Acad Sci USA 1993;90:8562-5.
18. Ross F P, Chappel J, Alvarez J I, et al. Interactions between the bone matrix proteins osteopontin and bone sialoprotein and the osteoclast integrin alpha v beta 3 potentiate bone resorption. J Biol Chem 1993;268:9901-7.
19. Mintz K P, Grzesik W J, Midura R J, Robey P G, Termine J D, Fisher L W. Purification and fragmentation of nondenatured bone sialoprotein: evidence for a cryptic, RGD-resistant cell attachment domain. J Bone Miner Res 1993;8:985-95.
20. Zhang J H, Tang J, Wang J, et al. Over-expression of bone sialoprotein enhances bone metastasis of human breast cancer cells in a mouse model. Int J Oncol 2003;23:1043-8.
21. Fedarko N S, Fohr B, Robey P G, Young M F, Fisher L W. Factor H binding to bone sialoprotein and osteopontin enables tumor cell evasion of complement-mediated attack. J Biol Chem 2000;275:16666-72.
22. Roach H I. Why does bone matrix contain non-collagenous proteins? The possible roles of osteocalcin, osteonectin, osteopontin and bone sialoprotein in bone mineralisation and resorption. Cell Biol Int 1994;18:617-28.
23. Stein G S, Lian J B, Owen T A. Relationship of cell growth to the regulation of tissue-specific gene expression during osteoblast differentiation. Faseb J 1990;4:3111-23.
24. Kondo H, Ohyama T, Ohya K, Kasugai S. Temporal changes of mRNA expression of matrix proteins and parathyroid hormone and parathyroid hormone-related protein (PTH/PTHrP) receptor in bone development. J Bone Miner Res 1997;12:2089-97.
25. Richards J S. New signaling pathways for hormones and cyclic adenosine 3′,5′-monophosphate action in endocrine cells. Mol Endocrinol 2001;15:209-18.
26. Liu A Y. Differentiation-specific increase of cAMP-dependent protein kinase in the 3T3-L1 cells. J Biol Chem 1982;257:298-306.
27. Schwartz D A, Rubin C S. Regulation of cAMP-dependent protein kinase subunit levels in Friend erythroleukemic cells. Effects of differentiation and treatment with 8-Br-cAMP and methylisobutyl xanthine. J Biol Chem 1983;258:777-84.
28. Li M, West J W, Numann R, Murphy B J, Scheuer T, Catterall W A. Convergent regulation of sodium channels by protein kinase C and cAMP-dependent protein kinase. Science 1993;261:1439-42.
29. Roesler W J, Vandenbark G R, Hanson R W. Cyclic AMP and the induction of eukaryotic gene transcription. J Biol Chem 1988;263:9063-6.
30. Boguslawski G, Hale L V, Yu X P, et al. Activation of osteocalcin transcription involves interaction of protein kinase A- and protein kinase C-dependent pathways. J Biol Chem 2000;275 :999-1006.
31. Servillo G, Della Fazia M A, Sassone-Corsi P. Coupling cAMP signaling to transcription in the liver: pivotal role of CREB and CREM. Exp Cell Res 2002;275:143-54.
32. Sassone-Corsi P. Coupling gene expression to cAMP signalling: role of CREB and CREM. Int J Biochem Cell Biol 1998;30:27-38.
33. Yang R, Gerstenfeld L C. Signal transduction pathways mediating parathyroid hormone stimulation of bone sialoprotein gene expression in osteoblasts. J Biol Chem 1996;271:29839-46.
34. Samoto H, Shimizu E, Matsuda-Honjyo Y, et al. Prostaglandin E2 stimulates bone sialoprotein (BSP) expression through cAMP and fibroblast growth factor 2 response elements in the proximal promoter of the rat BSP gene. J Biol Chem 2003;278:28659-67.
35. Gleave M, Hsieh J T, Gao C A, von Eschenbach A C, Chung L W. Acceleration of human prostate cancer growth in vivo by factors produced by prostate and bone fibroblasts. Cancer Res 1991;51:3753-61.
36. Kiyoshima T, Yamauchi M, Wong C, Jheon A, Ganss B, Sodek J. An L1 element disrupts human bone sialoprotein promoter: lack of tissue-specific regulation by distalless5 (D1x5) and runt homeodomain protein2 (Runx2)/core binding factor al (Cbfa1) elements. Gene 2002;299:205-17.
37. Ausubel F M, Brent R, Kingston R E, et al. Current protocols in molecular biology. New York: Greene Pub. Associates and Wiley-Interscience: J. Wiley; 1999.
38. Mahonen A, Pirskanen A, Keinanen R, Maenpaa P H. Effect of 1,25(OH)2D3 on its receptor mRNA levels and osteocalcin synthesis in human osteosarcoma cells. Biochim Biophys Acta 1990;1048:30-7.
39. Chijiwa T, Mishima A, Hagiwara M, et al. Inhibition of forskolin-induced neurite outgrowth and protein phosphorylation by a newly synthesized selective inhibitor of cyclic AMP-dependent protein kinase, N-[2-(p-bromocinnamylamino)ethyl]-5-isoquinolinesulfonamide (H-89), of PC12D pheochromocytoma cells. J Biol Chem 1990;265:5267-72.
40. Chung L W, Hsieh C L, Law A, et al. New targets for therapy in prostate cancer: modulation of stromal-epithelial interactions. Urology 2003;62:44-54.
41. Thalmann G N, Sikes R A, Devoll R E, et al. Osteopontin: possible role in prostate cancer progression. Clin Cancer Res 1999;5:2271-7.
42. Jacob K, Webber M, Benayahu D, Kleinman H K. Osteonectin promotes prostate cancer cell migration and invasion: a possible mechanism for metastasis to bone. Cancer Res 1999;59:4453-7.
43. Thomas R, True L D, Bassuk J A, Lange P H, Vessella R L. Differential expression of osteonectin/SPARC during human prostate cancer progression. Clin Cancer Res 2000;6:1140-9.
44. Yeung F, Chung L W. Molecular basis of co-targeting prostate tumor and stroma. J Cell Biochem Suppl 2002;38:65-72.
45. Lin D L, Tarnowski C P, Zhang J, et al. Bone metastatic LNCaP-derivative C4-2B prostate cancer cell line mineralizes in vitro. Prostate 2001;47:212-21.
46. Ou Y C, Chen J T, Yang C R, Ko J L, Hsieh Y S, Kao C. Expression of osteocalcin in prostate cancer before and after hormonal therapy. Anticancer Res 2003;23:3807-11.
47. Huang W C, Zhau H E, Chung L W. Invasive prostate cancer cells secret soluble protein factors that upregulate osteocalcin (OC) promoter activity: Implication to stroma desmoplastic response and cancer metastasis. J Urol 2003;169:154-4. (Abstr 597)
48. Xiao G, Jiang D, Gopalakrishnan R, Franceschi R T. Fibroblast growth factor 2 induction of the osteocalcin gene requires MAPK activity and phosphorylation of the osteoblast transcription factor, Cbfa1/Runx2. J Biol Chem 2002;277:36181-7.
49. Kim H J, Kim J H, Bae S C, Choi J Y, Ryoo H M. The protein kinase C pathway plays a central role in the fibroblast growth factor-stimulated expression and transactivation activity of Runx2. J Biol Chem 2003;278:319-26.
50. Alliston T, Choy L, Ducy P, Karsenty G, Derynck R. TGF-beta-induced repression of CBFA1 by Smad3 decreases cbfal and osteocalcin expression and inhibits osteoblast differentiation. Embo J 2001;20:2254-72.
51. Lian J B, Shalhoub V, Aslam F, et al. Species-specific glucocorticoid and 1,25-dihydroxyvitamin D responsiveness in mouse MC3T3-E1 osteoblasts: dexamethasone inhibits osteoblast differentiation and vitamin D down-regulates osteocalcin gene expression. Endocrinology 1997;138:2117-27.
52. Ogata Y, Yamauchi M, Kim R H, Li J J, Freedman L P, Sodek J. Glucocorticoid regulation of bone sialoprotein (BSP) gene expression. Identification of a glucocorticoid response element in the bone sialoprotein gene promoter. Eur J Biochem 1995;230:183-92.
53. Ogata Y, Nakao S, Kim R H, et al. Parathyroid hormone regulation of bone sialoprotein (BSP) gene transcription is mediated through a pituitary-specific transcription factor-1 (Pit-1) motif in the rat BSP gene promoter. Matrix Biol 2000;19:395-407.
54. Shimizu-Sasaki E, Yamazaki M, Furuyama S, Sugiya H, Sodek J, Ogata Y. Identification of a novel response element in the rat bone sialoprotein (BSP) gene promoter that mediates constitutive and fibroblast growth factor 2-induced expression of BSP. J Biol Chem 2001 ;276:5459-66.
55. Boudreaux J M, Towler D A. Synergistic induction of osteocalcin gene expression: identification of a bipartite element conferring fibroblast growth factor 2 and cyclic AMP responsiveness in the rat osteocalcin promoter. J Biol Chem 1996;271:7508-15.
56. Shimizu-Sasaki E, Yamazaki M, Furuyama S, Sugiya H, Sodek J, Ogata Y. Identification of FGF2-response element in the rat bone sialoprotein gene promoter. Connect Tissue Res 2003;44 Suppl 1:103-8.
57. Pfitzenmaier J, Quinn J E, Odman A M, et al. Characterization of C4-2 prostate cancer bone metastases and their response to castration. J Bone Miner Res 2003; 18:1882-8.
58. Letsch M, Schally A V, Szepeshazi K, Halmos G, Nagy A. Preclinical evaluation of targeted cytotoxic luteinizing hormone-releasing hormone analogue AN-152 in androgen-sensitive and insensitive prostate cancers. Clin Cancer Res 2003;9:4505-13.
59. Zayzafoon M, Abdulkadir S A, McDonald J M. Notch signaling and ERK activation are important for the osteomimetic properties of prostate cancer bone metastatic cell lines. J Biol Chem 2004;279:3662-70.

### Example 2

### β2M Overexpression in Human Prostate Cancer Cells Induces Explosive Growth and Osteoblastic Reactions in Mouse Bone

As described Supra in Example 1, the inventors recently discovered that the osteoblast-mimicking activity of prostate cancer cells, the ability of prostate cancer cells to synthesize and deposit OC and BSP, is controlled solely by a stretch of eight base nucleotides, the cyclic AMP responsive element (CRE), located at the human OC and BSP promoters (52). Further, the inventors found that Protein Kinase A (PKA) activation of a downstream CREB is responsible for osteomimicry in prostate cancer cells.

Among many potential factors that could activate the PKA signaling pathway, the inventors isolated and characterized a soluble factor secreted by prostate cancer, bone and inflammatory (macrophage) cells. This factor, β2M, is responsible for the activation of PKA in cultured cells and in situ. β2M, a well-known housekeeping gene, has an expected similar level of mRNA expression among all prostate cancer cell lines tested. Interestingly, the secreted form of β2M protein is markedly different between cell lines and the steady-state level of β2M protein correlated directly with the malignant potential of prostate cancer cells. As will be described below, this discovery led the inventors to test the biologic activity of β2M in prostate cancer cells. β2M overexpressing human prostate, breast, lung or renal cancer cells exhibited increased growth rates on both plastic dishes and as soft agar or matrigel colonies, and their growth closely correlated with the steady-state of β2M protein expression in the transfected cell clones. Human prostate cancer cells overexpressing β2M, when introduced in bone, showed explosive growth with a mixed histopathology of both osteoblastic and osteolytic reactions (see Results below). Sequence-specific β2M siRNA was found to knock down β2M expression by prostate cancer cells, and this treatment effectively abolished the growth of pre-existing prostate cancers in mouse femur or as bone powder xenografts, by inducing massive tumor cell death. These results established that β2M is an effective growth promoter for human prostate, breast, lung and renal cancer cells. The inventors demonstrate herein that in mouse bone or bone powder, β2M and its downstream signaling components are new and exciting potential therapeutic targets for the control of human prostate cancer skeletal metastasis. We propose to investigate the roles of β2M in accelerating human prostate, breast, lung and renal cancer growth and their subsequent dissemination to bone.

The inventors will evaluate β2M-mediated downstream cell signaling, particularly the activation of the vascular endothelial growth factor axis, in prostate cancer cells and cells in the tumor microenvironment. We will establish transgenic animal models for prostate cancer bone metastasis for prognosis and targeting. Novel strategies include the use of β2M siRNA and small molecules to target β2M-mediated osteomimicry in human prostate cancer bone metastasis. This same approach will then be applied to other solid tumor bone xenograft models, such as human breast, lung and renal cancers. It is believed that these approaches will ultimately be translated into the clinic to improve the survival of cancer patients with skeletal metastases.

Experimental Procedures

Cell Lines and Cell Culture

Human prostate cancer cell lines, LNCaP, C4-2B, DU145, PC3 and ARCaP, and a human osteosarcoma cell line, MG63 were cultured in T-medium (Life Technologies Inc., Rockville, Md.) supplemented with 5% fetal bovine serum and 1% penicillin/streptomycin as previously described {Gleave, 1991#35}. The cells were maintained at 37° C. in 5% CO₂. For CM collection, cells were cultured in T-medium with serum until 80% confluence. The cells were washed subsequently twice in PBS (10 mM phosphate buffer and 137 mM NaCl) and incubated in T-medium without serum. After two days additional incubation, CM were collected and centrifuged, stored at −20° C. until use.

Purification and Characterization of β2-Microglobulin

All purification procedures were performed at 4° C. unless stated otherwise. Total proteins from 100 ml of serum-free ARCaP CM were precipitated by 0-100% saturation of solid and oven-dried ammonium sulfate. After centrifugation, the precipitates were dissolved in 1 ml of PB [10 mM sodium phosphate, pH 7.6, containing 0.2 mM PMSF, 0.5 mM DTT and protease inhibitor cocktail (Roche Molecular Biochemicals, Indianapolis, Ind.)], and dialyzed against 500 ml of PB, twice, overnight. After centrifugation of the dialyzed solution, the supernatant was passed through Centricon Plus-20 (YM-30, membrane cut-off 30 k, Millipore Corp., Billerica, Mass.) to remove the proteins with high molecular mass (>30 kDa). One hundred μl of the protein filtrate (ca. 100 μg of total proteins) was loaded into an anion-exchange column (4×250 mm, ProPac Wax-10, Dionex, Sunnyvale, Calif.) preequilibrated with PB at a flow rate of 1 ml/min. The bound-form proteins were eluted with a liner gradient of 0-1 M NaCl. After analysis of hOC promoter activity {Huang, 2005#67} for each fraction, the purified protein fractions having promoter activity were collected, and analyzed by SDS-PAGE with silver staining (Invitrogen, Carlsbad, Calif.) and determined the N-terminal amino acid sequence of the homogenous protein by Edman degradation method {Edman, 1970#39} performed by Microchemical and Proteomics Facility, Emory University, GA.

RT-PCR

Total RNA was isolated from the confluent monolayer of cells using RNAZol B (Teltest Inc., Friendswood, Tex.). The total RNA was used as template for RT according to the manufacturer's instruction (Invitrogen). The oligonucleotide primer sets used for PCR analysis of cDNA are: β2M, 5′-ACGCGTCCGAAGCTTACAGCATTC-3′ (forward) and 5′-CCAAATGCGGCATCTAGAAACCTCCATG-3′ (reverse); OC, BSP and glyceraldehyde phosphate dehydrogenase (GAPDH) as previously described {Huang, 2005#67}, respectively. Thermal profile for β2M amplification is 30 cycles starting with denaturation 1 min at 94° C., followed by 1 min of annealing at 64° C. and 30 s of extension at 72° C. RT-PCR products were analyzed by 1.2% agarose gel electrophoresis. Quantity one-4.1.1 Gel Doc gel documentation software (Bio-Rad, Hercules, Calif.) was used for quantification of β2M mRNA expression normalized by GAPDH mRNA expression.

Western Blot Analysis and β2M ELISA

Western blotting was performed using the NOVEX system (Invitrogen). Primary antibody β2M (Santa Cruz Biotechnology, Inc., Santa Cruz, Calif.) was used at a 1:500 dilution, and secondary antibody (horseradish peroxidase-anti-rabbit antibody, Amersham Biosciences Corp., Piscataway, N.J.) was used at a 1:5,000 dilution. Detection of protein bands was performed with ECL Western Boltting Detection Reagents (Amersham Biosciences Corp.). The concentration of total proteins was determined by the Bradford method using Coomassie plus protein reagent (Pierce, Rockford, Ill.). β2M concentration was assayed by Quantikine IVD human β2M ELISA kit (R&D Systems, Inc., Minneapolis, Minn.) according to the manufacturer's instruction.

Plasmid Construction

For construction of expression vector of β2M, β2M cDNA inserting HindIII and XbaI sites was cloned by RT-PCR (see above, RT-PCR). After RT-PCR, β2M cDNA (427 bp) was subcloned into pcDNA 3.1 expression vector (Invitrogen). The empty pcDNA 3.1 expression vector was used as control (Neo). The hOC and hBSP promoter constructs have been described before {Huang, 2005#67}.

In Vitro Cell Proliferation Assay

LNCaP, C4-2B, DU145, PC3, MG63 and ARCaP cell lines were seeded in 96-well plates in T-medium containing 5% FBS, respectively. After 24 h incubation, media were replaced by fresh, serum-free T-medium and incubated for an additional 24 h. The cells were treated with β2M CM or Neo CM for 4-day incubation. Cell numbers were measured every 24 h using CellTiter 96® AQueous One Solution Cell Proliferation Assay (Promega, Madison, Wis.).

In Vivo Animal Study

All of the animal experiments were approved and performed in accordance with the institutional guidelines. Four weeks old male athymic nu/nu mice (NCl, Frederick, Md.) were inoculated into the s.c. space of the flank regions with 2×10⁶ cells or injected into bone marrow space of the femurs with 1×10⁶ cells of Neo or β2M-overexpressing C4-2B cells, respectively. Blood specimens were harvested for PSA assay once two weeks. Serum PSA levels were determined by microparticle ELISA using Abbott IMx machine (Abbott Laboratories, Abbott Park, Ill.).

β2M-siRNA and Anti-Prostate Tumor Study

The β2M- and control scramble-siRNA sequences were 5′-UUGCUAUGUGUCUGGGUUU(dT)(dT)-3′ and 5′-UUCAUGUGUCUGUGGUGUU(dT)(dT)-3′, respectively. For RNA delivery system, we used cationic liposome formulation, termed SN {Zou, 2002#68} to deliver β2M- and scramble-siRNA into cell lines or mice. The ratio of siRNA (μg) to SN (μl) was 1:24. To test prostate tumor growth inhibition by β2M-siRNA, four weeks old male athymic nu/nu mice were inoculated into the subcutaneous space of the chest regions with 2×10⁶ cells of PC3-Luc or C4-2 mixed with 20 mg of bone powder (kindly provided by Dr. A. H. Reddi, University of California, Davis, Sacramento, Calif.) and 35 μl of Matrigel matrix (BD Bioscience, Bedford Mass.), respectively. Three (PC3-Luc) or four (C4-2) weeks later, the tumor-bearing mice were randomly divided into two groups. The mice in the treatment group received intratumoral injection of SN-β2M-siRNA for three-time per week continuously four weeks, at a dose of 0.8 μg of siRNA mixed with 19.2 μl of SN for each mouse. The control group injected with the same dose of SN-scramble-siRNA. To assay of anti-tumor activity of β2M-siRNA therapeutic efficacy, the real-time bioluminescence image by the CCD camera cryogenically cooled IVIS system with analysis software (Xenogen Corp., Alameda Calif.) (Ref) and serum PSA levels were used to monitored PC3-Luc and C4-2 tumor burden in nude mice.

Statistical Analysis.

All statistical analyses were performed using Microsoft Excel software or kindly assisted by Dr. Jae K. Lee (University of Virginia, Charlottesville, Va.). Significant differences were analyzed using Student's t test and two-tailed distribution.

Results:

Evidence for the Role of β2-Microglobulin as a Novel Signaling and Mitogenic Factor Supporting the Growth of Human Prostate Cancer Cells:

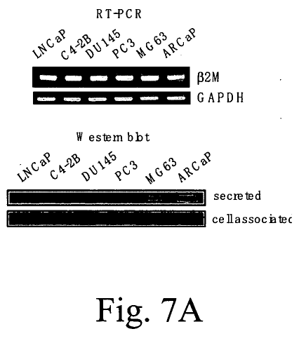

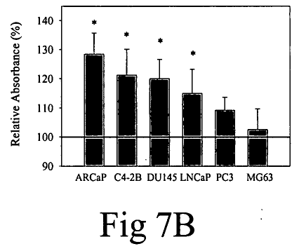

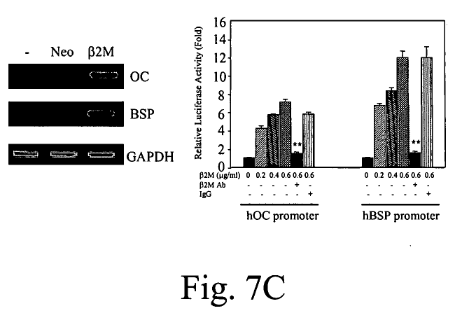

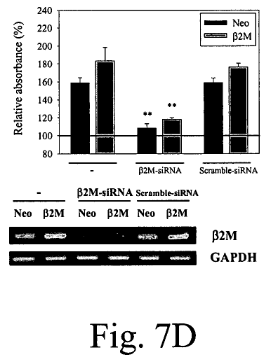

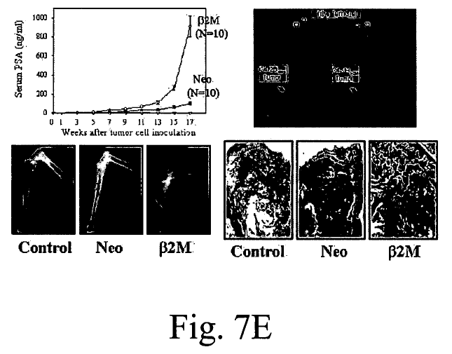

To isolate, purify and characterize soluble factors that contribute to osteomimicry and prostate cancer growth in bone, we monitored both OC and BSP promoter activity and their endogenous gene expression. We isolated a soluble protein factor with a molecular weight of 11.8 kDa, using ammonium sulfate precipitation, gel filtration, ion-exchange HPLC and N-terminal amino acid sequencing. This protein had complete identity with a known protein found in myeloma, β2-microglobulin (β2M), and appears to confer osteomimicry on prostate cancer cells. Although, as expected, the steady-state of β2M mRNA is similar among prostate cancer cell lines, secreted β2M protein correlated directly with the aggressiveness of prostate cancer cell lines in mice, with LNCAP as the least aggressive and ARCaP (109) as the most aggressive human prostate cancer cell line tested (FIG. 7A and (52)). Besides being a signaling molecule for osteomimicry, β2M was found to stimulate the growth of ALL human prostate cancer cell lines tested (FIG. 7B). The inventors studied 3 and 2 independent clones from β2M and neo, respectively, and found that C4-2B stably transfected with β2M clones showed marked enhancement of endogenous OC/BSP expression when compared to neo clones. Recombinant ⊖2M protein stimulated OC and BSP promoter activity, and this induction was selectively antagonized by anti-β2M antibody (FIG. 7C). β2M-mediated increases in prostate cancer cell growth in vitro can also be antagonized by the administration of anti-β2M but not by control anti-CREB antibody (data not shown). Likewise, β2M but not the control scramble-siRNA inhibited prostate cancer cell growth in vitro (FIG. 7D), suggesting that β2M is a potent mitogen. Remarkably, β2M-overexpressing C4-2B cells inoculated in mouse bone, but not subcutaneously, showed explosive tumor growth in hosts, greatly elevating serum PSA with mixed osteoblastic and osteolytic lesions (FIG. 7E). These results suggest that β2M-induced prostate tumor growth must be mediated by bone cells, due in part to the rich bone microenvironment resulting from increased bone turnover in the presence of prostate cancer cells. The inventors also observed a 2-fold increase of prostate cancer growth when C⁴-2B_{β2M} cells were injected subcutaneously in nude mice, compared to C⁴-²B_{Neo} cells. Tumors from the former were found to be more angiogenic and less necrotic (data not shown). The inventors' results expanded the previously known role of β2M as an essential factor for presenting and stabilizing MHC class I Ag on the cell surface of normal and cancer cells. The inventors' results reveal β2M as a mitogen for prostate cancer cell growth in vitro and tumor growth in vivo, particularly in bone.

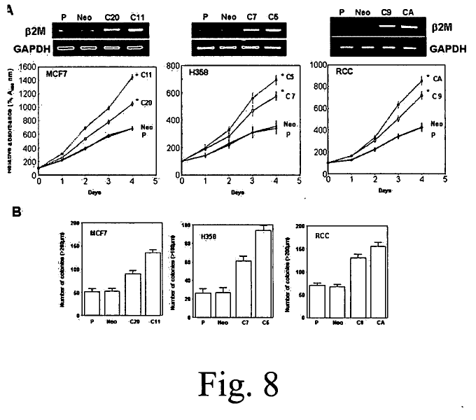

Because breast, lung and renal cancers are also known to metastasize to bone, we performed similar studies to test the possible roles of β2M in stimulating the growth of these human cancer cell lines in culture and in soft agars. FIG. 8 showed that the ability of human breast, lung and renal cancer growth on plastic dishes and in soft agars correlated positively with the levels of β2M expression by these cell lines. These results supported our hypothesis that β2M may be a mitogen that could play key roles in affecting cancer growth and dissemination to bone. Aim 1 will test this hypothesis using relevant animal models.

Discussion:

β2M, assayed by western blot and RIA, was found released by human prostate cancer cells in culture and in the urine of 101 patients with advanced prostate cancer (25). In this study, β2M levels in the conditioned media of human prostate cancer cell lines and primary cultures derived from distant metastasis as well as in the urine of patients with bone and visceral metastasis were higher than normal and also higher than those from patients with local/regional extensions of the disease. In a group of patients with bone metastasis (66 patients), high urine β2M was associated with significantly shortened survival. This group of investigators identified that in addition to 11.8 kDa β2M, a high molecular weight β2M immunoreactivity of approximately 38 kDa was found in the highly tumorigenic and bone-derived human prostate cancer cell line, PC-3, but not in the brain- and lymph node-derived DU-145 and LNCaP human prostate cancer cell lines. The approximately 38 kDa β2M was found in the urine of several prostate cancer patients but not healthy controls. The conditioned medium of a prostate small cell carcinoma cell line, NCl-H660, had high levels of chromogranin A and β2M, but prostate specific antigen was absent (25). These results support the inventors finding that the secretion of β2M by human prostate cancer cell lines correlated directly with their malignant potential in mice. In another study, β2M was shown to have direct mitogenic activity on prostate cancer cells (75). The inventors results support the concept that β2M has direct growth-promoting effects on prostate cancer cells, increases endothelial proliferation via increased production of VEGF (a β2M target gene), and improves the growth and survival of prostate cancer cells by inducing osteomimicry. Increased expression of OC (26-28), OPN (29, 30, 81), BSP (22, 29) and ON (32, 53) recruits osteoblasts, osteoclasts and marrow stromal cells and promotes the migration and invasiveness of tumor cells (82). β2M could have a dominant effect promoting tumor growth and bypassing any acquired immunity.

β2M-Mediated Growth of Prostate Cancer in Bone

Recent encouraging studies targeting the growth factor milieu and cancer cell interactions with cell surface receptors have interrupted inter- and intra-cellular signaling pathways, halted cancer growth and prolonged patient survival (83, 84). In breast cancer, erbB2/neu targeting with a herceptin antibody, Trastuzumab, either alone or with a vascular endothelial growth factor antibody, Avustin, resulted in improved patient survival with minimal host toxicity (85, 86). By interrupting erbB2/neu signaling, breast cancer cells became more sensitive to other growth factor (e.g. EGFR), hormone (e.g. an estrogen synthesis aromatase inhibitor) or cytotoxic therapy. When the tumors were treated with combinations of Trastusumab and Gefitinib (an EGFR Ab), Letrozole (an aromatase inhibitor) or Docetaxel (a cytotoxic chemotherapy), better tumor response was achieved than with a single agent. In lung cancer, targeting tumors with mutant EGFR with Gefitinib resulted in increased clinical response (87). A number of growth factor candidates, such as the IGF axis, EGFR, HER2/neu and PDGF-mediated prostate cancer growth pathways, could be attractive targets for therapeutic intervention in human prostate cancer (88, 89).

Thus, given the data so far, β2M appears to be an excellent new target for disrupting human prostate cancer growth in bone.

A systematic approach to interrupting β2M signaling is now being developed. The inventors have evidence that β2M is a mitogen and a signaling molecule supporting prostate cancer osteomimicry, critical to cancer growth and survival in bone. β2M antibody can abolish osteomimicry and β2M siRNA or ribozyme can effectively interrupt prostate cancer attachment to collagen IV, most likely by blockade of α1β1 and α2β1 integrins, inhibiting the proliferation of prostate cancer cells. The inventors now evaluate the effect of β2M antibody, β2M siRNA and ribozyme, and small molecules interrupting β2M-induced downstream signaling in prostate cancer cells and in prostate cancer xenograft models (see Examples 3 et seq below). The presence of β2M in human tissue and serum specimens obtained from prostate cancer patients will also be confirmed to assess the potential of β2M and its downstream signaling molecules as prognostic indicators and selective therapeutic targets (25). These approaches are extended to human breast, lung and renal cancer xenografts grown in mouse bone.

### Example 3

VEGF Axis in Prostate Cancer

Clinical data indicate that increased plasma levels of VEGF are associated with lymph node and skeletal metastases of prostate cancer (91-94). Radical prostatectomy lowers plasma VEGF levels in patients with prostate cancer, suggesting that the prostate gland itself or its signaling with the host may be a significant source of systemic VEGF (95). Moreover, bone microenvironment may modulate VEGF expression and secretion by cancer cells. Increased VEGF in the tumor microenvironment could alter the stromal cell-derived factor-i (SDF-1 or CXCL12)/CXCR4 axis which is known to regulate cancer cell homing to bone (96, 97). Increased VEGF can also modulate the expression of integrins αvβ3 and αvβ5 by LNCaP-C4-2 cells, affecting their attachment and migration on bone matrix proteins (98). Within the bone environment, De and coworkers (53) demonstrated that SPARC (secreted protein, acidic and rich in cysteine) engaged integrins which stimulated the growth of the C4-2 cells and resulted in further stimulation of VEGF production to support neoangiogenesis, favoring the development of metastatic prostate tumors in bone.

VEGF expression is regulated by oxygen tension through transcriptional activation by hypoxia-inducible factor 1 (HIF-1α) (99). In normoxia, the von Hippel-Lindau protein (pVHL) rapidly degrades HIF-1α by targeting it for the ubiquitin-proteasome pathway. In the absence of oxygen, HIF-1 binds to the hypoxia-response elements (HREs) within a promoter region of the VEGF gene, thereby activating VEGF expression (100). Other than hypoxia, growth factors, cytokines and hormones can regulate VEGF expression, mostly through the stabilization and/or increased expression of HIF-1α protein. These factors include EGF, TGF-α, TGF-β, keratinocyte growth factor, macrophage colony-stimulating factor (M-CSF) (101), IGF-1, FGF (102), PDGF, IL-1, IL-6 and IL-8 (103), growth hormone-releasing hormone (GHRH) (104), TSH, ACTH, 17β estradiol and androgen (105-108). In this experiement, we emphasize the coordinated VEGF regulation by β2M signaling via CREB and define the role of HIF1α in this process.

Consistent with these results, as shown below, the inventors found that β2M activated phospho-CREB and ATF-1 transcription factor in transfected prostate cancer cells to promote VEGF expression and signaling and support the growth of prostate tumors in mice (see below).

Results

β2M Activates the PKA-CREB-VEGF Axis in Human Prostate Cancer Cells:

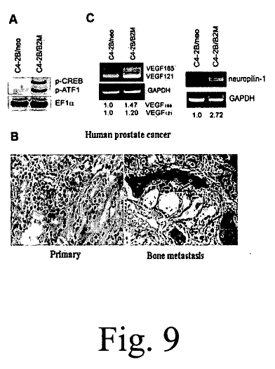

Phosphorylation of CREB/ATF1 in C4-2B cells is altered by ectopic expression of β2M (FIG. 9A), indicating that activation of the CREB pathway is a downstream event of β2M signaling. The inventors identified two important molecular pathways that may control the vital functions and survival of prostate cancer cells. First, PKA-CREB regulates VEGF expression by prostate cancer cells and the ingrowth of endothelium into the cancer tissues by promoting endothelial cell proliferation, motility, and vascular permeability. Second, PKA-CREB activation could mediate AR action to enhance the androgen-induced proliferative responses of prostate epithelial cells and their survival under suboptimal concentrations of androgen, as in castrated hosts. β2M could thus be a gate-keeper or switch that selects either AR or growth factor-mediated signaling for prostate cancer cells (see below). In the present sudy, the inventors focus on delineating the PKA-CREB-VEGF axis in prostate cancer cells to understand how this axis may be regulated by β2M signaling. Understanding this signaling pathway may help predict prostate cancer bone metastasis clinically and also provide a valuable therapeutic target for treating prostate cancer bone metastasis.

Aberrantly enhanced VEGF expression is associated with increased tumor growth and metastatic spread (110, 111). Human VEGF monomers have at least five different isoforms of 121, 145, 165, 189, and 206 amino acids. VEGF121 and VEGF 165 are the most abundant forms. VEGF165 is partially retained by cells while VEGF121 is completely released. VEGF165 is a more potent endothelial cell mitogen than VEGF121 (112, 113). Expression of both VEGF165 and VEGF121 is increased in β2M-overexpressing C4-2B cells (FIG. 9B). VEGF protein secretion was also found to be elevated in β2M-overexpressing (6.2% in total proteins of conditioned medium) versus neo- (3.9%) C4-2B cells. VEGF promoter contains CREs. Treatment with the PKA agonist Forskolin (10 μM) increased VEGF expression (data not shown). Transiently transfecting C4-2B cells with the expression plasmids encoding either wild type CREB (WT-CREB) or a mutated inactive form of CREB (K-CREB) increased VEGF expression in WT-CREB- but not K-CREB-expressing or control non-transfected cells, confirming that VEGF may be a target gene downstream from CREB signaling (data not shown).

VEGF binds with high affinity to the tyrosine kinase receptors VEGFR-1 (Flt-1) and R-2 (Flk-1m/KDR) expressed on the cell surface of endothelial cells (112). A third receptor, neuropilin-1 (NP-1), is primarily the coreceptor for VEGF165 (114). Though there are reports that some prostate cancer lines express Flt-1 and Flk-1 (115, 116), we could not detect signals of either receptor expression in C4-2B cells. However, NP-1 is found expressed in C4-2B cells (FIG. 9). Recent studies have shown that overexpression of both the VEGF165 isoform and NP-1 correlated with advanced prostate cancer and a high Gleason grade (117-119). Interestingly, NP-1 expression is higher in β2M-expressing C4-2B cells, suggesting a coordinated regulation of both VEGF and NP-1 expression in prostate cancer cells so that an autocrine loop is activated to support prostate cancer progression. In addition, VEGF could also have a paracrine function, regulating endothelial cell functions and subsequent neovascular sprouting (115). However, transient expression of wild type CREB or K-CREB (a mutant CREB) did not alter NP-1 mRNA expression in C4-2B cells, implying that β2M can regulate NP-1 transcription via certain CREB-independent pathway(s) (data not shown). The eventual goal will be to determine the mechanism by which an autocrine VEGF-NP-1 axis, activated by β2M signaling, supports prostate cancer cell growth, survival and migration in bone microenvironment.

Bioluminescence Imaging (BLI) of Prostate Cancer Metastasis in Bigenic and Immune-Compromised Mice:

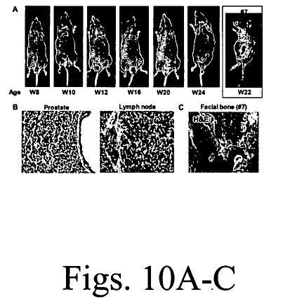

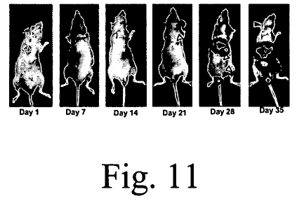

The inventors established the imaging of metastatic prostate cancer cells using a Xenogen CCD camera to detect bioluminescence in both transgenic animal models and luciferase (Luc) tagged human prostate cancer cells. The inventors successfully generated a supra-PSA driven Luc transgenic mouse (sPSA-Luc) (J Mol. Endocrinol. 2005, In Press), where homozygous sPSA-Luc male mice with FVB background crossed with a heterozygous TRAMP female mouse with C57BL/6]F1 (designated as TRAMP-Luc or a transgenic strain overexpressing T Ag in mouse prostate gland) were monitored at 2-week intervals for the appearance of metastatic prostate cancer using a Xenogen CCD camera between 8 and 24 weeks of age. Mice with visible tumor burdens displayed similar kinetic profiles of BLI. Light emission peaked in the lower abdomen, upper abdomen, chest and groin at 10 to 14 weeks, and then markedly decreased after week 16 (FIG. 10A). IHC staining of SV40-Tg confirmed the tumor distribution in imaged tissues including prostate gland and pelvic lymph node (FIG. 10B), and a low incidence of metastasis to jaw bone (FIG. 10C) in a TRAMP-Luc mouse at 18 weeks. Since no Luc-positive metastatic foci were detected in other organs, these results confirmed previous reports that AR is lost in TRAMP mouse prostate cancer cells upon disease progression (120). Another method of detecting metastatic prostate cancer cells in mice is the injection of Luc-tagged human prostate cancer cells via intracardiac route. 5×10⁴ PC3M-Luc cells were injected into the left ventricle and mice bearing human cancer cells were monitored by a Xenogen CCD camera. As shown in FIG. 11, a satisfactory injection of prostate cancer cells into the left ventricle was detectable within minutes by BLI (Day 1). In all mice, as early as 2 weeks after cell injection metastasis to various tissues was observed, including tumor metastases to liver, adrenal gland and left tibia (see arrows) (121).

### Example 4

Regression of Human Prostate Cancer Grown in Nude Mouse Femur or as Bone Powder Implants by Intralesional Administration of β2M siRNA Liposome Complex:

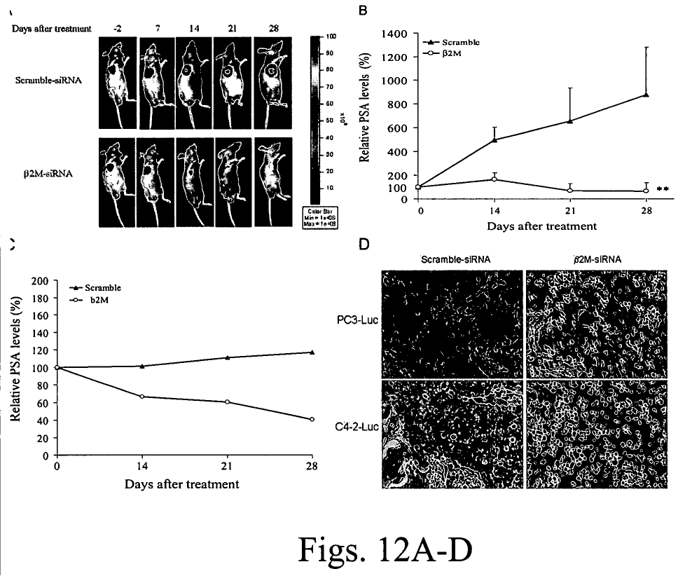

The inventors demonstrated effective siRNA lipsome delivery and αv integrin activity in mouse bone harboring human prostate cancer (121). Since β2M has been revealed as a mitogen and signal molecule in human prostate cancer cells, we questioned whether downregulating β2M using a siRNA approach would decrease the growth of pre-established prostate cancers in mouse bone. Two model systems were utilized. First, a bone powder model was used that was pioneered by Dr. Hari Reddi at UC Davis, where he and Charles Huggins in the 1970s found that an acellular rat bone powder preparation implanted subcutaneously in syngeneic or athymic animals recapitulated complete bone morphogenesis and cytodifferentiation, including the ability to form osteoclasts, osteoblasts, mineralized bone, bone marrow and red blood cells, by recruiting host cells (122, 123). Using this model, we found that PC3-Luc or C4-2-Luc grew in bone powder, forming highly interactive prostate cancer cell clusters with bone cells. Upon β2M siRNA-liposome treatment, prostate tumors were found to regress dramatically as assessed by Luc-imaging (PC3-Luc) or serum PSA (C4-2-Luc model) (FIGS. 12A and B). Similarly, siRNA-liposome was also found to be highly effective in antagonizing the growth of C4-2 prostate tumors in mouse tibia as revealed by serum PSA (FIG. 12C). These results were confirmed by tumor histomorphology in bone powder (FIG. 12D) where massive tumor cell death was observed. In this and other studies, liposome encapsulated with siRNA against αv integrin (121) or β2M produced no toxicity in host animals, as judged by the body weight of the mice and their level of physical activity. These results are consistent with the fact that β2M knockout mice develop mild level of autoimmune disease without major consequence on their organ development and postnatal growth. These promising pre-clinical studies prompted the inventors to use small-molecule β2M signal pathway interrupters and to evaluate the possible combined cytotoxic effects of β2M signal pathway interrupters (124) with other drugs approved for the treatment of hormone refractory human prostate cancer in mouse models of prostate cancer.

β2M Activation Alters Integrin Isotype Expression and Depresses AR-Mediated Signaling:

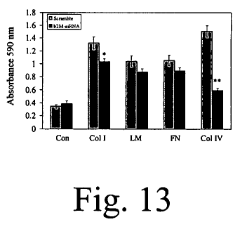

The inventors observed that when human prostate cancer cells were treated with β2M siRNA, cell attachment to ECM proteins was altered. We compared C4-2B cells, treated with either β2M siRNA or scramble siRNA, for their attachment to ECM proteins such as collagen I (Col I), laminin (LM), fibronectin (FN) and collagen IV (Col IV). Cell attachment to BSA coated wells served as an internal control (Con). FIG. 13 shows that the adhesion of C4-2B cells, transfected either with β2M or scramble siRNA, showed no significant difference in attachment to Col I, LM and FN. However, decreased attachment of C4-2B cells to the basement membrane Col IV, known to play a role in supporting prostate cancer growth and survival (124, 125), was demonstrated. These results suggest that β2M could affect α1β1 and α2β1 integrin expression on cell surfaces, thus revealing another potential consequence of β2M targeting in prostate cancer cells that ultimately will affect prostate cancer cell attachment to collagen matrices, growth and survival in bone (126).

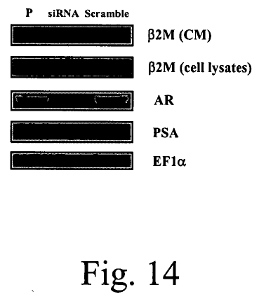

Another exciting consequence of targeting β2M signaling was the dramatic downregulation of AR and PSA expression in prostate cancer cells. FIG. 14 shows that AR and PSA protein expression, as assessed by western blots, was abolished in β2M-siRNA but not in parental and scramble-siRNA infected C4-2B cells. These results are not explained by cell selection since β2M-siRNA transfected cells were selected by antibiotic resistance and not single-cell cloned. Despite the marked decrease of AR in β2M-siRNA C4-2B cells, we found that β2M-siRNA-liposome treated prostate cancer, when maintained in mice, continued to synthesize and secrete PSA, suggesting an incomplete antagonism of AR. Possibly androgen or other growth factors and cytokines induce PSA production via residual AR in these cells. These results have important clinical implications: 1) downregulating β2M expression could effectively eliminate or attenuate AR signaling, removing one support for prostate cancer growth and survival in bone (31, 127); and 2) downregulating β2M expression could also decrease VEGF signaling (see above) and block AR from supporting prostate cancer cell survival (128-130), more effectively controlling prostate cancer growth in bone.

D. Research Design and Methods

Overall approaches and strategies: The inventors have established supra that β2M is a growth and intracellular signaling molecule contributing to cancer growth and survival in bone. The roles of β2M in prostate cancer bone metastasis are delineated as follows. A series of biologic studies will determine if overexpression of β2M increases the ability of human prostate, breast, lung and renal cancers to disseminate to bone or increases their growth as bone xenografts or in bone powder. Since β2M is an important signaling molecule, its role in regulating VEGF and NP-1 receptor gene expression downstream from β2M-mediated signaling in a CREB-dependent and independent manner are examined in detail. Taking advantage of the inventors success in building a transgenic mouse imaging model, the roles of β2M both as a growth and a signaling molecule in supporting prostate cancer cell growth and survival in mouse bone will be evaluated using non-invasive luciferase imaging to follow the growth and shrinkage of prostate tumors in bone. Transgenic mouse models are used to test the hypothesis that β2M and its signaling components may be new prognostic biomarkers that can predict prostate cancer bone metastasis and that this signaling pathway can be targeted to treat pre-established human prostate cancer in mouse bone. Finally, the inventors explore both β2M siRNA and other promising small molecules to block β2M-mediated signaling in prostate cancer cells in non-transgenic mouse models, determining their therapeutic effects on human prostate tumors either as single agents or in combination with other cytotoxic drugs, either subcutaneously as bone powder xenografts or directly as bone xenografts. The possibility is also tested that β2M-mediated signaling controls prostate cancer dissemination to bone. If so, targeting the β2M-mediated signaling pathway with β2M siRNA or small molecules could prevent prostate cancer bone metastasis and shrink previously established prostate tumors in bone.

These studies are based on promising preliminary investigations. a) β2M induces osteomimicry in human prostate cancer cells by activating the PKA signaling pathway (52). b) β2M increases VEGF, a VEGF receptor (NP-1), and AR expression and promotes the growth and survival of human prostate cancer cells by activating PKA and pCREB. c) β2M provokes potent growth-stimulatory effects on human prostate, breast, lung and renal cancer cells on plastic dishes and in soft agars in vitro and stimulates prostate tumor growth in mouse bone. d) By blocking β2M signaling using anti-β2M antibody, β2M siRNA or promising small molecules, the inventors greatly attenuated the growth of prostate cancer cells in vitro and tumor growth in bone. e) β2M activates pCREB and its downstream target genes, known to be involved in prostate cancer growth and survival. Representative examples of Putative cAMP/PKA downstream genes in prostate cancer cells include those listed in Table 1 below.

TABLE 1

| | |
|---|---|
| b2-Adrenergic receptor | STAT1 |
| VEGF | b-Catenin |
| STAT3 | G protein-coupled receptor 56 |
| Glutathione peroxisase, | IGFBP3 |
| PDGF b peptide | IGF2R |
| ADAM17 | Heat shock 70 kDa protein 4 |
| IL-8 receptor b | ADAM15 |
| b2M, IGF2 | Vimentin |
| PSA | IGFBP2 |
| Tumor protein D52 | IGF1 |
| CREB-like2 | Phosphodiesterase 3A. |

These results are consistent with published data from the inventors' laboratory (17, 26, 52, 131) (23) and others (28, 29, 32, 53, 81) strongly supporting the idea that aberrant expression and response to β2M increases the expression of highly restrictive osteoblast associated proteins, OC and BSP, in prostate cancer cells and tissues. This and other roles of β2M could decisively determine the fate of prostate cancer cells in bone. Since β2M in serum is higher in prostate cancer patients than age-matched normal controls (25) and serum β2M and its target gene VEGF were also found to be higher in men with clinically documented bone metastasis than with localized disease, the concept that β2M-mediated signaling could predict cancer bone metastasis using animal models will also be tested. Likewise, β2M-mediated signaling as a potential therapeutic target will be explored for the treatment of prostate cancer bone metastasis. These approaches, where appropriate, are then also applied to other human cancers with the goal of examining if the same molecular mechanism may also determine bone metastases of breast, lung and renal cancers. The inventors have already established stable β2M-expressing clones of human breast, lung and renal cancers (see FIG. 14) and are familiar with the use of different animal models to study the roles of β2M in promoting cancer growth in mouse bone and targeting β2M.

### Example 5

Purpose:

To determine if enhanced β2M expression and signaling increases human prostate, breast, lung and renal cancer bone metastasis.

Objectives and rationales:

Since bone metastases from prostate, lung, breast and renal cancers constitute the bulk of cancer deaths and cancer associated morbidity in the United States and world-wide, it is important to seek new molecular targets with prognostic and treatment value to reduce mortality and improve the quality of life and overall survival of patients with bone metastases. As outlined above in Example 2, there is a high likelihood that β2M-mediated signaling determines the growth and survival of human prostate cells in mouse bone and control the dissemination of solid tumors, such as prostate, breast, lung and renal cancers, to the skeleton. This hypothesis is tested in immune-compromised athymic mouse models with cancer cells inoculated either intracardiacally or intratibially. The inventors have extensive experience with these techniques for the assessment of tumor growth and dissemination in mice. In addition, the inventors have already established baseline information suggesting that like human prostate cancer cells, human breast, lung and renal cancer cells stably expressing β2M also had increased growth on plastic dishes and as soft agar colonies (FIG. 14 supra). Since tumor cell growth as soft agar colonies often reflects their ability to grow in mice, the inventors will test the hypothesis that increased β2M-mediated signaling in human prostate, breast, lung and renal cancers confers the ability to grow in mouse bone as xenografts or disseminate to mouse bone upon intracardiac tumor cell administration. If this proves to be true, the inventors propose to test whether β2M and its signaling components are prognostic biomarkers predictive of cancer bone metastases and are suitable new molecular targets that can be targeted to treat prostate cancer bone metastases as outlined below.

Methods:

β2M-(high, intermediate and low expressing clones) and neo-transfected human prostate (C4-2B), lung (H358), breast (MCF7) and renal (Rcc) cancer cells are selected and confirmed prior to use for the proposed studies. β2M mRNA and protein expression are determined by RT-PCR and ELISA, respectively with the established procedures in the laboratory. The growth and metastasis of these human prostate cancer cell lines in mice will be tested in three models (15 mice/group/tumor type): 1. direct injection of 1×10⁶ cells intrafemorally in mouse bone and the growth and survival of human prostate, lung, breast and renal cancers in mouse bone will be monitored bi-weekly and non-invasively by radiographic methods; 2. direct injection of 1×10⁶ cells into previously implanted bone powder for the evaluation of cancer cell growth and interaction in situ with newly formed bone; and 3. direct injection of 1×10⁶ cells into the left ventricle of mice and the metastatic spread of the cancer cells will be monitored as described above. The above described model 1 and 2 allow the inventors to assess the ability of cancer cells to grow, survive and colonize in bone microenvironment. The difference of model 1 and 2 is represented by adult and newly formed bone, respectively. The advantages of model 2 are that tumor growth in bone can be followed conveniently by luciferase imaging, multiple implantations of tumors are possible, and the tumor growth in bone powder can be easily assessed in a time-dependent manner. The advantage of model 1 is that the growth of tumor as bone xenografts may mimic closely cancer cell growth in bone. Model 3 has the advantage to study the multi-step nature of cancer growth and dissemination to bone although a longer latent period may be expected. If cancer growth and dissemination to bone is closely mimicked by β2M protein expression, the inventors will stably tag selected β2M expressed cancer cells with luciferase gene for a more detailed monitoring of time dependent spread of cancer cells to bone through intracardiac injection and monitoring cancer cells under a CCD camera as described (such as time course, location of the skeleton, tumor-stroma interaction). The inventors will confirm the growth, dissemination, and local bone osteoblastic and osteolytic reactions of cancer cells in bone by histopathology and IHC (e.g. expression of growth, apoptosis, osteoblastic and osteolytic biomarkers and differentiation-related genes).

Results:

A direct correlation is expected between the steady state level of β2M expression and the ability of cancer cells to colonize in bone (model 1 and 2). This will also reflect in the ability of high β2M expressing cancer cells to metastasize more frequently with short latent period to bone (model 3). Time course study of cancer cell growth in bone will reveal the infiltration of host inflammatory cells to bone and bone powder and this reaction is correlated also with the level of β2M expression in tissues. A concordance is expected between cancer growth in bone and the intensities of their radiographic and luciferase imaging. Histopathologic and IHC data will support both the osteoblastic and osteolytic lesions can be found in cancer cell growth in bone with relatively more osteoblastic reactions for prostate cancer cells and more osteolytic actions for breast, lung and renal cancer cells. The infiltrating lymphocytes, mast cells and macrophages in cancer specimens will be observed in each instance. A positive correlation between the status of these infiltrating cells, β2M expression in cancers or cancer metastases, and the growth and extent of cancer metastasis in mice will be observed.

### Example 6

Purpose:

To characterize and validate β2M-mediated downstream signaling components, in particular, VEGF signaling, in prostate cancer cells and surrounding cells in the microenvironment.

Objectives and Rationales:

Considerable evidence supports the idea of identifying men with potentially aggressive forms of prostate cancer and offering them early therapy rather than simply detecting the disease and offering general therapy. Clinically it is a rational goal to improve our understanding of prostate cancer biology, develop improved biomarkers and validate them in clinical samples. β2M is a mitogen with signal functions in addition to its well-known roles regulating host-acquired immune responses through the presentation of MHC to cell surfaces. The detailed molecular mechanism, however, by which β2M affects cancer cell growth and survival remains elusive. The objectives of this experiment are to: 1) to clarify the roles of β2M as a mitogen and signal molecule; 2) to identify and validate genes downstream from the β2M signaling pathway in cultured human prostate cancer cells and animal models; and 3) to determine the functional roles of β2M signaling in mediating the expression of VEGF and its receptor, NP-1, in human prostate cancer cells. The experiments are designed to test the involvement of CREB and HIF1α activation in modulating the VEGF axis for the growth and dissemination of human prostate cancer cells to mouse bone. By increasing the understanding of β2M mediated downstream signaling in prostate cancer cells, we hope to design more rational and effective targeting strategies to treat human prostate cancer bone metastasis. The hypothesis being tested is that β2M has a special role in promoting prostate cancer growth and colonization in bone through activation of the VEGF axis. The VEGF receptor, neuropilin 1 (NP-1), on the cancer cell surface may respond to VEGF secreted by cancer cells (autocrine loop) or cells in the cancer microenvironment such as endothelial cells, marrow stromal cells, platelets (which were found to sequester VEGF) and bone cells including osteoblasts and osteoclasts (paracrine loop), that could promote cancer cell growth, survival and migration to bone. Special emphases of this experiment are to: 1) identify the downstream β2M target genes in prostate cancer cells and cells in the cancer microenvironment; 2) explore the use of β2M and its downstream gene expression as prognostic biomarkers for prostate cancer bone metastasis; and 3) evaluate the mechanistic roles of β2M in the VEGF axis.

Methods:

To Determine and Validate the Downstream β2M Target Genes in Prostate Cancer Cells and the Cancer Microenvironment:

There are four potential pathways by which β2M can promote PKA activation in prostate cancer cells. 1) β2M can signal through the MHC class I complex, via yet-unidentified intracellular signaling components, to activate PKA. 2) β2M binds to its seven transmembrane G protein receptor or a putative seven transmembrane β2M receptor and induces intracellular cAMP accumulation and downstream PKA signaling including the activation of CREB. 3) β2M binds to a yet-unidentified β2M receptor and signals cells via PKA. 4) β2M functions directly as an intracellular signaling molecule by complexing with intracellular protein(s) without the prerequisite of membrane receptor-based activation. Further defining how β2M serves as a signaling and growth-promoting molecule will help identify useful new mechanism-based biomarkers to prognose bone metastasis and therapeutic response of prostate tumors grown in mice and design β2M-based targeting strategies to combat prostate cancer growth in the mouse skeleton.

1) Identification of β2M downstream target genes in established prostate cancer and bone stromal cell lines. This approach exploits data from Experiment 2 validating the expression of β2M target genes in cultured human prostate cancer cells. Where appropriate, the expression of β2M and its target genes in breast, lung and renal cancer cell lines and tumor specimens harvested from mice with cancer cells either grown in bone, bone powder or metastasized to the skeleton with defined levels of β2M expression will also be confirmed.

To identify and validate β2M target genes in prostate cancer cells, cells in the prostate cancer microenvironment, and in tissue specimens harvested from animal models, three specific studies are performed:

i) Searching for Global Gene Expression Differences Among Various C4-2 B Prostate Cancer Sublines that Express Different Levels of β2M, such as C4-2B_{Neo}, C4-2B_{β2M}, C4-2B_{siβ2m} (C4-2B Cells Stably Transfected with β2M siRNA) and Parental C4-2B and Validating These Genes in a Variety of Other Human Prostate Cancer Cell Lines.

The inventors have conducted a pilot microarray study using the currently established PO-1 Core Facility in collaboration with Dr. Peter Nelson at the Fred Hutchison Cancer Institute to compare global gene expression differences between C4-2B_{Neo}, C4-2B_{β2M}, and C4-2B_{siβ2M}. The C4-2B cell line was chosen because it has been shown to respond to β2M-induced growth and gene expression in culture and as xenografts in mouse hosts. This cell line is widely used by the prostate cancer research community since it represents an androgen-independent, AR/PSA positive and bone metastatic variant that compares well with parental LNCaP cells and patient specimens (47). Data analyzed by Dr. Jae Lee of the University of Virginia after background subtraction, correction of inter- and intra-assay variations and statistical analyses, revealed that at least 20 cAMP/PKA signaling downstream genes were regulated by β2M in C4-2B_{β2M} cells (Representative examples of Putative cAMP/PKA downstream genes in prostate cancer cells include for example, and not by way of limitation, b2-Adrenergic receptor, VEGF, STAT3, Glutathione peroxiase, PDGF b peptide, ADAM17, IL-8 receptor b, b2M, IGF2, PSA, Tumor protein D52, CREB-like2, STAT1, b-Catenin, G protein-coupled receptor 56, IGFBP3, IGF2R, Heat shock 70 kDa protein 4, ADAM15, Vimentin, IGFBP2, IGF1, and Phosphodiesterase 3A). The expression of these genes induced by β2M will be evaluated in other human prostate cancer cell lines (LNCaP, C4, C4-2, PC-3, DU145 and ARCaP). Cells treated with recombinant β2M (0.6 □g/ml) or vehicle will be harvested 24 h and 48 h after treatment. Selected gene expression in β2M-treated and untreated cells will be determined and compared at both mRNA and protein levels by quantitative RT-PCR (qRT-PCR) and western blotting analysis and/or ELISA, respectively. Each experiment will be carried out in triplicate.

ii) Searching for Genes Activated Upon β2M-Mediated cAMP/PKA Signaling in Cells in the Prostate Cancer Microenvironment.

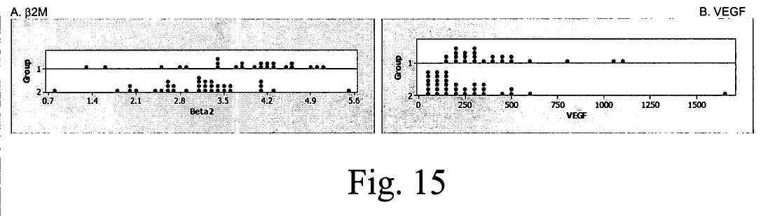

The hypothesis to be tested is that some β2M target genes may also be expressed by cells in the cancer microenvironment. It has been shown that human platelets and bone cells express OC and most of these cell types also express the secreted form of β2M. Defining β2M target gene expression in these cells will allow the inventors to understand the possible “field effects” of β2M that regulate not only cancer cells but also cells in the cancer microenvironment, and to exploit the potential of β2M as a target for both cancer cells and cancer-supporting cells in the microenvironment (e.g., β2M-mediated osteomimicry could control “reactive” stroma (43, 48, 132-135)). Pathway-focused microarray studies are conducted to define β2M-mediated cAMP/PKA signaling in human bone, endothelial, and inflammatory cells with a specific focus on CREB target gene expression. Since β2M has been shown to activate PKA and CREB, CREB target genes are probably activated upon the activation of PKA by β2M during human prostate cancer progression. As stated above and supported by the Results presented supra, upon β2M activation the inventors observed that serine 133 in CREB was phosphorylated both in vitro and in clinical prostate cancer bone metastasis specimens (FIG. 15). This activation, in reality, will result in only a fraction of 4,000 CREB target genes being actually activated in a context-dependent manner upon the stimulation of cAMP/PKA pathway. For this reason, Custom Oligo GEArray® focused DNA microarrays will be created, based on published work and the assessable database for CREB target genes (http://natural.salk.edu/CREB) and stromal cells (http://stromalcell.princeton.edu), through the service of Superarray, Bioscience Corporation (Frederick, Md.). Human bone stromal cells including osteoblasts (Kees II, a normal immortalized human osteoblast cell line), bone marrow stromal cells (HS-27A and HS-5), bone marrow associated normal endothelial cells (HBME, (136) and BMEC (137)), osteoclasts (FLG 29.1), and macrophages (MD) will be treated with recombinant β2M (0.6 □g/ml) or vehicle for 48 h and RNA will be extracted for microarray analysis according to the manufacturer's protocol. Each experiment is carried out in triplicate and the data are analyzed by a biostatistical core supported by a PO-1 grant. Once the differential gene expressions have been defined, a database of β2M-target genes for human prostate cancer and stromal cells in the tumor microenvironment willl be constructed.

iii) Validating β2M Target Genes in Experimental Animal Models

Although β2M-overexpressed C4-2B prostate cancer cells grow faster than neo-transfected prostate cancer cells subcutaneously, markedly enhanced growth of prostate cancer cells in bone was observed both at the gross morphologic and histomorphologic levels with elevated serum PSA. These results suggest β2M signaling may be different in prostate tumors grown in bone compared to subcutaneous sites, with human prostate cancer cells gaining particular growth advantages in bone. To address this question, the expression level of candidate genes in human prostate cancer cells grown in bone versus subcutaneously is compared. The gene expression profiles in prostate cancer cells grown in bone powder, which showed extensive interaction with the newly formed mouse bone surrounding cancer cells at the histopathologic and immunohistochemical (IHC) levels, is evaluated. Cancer and stromal cell components from excised tumors are isolated using the inventors previously established protocol (47). Each cell component is subjected to qRT-PCR and western blot analyses in triplicate to confirm the gene profile obtained from IHC in tumor specimens. Additional parameters such as cell proliferation and apoptosis will be assessed by Ki67 staining and TUNEL assays in tumor specimens harvested from mice.

2) Correlation of β2M and Its Target Gene Expression in Serum and Marrow Aspirate Specimens Isolated from Transgenic Mice and from Mice Inoculated with Human Prostate Cancer Cells.

In view of the fact that β32M signaling involves downstream activation of CREB, the following factors are evaluated for analysis for correlation with disease progression: β2M, OC, BSP, OPN, PSA, VEGF NP-1, vimentin and RANKL (the latter two are the epithelial to mesenchymal transition or EMT biomarkers the inventors identified in prostate cancer cell models, (138)) and integrin receptors α1β1 and α2β1. These markers will be expanded from those outlined above as the research is being conducted. Serum and bone marrow aspirates are obtained from transgenic and non-transgenic mice harboring prostate cancer either at primary or metastatic sites. The protein levels of these biomarkers are assessed quantitatively by ELISA and western blot assays using the appropriate kits from commercial sources. A preliminary trial was conducted using approved IRB protocols at the Department of Urology at Emory University School of Medicine (Dr. Fray Marshall) and Louisiana State University Cancer Center in New Orleans (Dr. Oliver Sartor) to assess if two biomarkers related to β2M signaling, β2M itself and one of its target genes, VEGF, can differentiate patients with or without prostate cancer bone metastases. FIG. 15 shows the results of a double blind study where the correlation of β2M and VEGF expression in the serum of prostate cancer patients with bone metastasis (n=24) or with prostate confined tumors (n=34) were dot-plotted. Based on the first set of statistical analyses, the inventors found significant correlation (p-value=0.013) between β2M and VEGF in prostate cancer patients with clinically diagnostic bone metastasis but not in patients with prostate-confined tumors. Also there was a significant differential expression of β2M (p-value=0.0015) and VEGF (p-value=0.0017) between cancer patients with bone metastasis and patients with localized tumors (FIG. 15), suggesting that β2M and one of its target genes, VEGF, are promising diagnostic biomarkers for prostate cancer bone metastasis. This is a small but encouraging trial assaying only a few biomarkers related to β2M downstream signaling. Before further clinical investigation, we intend to clarify the roles of β2M and its downstream signaling components as prognostic biomarkers for prostate cancer bone metastasis using mouse models bearing either mouse or human tumors (see below).

3) Assessment of VEGF Axis Downstream from β2M Signaling in Prostate Cancer Cells: A Mechanistic Study.

The data presented above in Experiement 3 suggest that phosphorylation of CREB/ATF1 in C4-2B cells is altered by ectopic expression of β2M (FIG. 9A). These results imply that activation of CREB pathway is a downstream event of β2M signaling. The inventors have thus identified an important molecular pathway in which the activation of PKA-CREB upregulates VEGF expression by prostate cancer cells resulting in increased prostate cancer growth through autocrine stimulation and the ingrowth of endothelium into the cancer tissues through paracrine stimulation. At the transcriptional level, the optimal control of the VEGF promoter requires a large transcriptional complex that is dynamically coordinated by HIF-1 and its coactivators including CBP/p300 (139), STAT3 (140, 141), AP-1 (142), SRC-1 (143) and Redox effector factor-1/apurinic/apyrimidinic endonuclease (Ref-1/Ape) (144, 145). Sp1 is also involved in Akt-mediated induction of VEGF expression through a HIF-1-independent mechanism (146). The inventors propose herein a novel signaling pathway by which β2M activates the PKA-CREB-VEGF axis to modulate VEGF expression and survival of prostate cancer cells (See FIG. 18). The inventors hypothesize that β2M-induced activation of PKA-CREB signaling recruits certain transcriptional factors, for example, and not by way of limitation, CBP/p300, or STAT3, or AR, to form an active transcriptional complex and bind to the VEGF promoter, thereby regulating VEGF expression and subsequently affecting prostate cancer cell proliferation via an autocrine loop consisting of VEGF and its co-receptor neuropilin-1 (NP-1).

To investigate the roles of β2M in affecting the VEGF axis, the following studies are conducted:

i. Investigate the Role of the β2M-PKA-CREB Axis in the Regulation of VEGF Expression in Prostate Cancer Cells.

This experiment tests the hypothesis that β2M induces transcriptional activation of VEGF, presumably through distinct mechanisms, i.e. HIF-1-dependent and HIF-1-independent signaling.

HIF-1-Dependent Pathway:

Cytoplasmic and nuclear proteins are collected from C4-2B cells pre-treated for various times with conditioned medium (CM) obtained from C4-2B β2M or C4-2B neo (control), or forskolin (positive control), H-89 (negative control), and DMSO (control). The inventors' previous studies have confirmed that the effects of C4-2B β2M are equivalent to recombinant β2M in OC and BSP promoter activation and these effects can be completely blocked by anti-β2M antibody. The inventors chose CM rather than recombinant β2M for this experiment to understand the biologic effects of secreted β2M on prostate cancer cells. Western blotting analysis will be performed to compare the nuclear presence of pCREB, CBP/p300 and HIF-1. These experiments will show whether increased CREB phosphorylation induced by β2M signaling promotes the formation of a transcriptional complex including the recruitment of HIF-1 and CBP into the cell nucleus.

The commercially available reporter construct (ATCC) pVEGF-KpnI contains a 2.65 kb VEGF promoter fragment. The VEGF luciferase reporter minus the HRE site will be constructed as previously described (100, 147). C4-2B cells will be transiently transfected with the full-length pVEGF-KpnI and the HRE minus luciferase reporters. The effects of conditioned medium from C4-2B β2M or C4-2B neo will be added to test the induction of VEGF transcriptional activity. Forskolin and H-89, expression vectors for wild-type CREB (WT-CREB), dominant-negative CREB (K-CREB) or empty vector (control) will also be introduced to examine their effects on VEGF luc reporter activity in a time- and dose-dependent manner. Parallel EMSA and supershift will be performed to assess the binding of the transcription factor complexes to VEGF promoter.

HIF-1-independent pathway: β2M signaling may induce VEGF transcription in HIF-1-independent manner. In this experiment, three possible models will be examined: a) β2M induces the direct binding of phosphorylated CREB protein to the putative CRE sites within VEGF promoter; b) β2M promotes formation of VEGF transcriptional complex by recruiting CBP/p300, SRC-1, or AR (in AR-positive cancer cells) into the nucleus; or c) increased nuclear presence of CBP/p300 facilitates nuclear translocation of STAT3 and induces the acetylation of STAT3 at lysine 685 in the C-terminal transcriptional activation domain of STAT3, which in turn stimulates the binding of STAT3 to VEGF promoter and transacting activity.

a) Determination of Whether CREB Directly Binds to the Putative CRE Sites in VEGF Promoter:

Computer analysis of VEGF promoter region reveals two putative CRE sites located upstream (-4940--4957, and -5625 --5632) of the transcriptional start site of the human VEGF gene. Therefore, activation of CREB signaling may directly regulate VEGF transcription by binding to these CRE sites in an HIF-1-independent manner. Currently available pVEGF-KpnI reporter does not contain the putative CRE sites. To gain a better understanding of β2M regulation of VEGF expression, the 5′-flanking region of human VEGF gene is cloned using PCR from FISH-confirmed clones in RPCI-1 library (http://bacpac.chori.org), and inserted into a pGL3-basic plasmid. Deletion mutants for the two putative CRE sites are constructed using the methods outlined in Example 1.

C4-2B cells will be transiently transfected with the full-length pGL3-VEGF and CRE minus luciferase reporters. The effects of conditioned medium from C4-2B β2M or C4-2B neo will be added to test the induction of VEGF transcription activity. Forskolin and H-89, expression vectors for wild-type CREB (WT-CREB), dominant-negative CREB (K-CREB) or empty vector (control) will also be introduced to examine their effects on VEGF reporters in a time- and dose-dependent manner.

To determine if CREB protein could directly bind to the putative CRE sites in the VEGF promoter in vivo, chromatin immunoprecipitation (ChIP) assays will be performed. This technique allows the detection of specific genomic DNA sequences associated with a particular transcription factor in intact cells. After the same treatment described above, nuclear proteins from C4-2B cells will be immunoprecipitated with a CREB antibody followed by PCR using oligonucleotide primers that amplify two ˜300-bp regions spanning the putative CRE sites within VEGF promoter.

b) Determination of Whether Phosphorylated CREB Indirectly Regulates VEGF Expression by Recruiting Certain Transcriptional Factors to VEGF Promoter:

This experiment will test the model that β2M-induced CREB phosphorylation facilitates nuclear translocation of CBP/p300, which in turn recruits other binding partners such as SRC-1 or AR to the promoter region of various genes, thus initiating VEGF transcription (148-150).

Cytoplasmic proteins and nuclear proteins are collected from C4-2B cells as described above. Western blotting analysis will be performed to compare the nuclear presence of pCREB, CBP/p300, SRC-1 and AR. CHIP assays will be performed to examine the binding of these transcriptional factors to the VEGF promoter.

c) Determination of Whether β2M Induces Both Nuclear Translocation and Acetylation of STAT3 to Activate VEGF Transcription:

Activated STAT3 forms dimers and translocates into the nucleus to induce VEGF. Recently it was shown that STAT3 is acetylated by its coactivator, CBP/p300, providing a novel regulatory mechanism for STAT3 activation (151). The possibility will be examined that β2M signaling induces VEGF expression via a STAT3-dependent pathway. Western blotting analysis will be performed to examine the presence of STAT3 in the nucleus isolated from C4-2B cells pre-treated by various conditions as described above. In vivo acetylation of STAT3 will be analyzed using a protocol described by Wang et al (151). Finally, EMSA and CHIP experiments will be performed to examine the binding of STAT3 to the STAT3-binding region in VEGF promoter using previously described methods (141, 151).

ii. Investigate VEGF-NP1 Signaling in Regulating Prostate Cancer Cell Proliferation and Migration.

VEGF appears to have several functions during bone formation and turnover. VEGF is critical for chondrocyte survival (152-154) and regulates cartilage angiogenesis and maturation during endochondral ossification (155, 156). In vivo and in vitro studies have revealed that VEGF may regulate bone formation through a direct effect on osteoblasts (156-158). VEGF also has a functional role for recruitment of osteoprogenitor cells in the course of endochondral bone formation or remodeling, a dose-dependent chemoattractive effect dependent on VEGFR-1, and possibly, neuropilins (112). Collectively this suggests that VEGF is involved in the regulation of the bone microenvironment, which is important in determining the tropism of cancer cells during bone metastasis.

VEGF binds with high affinity to the tyrosine kinase receptors VEGFR-1 (Flt-1) and R-2 (Flk-1/KDR) expressed on the cell surface of endothelial cells (112) A third receptor, NP-1, is primarily the co-receptor for VEGF165 (114). VEGF receptors are expressed by various tumor cells including prostate cancer (115). It is therefore possible that VEGF acts as a multifunctional cytokine in tumor progression, capable of promoting angiogenesis and autocrine regulation of tumor growth. Reportedly, some prostate cancer cell lines, including C4-2B, express Flt-1 and Flk-1 (115, 116). The inventors' data supra show that NP-1 is expressed by C4-2B cells. Interestingly, NP-1 expression is higher in β2M-expressing C4-2B cells, suggesting the possible role of β2M signaling which increases NP-1 expression.

NP-1 is a type 1 membrane protein that binds the axon guidance factors belonging to the class-3 semaphorin family. The importance of NP-1 as a functional receptor in neurobiology is well established, but its role in tumor biology has only recently been recognized. In endothelial cells, NP-1 is a co-receptor for VEGF and regulates VEGFR-2-dependent angiogenesis. Gene-targeting studies documenting embryonic lethality in NP-1 null mice suggest a critical role for NP-1 in vascular development. In mature endothelial cells, NP-1 has a dual role as an enhancer of VEGF activity, by regulating VEGFR-2 activity and as a mediator of endothelial cell adhesion to extracellular matrix proteins independently of VEGFR-2. NP-1 alone can also mediate endothelial cell migration and adhesion to extracellular matrix proteins independent of VEGFR-2. Several distinct signaling pathways, including PI-3 kinase, RhoA, and G proteins are involved in these processes (159-161). A recent study showed that NP-1 binds a subset of heparin-binding proteins (such as FGF-2, FGF-4 and HGF) with higher affinity than VEGF, and augments the growth stimulatory activity of FGF-2 on HUVECs (162). Given the importance of these heparin-binding growth factors in tumor progression and metastasis (17, 163), it is well worth investigating the role of NP-1 in tumor-microenvironment interaction during bone metastasis.

Given the importance of VEGF and NP-1 as a potential autocrine loop in prostate cancer bone metastasis, a coordinative regulation of both VEGF and NP-1 expression in prostate cancer cells will be investigated.

a) VEGF and NP-1 in the Regulation of Prostate Cancer Cell Proliferation and Migration

The possible involvement of autocrine signaling in prostate cancer cell proliferation and migration is be examined in the following in vitro experiments: 1) addition of neutralizing antibodies to VEGF and NP-1 to C4-2B cells; 2) addition of recombinant VEGF and ectopic expression of human NP-1 in C4-2B cells; 3) treatment of C4-2B cells with siRNA for VEGF and NP-1 (Santa Cruz). These data will elucidate whether VEGF can regulate C4-2B cell proliferation and migration in an NP-1-dependent manner.

b) Mechanisms Underlying VEGF-NP-1 Regulation of Prostate Cancer Progression

It has been shown that NP-1 is associated with activation of various intracellular signaling pathways, including PI-3K/Akt, MAPK (145, 164), G protein and small GTPase proteins (RhoA, Rac1). Western blotting analysis is performed to examine the activation of these pathways in C4-2B cells treated with recombinant VEGF, neutralizing antibodies to VEGF and NP-1, overexpressing NP-1, or treated with siRNAs to VEGF and NP-1. Inhibitors to these pathways, i.e., LY294002 (PI-3 kinase), MEK-1 (PD98059 or U0126), and pertussis toxin (G proteins), will be used to treat C4-2B cells to examine their effects on cell proliferation and migration induced by recombinant VEGF and/or ectopic expression of NP-1.

c) VEGF as a Modulator for NP-1-Dependent Prostate Cancer Cell Proliferation and Migration

Previous studies on the nervous system have shown that the axon guidance molecules are involved in the regulation of neural cell proliferation and migration (165, 166). Sema3A, a soluble member of the semaphoring superfamily, acts as a repellent guidance cue for migrating neural progenitor cells and induces apoptosis in an NP-1-dependent manner (167, 168). VEGF165 functions as an antagonist for sema3A and inhibits the binding of sema3A to NP-1 and vice versa (167, 169, 170). It is therefore possible that VEGF regulates the migration of cancer cells via direct interaction with NP-1 or suppression of sema3A-NP-1 interaction (171-173). In fact, it has been shown that a balance between two opposing autocrine loops of VEGF-NP-1 and Sema3A-NP-1 regulates the chemotaxis of breast carcinoma cells (174). It is interesting to note that Sema3A-NP-1 signaling is present in bone and seems to precede or coincide at the temporal and spatial level with the invasion of bone by blood vessels and nerve fibers (175). Another semaphoring 3 member, Sema3B, has been recently shown to induce apoptosis in lung and breast cancer, whereas VEGF165 antagonizes this effect (176).

The hypothesis that VEGF may antagonize the inhibitory effects of Sema3A on cancer cell proliferation and migration by competitive binding to NP-1 will be evaluated. Endogenous VEGF and Sema3 (or Sema 3B) compete for NP-1 binding, and the ratio of the concentration of these proteins in prostate cancer cells is a critical determinant of their chemotactic rate. The relative amounts of Sema3A (or Sema3B) and VEGF protein will be measured in various prostate cancer cell lines with different metastatic abilities using RT-PCR and western blotting analysis. These data will provide a possible index for determining metastatic potentials of prostate cancer cells. Next, C4-2B cells overexpressing NP-1 or treated with siRNA to NP-1 will be used to test the effects of the balance of recombinant Sema3A vs. VEGF on cell proliferation and migration. Intracellular signaling will be studied using the methods described above. IHC staining will be conducted in tissues harvested from mice to determine if β2M signaling affects the levels of these protein expressions.

Results:

A positive correlation is expected between β2M signaling and the status of activation of PKA, pCREB, osteomimicry, and the activation of VEGF and AR-related growth and survival signaling pathways. An autocrine loop of VEGF-NP-1 activated by β2M signaling may be demonstrated in LNCaP lineage cells whereas paracrine loop of VEGF-VEGFR may be activated by β2M signaling in cancer and endothelial interaction model. Additional biomarkers associated with β2M signaling, revealed by microarray (sss Table 1), and related to the VEGF-NP-1 pathway will be confirmed in animal models. Upon activation of the β2M signaling pathway, we expect an elevation of the serum markers reflecting increased osteomimicry (OC, BSP, OPN), activated VEGF-NP-1 (increased levels of agonists and decreased their competitors, Sema3A and 3B) and increased AR signaling and EMT (PSA, VEGF are AR target genes whereas vimentin and RANKL are EMT biomarkers), gained integrin receptors (α1β1 and α2β1) and other targeted molecules (see Table 1). Serum or bone marrow specimens harvested from mice may reveal correlative values of these markers in predicting prostate cancer skeletal metastases at the time before radiographic or PSA prediction of prostate cancer bone metastasis. Tissue distribution and subcellular localization of the markers could yield significant information on the activation status of signaling molecules (e.g. pCREB and its recruited protein complexes in the cell nucleus), thus increasing the predictive values of the biomarkers identified. Increased β2M signaling and its downstream activity could indicate poor prognosis. While overlapping of β2M and VEGF has been observed in serum samples obtained from men with and without bone metastasis (FIG. 15), using animal models described below it will be possible to discern the possibility of some biomarkers downstream from β2M signaling may be able to differentiate cancer grown in bone or at visceral organs before radiographic evidence of bone metastasis (i.e. predicting tumors in bone when they are very small in size). If this is true, this would have remarkable value in clinical applications since currently there is no serum biomarkers that can be used to predict patients with small foci of bone metastasis.

A convergence is also expected between β2M signaling, the activation of CREB downstream target genes including VEGF, and AR signaling. In AR-negative prostate cancer cell lines (PC3/DU145, AR negative), genes that are related to CREB, but not AR downstream genes, will be correlated with β2M activation. β2M-induced growth of prostate cancer in bone, but not in subcutaneous space, is related exquisitely with the interaction between β2M-mediated signaling and cells in bone marrow, including osteoblasts, osteoclasts, marrow stromal cells, endothelial cells and inflammatory cells. It is important to understand the molecular basis of bone metastasis, a dominant phenotype of clinical human prostate cancer. There may be a “vicious cycle” between the signaling components mediated by altered growth factor, ECM milieu and increased bone turnover (e.g. through RANKL-RANK interaction) in response to β2M that favors prostate cancer bone colonization.

### Example 7

Purpose:

To establish animal models with altered β2M status for the evaluation of β2M effects on prostate cancer growth and metastasis to bone and to employ this model as a tool to screen for compounds that will block β2M signaling in prostate cancer skeletal metastases.

Objectives:

Since no transgenic mouse models yield an acceptable incidence of bone metastasis for studying the biology and targeting of prostate cancer, the objectives of this experiment are to develop a highly effective bone metastasis mouse model and use this model to test agents targeting prostate cancer metastasis to bone. This study will also confirm that β2M is a mitogen and a signaling molecule and will allow us to investigate the molecular basis of the osteomimicry observed in prostate cancer bone metastasis in live mice.

While β2M is expressed by many organs in the body, β2M is contributed locally primarily by the circulating lymphocytes (60). In the bone microenvironment, β2 production is assessed by ELISA to be produced by macrophage, osteoblasts, and marrow stromal cells. There appears to be significant homology in β2M between human and mouse (80% homology) and mouse β2M may have similar biological functions to human β2M, as supported by the observation that human bone marrow engrafts in mouse bone were greatly attenuated in strains that had β2M knockout by genetic mutation of mouse β2M genes (177). Since β2M-overexpressed human prostate cancer cells grow explosively in bone, and grow favorably subcutaneously (see preliminary results), we will test the effect of mouse host-derived β2M in promoting human prostate tumor growth in live mice (see Table 2 below).

TABLE 2
Two sets of animal models will be proposed for the study of b2M in
prostate cancer bone metastasis in vivo.

| | | |
|---|---|---|
| Strain | b2M status | Tumor cells |

Model I: Immune-compromised Mice

| | | |
|---|---|---|
| Col I-h b2M | human b2M overexpressed | Implanted C4-2-Luc and |
| | in bone | PC3-Luc cells |
| Balb/c nu/nu | normal level of mouse b2M | Implanted C4-2-Luc and |
| | | PC3-Luc cells |
| NOD/β2M | null | Implanted C4-2-Luc and |
| Null^{SCID} | | PC3-Luc cells |

Model II: Immune-competent Mice

| | | |
|---|---|---|
| PTEN^{null}-Luc | normal level of mouse b2M | Spontaneous prostate |
| | | cancer development with |
| | | imaging capacity |
| b2M-Luc | mouse b2M overexpressed | Normal prostate with |
| | in bone | imaging capacity |
| b2M-PTEN^{null}- | mouse b2M overexpressed | Increased prostate cancer |
| Luc | in bone | development with imaging |
| | | capacity |

Two experiments will be conducted. First, β2M expression will be increased in a transgenic mouse model by delivering β2M to bone using a collagen 1 promoter obtained from Dr. B. deCrombrugge (UT MD Anderson Cancer Center). This conditionally β2M-overexpressing mouse strain will be used as a host for testing the idea that β2M supports prostate cancer bone colonization in transgenic mice. To accomplish this goal, β2M-overexpressing founder mice will be crossed with PTEN knockout mice created by Dr. Hong Wu of UCLA (178) and the incidence of bone metastasis will be evaluated. PTEN knockout has advantages over TRAMP mice; it develops both primary and metastatic adenocarcinoma, and the tumor cells retain AR (178). If this cross yields promising findings defining the roles of β2M in prostate cancer bone metastasis, the investigation will be expanded into the use of supra-PSA-luciferase transgenic mice created in the inventors laboratory to generate a bigenic metastatic model with imaging capability. Luciferase imaging can visualize bone and is a convenient non-invasive approach for the detection of prostate cancer bone metastases.

The direct effects of μ2M in promoting human prostate tumor growth in immune-compromised mice are tested by generating a transgenic human recombinant β2M conditionally overexpressed mouse strain with SCID background to test the hypothesis that human prostate cancer growth and metastasis to bone may be facilitated by overexpression of human recombinant β2M in mouse bone. The growth and metastasis of the human prostate cancer cells in these mice will be investigated by directly injecting human prostate cancer cells in mouse tibia or by inoculating prostate cancer cells either orthotopically or intracardiacally using methods established previously by the inventors laboratory (179). In the experimental design, caution must be exercised when using β2M knockout mice as hosts for this study to test if the growth of human prostate tumors in this mouse strain (which is available commercially) may be compromised, because β2M has two confounding activities, one to stimulate tumor cell growth and survival in bone and the other to boost host immune activity due to enhanced MHC presentation. Using β2M knockout mice as hosts might result in either decreased or increased prostate tumor growth in mice depending on the balance of these two actions of β2M.

Methods:

This experiment establishes conditionally β2M-overexpressing transgenic mouse strains with overexpression of human or mouse β2M in the bone. To generate conditionally β2M-overexpressing transgenic mouse strains, the fragment of mouse collagen I promoter-human β2M expression cassette is purified from its respective plasmid and then microinjected into the pronuclei of fertilized eggs of Balb c/nu/nu males and Balb c/nu/+ female mice (Jackson Laboratory). The offspring from this mating are subjected to genotyping (β2M+) and phenotyping (nude and serum β2M+) and then used as hosts for the growth of human prostate tumors. After establishment of founders, genotyping and phenotyping of offspring will be accomplished by PCR of purified tail DNA and serum β2M ELISA, respectively. At least three transgenic lines are established to avoid the non-specific effects cased by the position of the transgenes inserted. Once these lines are established, a determination is then be made if host β2M has a role in determining the ability of human prostate cancer cells to metastasize to bone and soft tissues by inoculating two human prostate cancer cell lines stably transfected with luciferase gene (2×10⁶ PC-3Luc or C4-2B-Luc cells per mice) in hosts (12 mice/group) with different β2M backgrounds, including hosts expressing recombinant human β2M, mouse β2M or lacking or expressing non-functional β2M (such as the commercially available NOD/β2M Null^{SCID} strain). The metastatic potential of these human cells for bone is determined via two routes of tumor cell inoculation, intracardiac (which is the conventional route for generating bone and visceral organ metastases) versus orthotopic (which is the conventional route of generating lymph node, bone and visceral organ metastases), to determine the latency and incidence of tumor formation at different metastatic organ sites. Tumor cell growth in mice is followed by non-invasive luciferase imaging under a CCD camera and by serum PSA (for C4-2B tumors only). Possible correlation of β2M expression in host tissues and prostate cancer metastases at various anatomical sites is then determined.

The same procedure will then be employed to generate recombinant mouse β2M-overexpressing mice in an immune-competent B6 background strain. The resultant mouse strain is characterized and the high β2M expressing founder mice are selected and crossed with the PTEN knockout strain (PTEN^{null}) purchased from the Jackson Laboratory (Bar Harbor, Me.) to generate a bigenic β2M-PTEN^{null 1} strain. PTEN knockout mice were shown to harbor prostate cancer soft tissue metastases with a low incidence of bone metastasis. The tumor model could be superior to the TRAMP model for our purposes because the prostate adenocarcinoma developed are AR positive (TRAMP mice developed largely AR-negative tumors with neuroendocrine phenotype as found in the latter stages of tumor progression). The β2M-PTEN^{null} bigenic strain will be further crossed with the inventors' supra PSA promoter-luciferase mouse strain (sPSA-Luc) to generate a new prostate cancer metastatic mouse model (β2M- PTEN^{null}-Luc) with imaging capability. A PTEN^{null}-Luc strain generated by crossing the PTEN^{null} to sPSA-Luc strain is used as control group. These novel mouse strains could be invaluable laboratory tools to study the biology and molecular targeting of prostate cancer bone and visceral organ metastases. Twelve β2M- PTEN^{null}-Luc males and an equal number of PTEN^{null}-Luc males is followed for tumor development and distant metastasis by measuring bioluminescence at 1-week intervals from 8 to 36 weeks of age. Mouse serum (25 μl) is obtained at monthly intervals to assay for β2M, OC, VEGF, and RANKL (5 μl/assay) and data will be correlated with onset and extent of bone metastasis. All mice are sacrificed at 35 weeks and bioluminescence distribution in tissues will be confirmed by histopathological analysis of cancer cells. Serum is obtained and subjected to more extensive tests for other potential biomarkers associated with osteomimicry and β2M signaling (see Table 1). The latency and the incidence of tumor formation at different metastatic organ sites will be compared between the β2M- PTEN^{null}-Luc and PTEN^{null}-Luc groups. These mice are employed for the assessment of β2M interrupters.

Results:

It is expected that prostate cancer cells will metastasize to sites with increasing β2M accumulation. A lower incidence of prostate cancer metastasis to bone and soft tissues may be expected in mice with non-functional β2M mutant (or β2M null) expression. Tumor metastases to different anatomical sites may differ depending on the route of tumor cell inoculation (179). The extent of metastases can be determined by the Xenogen machine under a CCD camera. A positive correlation may be observed between β2M expression and cancer metastases. Further, animals harboring prostate cancer bone metastasis could express high levels of serum surrogate biomarkers related to the β2M signaling pathway; overexpression of β2M may increase osteomimicry, allow prostate cancer adhesion to bone-like matrix proteins, OC, OPN and BSP and collagen IV integrin receptors, and overexpression by the host cells of VEGF and AR and their downstream target genes including growth and survival related genes and EMT associated genes (Table 1) that will eventually increase tumor cell growth and survival in bone. A series of assays for mouse genes will be developed. An increased incidence of bone and soft tissue metastases is expected in the bigenic mouse strain overexpressing β2M with a PTEN knockout background. Luciferase imaging is expected to be especially helpful in the detection of prostate cancer bone and soft tissue metastases, and such metastases can be confirmed by histomorphology, IHC and biomarkers (serum β2M, OC, VEGF, RANKL) associated with activation by β2M signaling in mice. Though PTEN knockout mice have a short lifespan (˜20 months), this strain of mouse has been invaluable for observing prostate cancer growth and distant metastases. It is possible that bigenic mice could also have shorter than normal lifespan, thus narrowing the window of opportunity for observing tumor metastases. If so, alternative approaches will be explored to determine the role of β2M in prostate cancer bone metastasis, delivering recombinant β2M through the implantation of prostate cancer cells stably expressing β2M. IHC evidence suggests that such approaches result in human β2M accumulation in mouse bone. This animal model can thus be used to evaluate the bone-homing potential of human prostate cancer cells injected either orthotopically or intracardiacally (17).

Since other bone and soft tissue derived markers have been associated with cancer metastasis (including SDF-1, CRCX4 (180), expression of sonic hedgehog (181), Wnt signaling pathways (182), and the presence of abundant heparin bound growth factors (183, 184)), the expression of these potential markers by IHC, and ELISA in serum specimens harvested from mice will alternatively be evaluated before drawing correlations between β2M and the expression of osteomimicry related and unrelated factors that are known to support prostate cancer growth and metastases to bone and visceral organs.

Because conditionally overexpressed β2M was restricted to bone, increase MHC presentation in mouse prostate tumor is not expected. The overexpression of β2M by mouse bone will potentially enhance an autoimmune reaction against host antigens but probably will not increase host antitumor immunity. Increased β2M expression in mouse bone could support the growth of prostate tumor with PTEN knockout background through induced osteomimicry which promotes cell growth, attachment to extracellular substratum and survival in bone. This could result in efficient prostate cancer bone metastasis. Cancer cells are often deficient in MHC class I presentation despite elevated serum β2M and thus the tight coupling between β2M and MHC shown in normal cells may be lost in cancer cells.

### Example 8

Purpose:

To test β2M targeting by the use of β2M sequence-specific siRNA or ribozyme and small molecules that can interfere with β2M-mediated cell signaling.

β2M is available in circulation and also produced locally. Preliminary results suggest that locally produced, rather than circulating β2M is responsible for prostate cancer growth in bone. This conclusion is derived as follows. β2M is expressed prevalently in prostate cancer cells and the surrounding inflammatory lymphocytes and bone- and prostate-derived stromal cells. In response to β2M, prostate cancer cells in primary or metastatic sites exhibit osteomimicry by expressing highly restricted bone proteins such as OC, BSP, OPN and RANKL, normally expressed by osteoblasts (17). While β2M immunostaining was strong in prostate cancer bone metastasis, normal bone marrow without prostate cancer cells failed to reveal intense β2M immunostaining (data not shown). These results imply that β2M local expression (and accumulation) surrounding cancer cells contributes to the osteomimicry of prostate cancer cells in bone. Further, locally produced β2M and its induced osteomimicry may be responsible for prostate cancer growth and survival at metastatic bone sites. The inventors' results support this concept. By knockdown of β2M, pre-existing prostate cancer cells grown as xenografts in bone powder or as femur implants were markedly depressed as assessed by luciferase imaging and serum PSA and showed massive cell death as evaluated by histopathology. This observation will be extended by comparing the efficacy of gene-based and small molecule-based β2M interrupters or interfering compounds.

The search for small molecules that specifically targets β2M signaling is initially focused on G protein-coupled receptor (GPCR)-specific signaling. These receptors are the target of >50% of the current therapeutic agents on the market, including more than a quarter of the 100 top-selling drugs. GPCR Bradykinin (BK) antagonists as well as their bisphosphonate (BP) conjugate mimetics may be screened using the methods described supra for the inhibition of β2M-mediated prostate cancer cell growth.

Significant progress searching for gene-based targeting has been made. In particular, β2M siRNA has been delivered successfully to pre-existing prostate cancers and induced massive tumor cell death using intra-lesional injection of liposomes mixed with β2M siRNA. This form of gene therapy can be improved by using intravenous (IV) β2M siRNA complex with liposome with the expression of β2M siRNA or ribozyme controlled by tissue-specific and tumor-restrictive promoters (185), such as OC (expressed in both cancer and bone cells) or supra PSA (expressed by prostate cancer cells). Similar approaches have been used successfully by others and are currently being pursued for a breast cancer clinical trial at UTMD Anderson Center (Mien-Chie Hung, personal communication). Liposome delivery through IV infusion has been shown to be quite effective in other studies (186-188). Improved liposome targeting of tumor cells can be achieved by antibody or other targeting ligands such as RGD peptide, folic acid, PSMA, growth factors, cytokines, or aptamers, conjugated to the liposomes (189).

### Example 9

β2M Knockout Tumors:

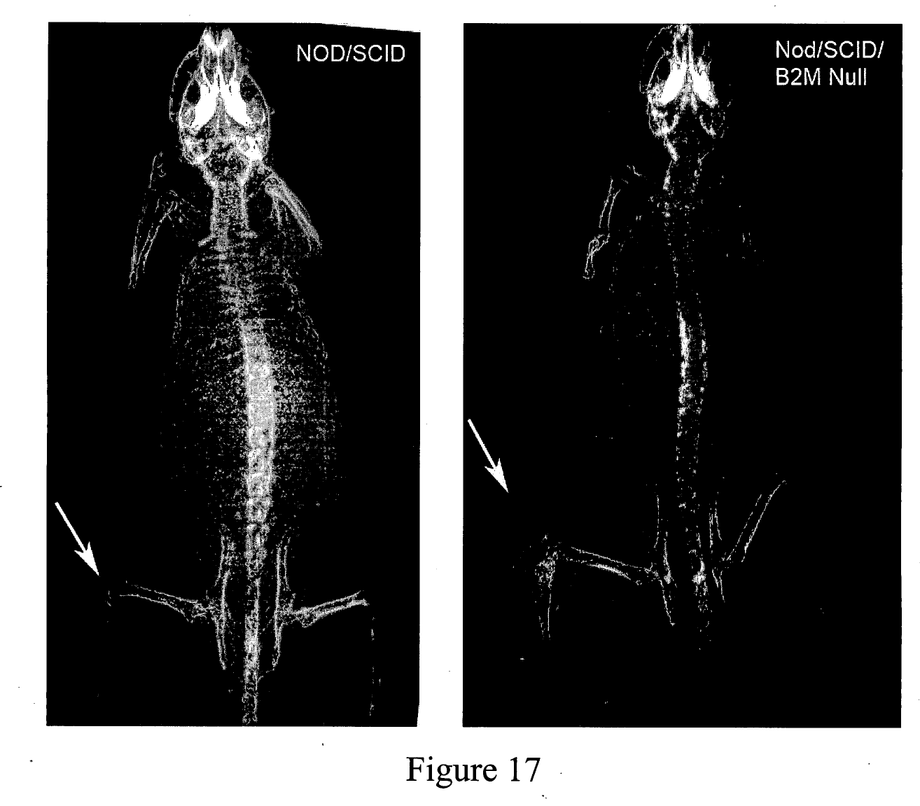

This experiment demonstrates that human prostate cancer cells grew far better in β2M knockout mice than that of the wild type mice. In this expeiment, mice inoculated with one million human prostate cancer cells in bone were found to develop osteoblastic/osteolytic mixed tumors in β2M knockout SCID mice in less than 2 months (FIG. 17B). Similar injection of human prostate cancer cells in bone in control wild-type SCID mice only developed very small tumors (FIG. 17A). These results suggest that by antagonizing osteomimicry through β2M knockdown of the hosts will increase the colonization of foreign cells including but not limited to bone marrow and stem cell transfer to the recepient hosts.

Since mice with greatly decreased osteomimicry as a result of β2M knockout survived and with minimal manifestation of any serious disease, a transient decrease of osteomimicry will result in minimal toxicities to the mouse host. The inventors suggest that since cancer and benign cells may dependent upon osteomimicry to maintain their calcification and mineralization potential, decreased osteomimicry could cause massive cell death and decreased growth potential of these cells.

Throughout this application various publications and patents are referenced. The disclosures of these publications and patents in their entireties are hereby incorporated by reference into this application in order to more fully describe the state of the art to which this invention pertains.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

APPENDIX A

| | | b2M Target Genes | |
|---|---|---|---|
| b2M Target Genes (increased) | Function | (decreased) | Function |
| b2-Adrenergic receptor | cell growth | IGF2R | cell growth |
| VEGF | cell growth, cell | Heat shock 70 kDa | |
| | cycle | protein 4 | |
| STAT3 | cell mobility | ADAM15 | cell adhsion |
| Glutathione peroxiase | oxidative stress | Vimentin | EMT marker |
| PDGF b peptide | | IGFBP2 | cell growth |
| ADAM17 | | IGF1 | cell growth |
| IL-8 receptor b | cell growth, mobility | Phosphodiesterase 3A | |
| b2M | cell growth, survival | | |
| IGF2 | cell growth | | |
| PSA | prostate cancer | | |
| | progression | | |
| Tumor protein D52 | | | |
| CREB-like2 | cell growth, survival | | |
| STAT1 | apoptosis | | |
| b-Catenin | cell adhesion | | |
| G protein-coupled | cell growth, survival | | |
| receptor 56 | | | |
| IGFBP3 | cell growth | | |

APPENDIX B
A Partial List of GPCRs Antagonists in Cancer

| GPCR | antagonist | References |
|---|---|---|
| α1-adrenoceptor | Doxazosin, terazosin, tamsulosin | (Tahmatzopoulos & Kyprianou, |
| | | 2004; Tahmatzopoulos et al., |
| | | 2004) |
| β₂-Adrenergic Receptor | ICI 118,551 | (Chen et al., 1999; Kasbohm et |
| | | al., 2005; Plummer et al., 2005) |
| Bradykinin receptor | B-9870, BKM-570, etc | (Barki-Harrington & Daaka, |
| | | 2001; Bironaite et al., 2004; Kue |
| | | & Daaka, 2000; Yan et al., 2005) |
| CXCR4 | AMD 3100 | (De Clercq, 2003; Rubin et al., |
| | | 2003) |
| Endothelin A Receptor | ZD4054 (AstraZeneca) | (Lassiter & Carducci, 2003; |
| (ETA) | | Morris et al., 2005) |
| Gonadotropin-releasing- | Abarelix, Antarelix, Cetrorelix, | (Doehn & Jocham, 2000; Huirne |
| hormone-receptor | Ganirelix acetate, Lturelix | & Lambalk, 2001) |
| Lysophosphatidic acid | Ki16425 | (Daaka, 2002; Kue & Daaka, |
| receptor | | 2000; Kue et al., 2002; Li et al., |
| | | 2005; Ohta et al., 2003; Raj et |
| | | al., 2002; Raj et al., 2004; Wang |
| | | et al., 2004) |
| Leukotriene B4 receptor | LY293111 | (Ding et al., 2005; Hennig et al., |
| | | 2005) |
| Platelet activation factor | Y-24180 | (Jan & Chao, 2004) |
| receptor | | |
| Prostaglandin E(2) | indomethacin | (Chang et al., 2004; Ohshiba et |
| | | al., 2003) |
| Broad-spectrum G | Substance P analogues | (Guha et al., 2005; Hennig et al., |
| protein-coupled receptor | | 2004) |
| (GPCR) antagonists | | |

Antiandrogens

Steroidal androgens

| Name | Reference |
|---|---|
| Cyproterone | (de Voogt, 1992; el Etreby et al., 1987; |
| acetate | Varenhorst et al., 1982) |

Nonsteroidal antiandrogens

| Name | Reference |
|---|---|
| Bicalutamide | (Iversen et al., 2001; Iversen et al., 2000; See et |
| | al., 2002; Tyrrell et al., 1998) |
| Flutamide | (Brogden & Clissold, 1989; Sogani & Whitmore, |
| | 1988) |
| Nilutamide | (Davis et al., 2005) |

Small-molecule VEGF antagonist and inhibitors

| Name | Reference |
|---|---|
| CP-547,632 | (Beebe et al., 2003) |
| PTK787/ZK22584 | (Drevs et al., 2002) |
| SU5416 | (Shaheen et al., 1999) |
| SU6668 | (Fabbro & Manley, 2001) |
| SU11248 | (Sakamoto, 2004) |
| Thalidomide | (Baidas et al., 2000; Eisen et al., 2000; Eisen, |
| | 2000) |
| ZD6474 | (Ciardiello et al., 2004; Ciardiello et al., 2003) |

PKA/CREB inhibitors

| Target | Inhibitor | Reference |
|---|---|---|
| PKA | H-89 | (Graziani et al., 2002; Kaufmann et al., |
| | | 2002; Manna & Frazier, 2004) |
| | KT5720 | (Caraglia et al., 2002; Kim et al., 2002) |
| | PKA inhibitor | (Cvijic et al., 2000; Graziani et al., 2002; |
| | peptide | Kaufmann et al., 2002; Manna & Frazier, |
| | | 2004) |
| CREB | K-CREB (a | Lung cancer (Linnerth et al., 2005); |
| | dominant negative | melanoma (Aucoin et al., 2004; Jean et |
| | construct to | al., 1998; Xie et al., 1997); gastric cancer |
| | CREB | (Pradeep et al., 2004); hepatocellular |
| | | carcinoma (Abramovitch et al., 2004); |
| | | acute myeloid leukemia (Kinjo et al., |
| | | 2005; Shankar et al., 2005) |
| | single chain Fv | Melanoma (Jean & Bar-Eli, 2001; Tellez |
| | fragment | et al., 2004) |

### REFERENCES CITED FOR APPENDIX B

Abramovitch R, Tavor E, Jacob-Hirsch J, Zeira E, Amariglio N, Pappo O, Rechavi G, Galun E and Honigman A. (2004). Cancer Res, 64, 1338-46.
Aucoin R, Reiland J, Roy M and Marchetti D. (2004). J Cell Biochem, 93, 215-23.
Baidas S M, Winer E P, Fleming G F, Harris L, Pluda J M, Crawford J G, Yamauchi H, Isaacs C, Hanfelt J, Tefft M, Flockhart D, Johnson M D, Hawkins M J, Lippman M E and Hayes D F. (2000). J Clin Oncol, 18, 2710-7.
Barki-Harrington L and Daaka Y. (2001). J Urol, 165, 2121-5.
Beebe J S, Jani J P, Knauth E, Goodwin P, Higdon C, Rossi A M, Emerson E, Finkelstein M, Floyd E, Harriman S, Atherton J, Hillerman S, Soderstrom C, Kou K, Gant T, Noe M C, Foster B, Rastinejad F, Marx M A, Schaeffer T, Whalen P M and Roberts W G. (2003). Cancer Res, 63, 7301-9.
Bironaite D, Gera L and Stewart J M. (2004). Chem Biol Interact, 150, 283-93.
Brogden R N and Clissold S P. (1989). Drugs, 38, 185-203.
Caraglia M, Marra M, Giuberti G, D'Alessandro A M, Beninati S, Lentini A, Pepe S, Boccellino M and Abbruzzese A. (2002). J Biochem (Tokyo), 132, 45-52.
Chang S H, Liu C H, Conway R, Han D K, Nithipatikom K, Trifan O C, Lane T F and Hla T. (2004). Proc Natl Acad Sci USA, 101, 591-6.
Chen T, Cho R W, Stork P J and Weber M J. (1999). Cancer Res, 59, 213-8.
Ciardiello F, Bianco R, Caputo R, Caputo R, Damiano V, Troiani T, Melisi D, De Vita F, De Placido S, Bianco A R and Tortora G. (2004). Clin Cancer Res, 10, 784-93.
Ciardiello F, Caputo R, Damiano V, Caputo R, Troiani T, Vitagliano D, Carlomagno F, Veneziani B M, Fontanini G, Bianco A R and Tortora G. (2003). Clin Cancer Res, 9, 1546-56.
Cvijic M E, Kita T, Shih W, DiPaola R S and Chin K V. (2000). Clin Cancer Res, 6, 2309-17.
Daaka Y. (2002). Biochim Biophys Acta, 1582, 265-9.
Davis N B, Ryan C W, Stadler W M and Vogelzang N J. (2005). BJU Int, 96, 787-90.
De Clercq E. (2003). Nat Rev Drug Discov, 2, 581-7.
de Voogt H J. (1992). Prostate Suppl, 4, 91-5.
Ding X Z, Talamonti M S, Bell R H, Jr. and Adrian T E. (2005). Anticancer Drugs, 16, 467-73.
Doehn C and Jocham D. (2000). Curr Opin Mol Ther, 2, 579-85.
Drevs J, Muller-Driver R, Wittig C, Fuxius S, Esser N, Hugenschmidt H, Konerding M A, Allegrini P R, Wood J, Hennig J, Unger C and Marme D. (2002). Cancer Res, 62, 4015-22.
Eisen T, Boshoff C, Mak I, Sapunar F, Vaughan M M, Pyle L, Johnston S R, Ahem R, Smith I E and Gore M E. (2000). Br J Cancer, 82, 812-7.
Eisen T G. (2000). Oncology (Williston Park), 14, 17-20.
el Etreby M F, Habenicht U F, Louton T, Nishino Y and Schroder H G. (1987). Prostate, 11, 361-75.
Fabbro D and Manley P W. (2001). Curr Opin Investig Drugs, 2, 1142-8.
Graziani G, Tentori L, Portarena I, Barbarino M, Tringali G, Pozzoli G and Navarra P. (2002). Endocrinology, 143, 807-13.
Guha S, Eibl G, Kisfalvi K, Fan R S, Burdick M, Reber H, Hines O J, Strieter R and Rozengurt E. (2005). Cancer Res, 65, 2738-45.
Hennig R, Ding X Z, Tong W G, Witt R C, Jovanovic B D and Adrian T E. (2004). Cancer Lett, 210, 41-6.
Hennig R, Ventura J, Segersvard R, Ward E, Ding X Z, Rao S M, Jovanovic B D, Iwamura T, Talamonti M S, Bell R H, Jr. and Adrian T E. (2005). Neoplasia, 7, 417-25.
Huirne J A and Lambalk C B. (2001). Lancet, 358, 1793-803.
Iversen P, Melezinek I and Schmidt A. (2001). BJU Int, 87, 47-56.
Iversen P, Tyrrell C J, Kaisary A V, Anderson J B, Van Poppel H, Tammela T L, Chamberlain M, Carroll K and Melezinek I. (2000). J Urol, 164, 1579-82.
Jan C R and Chao Y Y. (2004). Cell Signal, 16, 959-65.
Jean D and Bar-Eli M. (2001). Crit Rev Immunol, 21, 275-86.
Jean D, Harbison M, McConkey D J, Ronai Z and Bar-Eli M. (1998). J Biol Chem, 273, 24884-90.
Kasbohm E A, Guo R, Yowell C W, Bagchi G, Kelly P, Arora P, Casey P J and Daaka Y. (2005). J Biol Chem, 280, 11583-9.
Kaufmann R, Junker U, Junker K, Nuske K, Ranke C, Zieger M and Scheele J. (2002). Cancer Lett, 180, 183-90.
Kim M S, Cheong Y P, So H S, Lee K M, Son Y, Lee C S, Yun J S and Park R. (2002). Biochem Biophys Res Commun, 291, 663-8.
Kinjo K, Sandoval S, Sakamoto K M and Shankar D B. (2005). Cell Cycle, 4, 1134-5.
Kue P F and Daaka Y. (2000). J Urol, 164, 2162-7.
Kue P F, Taub J S, Harrington L B, Polakiewicz R D, Ullrich A and Daaka Y. (2002). Int J Cancer, 102, 572-9.
Lassiter L K and Carducci M A. (2003). Semin Oncol, 30, 678-88.
Li S, Huang S and Peng S B. (2005). Int J Oncol, 27, 1329-38.
Linnerth N M, Baldwin M, Campbell C, Brown M, McGowan H and Moorehead R A. (2005). Oncogene.
Manna P P and Frazier W A. (2004). Cancer Res, 64, 1026-36.
Morris C D, Rose A, Curwen J, Hughes A M, Wilson D J and Webb D J. (2005). Br J Cancer, 92, 2148-52.
Ohshiba T, Miyaura C and Ito A. (2003). Biochem Biophys Res Commun, 300, 957-64.
Ohta H, Sato K, Murata N, Damirin A, Malchinkhuu E, Kon J, Kimura T, Tobo M, Yamazaki Y, Watanabe T, Yagi M, Sato M, Suzuki R, Murooka H, Sakai T, Nishitoba T, Im D S, Nochi H, Tamoto K, Tomura H and Okajima F. (2003). Mol Pharmacol, 64, 994-1005.
Plummer H K, 3rd, Dhar M S, Cekanova M and Schuller H M. (2005). BMC Cancer, 5, 104.
Pradeep A, Sharma C, Sathyanarayana P, Albanese C, Fleming J V, Wang T C, Wolfe M M, Baker K M, Pestell R G and Rana B. (2004). Oncogene, 23, 3689-99.
Raj G V, Barki-Harrington L, Kue P F and Daaka Y. (2002). J Urol, 167, 1458-63.
Raj G V, Sekula J A, Guo R, Madden J F and Daaka Y. (2004). Prostate, 61, 105-13.
Rubin J B, Kung A L, Klein R S, Chan J A, Sun Y, Schmidt K, Kieran M W, Luster A D and Segal R A. (2003). Proc Natl Acad Sci USA, 100, 13513-8.
Sakamoto K M. (2004). Curr Opin Investig Drugs, 5, 1329-39.
See W A, Wirth M P, McLeod D G, Iversen P, Klimberg I, Gleason D, Chodak G, Montie J, Tyrrell C, Wallace D M, Delaere K P, Vaage S, Tammela T L, Lukkarinen O, Persson B E, Carroll K and Kolvenbag G J. (2002). J Urol, 168, 429-35.
Shaheen R M, Davis D W, Liu W, Zebrowski B K, Wilson M R, Bucana C D, McConkey D J, McMahon G and Ellis L M. (1999). Cancer Res, 59, 5412-6.
Shankar D B, Cheng J C and Sakamoto K M. (2005). Cancer.
Sogani P C and Whitmore W F, Jr. (1988). Cancer Treat Res, 39, 131-45.
Tahmatzopoulos A and Kyprianou N. (2004). Prostate, 59, 91-100.
Tahmatzopoulos A, Rowland R G and Kyprianou N. (2004). Expert Opin Pharmacother, 5, 1279-85.
Tellez C, Jean D and Bar-Eli M. (2004). Methods, 34, 233-9.
Tyrrell C J, Kaisary A V, Iversen P, Anderson J B, Baert L, Tammela T, Chamberlain M, Webster A and Blackledge G. (1998). Eur Urol, 33, 447-56.
Varenhorst E, Wallentin L and Carlstrom K. (1982). Scand J Urol Nephrol, 16, 31-6.
Wang Q, Liu M, Kozasa T, Rothstein J D, Stemweis P C and Neubig R R. (2004). J Biol Chem, 279, 28831-4.
Xie S, Price J E, Luca M, Jean D, Ronai Z and Bar-Eli M. (1997). Oncogene, 15, 2069-75.
Yan F, Mosier P D, Westkaemper R B, Stewart J, Zjawiony J K, Vortherms T A, Sheffler D J and Roth B L. (2005). Biochemistry, 44, 8643-51.

### REFERENCES CITED

1. Jemal, A., Murray, T., Ward, E., Samuels, A., Tiwari, R. C., Ghafoor, A., Feuer, E. J., and Thun, M. J. Cancer statistics, 2005. CA Cancer J Clin, 55: 10-30, 2005.
2. Oh, W. K. and Kantoff, P. W. Management of hormone refractory prostate cancer: current standards and future prospects. J Urol, 160: 1220-1229, 1998.
3. Pagliaro, L. C., Delpassand, E. S., Williams, D., Millikan, R. E., Tu, S. M., and Logothetis, C. J. A Phase I/II study of strontium-89 combined with gemcitabine in the treatment of patients with androgen independent prostate carcinoma and bone metastases. Cancer, 97: 2988-2994, 2003.
4. Saad, F., Gleason, D. M., Murray, R., Tchekmedyian, S., Venner, P., Lacombe, L., Chin, J. L., Vinholes, J. J., Goas, J. A., and Chen, B. A randomized, placebo-controlled trial of zoledronic acid in patients with hormone-refractory metastatic prostate carcinoma. J Natl Cancer Inst, 94: 1458-1468, 2002.
5. Friedland, J. Local and systemic radiation for palliation of metastatic disease. Urol Clin North Am, 26: 391-402, x, 1999.
6. Hsieh, C. L., Gardner, T. A., Miao, L., Balian, G., and Chung, L. W. Cotargeting tumor and stroma in a novel chimeric tumor model involving the growth of both human prostate cancer and bone stromal cells. Cancer Gene Ther, 11: 148-155, 2004.
7. Knerr, K., Ackermann, K., Neidhart, T., and Pyerin, W. Bone metastasis: Osteoblasts affect growth and adhesion regulons in prostate tumor cells and provoke osteomimicry. Int J Cancer, 111: 152-159, 2004.
8. Bagi, C. M. Targeting of therapeutic agents to bone to treat metastatic cancer. Adv Drug Deliv Rev, 57: 995-1010, 2005.
9. Smith, M. R. Osteoclast-targeted therapy for prostate cancer. Curr Treat Options Oncol, 5: 367-375, 2004.
10. Fierro, F. A., Sierralta, W. D., Epunan, M. J., and Minguell, J. J. Marrow-derived mesenchymal stem cells: role in epithelial tumor cell determination. Clin Exp Metastasis, 21: 313-319, 2004.
11. Nyambo, R., Cross, N., Lippitt, J., Holen, I., Bryden, G., Hamdy, F. C., and Eaton, C. L. Human bone marrow stromal cells protect prostate cancer cells from TRAIL-induced apoptosis. J Bone Miner Res, 19: 1712-1721, 2004.
12. Yoneda, T. and Hiraga, T. Crosstalk between cancer cells and bone microenvironment in bone metastasis. Biochem Biophys Res Commun, 328: 679-687, 2005.
13. Neri, D. and Bicknell, R. Tumour vascular targeting. Nat Rev Cancer, 5: 436-446, 2005.
14. Teoh, G. and Anderson, K. C. Interaction of tumor and host cells with adhesion and extracellular matrix molecules in the development of multiple myeloma. Hematol Oncol Clin North Am, 11: 27-42, 1997.
15. Cooper, C. R., Chay, C. H., Gendernalik, J. D., Lee, H. L., Bhatia, J., Taichman, R. S., McCauley, L. K., Keller, E. T., and Pienta, K. J. Stromal factors involved in prostate carcinoma metastasis to bone. Cancer, 97: 739-747, 2003.
16. Lee, H. L., Pienta, K. J., Kim, W. J., and Cooper, C. R. The effect of bone-associated growth factors and cytokines on the growth of prostate cancer cells derived from soft tissue versus bone metastases in vitro. Int J Oncol, 22: 921-926, 2003.
17. Koeneman, K. S., Yeung, F., and Chung, L. W. Osteomimetic properties of prostate cancer cells: a hypothesis supporting the predilection of prostate cancer metastasis and growth in the bone environment. Prostate, 39: 246-261, 1999.
18. Lin, D. L., Tarnowski, C. P., Zhang, J., Dai, J., Rohn, E., Patel, A. H., Morris, M. D., and Keller, E. T. Bone metastatic LNCaP-derivative C4-2B prostate cancer cell line mineralizes in vitro. Prostate, 47: 212-221, 2001.
19. Brown, L. F., Papadopoulos-Sergiou, A., Berse, B., Manseau, E. J., Tognazzi, K., Perruzzi, C. A., Dvorak, H. F., and Senger, D. R. Osteopontin expression and distribution in human carcinomas. Am J Pathol, 145: 610-623, 1994.
20. Carlinfante, G., Vassiliou, D., Svensson, O., Wendel, M., Heinegard, D., and Andersson, G. Differential expression of osteopontin and bone sialoprotein in bone metastasis of breast and prostate carcinoma. Clin Exp Metastasis, 20: 437-444, 2003.
21. Curatolo, C., Ludovico, G. M., Correale, M., Pagliarulo, A., Abbate, I., Cirrillo Marucco, E., and Barletta, A. Advanced prostate cancer follow-up with prostate-specific antigen, prostatic acid phosphatase, osteocalcin and bone isoenzyme of alkaline phosphatase. Eur Urol, 21 Suppl 1: 105-107, 1992.
22. Waltregny, D., Bellahcene, A., Van Riet, I., Fisher, L. W., Young, M., Fernandez, P., Dewe, W., de Leval, J., and Castronovo, V. Prognostic value of bone sialoprotein expression in clinically localized human prostate cancer. J Natl Cancer Inst, 90: 1000-1008, 1998.
23. Yeung, F., Law, W. K., Yeh, C. H., Westendorf, J. J., Zhang, Y., Wang, R., Kao, C., and Chung, L. W. Regulation of human osteocalcin promoter in hormone-independent human prostate cancer cells. J Biol Chem, 277: 2468-2476, 2002.
24. Zhang, J., Dai, J., Qi, Y., Lin, D. L., Smith, P., Strayhorn, C., Mizokami, A., Fu, Z., Westman, J., and Keller, E. T. Osteoprotegerin inhibits prostate cancer-induced osteoclastogenesis and prevents prostate tumor growth in the bone. J Clin Invest, 107: 1235-1244, 2001.
25. Abdul, M. and Hoosein, N. Changes in beta-2 microglobulin expression in prostate cancer. Urol Oncol, 5: 168-172, 2000.
26. Chung, L. W., Hsieh, C. L., Law, A., Sung, S. Y., Gardner, T. A., Egawa, M., Matsubara, S., and Zhau, H. E. New targets for therapy in prostate cancer: modulation of stromal-epithelial interactions. Urology, 62: 44-54, 2003.
27. Matsubara, S., Wada, Y., Gardner, T. A., Egawa, M., Park, M. S., Hsieh, C. L., Zhau, H. E., Kao, C., Kamidono, S., Gillenwater, J. Y., and Chung, L. W. A conditional replication-competent adenoviral vector, Ad-OC-E1a, to cotarget prostate cancer and bone stroma in an experimental model of androgen-independent prostate cancer bone metastasis. Cancer Res, 61: 6012-6019, 2001.
28. Ou, Y. C., Chen, J. T., Yang, C. R., Ko, J. L., Hsieh, Y. S., and Kao, C. Expression of osteocalcin in prostate cancer before and after hormonal therapy. Anticancer Res, 23: 3807-3811, 2003.
29. Fedarko, N. S., Jain, A., Karadag, A., Van Eman, M. R., and Fisher, L. W. Elevated serum bone sialoprotein and osteopontin in colon, breast, prostate, and lung cancer. Clin Cancer Res, 7: 4060-4066, 2001.
30. Thalmann, G. N., Sikes, R. A., Devoll, R. E., Kiefer, J. A., Markwalder, R., Klima, I., Farach-Carson, C. M., Studer, U. E., and Chung, L. W. Osteopontin: possible role in prostate cancer progression. Clin Cancer Res, 5: 2271-2277, 1999.
31. Roodman, G. D. Mechanisms of bone metastasis. N Engl J Med, 350: 1655-1664, 2004.
32. Jacobs, S. C. Spread of prostatic cancer to bone. Urology, 21: 337-344, 1983.
33. Pinski, J. and Dorff, T. B. Prostate cancer metastases to bone: pathophysiology, pain management, and the promise of targeted therapy. Eur J Cancer, 41: 932-940, 2005.
34. Tu, S. M., Millikan, R. E., Mengistu, B., Delpassand, E. S., Amato, R. J., Pagliaro, L. C., Daliani, D., Papandreou, C. N., Smith, T. L., Kim, J., Podoloff, D. A., and Logothetis, C. J. Bone-targeted therapy for advanced androgen-independent carcinoma of the prostate: a randomised phase II trial. Lancet, 357: 336-341, 2001.
35. Fisher, M. The endothelin-1 antagonist, atrasentan, improves time to progression and quality of life in hormone-refractory prostate cancer. Clin Prostate Cancer, 1: 79-80, 2002.
36. Lee, D. Clinical trials of atrasentan in hormone-refractory prostate cancer. Clin Prostate Cancer, 2: 84-86, 2003.
37. Dahut, W. L., Gulley, J. L., Arlen, P. M., Liu, Y., Fedenko, K. M., Steinberg, S. M., Wright, J. J., Parnes, H., Chen, C. C., Jones, E., Parker, C. E., Linehan, W. M., and Figg, W. D. Randomized phase II trial of docetaxel plus thalidomide in androgen-independent prostate cancer. J Clin Oncol, 22: 2532-2539, 2004.
38. Bindra, R. S. and Glazer, P. M. Genetic instability and the tumor microenvironment: towards the concept of microenvironment-induced mutagenesis. Mutat Res, 569: 75-85, 2005.
39. Macaluso, M., Paggi, M. G., and Giordano, A. Genetic and epigenetic alterations as hallmarks of the intricate road to cancer. Oncogene, 22: 6472-6478, 2003.
40. Mueller, M. M. and Fusenig, N. E. Friends or foes—bipolar effects of the tumour stroma in cancer. Nat Rev Cancer, 4: 839-849, 2004.
41. Kurose, K., Hoshaw-Woodard, S., Adeyinka, A., Lemeshow, S., Watson, P. H., and Eng, C. Genetic model of multi-step breast carcinogenesis involving the epithelium and stroma: clues to tumour-microenvironment interactions. Hum Mol Genet, 10: 1907-1913, 2001.
42. Thalmann, G. N., Anezinis, P. E., Chang, S. M., Zhau, H. E., Kim, E. E., Hopwood, V. L., Pathak, S., von Eschenbach, A. C., and Chung, L. W. Androgen-independent cancer progression and bone metastasis in the LNCaP model of human prostate cancer. Cancer Res, 54: 2577-2581, 1994.
43. Tuxhorn, J. A., Ayala, G. E., Smith, M. J., Smith, V. C., Dang, T. D., and Rowley, D. R. Reactive stroma in human prostate cancer: induction of myofibroblast phenotype and extracellular matrix remodeling. Clin Cancer Res, 8: 2912-2923, 2002.
44. Micke, P. and Ostman, A. Tumour-stroma interaction: cancer-associated fibroblasts as novel targets in anti-cancer therapy? Lung Cancer, 45 Suppl 2: S163-175, 2004.
45. Smolle, J., Hofmann-Wellenhof, R., and Fink-Puches, R. Melanoma and stroma: an interaction of biological and prognostic importance. Semin Cutan Med Surg, 15: 326-335, 1996.
46. Reubi, J. C., Horisberger, U., and Laissue, J. High density of somatostatin receptors in veins surrounding human cancer tissue: role in tumor-host interaction? Int J Cancer, 56: 681-688, 1994.
47. Wu, H. C., Hsieh, J. T., Gleave, M. E., Brown, N. M., Pathak, S., and Chung, L. W. Derivation of androgen-independent human LNCaP prostatic cancer cell sublines: role of bone stromal cells. Int J Cancer, 57: 406-412, 1994.
48. Sung, S. Y. and Chung, L. W. Prostate tumor-stroma interaction: molecular mechanisms and opportunities for therapeutic targeting. Differentiation, 70: 506-521, 2002.
49. Gleave, M. E., Hsieh, J. T., von Eschenbach, A. C., and Chung, L. W. Prostate and bone fibroblasts induce human prostate cancer growth in vivo: implications for bidirectional tumor-stromal cell interaction in prostate carcinoma growth and metastasis. J Urol, 147: 1151-1159, 1992.
50. Wong, Y. C. and Wang, Y. Z. Growth factors and epithelial-stromal interactions in prostate cancer development. Int Rev Cytol, 199: 65-116, 2000.
51. Chung, L. W., Gleave, M. E., Hsieh, J. T., Hong, S. J., and Zhau, H. E. Reciprocal mesenchymal-epithelial interaction affecting prostate tumour growth and hormonal responsiveness. Cancer Surv, 11: 91-121, 1991.
52. Huang, W. C., Xie, Z., Konaka, H., Sodek, J., Zhau, H. E., and Chung, L. W. Human osteocalcin and bone sialoprotein mediating osteomimicry of prostate cancer cells: role of cAMP-dependent protein kinase A signaling pathway. Cancer Res, 65: 2303-2313, 2005.
53. De, S., Chen, J., Narizhneva, N. V., Heston, W., Brainard, J., Sage, E. H., and Byzova, T. V. Molecular pathway for cancer metastasis to bone. J Biol Chem, 278: 39044-39050, 2003.
54. Dorai, T., Dutcher, J. P., Dempster, D. W., and Wiernik, P. H. Therapeutic potential of curcumin in prostate cancer—V: Interference with the osteomimetic properties of hormone refractory C4-2B prostate cancer cells. Prostate, 60: 1-17, 2004.
55. Fizazi, K., Yang, J., Peleg, S., Sikes, C. R., Kreimann, E. L., Daliani, D., Olive, M., Raymond, K. A., Janus, T. J., Logothetis, C. J., Karsenty, G., and Navone, N. M. Prostate cancer cells-osteoblast interaction shifts expression of growth/survival-related genes in prostate cancer and reduces expression of osteoprotegerin in osteoblasts. Clin Cancer Res, 9: 2587-2597, 2003.
56. Thomas, R., True, L. D., Bassuk, J. A., Lange, P. H., and Vessella, R. L. Differential expression of osteonectin/SPARC during human prostate cancer progression. Clin Cancer Res, 6: 1140-1149, 2000.
57. Zayzafoon, M., Abdulkadir, S. A., and McDonald, J. M. Notch signaling and ERK activation are important for the osteomimetic properties of prostate cancer bone metastatic cell lines. J Biol Chem, 279: 3662-3670, 2004.
58. Inoue, H., Nishimura, K., Oka, D., Nakai, Y., Shiba, M., Tokizane, T., Arai, Y., Nakayama, M., Shimizu, K., Takaha, N., Nonomura, N., and Okuyama, A. Prostate cancer mediates osteoclastogenesis through two different pathways. Cancer Lett, 223: 121-128, 2005.
59. Keller, E. T. and Brown, J. Prostate cancer bone metastases promote both osteolytic and osteoblastic activity. J Cell Biochem, 91: 718-729, 2004.
60. Solheim, J. C. Class I MHC molecules: assembly and antigen presentation. Immunol Rev, 172: 11-19, 1999.
61. Rajkumar, S. V. and Greipp, P. R. Prognostic factors in multiple myeloma. Hematol Oncol Clin North Am, 13: 1295-1314, xi, 1999.
62. Nissen, M. H., Bjerrum, O. J., Plesner, T., Wilken, M., and Rorth, M. Modification of beta-2-microglobulin in sera from patients with small cell lung cancer: evidence for involvement of a serine protease. Clin Exp Immunol, 67: 425-432, 1987.
63. Rasmuson, T., Grankvist, K., and Ljungberg, B. Serum beta 2-microglobulin and prognosis of patients with renal cell carcinoma. Acta Oncol, 35: 479-482, 1996.
64. Mavridis, A. K., Tsiara, S., Makis, A., Chaidos, A., Christou, L., Seferiadis, K., and Bourantas, K. L. Interleukins, TNF-alpha and beta-2M in patients with B cell chronic lymphocytic leukemia. J Exp Clin Cancer Res, 17: 445-448, 1998.
65. Sadamori, N., Mine, M., Hakariya, S., Ichiba, M., Kawachi, T., Itoyama, T., Nakamura, H., Tomonaga, M., and Hayashi, K. Clinical significance of beta 2-microglobulin in serum of adult T cell leukemia. Leukemia, 9: 594-597, 1995.
66. Gressner, A. M. and Neu, H. H. N-terminal procollagen peptide and beta 2-microglobulin in synovial fluids from inflammatory and non-inflammatory joint diseases. Clin Chim Acta, 141: 241-245, 1984.
67. Krejsek, J., Slezak, R., Kopecky, O., Derner, V., and Andrys, C. Elevation of serum soluble intercellular adhesion molecule-1 (sICAM-1) and beta-2-microglobulin in Sjogren's syndrome. Clin Rheumatol, 16: 149-153, 1997.
68. Par, A. and Falus, A. Serum beta 2-microglobulin (beta 2m) and anti-beta 2m antibody in chronic hepatitis. Acta Med Hung, 43: 343-349, 1986.
69. York, I. A. and Rock, K. L. Antigen processing and presentation by the class I major histocompatibility complex. Annu Rev Immunol, 14: 369-396, 1996.
70. Bubenik, J. MHC class I down-regulation: tumour escape from immune surveillance? (review). Int J Oncol, 25: 487-491, 2004.
71. Foss, F. M. Immunologic mechanisms of antitumor activity. Semin Oncol, 29: 5-11, 2002.
72. Waller, E. K., Giver, C. R., Rosenthal, H., Somani, J., Langston, A. A., Lonial, S., Roback, J. D., Li, J. M., Hossain, M. S., and Redei, I. Facilitating T-cell immune reconstitution after haploidentical transplantation in adults. Blood Cells Mol Dis, 33: 233-237, 2004.
73. Kennedy, M. S., Lui, V. K., and Ragab, A. H. Bone marrow transplantation. Am J Med Technol, 45: 205-210, 1979.
74. Greipp, P. Prognostic indicators for the diagnosis of multiple myeloma. Oncology (Williston Park), 18: 558-560, 2004.
75. Rowley, D. R., Dang, T. D., McBride, L., Gerdes, M. J., Lu, B., and Larsen, M. Beta-2 microglobulin is mitogenic to PC-3 prostatic carcinoma cells and antagonistic to transforming growth factor beta 1 action. Cancer Res, 55: 781-786, 1995.
76. Tran, M., Rutecki, G. W., and Sprague, S. M. The pathogenesis of beta(2)-microglobulin-induced bone lesions in dialysis-related amyloidosis. Semin Dial, 14: 131-133, 2001.
77. Xie, J. and Yi, Q. Beta2-microglobulin as a potential initiator of inflammatory responses. Trends Immunol, 24: 228-229; author reply 229-230, 2003.
78. Shen, L., Chiang, A. K., Liu, W. P., Li, G. D., Liang, R. H., and Srivastava, G. Expression of HLA class I, beta(2)-microglobulin, TAP1 and IL-10 in Epstein-Barr virus-associated nasal NK/T-cell lymphoma: Implications for tumor immune escape mechanism. Int J Cancer, 92: 692-696, 2001.
79. Tsai, C. Y., Wu, T. H., Yu, C. L., Lu, J. Y., and Tsai, Y. Y. Increased excretions of beta2-microglobulin, IL-6, and IL-8 and decreased excretion of Tamm-Horsfall glycoprotein in urine of patients with active lupus nephritis. Nephron, 85: 207-214, 2000.
80. Centrella, M., McCarthy, T. L., and Canalis, E. Beta 2-microglobulin enhances insulin-like growth factor I receptor levels and synthesis in bone cell cultures. J Biol Chem, 264: 18268-18271, 1989.
81. Corey, E., Brown, L. G., Kiefer, J. A., Quinn, J. E., Pitts, T. E., Blair, J. M., and Vessella, R. L. Osteoprotegerin in prostate cancer bone metastasis. Cancer Res, 65: 1710-1718, 2005.
82. Chung, L. W., Baseman, A., Assikis, V., and Zhau, H. E. Molecular insights into prostate cancer progression: the missing link of tumor microenvironment. J Urol, 1 73: 10-20, 2005.
83. Giaccone, G. HER1/EGFR-targeted agents: predicting the future for patients with unpredictable outcomes to therapy. Ann Oncol, 16: 538-548, 2005.
84. Richter, M. and Zhang, H. Receptor-targeted cancer therapy. DNA Cell Biol, 24: 271-282, 2005.
85. Emens, L. A. Trastuzumab: targeted therapy for the management of HER-2/neu-overexpressing metastatic breast cancer. Am J Ther, 12: 243-253, 2005.
86. Pegram, M. D. and Reese, D. M. Combined biological therapy of breast cancer using monoclonal antibodies directed against HER2/neu protein and vascular endothelial growth factor. Semin Oncol, 29: 29-37, 2002.
87. Herbst, R. S., Fukuoka, M., and Baselga, J. Gefitinib—a novel targeted approach to treating cancer. Nat Rev Cancer, 4: 956-965, 2004.
88. Arora, A. and Scholar, E. M. Role of Tyrosine Kinase Inhibitors in Cancer Therapy. J Pharmacol Exp Ther, 2005.
89. Strother, J. M., Beer, T. M., and Dreicer, R. Novel cytotoxic and biological agents for prostate cancer: where will the money be in 2005? Eur J Cancer, 41: 954-964, 2005.
90. Edlund, M., Miyamoto, T., Sikes, R. A., Ogle, R., Laurie, G. W., Farach-Carson, M. C., Otey, C. A., Zhau, H. E., and Chung, L. W. Integrin expression and usage by prostate cancer cell lines on laminin substrata. Cell Growth Differ, 12: 99-107, 2001.
91. Duque, J. L., Loughlin, K. R., Adam, R. M., Kantoff, P. W., Zurakowski, D., and Freeman, M. R. Plasma levels of vascular endothelial growth factor are increased in patients with metastatic prostate cancer. Urology, 54: 523-527, 1999.
92. Shariat, S. F., Anwuri, V. A., Lamb, D. J., Shah, N. V., Wheeler, T. M., and Slawin, K. M. Association of preoperative plasma levels of vascular endothelial growth factor and soluble vascular cell adhesion molecule-I with lymph node status and biochemical progression after radical prostatectomy. J Clin Oncol, 22: 1655-1663, 2004.
93. Chen, J., De, S., Brainard, J., and Byzova, T. V. Metastatic properties of prostate cancer cells are controlled by VEGF. Cell Commun Adhes, 11: 1-11, 2004.
94. George, D. J., Halabi, S., Shepard, T. F., Vogelzang, N. J., Hayes, D. F., Small, E. J., and Kantoff, P. W. Prognostic significance of plasma vascular endothelial growth factor levels in patients with hormone-refractory prostate cancer treated on Cancer and Leukemia Group B 9480. Clin Cancer Res, 7: 1932-1936, 2001.
95. George, D. J., Regan, M. M., Oh, W. K., Tay, M. H., Manola, J., Decalo, N., Duggan, S., Dewolf, W. C., Kantoff, P. W., and Bubley, G. J. Radical prostatectomy lowers plasma vascular endothelial growth factor levels in patients with prostate cancer. Urology, 63: 327-332, 2004.
96. Sun, Y. X., Schneider, A., Jung, Y., Wang, J., Dai, J., Wang, J., Cook, K., Osman, N. I., Koh-Paige, A. J., Shim, H., Pienta, K. J., Keller, E. T., McCauley, L. K., and Taichman, R. S. Skeletal localization and neutralization of the SDF-1 (CXCL12)/CXCR4 axis blocks prostate cancer metastasis and growth in osseous sites in vivo. J Bone Miner Res, 20: 318-329, 2005.
97. Wang, J., Wang, J., Sun, Y., Song, W., Nor, J. E., Wang, C. Y., and Taichman, R. S. Diverse signaling pathways through the SDF-1/CXCR4 chemokine axis in prostate cancer cell lines leads to altered patterns of cytokine secretion and angiogenesis. Cell Signal, 2005.
98. Byzova, T. V., Goldman, C. K., Pampori, N., Thomas, K. A., Bett, A., Shattil, S. J., and Plow, E. F. A mechanism for modulation of cellular responses to VEGF: activation of the integrins. Mol Cell, 6: 851-860, 2000.
99. Wang, G. L., Jiang, B. H., Rue, E. A., and Semenza, G. L. Hypoxia-inducible factor 1 is a basic-helix-loop-helix-PAS heterodimer regulated by cellular O2 tension. Proc Natl Acad Sci USA, 92: 5510-5514, 1995.
100. Forsythe, J. A., Jiang, B. H., Iyer, N. V., Agani, F., Leung, S. W., Koos, R. D., and Semenza, G. L. Activation of vascular endothelial growth factor gene transcription by hypoxia-inducible factor 1. Mol Cell Biol, 16: 4604-4613, 1996.
101. Okazaki, T., Ebihara, S., Takahashi, H., Asada, M., Kanda, A., and Sasaki, H. Macrophage colony-stimulating factor induces vascular endothelial growth factor production in skeletal muscle and promotes tumor angiogenesis. J Immunol, 174: 7531-7538, 2005.
102. Shi, Y. H., Wang, Y. X., Bingle, L., Gong, L. H., Heng, W. J., Li, Y., and Fang, W. G. In vitro study of HIF-1 activation and VEGF release by bFGF in the T47D breast cancer cell line under normoxic conditions: involvement of PI-3K/Akt and MEK1/ERK pathways. J Pathol, 205: 530-536, 2005.
103. Mizukami, Y., Jo, W. S., Duerr, E. M., Gala, M., Li, J., Zhang, X., Zimmer, M. A., Iliopoulos, O., Zukerberg, L. R., Kohgo, Y., Lynch, M. P., Rueda, B. R., and Chung, D. C. Induction of interleukin-8 preserves the angiogenic response in HIF-1alpha-deficient colon cancer cells. Nat Med, 2005.
104. Letsch, M., Schally, A. V., Busto, R., Bajo, A. M., and Varga, J. L. Growth hormone-releasing hormone (GHRH) antagonists inhibit the proliferation of androgen-dependent and -independent prostate cancers. Proc Natl Acad Sci USA, 100: 1250-1255, 2003.
105. Mueller, M. D., Vigne, J. L., Minchenko, A., Lebovic, D. I., Leitman, D. C., and Taylor, R. N. Regulation of vascular endothelial growth factor (VEGF) gene transcription by estrogen receptors alpha and beta. Proc Natl Acad Sci USA, 97: 10972-10977, 2000.
106. Kanda, N. and Watanabe, S. 17beta-estradiol enhances vascular endothelial growth factor production and dihydrotestosterone antagonizes the enhancement via the regulation of adenylate cyclase in differentiated THP-1 cells. J Invest Dermatol, 118: 519-529, 2002.
107. Stewart, R. J., Panigrahy, D., Flynn, E., and Folkman, J. Vascular endothelial growth factor expression and tumor angiogenesis are regulated by androgens in hormone responsive human prostate carcinoma: evidence for androgen dependent destabilization of vascular endothelial growth factor transcripts. J Urol, 165: 688-693, 2001.
108. Mabjeesh, N. J., Willard, M. T., Frederickson, C. E., Zhong, H., and Simons, J. W. Androgens stimulate hypoxia-inducible factor 1 activation via autocrine loop of tyrosine kinase receptor/phosphatidylinositol 3′-kinase/protein kinase B in prostate cancer cells. Clin Cancer Res, 9: 2416-2425, 2003.
109. Zhau, H. Y., Chang, S. M., Chen, B. Q., Wang, Y., Zhang, H., Kao, C., Sang, Q. A., Pathak, S. J., and Chung, L. W. Androgen-repressed phenotype in human prostate cancer. Proc Natl Acad Sci USA, 93: 15152-15157, 1996.
110. Ferrara, N. Vascular endothelial growth factor as a target for anticancer therapy. Oncologist, 9 Suppl 1: 2-10, 2004.
111. Kaushal, V., Mukunyadzi, P., Dennis, R. A., Siegel, E. R., Johnson, D. E., and Kohli, M. Stage-specific characterization of the vascular endothelial growth factor axis in prostate cancer: expression of lymphangiogenic markers is associated with advanced-stage disease. Clin Cancer Res, 11: 584-593, 2005.
112. Ferrara, N., Gerber, H. P., and LeCouter, J. The biology of VEGF and its receptors. Nat Med, 9: 669-676, 2003.
113. Keyt, B. A., Berleau, L. T., Nguyen, H. V., Chen, H., Heinsohn, H., Vandlen, R., and Ferrara, N. The carboxyl-terminal domain (111-165) of vascular endothelial growth factor is critical for its mitogenic potency. J Biol Chem, 271: 7788-7795, 1996.
114. Soker, S., Takashima, S., Miao, H. Q., Neufeld, G., and Klagsbrun, M. Neuropilin-1 is expressed by endothelial and tumor cells as an isoform-specific receptor for vascular endothelial growth factor. Cell, 92: 735-745, 1998.
115. Ferrer, F. A., Miller, L. J., Lindquist, R., Kowalczyk, P., Laudone, V. P., Albertsen, P. C., and Kreutzer, D. L. Expression of vascular endothelial growth factor receptors in human prostate cancer. Urology, 54: 567-572, 1999.
116. Steiner, H., Berger, A. P., Godoy-Tundidor, S., Bjartell, A., Lilja, H., Bartsch, G., Hobisch, A., and Culig, Z. An autocrine loop for vascular endothelial growth factor is established in prostate cancer cells generated after prolonged treatment with interleukin 6. Eur J Cancer, 40: 1066-1072, 2004.
117. Gagnon, M. L., Bielenberg, D. R., Gechtman, Z., Miao, H. Q., Takashima, S., Soker, S., and Klagsbrun, M. Identification of a natural soluble neuropilin-1 that binds vascular endothelial growth factor: In vivo expression and antitumor activity. Proc Natl Acad Sci USA, 97: 2573-2578, 2000.
118. Latil, A., Bieche, I., Pesche, S., Valeri, A., Fournier, G., Cussenot, O., and Lidereau, R. VEGF overexpression in clinically localized prostate tumors and neuropilin-1 overexpression in metastatic forms. Int J Cancer, 89: 167-171, 2000.
119. Miao, H. Q., Lee, P., Lin, H., Soker, S., and Klagsbrun, M. Neuropilin-1 expression by tumor cells promotes tumor angiogenesis and progression. Faseb J, 14: 2532-2539, 2000.
120. Kaplan-Lefko, P. J., Chen, T. M., Ittmann, M. M., Barrios, R. J., Ayala, G. E., Huss, W. J., Maddison, L. A., Foster, B. A., and Greenberg, N. M. Pathobiology of autochthonous prostate cancer in a pre-clinical transgenic mouse model. Prostate, 55: 219-237, 2003.
121. Bisanz, K., Yu, J., Edlund, M., Spohn, B., Hung, M. C., Chung, L. W., and Hsieh, C. L. Targeting ECM-Integrin Interaction with Liposome-Encapsulated Small Interfering RNAs Inhibits the Growth of Human Prostate Cancer in a Bone Xenograft Imaging Model. Mol Ther, 12: 634-643, 2005.
122. Reddi, A. H. Bone matrix in the solid state: geometric influence on differentiation of fibroblasts. Adv Biol Med Phys, 15: 1-18, 1974.
123. Reddi, A. H. and Huggins, C. B. Cyclic electrochemical inactivation and restoration of competence of bone matrix to transform fibroblasts. Proc Natl Acad Sci USA, 71: 1648-1652, 1974.
124. Nagakawa, O., Akashi, T., Hayakawa, Y., Junicho, A., Koizumi, K., Fujiuchi, Y., Furuya, Y., Matsuda, T., Fuse, H., and Saiki, I. Differential expression of integrin subunits in DU-145/AR prostate cancer cells. Oncol Rep, 12: 837-841, 2004.
125. Pfohler, C., Fixemer, T., Jung, V., Dooley, S., Remberger, K., and Bonkhoff, H. In situ hybridization analysis of genes coding collagen IV alpha1 chain, laminin beta1 chain, and S-laminin in prostate tissue and prostate cancer: increased basement membrane gene expression in high-grade and metastatic lesions. Prostate, 36: 143-150, 1998.
126. Eble, J. A., Golbik, R., Mann, K., and Kuhn, K. The alpha 1 beta 1 integrin recognition site of the basement membrane collagen molecule [alpha 1 (IV)]2 alpha 2(IV). Embo J, 12: 4795-4802, 1993.
127. Heinlein, C. A. and Chang, C. Androgen receptor in prostate cancer. Endocr Rev, 25: 276-308, 2004.
128. Quinn, D. I., Henshall, S. M., and Sutherland, R. L. Molecular markers of prostate cancer outcome. Eur J Cancer, 41: 858-887, 2005.
129. Grossmann, M. E., Huang, H., and Tindall, D. J. Androgen receptor signaling in androgen-refractory prostate cancer. J Natl Cancer Inst, 93: 1687-1697, 2001.
130. Huang, H. and Tindall, D. J. The role of the androgen receptor in prostate cancer. Crit Rev Eukaryot Gene Expr, 12: 193-207, 2002.
131. Koeneman, K. S., Kao, C., Ko, S. C., Yang, L., Wada, Y., Kallmes, D. F., Gillenwater, J. Y., Zhau, H. E., Chung, L. W., and Gardner, T. A. Osteocalcin-directed gene therapy for prostate-cancer bone metastasis. World J Urol, 18: 102-110, 2000.
132. Condon, M. S. The role of the stromal microenvironment in prostate cancer. Semin Cancer Biol, 15: 132-137, 2005.
133. Edlund, M., Sung, S. Y., and Chung, L. W. Modulation of prostate cancer growth in bone microenvironments. J Cell Biochem, 91: 686-705, 2004.
134. Tuxhorn, J. A., Ayala, G. E., and Rowley, D. R. Reactive stroma in prostate cancer progression. J Urol, 166: 2472-2483, 2001.
135. Tuxhorn, J. A., McAlhany, S. J., Dang, T. D., Ayala, G. E., and Rowley, D. R. Stromal cells promote angiogenesis and growth of human prostate tumors in a differential reactive stroma (DRS) xenograft model. Cancer Res, 62: 3298-3307, 2002.
136. Lehr, J. E. and Pienta, K. J. Preferential adhesion of prostate cancer cells to a human bone marrow endothelial cell line. J Natl Cancer Inst, 90: 118-123, 1998.
137. Bironaite, D., Siegel, D., Moran, J. L., Weksler, B. B., and Ross, D. Stimulation of endothelial IL-8 (eIL-8) production and apoptosis by phenolic metabolites of benzene in HL-60 cells and human bone marrow endothelial cells. Chem Biol Interact, 149: 37-49, 2004.
138. Rubin, J., Chung, L. W., Fan, X., Zhu, L., Murphy, T. C., Nanes, M. S., and Rosen, C. J. Prostate carcinoma cells that have resided in bone have an upregulated IGF-I axis. Prostate, 58: 41-49, 2004.
139. Arany, Z., Huang, L. E., Eckner, R., Bhattacharya, S., Jiang, C., Goldberg, M. A., Bunn, H. F., and Livingston, D. M. An essential role for p300/CBP in the cellular response to hypoxia. Proc Natl Acad Sci USA, 93: 12969-12973, 1996.
140. Niu, G., Wright, K. L., Huang, M., Song, L., Haura, E., Turkson, J., Zhang, S., Wang, T., Sinibaldi, D., Coppola, D., Heller, R., Ellis, L. M., Karras, J., Bromberg, J., Pardoll, D., Jove, R., and Yu, H. Constitutive Stat3 activity up-regulates VEGF expression and tumor angiogenesis. Oncogene, 21: 2000-2008, 2002.
141. Wei, D., Le, X., Zheng, L., Wang, L., Frey, J. A., Gao, A. C., Peng, Z., Huang, S., Xiong, H. Q., Abbruzzese, J. L., and Xie, K. Stat3 activation regulates the expression of vascular endothelial growth factor and human pancreatic cancer angiogenesis and metastasis. Oncogene, 22: 319-329, 2003.
142. Salnikow, K., Kluz, T., Costa, M., Piquemal, D., Demidenko, Z. N., Xie, K., and Blagosklonny, M. V. The regulation of hypoxic genes by calcium involves c-Jun/AP-1, which cooperates with hypoxia-inducible factor 1 in response to hypoxia. Mol Cell Biol, 22: 1734-1741, 2002.
143. Carrero, P., Okamoto, K., Coumailleau, P., O'Brien, S., Tanaka, H., and Poellinger, L. Redox-regulated recruitment of the transcriptional coactivators CREB-binding protein and SRC-1 to hypoxia-inducible factor 1alpha. Mol Cell Biol, 20: 402-415, 2000.
144. Ziel, K. A., Campbell, C. C., Wilson, G. L., and Gillespie, M. N. Ref-1/Ape is critical for formation of the hypoxia-inducible transcriptional complex on the hypoxic response element of the rat pulmonary artery endothelial cell VEGF gene. Faseb J, 18: 986-988, 2004.
145. Gray, M. J., Wey, J. S., Belcheva, A., McCarty, M. F., Trevino, J. G., Evans, D. B., Ellis, L. M., and Gallick, G. E. Neuropilin-1 suppresses tumorigenic properties in a human pancreatic adenocarcinoma cell line lacking neuropilin-1 coreceptors. Cancer Res, 65: 3664-3670, 2005.
146. Pore, N., Liu, S., Shu, H. K., Li, B., Haas-Kogan, D., Stokoe, D., Milanini-Mongiat, J., Pages, G., O'Rourke, D. M., Bernhard, E., and Maity, A. Sp1 is involved in Akt-mediated induction of VEGF expression through an HIF-1-independent mechanism. Mol Biol Cell, 15: 4841-4853, 2004.
147. Shi, Q., Le, X., Abbruzzese, J. L., Peng, Z., Qian, C. N., Tang, H., Xiong, Q., Wang, B., Li, X. C., and Xie, K. Constitutive Sp1 activity is essential for differential constitutive expression of vascular endothelial growth factor in human pancreatic adenocarcinoma. Cancer Res, 61: 4143-4154, 2001.
148. Agoulnik, I. U., Vaid, A., Bingman, W. E., 3rd, Erdeme, H., Frolov, A., Smith, C. L., Ayala, G., Ittmann, M. M., and Weigel, N. L. Role of SRC-1 in the Promotion of Prostate Cancer Cell Growth and Tumor Progression. Cancer Res, 65: 7959-7967, 2005.
149. Culig, Z., Comuzzi, B., Steiner, H., Bartsch, G., and Hobisch, A. Expression and function of androgen receptor coactivators in prostate cancer. J Steroid Biochem Mol Biol, 92: 265-271, 2004.
150. Culig, Z., Steiner, H., Bartsch, G., and Hobisch, A. Mechanisms of endocrine therapy-responsive and -unresponsive prostate tumours. Endocr Relat Cancer, 12: 229-244, 2005.
151. Wang, R., Cherukuri, P., and Luo, J. Activation of Stat3 sequence-specific DNA binding and transcription by p300/CREB-binding protein-mediated acetylation. J Biol Chem, 280: 11528-11534, 2005.
152. Maes, C., Stockmans, I., Moermans, K., Van Looveren, R., Smets, N., Carmeliet, P., Bouillon, R., and Carmeliet, G. Soluble VEGF isoforms are essential for establishing epiphyseal vascularization and regulating chondrocyte development and survival. J Clin Invest, 113: 188-199, 2004.
153. Zelzer, E., Mamluk, R., Ferrara, N., Johnson, R. S., Schipani, E., and Olsen, B. R. VEGFA is necessary for chondrocyte survival during bone development. Development, 131: 2161-2171, 2004.
154. Zelzer, E. and Olsen, B. R. Multiple roles of vascular endothelial growth factor (VEGF) in skeletal development, growth, and repair. Curr Top Dev Biol, 65: 169-187, 2005.
155. Maes, C., Carmeliet, P., Moermans, K., Stockmans, I., Smets, N., Collen, D., Bouillon, R., and Carmeliet, G. Impaired angiogenesis and endochondral bone formation in mice lacking the vascular endothelial growth factor isoforms VEGF164 and VEGF188. Mech Dev, 111: 61-73, 2002.
156. Zelzer, E., McLean, W., Ng, Y. S., Fukai, N., Reginato, A. M., Lovejoy, S., D'Amore, P. A., and Olsen, B. R. Skeletal defects in VEGF(120/120) mice reveal multiple roles for VEGF in skeletogenesis. Development, 129: 1893-1904, 2002.
157. Midy, V. and Plouet, J. Vasculotropin/vascular endothelial growth factor induces differentiation in cultured osteoblasts. Biochem Biophys Res Commun, 199: 380-386, 1994.
158. Deckers, M. M., Karperien, M., van der Bent, C., Yamashita, T., Papapoulos, S. E., and Lowik, C. W. Expression of vascular endothelial growth factors and their receptors during osteoblast differentiation. Endocrinology, 141: 1667-1674, 2000.
159. Cai, H. and Reed, R. R. Cloning and characterization of neuropilin-1-interacting protein: a PSD-95/Dlg/ZO-1 domain-containing protein that interacts with the cytoplasmic domain of neuropilin-1. J Neurosci, 19: 6519-6527, 1999.
160. Wang, L., Zeng, H., Wang, P., Soker, S., and Mukhopadhyay, D. Neuropilin-1-mediated vascular permeability factor/vascular endothelial growth factor-dependent endothelial cell migration. J Biol Chem, 278: 48848-48860, 2003.
161. Murga, M., Fernandez-Capetillo, O., and Tosato, G. Neuropilin-1 regulates attachment in human endothelial cells independently of vascular endothelial growth factor receptor-2. Blood, 105: 1992-1999, 2005.
162. West, D. C., Rees, C. G., Duchesne, L., Patey, S. J., Terry, C. J., Turnbull, J. E., Delehedde, M., Heegaard, C. W., Allain, F., Vanpouille, C., Ron, D., and Fernig, D. G. Interactions of multiple heparin binding growth factors with neuropilin-1 and potentiation of the activity of fibroblast growth factor-2. J Biol Chem, 280: 13457-13464, 2005.
163. Jankowski, K., Kucia, M., Wysoczynski, M., Reca, R., Zhao, D., Trzyna, E., Trent, J., Peiper, S., Zembala, M., Ratajczak, J., Houghton, P., Janowska-Wieczorek, A., and Ratajczak, M. Z. Both hepatocyte growth factor (HGF) and stromal-derived factor-1 regulate the metastatic behavior of human rhabdomyosarcoma cells, but only HGF enhances their resistance to radiochemotherapy. Cancer Res, 63: 7926-7935, 2003.
164. Wey, J. S., Gray, M. J., Fan, F., Belcheva, A., McCarty, M. F., Stoeltzing, O., Somcio, R., Liu, W., Evans, D. B., Klagsbrun, M., Gallick, G. E., and Ellis, L. M. Overexpression of neuropilin-1 promotes constitutive MAPK signalling and chemoresistance in pancreatic cancer cells. Br J Cancer, 93: 233-241, 2005.
165. McAllister, A. K. Conserved cues for axon and dendrite growth in the developing cortex. Neuron, 33: 2-4, 2002.
166. He, Z., Wang, K. C., Koprivica, V., Ming, G., and Song, H. J. Knowing how to navigate: mechanisms of semaphorin signaling in the nervous system. Sci STKE, 2002: RE1, 2002.
167. Bagnard, D., Vaillant, C., Khuth, S. T., Dufay, N., Lohrum, M., Puschel, A. W., Belin, M. F., Bolz, J., and Thomasset, N. Semaphorin 3A-vascular endothelial growth factor-165 balance mediates migration and apoptosis of neural progenitor cells by the recruitment of shared receptor. J Neurosci, 21: 3332-3341, 2001.
168. Gavazzi, I. Semaphorin-neuropilin-1 interactions in plasticity and regeneration of adult neurons. Cell Tissue Res, 305: 275-284, 2001.
169. Giger, R. J., Urquhart, E. R., Gillespie, S. K., Levengood, D. V., Ginty, D. D., and Kolodkin, A. L. Neuropilin-2 is a receptor for semaphorin IV: insight into the structural basis of receptor function and specificity. Neuron, 21: 1079-1092, 1998.
170. Miao, H. Q., Soker, S., Feiner, L., Alonso, J. L., Raper, J. A., and Klagsbrun, M. Neuropilin-1 mediates collapsin-1/semaphorin III inhibition of endothelial cell motility: functional competition of collapsin-1 and vascular endothelial growth factor-165. J Cell Biol, 146: 233-242, 1999.
171. Comoglio, P. M. and Trusolino, L. Invasive growth: from development to metastasis. J Clin Invest, 109: 857-862, 2002.
172. Tamagnone, L. and Comoglio, P. M. To move or not to move? Semaphorin signalling in cell migration. EMBO Rep, 5: 356-361, 2004.
173. Neufeld, G., Shraga-Heled, N., Lange, T., Guttmann-Raviv, N., Herzog, Y., and Kessler, O. Semaphorins in cancer. Front Biosci, 10: 751-760, 2005.
174. Bachelder, R. E., Lipscomb, E. A., Lin, X., Wendt, M. A., Chadborn, N. H., Eickholt, B. J., and Mercurio, A. M. Competing autocrine pathways involving alternative neuropilin-1 ligands regulate chemotaxis of carcinoma cells. Cancer Res, 63: 5230-5233, 2003.
175. Gomez, C., Burt-Pichat, B., Mallein-Gerin, F., Merle, B., Delmas, P. D., Skerry, T. M., Vico, L., Malaval, L., and Chenu, C. Expression of Semaphorin-3A and its receptors in endochondral ossification: Potential role in skeletal development and innervation. Dev Dyn, 2005.
176. Castro-Rivera, E., Ran, S., Thorpe, P., and Minna, J. D. Semaphorin 3B (SEMA3B) induces apoptosis in lung and breast cancer, whereas VEGF165 antagonizes this effect. Proc Natl Acad Sci USA, 101: 11432-11437, 2004.
177. Adams, G. B., Chabner, K. T., Foxall, R. B., Weibrecht, K. W., Rodrigues, N. P., Dombkowski, D., Fallon, R., Poznansky, M. C., and Scadden, D. T. Heterologous cells cooperate to augment stem cell migration, homing, and engraftment. Blood, 101: 45-51, 2003.
178. Wang, S., Gao, J., Lei, Q., Rozengurt, N., Pritchard, C., Jiao, J., Thomas, G. V., Li, G., Roy-Burman, P., Nelson, P. S., Liu, X., and Wu, H. Prostate-specific deletion of the murine Pten tumor suppressor gene leads to metastatic prostate cancer. Cancer Cell, 4: 209-221, 2003.
179. Wu, T. T., Sikes, R. A., Cui, Q., Thalmann, G. N., Kao, C., Murphy, C. F., Yang, H., Zhau, H. E., Balian, G., and Chung, L. W. Establishing human prostate cancer cell xenografts in bone: induction of osteoblastic reaction by prostate-specific antigen-producing tumors in athymic and SCID/bg mice using LNCaP and lineage-derived metastatic sublines. Int J Cancer, 77: 887-894, 1998.
180. Orimo, A., Gupta, P. B., Sgroi, D. C., Arenzana-Seisdedos, F., Delaunay, T., Naeem, R., Carey, V. J., Richardson, A. L., and Weinberg, R. A. Stromal fibroblasts present in invasive human breast carcinomas promote tumor growth and angiogenesis through elevated SDF-1/CXCL12 secretion. Cell, 121: 335-348, 2005.
181. Sheng, T., Li, C., Zhang, X., Chi, S., He, N., Chen, K., McCormick, F., Gatalica, Z., and Xie, J. Activation of the hedgehog pathway in advanced prostate cancer. Mol Cancer, 3: 29, 2004.
182. Chesire, D. R. and Isaacs, W. B. Beta-catenin signaling in prostate cancer: an early perspective. Endocr Relat Cancer, 10: 537-560, 2003.
183. Guvakova, M. A., Yakubov, L. A., Vlodavsky, I., Tonkinson, J. L., and Stein, C. A. Phosphorothioate oligodeoxynucleotides bind to basic fibroblast growth factor, inhibit its binding to cell surface receptors, and remove it from low affinity binding sites on extracellular matrix. J Biol Chem, 270: 2620-2627, 1995.
184. Jin, C., Wang, F., Wu, X., Yu, C., Luo, Y., and McKeehan, W. L. Directionally specific paracrine communication mediated by epithelial FGF9 to stromal FGFR3 in two-compartment premalignant prostate tumors. Cancer Res, 64: 4555-4562, 2004.
185. Hsieh, C. L. and Chung, L. W. New prospectives of prostate cancer gene therapy: molecular targets and animal models. Crit Rev Eukaryot Gene Expr, 11: 77-120, 2001.
186. Sakurai, F., Terada, T., Maruyama, M., Watanabe, Y., Yamashita, F., Takakura, Y., and Hashida, M. Therapeutic effect of intravenous delivery of lipoplexes containing the interferon-beta gene and poly I: poly C in a murine lung metastasis model. Cancer Gene Ther, 10: 661-668, 2003.
187. Sioud, M. and Sorensen, D. R. Cationic liposome-mediated delivery of siRNAs in adult mice. Biochem Biophys Res Commun, 312: 1220-1225, 2003.
188. Wang, X. P., Yazawa, K., Templeton, N. S., Yang, J., Liu, S., Li, Z., Li, M., Yao, Q., Chen, C., and Brunicardi, F. C. Intravenous delivery of liposome-mediated nonviral DNA is less toxic than intraperitoneal delivery in mice. World J Surg, 29: 339-343, 2005.
189. Noble, C. O., Kirpotin, D. B., Hayes, M. E., Mamot, C., Hong, K., Park, J. W., Benz, C. C., Marks, J. D., and Drummond, D. C. Development of ligand-targeted liposomes for cancer therapy. Expert Opin Ther Targets, 8: 335-353, 2004.
190. Gianni, L., Kearns, C. M., Giani, A., Capri, G., Vigano, L., Lacatelli, A., Bonadonna, G., and Egorin, M. J. Nonlinear pharmacokinetics and metabolism of paclitaxel and its pharmacokinetic/pharmacodynamic relationships in humans. J Clin Oncol, 13: 180-190, 1995.

What is claimed is:

1. A method for interfering with the osteomimetic properties of a cell comprising introducing into a cell of a subject in need thereof of an osteomimecry interfering compound, wherein said compound prevents or ameliorates the expression of the osteomimetic properties of said cell.

2. A method for interfering with the osteomimetic properties of a prostate cancer cell comprising introducing into a prostate cancer cell of a subject in need thereof of an osteomimecry interfering compound, wherein said compound prevents or ameliorates the expression of the osteomimetic properties of said prostate cancer cell.

3. The method according to claim 2, wherein said osteomimicry interrfering compound inhibits one or more determinants governing prostate cancer bone colonization wherein said determinants comprise prostate cancer cell adhesion, extravasation, migration, and interaction with bone cells or a combination thereof.

4. The method according to claims 1-3, wherein said osteomimicry interrfering compound interferes with the ability of the cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL), and/or to increase calcification, mineralization and bone turnover through the expression of genes normally restricted to osteoblasts and through epithelial to mesenchymal transition (EMT).

5. The method according to any of claims 1-4, wherein said osteomimicry interrfering compound comprises a beta 2 microglobulin (β2M) siRNA, a β2M antibody, a GPCR antagonist, a PKA/CREB signal activation interrupter, a selective agent that interferes with β2M/PKA/CREB signaling, a selective agent that interferes with CREB transcription, phosphorylation and complex formation, a selective agent that interferes with β2M complex formation with either an intracellular protein or a membrane receptor or any combination thereof.

6. The method according to any of claims 1-5, wherein said osteomimicry interfering compound is administered in combination with one or more antagonists, one or more anti-angiogenic agents, one or more cytotoxic drugs, or any combination thereof.

7. A method for treating or ameliorating an osteotropic-related cancer or other proliferative disorder comprising introducing into a cell of said cancer or other proliferative disorder of a subject in need thereof an osteomimecry interfering compound, wherein said compound interferes with the osteomimetic potential of said cell, prevents its growth, abrogates its supportive blood vessels, eliminates the survival androgen receptor signaling and causes massive cell death in pre-existing cancer or any combination thereof

8. The method of claim 7, wherein said cancer or other proliferative disorder is selected from the group consisting of osteosarcoma, prostate, breast, colon, lung, brain, multiple myeloma, thyroid, melanoma or any other disease or disorder with calcification potential.

9. The method according to claims 7-8, wherein said osteomimicry interrfering compound interferes with the ability of the cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL), and/or to increase calcification, mineralization and bone turnover through the expression of genes normally restricted to osteoblasts and through epithelial to mesenchymal transition (EMT).

10. The method according to claims 7-9, wherein said compound a beta 2 microglobulin (β2M) siRNA, a β2M antibody, a GPCR antagonist, a PKA/CREB signal activation interrupter, a selective agent that interferes with β2M /PKA/CREB signaling, a selective agent that interferes with CREB transcription, phosphorylation and complex formation, a selective agent that interferes with β2M complex formation with either an intracellular protein or a membrane receptor or any combination thereof.

11. The method according to claims 7-10, wherein said osteomimicry interfering compound is administered in combination with one or more antagonists, one or more anti-angiogenic agents, one or more cytotoxic drugs, or any combination thereof.

12. A method for treating and/or ameliorating an osteotropic-related disease or proliferative disorder comprising introducing into osteotropic cells of a subject in need thereof a vector comprising an osteomimecry interfering regulatory region sequence, or transcriptionally active fragment thereof of one or more osteomimecry target genes including, but not limited to, genes that are related to or downstream from the VEGF axis, AR axis, GPCR axis, PKA/CREB axis, genes depicted in Appendix A, or any combination thereof, wherein said osteomimecry interfering regulatory region sequence can regulate the activity or activities of one or more of said genes by interfering with the osteomimetic potential of said osteotropic cells.

13. The method of claim 12, wherein said cancer or other proliferative disorder is selected from the group consisting of osteosarcoma, prostate, breast, colon, lung, renal, brain, multiple myeloma, thyroid, melanoma or any other disease consisting of benign prostate hyperplasis, vascular plaque formation in cardiovascular conditions or disorders with excessive calcification and mineralization potential, or any combination thereof.

14. The method of claim 13, wherein said osteotropic-related disease or proliferative disorder comprises increased bone turnover through enhanced interaction between RANK and RANKL such as osteoporosis and increased cancer bone colonization through enhanced osteomimicry and recruitment of host cells that promote osteoclastogenesis and osteoblastogenesis.

15. A method for identifying a compound which modulates the osteomimetic potential of a cell comprising: (a) contacting a test compound to a cell that exhibits osteomimetic potential; (b) measuring expression of one or more osteomimetic gene products in the cell; and (c) comparing the level of expression of one or more osteomimetic gene products in the cell in the presence of the test compound to a level of expression of one or more osteomimetic gene products in the cell in the absence of the test compound; wherein, if the level of the expression of one or more osteomimetic gene products in the cell in the presence of the test compound differs from the level of expression of the one or more osteomimetic gene products in the cell in the absence of the test compound, a compound that modulates expression of the one or more osteomimetic gene products is identified.

16. The method of claim 15, wherein the cell that exhibits osteomimetic potential comprises a cancer cell from osteosarcoma, prostate, breast, colon, lung, brain, multiple myeloma, thyroid, melanoma or any other known disease or disorder with osteomimetic or calcification potential.

17. The method according to claims 15-16, wherein said test compound comprises an osteomimicry interrfering compound that interferes with the ability of the cancer cells to express highly restricted bone-like proteins comprising one or more of osteocalcin (OC), bone sialoprotein (BSP), SPARC/osteonectin (ON), osteopontin (OPN) and the receptor activator of NF-κB ligand (RANKL), and/or to increase bone turnover through Epithelial to Mesenchymal Transition (EMT).

18. The method according to claims 15-17, wherein said test compound comprises a b2m siRNA, a b2m antibody, a GPCR antagonist, a PKA/CREB signal activation interrupter, a selective agent that interferes with b2m/PKA/CREB signaling, a selective agent that interferes with CREB transcription factor and complex formation, or any combination thereof.

19. The method according to claims 15-18, wherein said osteomimicry interrfering compound is administered in combination with one or more antagonists, one or more anti-angiogenic agents, one or more cytotoxic drugs, or any combination thereof.


SEQUENCE LISTING (6 sequences)

SEQ ID NO: 1 - DNA, 946 bp, Homo Sapiens
gagctcggatccactagtaacggccgccagtgtgctggaattccccttctgcagggtcaggaggagaatcgtggggccaggagggcagaggcacactccatcttcgtgctcctcacaggccctgctccctgcctgctaagacacagggaagggggcccccacctcagtgcctccctcccttccctgtgcctgtgtacctggcagtcacagccacctggcgtgtcccagaaaccaaccggctgacctcatctcctgcccggccccacctccattggctttggcttttggcgtttgtgctgcccgaccctttctcctgtccggatgcgcagggcagggctgagccgtcgagctgcacccacagcaggctgcctttggtgactcaccgggtgaacgggggcattgcgaggcatcccctccctgggtttggctcctgcccacggggctgacagtagaaatcacaggctgtgagacagctggagcccagctctgcttgaacctattttaggtctctgatccccgcttcctctttagactcccctagagctcagccagtgctcaacctgaggctgggggtctctgaggaagagtgagttggagctgaggggtctggggctgtcccctgagagaggggccagaggcagtgtcaagagccgggcagtctgattgtggctcaccctccatcactcccagggcccctggcccagcagccgcagctcccaaccacaatatcctttggggtttggcctacggagctggggcggatgaccccaaatagccctggcagattccccctagacccgcccgcaccatggtcaggcatgcccctcctcatcgctgggcacagcccagagggtataaacagtgctggaggctggcggggcaggccagctgagtcctgagcagcaaagggcgaattctgcagatatccatcacactggcggccgct

SEQ ID NO: 2 - DNA, 1468 bp, Homo Sapiens
gtggcacatatacaccatggaatactatgcagccataaaaaatgatgagttcatgtcctttgtggggacatgtatgaaattggaaaccatcattctcagtaaactatcacaagaacaaaaaaccaaacaccacatattctcactcataggtgggaattgaacaatgagatcacatggacacaggaaggggaacatcacactctggggactgttgtggggtggggggagtaaggggagggatagcattgggagatatacctaatgctagatgatgagttagtgggtgcagcacaccagcatggcacatgtgtacgtatgtaactaacctgcacacaatgtgcacatgtaccctaaaacttaaagtataataataaaaaaaattaagagaaaaaaagaaaaaaaatgatattcattaatttttgatttctcaagcagacttcgcaactggaggaagaataaaatgactagactaggagaatatgcaaactattaagctagatttccctttataaattaaaaaattagtactttagtttatcaatccattctttgtggtgttggtttcatgaatcatttcaaaaacaatggatcactcctgctagctctagtcattttgttattctcataggaaaaaaattaaatatgaaaatgaatagaaaagatatatatagaagcccaagaaaaatcagctgacctcacatgcacgacaggaaggccacataaatggacaatatacagagatttaatttacaaaacaaaatataaaatctgcctctcagtggtatgattctcaaaagttctaacttttatactcagcatcatgttttagcaactatatgttacaaagtctgaccgacttaatcatatcaactttaatttatgagtcaatgaagtatatttcaggaggaaacatcaaatgatattaaaatattgatggttcatctgctggtttcccttattatttagtttttctttctttttttagctaaactaatgtaaaagttatatctaatgacagcaagctttcctttctttcgacatagtgaaaacttgtgtaattatgaaatttttaaaaggttaaagcctttgttatttattttaattcaaatccagtatattattatacatattcggagcccaaactattcatcttcatctaaaccttcaattaaattccacaatgcaaacctcttggctctagaatcacgtttcttgtttattcaactgagcctgtgtcttgaaaaagtgttgaagtttgggggttttctggtgagaatccacgttctgacatcaccttggtcgtgacagtgattggctgttggaaggcaaagaagagtttatagccagcaagagcaagtgaatgagtgagtgagagggcagaggaaatactcaatctgtgccactcactgccttgagcctgcttcctcactccaggactgccagagggtaagatttaatagaacaacgg

SEQ ID NO: 3 - DNA, 2200 bp, Homo Sapiens
  unsure N position 495, 573: a or g or c or t
gggaaggagacaatagtgtcaacttgggattgcctaaggcaacaacagagcaaaacaagaacgctttggttctctgggtctctgtccctgattgcatagcgggtcattgttgggaaatatttcctcacctggcattccaagaaatggtgagctccacagctgtatatagtcctgtcattaaatacaggagtgttctatcccgctggaattaagaaaattggtagaaccagattgtggtctgaaatcttttttcagaaatgctgccatcgtgtggcactgcggagctatgaccagaagagtcctgtaaagggtcgtatggttcatctcaagatggctgggctccagcataatctattcctataattaattctagcttcatattgaatcattcccgtgggcacagagtaaactacagtaaatcctgtggaaattttgttgtttttagaattttcggacttccctccactaaattgacaacatgacacgcttatgcgngtatgtttaaaggaaaaaaatagtttttagaagcagaaaaaagaagtctattttgcaactttataatctgtgtgcttnctattttatagagatagtcgtcatcttacttattaaaatgggtgcttattacctacaaaccaatcatatcaattcatctggaatacatccaatttaagggagacatatttccccctaccaaatgttcatgaaacctatgaattagctatacactatcactgcaagacattatttaatctatatttatattaaaagtaatatttggcaaaaggaagctgacactttaggactaataaaaaccacaattacttttgcagcaacctaataataaataggaccatttatttttcatctcaattacacacaagtcttaacaataaaggtgtaaggtaaataaatagtgcaatctgcatttcacaactgagaagcaaatgaagataagtaatctcaaggcaatattaaatattttaaaaggacccagagctctgctatccctgaattctgctctaatattcggactttccctgtaattttctttcattcagacaccttttaaatacctagtaaagtgttttttaatacagaaatttttaaaaatgtttttctttttaagtggcctactttacataccttgggagaaaaactagaaaaaaagatgattccaaaatcgaatctgttcctttagaaatgtgcaaaatttccttattgatgcatacaatttaaagatcttacgtctactctcattttaataacctgttcttttaaaggacattacaattcgtgactgcctgcccctcttaaaaatttcataatagttaacacacatatagtccttaagatacgcagagcatttgcatctaatatgtgctaagcattgctagtttaacatactaattcatttaaacccctcaaaaaccccatgacctaggtaatagtattgcatttcatggatgagggaacaaggataggtaggctgggcgatttgcccaaggttgcacaggtcagcagtgacacagcggaattcagaaccacggtctggctcctgaagcagccctctcaagcagtcatccttctctcagtcagaaactgctttacttctgcaacatctagaataaattaccattcttctatttcatatagaattttatattttaatgtcactagtgccatttgtctaagtaacaagctactgcatactcgaaatcacaaagctaagcttgagtagtaaaggacagaggcaagttttctgaactccttgcaggcttgaacaatagccttctggctcttcaataagtacaatcatacaggcaagagtggttgcagatattacctttatgttacttaaaccgaaagaaacaaaaatccattgtatttaattttacattaatgtttttccctactttctccctttttcatgggatccctaagtgctcttcctggatgctgaatgcccatcccgtaaatgaaaaagctagttaatgatattgtacataagtaatgttttaactgtagattgtgtgtgtgcgtttttggtttttttttgttttaaccacaaaaccagagggggaagtgtgggagcaggtgggctgggcagtggcagaaaacctcatgacacaatctctccgcctccctgtgttggtggaggatgtctgcagcagcatttaaattctgggagggcttggttgtc

SEQ ID NO: 4 - DNA, 2313 bp, Homo Sapiens
gaattccttgtactttttttcccttctcagttctgcacttaactcgtctaaaaaaattaaaaaagaatttaagaaaccacaaagctaagctgggtgcggtggctcacgcctgtaatcctagcactttgggaagccaaggcattcggattgcccaagctcaggagttcgagaccagcctgggcaacatgttgaaaccccatttctactaaaaatacaataaattagctgggtgttgtggcatgtgcgcctgtaatcccagctactctggaggctgaggcgcgataattgcttgaacccgggaggcagaggttgcagtgagccgaaatcataccactgcactccagcctgggcgacagagtgagtgagactctgtctcaaaacaaaacaaaacaaacaaacaaaaaaaccggaaaccaacaaaactttttgaggaacaaagggaaccaggtattttattaattctcatacctccagagtgttaggcacaaaataaacattcaaccaagacctgttgcactgagcagttcatatataacaggagtgacccaagttgaaacgtagaatcagccctctcataccactttttgccaggtgatcataggcaagttacttagcatctatgtttccttattattaaaatggtcataattacaatgcctaagataagggggttgctgtgaagattattaaatcctcagtaaactttggctattgttactcctatgattatcatcaatatcatcaattaccttatctgttcaatactggtggcacaggtccaccagctagatgtctaatcccttatgtgtctattagtggtacaagtggagtttgagtgggatttttttttttttttttaagaccagttccaaatcatcaaggatgataccactagtagcagcttgtcttgtctgtacagtggtaagtcctggccttgcctttgtggcaaatacaacccccttgaattgcttggcccttctcagcattgcctaatattagggaggactcctgtaaagctcactggttagaagatcaagacacttgggcctggttctgcccctgggggccattgggtaattccttggagtctccaggcctcacttgccctctgaacaagaaagaggcctgttctggtcatccctccagcctgtccagccctggcactctgtgagtcggtttaggcagcagccccggaacagatgaggcaggcagggttgggacgtttggtcaggacagcccaccgcaaaaagaggaggaaagaaatgaaagacagagacagctttggctatgggagaaggaggaggccgggggaaggaggagacaggaggaggagggaccacggggtggaggggagatagacccagcccagagctctgagtggtttcctgttgcctgtctctaaacccctccacattcccgcggtccttcagactgcccggagagcgcgctctgcctgccgcctgcctgcctgccactgaggtatgtgtgacccccgcccagcctttcccttctatagttgcaccaaccccgacacccccgttcacgccgtcagctcgtgtgcaagggagggaagctctgctgaggatgcgcctctcctcccggctccatcacggctccccttaagagcatggccctcggtcctgtctgcctgttgcttttcagaaggtggactcactgtgtaactttgtcttcccttacaggtttacaggaaaataatctcactatgttcttcgggggagcattttctcactctctgtttttctctgtgtctgtctctggtttcagaggctgcctgcctgtcctctttgctccctttgcaaatgtggcagcctcctcctttcctgggaatctgatcccatcacagctgccacagggacctggccagcaaccggagtctgtcctccagatctcggtcaggggttctgttttccaaaaagggactttgcagaacaatcagttgatctctgaaagggaaagggggaggcttcaccattaatccacacctctgggaagcttctgttttcctctaattctcctcactcccaaacaccaccttccgtccccccaatacacaaatttcagcaccattctgcctgaaatggcaccatcacaacctcagtcttgggttaggtgttgttcctgtcctgagttccttgggatggtaaacacaggcagtagcccttagtttatctagatctgaaaacccagacatcagatatcgtcaaccaagacatgggtgtaatgggaggtggagtgtgctgggggagatattctcagaagggggaaagggggaagggaagagggagagaattc

SEQ ID NO: 5 - DNA, 4255 bp, Homo Sapiens
acaccaaatatttataaatataactcacacaaataaaacctctttggtgttctcaaaattttgaagaatgtaaaaggtttgaaaattgctgatctagcaaatgactgaacatgaacagctatagtatttgtacctgcccagcagtgcagcaattccttatccttctcatatctgcactttaattttcctttgacaaatatctctccctcctctcagcccatgacatgaggttcacatggggttaacttaattccctggctcaaaggaaaggtattaaattcagacttgtatccaaccattcctgaagctagacttagccctatttttcaataacatgaaccaatcaattttcacatgagtccaaaataattctatgttaatacactaaggtactaggaaatatagtttgagaaatgttgatccaaacattgtgttatttacagtggagtattgacataaactttgaatcttcaaatatgttctggtgtcttggcatctcttaatacctattagcttacaaggctttcactcaactattttataattttgataatgacttaattgattagttgatatattgttaaaataaatatattaatgaatttatgataaataaggcagataaataagacatgcaattaggaagacatgttaaacaaattgttataataatacaatcactctcagcttaggatagctcctggccactttctctctgggtggtttttactctgggagtagtttaaatcattatctagtagtagtttaaagcattatctttgcctaagagctttcgctgactccccacatttgcattgtactaagagttttctctgactccccacataggtctagaccctagtattataagattctcattgtacttgcactttgccttcaaagtactaatcacggttttgttagtgatttgtgtgatgatttgttgaatctttttttttttcccactagggtgtaagccccatgttccatcttgatcaccatgtttctagcccagtgctggcatatagtgggttctcactaatatatctgtagagtaaatgaagaaatgcatgagtgacatgacaggagaatttaaggatgccatgggagcataaaacagagggagccacctgggtgaggagagctgagaaagacttctggagaggcgacatttgagctgagaaaggaaagacaagtgggagagtcctccaggtgtagaagttggagagatgagcgctccagttaggtagtatttgaagctgatgtagaaaaggagtcttgagccagcttgtgaaggactattggagagttttatttttatttttatcttttttttaatttttgagacagaatcttgctttgtctcccaggctggagtgcagtggcatgattgtagcttactgcagcttcgacctcctgggctcaaacaatccacctatctcagccttctgagtaactgggaccagagatgtgcaccaaaatgcctggctaatttgttcattttttgtaaagatagggtctccctatgttccccaggctattctccatctcctgggctccagtgatcctcacgcctcggccacccaaagtgctgggattatagaagtgaaccactgcgcctggcctattgaaggtttttaatcttcagagtttcgactttatcaacaacacttagaagccaccaaagaattgcaggtatggaaatgacatatacttttgcttttagaagaaaatcctgatcagtgtgcacagaattcttcagggggcaagtgtgattcattctgataagatatagcatggcttagactgggagactggcagaggctttgaagatttctttgctcaaattttattcagcaagtatttaccatgcacctactatagcaggcaacatttttaggaaatggtgaatgttacagaggtgaataatacagcaagagtcgttgaacatatggagtttatctattagttggggagtgaatgttgacaaaggaataagtaaatacataggcaagaaagatacattacctgtgaaacagcagcaggtagactgacagtggagtatctaatacagcctatggaagccagaagatagtgggatgacatttttggagtactagtagaaatgtcatatgaagaactctgtaggaatgtaacatacggtcccatatatgaagctcctgggtcaagtatacctgaacataattcagggatttgagggactttcttgtaacctgaggatcaagatgtcaaggaattaaaaacatgtataaaacattgttgtataaaaacccattaaaaagaatggaagacactatagtaaaatcattgtgggtttagttgttataacacattttaaaaatctttgatcccaatcaatatttataagaaagaagaaatatggaattatttcctgagtcaaggagcagggagagaatgaggaagaagaggaggaggaggagggggaggaggagacaataaacctacttcccaaagttaacaaacaaaaagtgggaagaggtcaaagactacaaggagtagaattaacgtcaattgtttctatgtttgagtctgaaaattttttgtcccttctccaccaacctatatattgatacacatataaatgctaaaggcatttttgaatttgaacagatcattttctttgtatggctgcctttaaaaaaaattcaacctggtcactcttcctcaacatttactgaggtctaagtgttcaatttagaacacatgctttaataactcagagacctgtcatttgtcacaaatcttgcctagagaaatactcattagcgaattaggcagaaagaggatgcaaaataaaaaggcacagtagtcccctgatatccatggaagactggttccaggacaccaccaaacccctccccgcaaataccaaaatccatggatgttcaagtttcttaacatatcatggcatagtatttgcatttaacctacacacatcctcttgtacacttgaaattatctttagattatttataatacttaatagaatgtaaatgctatgtaactagttgtgtatcatttaggaaatgatcacaagaaaaaaagtctacagatgttagtccagacacagccatcctttttttttttttcaaatatttttgatctgtggttcattgcatccacagatgtggaacccatggatactgtgggctaactgtattaataaaaaagtggaaacatcctaagtttcatgggtgtttaaattggtcagcaacttccttctgaagaagtatcagaatttgtgagcaatgttaatatttttgttttctcactaagagccacagttctgaatagaggtttttaaaaagccctagcaaggtttctttagcaatgaaactaacatttaactgtatcatcagcttcgtgttacatctctttcctgactgttgggtgagccctcctcggatgcttgcttctggctacacgcccctttacccttttctctgcactgttttcatctttataaagtcagagttggtgtctataggctctctactgccacattcaagacctgcctcgctcaatgtcaccttcaagatgcagaaatagggatttgggaaggggattgtgaaattttcgaagtcttccaaaatactttgagaaactatatttggaagcactttggggggagaggttggacaggaagggtcttcagagatcatcaaatttaactttctaaatcctaaggaggaaaccgagactccaggatgtgaagtcccttctctaccaaactagaatggatgcaggaggaatgtctgaggtgcaatccttatcctttagcaaaggtgtcctctgcgtcttctttaacccatctcttggacctccagaaagacagctgaggatggcaaggggagtctggaaccactggagtagcccccagcctcctccttggagggcccccatgaaggaggcccttcagtgacagagattgagagagagggagggcgaaaggaaggaaggggagccagaggtgggagtggaagaggcagcctcgcctggggctgattggctcccgaggccagggctctccaagcggtttataagagttggggctgccgggcgccctgcccgctcgcccgcgcgccccaggacccaaagccgggctccaagtcggcgccccacgtcgaggctccgccgcagcctccggagttggccgcagacaagaaggggagggagcgggagagggaggagagctccgaagcgagagggccga

SEQ ID NO: 6 - DNA, 6905 bp, Homo Sapiens
tctagaaaataattcccaatattgaatcccaaagaattcaacatttgggctgtcgtttgaaagataagttgaatttggtcatgaaggaagagaggggggatacaatttcagtaaaaggtaacagcaaggtccaaagacagtcaggtcttcagtagtatggagtatattcagagggagccaagatgtctgatgtgaactaaaaagattggtggttggtaggaggaagaggtgtgagaagaggctgtaaagaaaaattgaaacttgattgtgatggactttaaaggctaggctatgggacttggacatgaatctgcaggccagtgtttgcagactggcgcccataactgtctatcacagcaacacagacatgtgttgtttggcctgcagaggtttggcctgcatgatgattttaaaccatctgaattagtagccatcattttcaaaaatcaagagatgccacattaaaatatggaatgctgctgttcttgaaaataatgaaacatctggaacattgaggccacattcctgactgacagcaatcagttggagctgcgtagtgactgcccactttacatggggcatctgatccctagtcgattacagctgccaccacttccctttatctctctaataccaagctcttttcactcatttttgttacttaagagatatttgggtttgaaacctctgatgcaggtaattgagggttatagagcagaggacagatgctatcagagttgtcttttaagaaagaaccctctgttcttcattttgttgaagatagcctggaagagggcagccaggggagaagttagggctggagctatgagaaagcataagatgagatgatggcttcaacattgaggacagaaagaatattgagatgagaaagtagtccatataagcatctatgcaaaggaaatagcagatgtcctcaaatcagcagaggcaacaactctgaaagtttattcataagcccctcttttcatctccaatccagttcaaatgtaattatttaaattgttcttcactctccttcctggatcatgaatgagctccttaaatgcagggtccacagtgtcctattcatcagtgaattccaagtgcctagcacagagcctggcaaatagtaaatgcttaacaaatattcgttcagtgcatgaattggagtgattctctactttgcctcataagttgaaaaaaggtttattacatacctaaatatgctgaaatcacagggcatttggcaaccccccaaaaccaaaactcccagtttggaaacagaattttaattctgtgaaaataaaatccattcatttattcaaaaaatatttattaaacaatgaccatgtccacaccaggctgagtcctaaggattcaatgatgaacaaaaaccaacatgattcctgctcttaggaaacatacagttcagtgaggaaaacagattgtgagaagtcctccaacaaatactgggtgctattaaaatatattaaaaggtgagtgggtgagggacttgagctagcctaggtggttcaggaagtcttcctggatgtgctgatatgcataggcattaactagataaatagagagaaggatgaaccaacattgcaggtagagggaacagaatatgcaaaggcaggaaggattatggagtcgttggaggacctgaataaaggcccagtgtaagtggatctcagaaaacaggaggaaaggtgtatgagatgagatcagagaggcagatcatgtggggtatggttaatgttttggacttttctattaagagcaatggggagacagtgacaggacttaaacggggaaataatatgaccagattaaactttctaaaaaaccctctatgcaaatatatattgagagttaattattgacaaagattcaaaggcaacaaagtggagagagaatagtattttcaaaaaatggtgccaaaacaataggacatctatattaaaagttgggtatctgtctacaaaacttaattcaaaatggatcacagacctaaatgtaaaactgaaagctatacaacttctggaaggaaaacacagatgggaatctgtgtgatcttgagtttgaaaatgatttattatatctgacaccataatccgtaagttaacataattcataagtgaacaaagtgatgaactggacttcatcagaatttaaaatgtttgtgcttcaaaagacactggtatgataatgaagacaaactacagataagatattgttgaatcatatttctgataaaggaattgtggctcagaatacataactctaaacccccataataaattacaagtagcccaattaaaaaaaaaaaaagagaaaaaatttacagtcttcatcaaagaaagtatcaattgtaaaataagcacatgaaaaatgctctgcatctttattcatggggggatgaaataaaaattaaatgggaaagacacctctaattagaatactaaaattaaaaagactgaccataccaagtattggtgaagtggaaatgtaaaatgatacaatcaacttaggtagatgatttggaagtttcttacaaaagtaggtgtatacctaccctgtgactcacccattccatggctaagtatttacctgagagaaatgaaagaatacatccatacaaagatgtttatacaaatatttatagcagttttatttgtagtagccccaaactgaaaagaacccaaatgtccatcaaaagtgaatggataaacaaagcgtggtacagcaatgcaatagaatactacttagcaataaagaagaatgagctagtgatatacataacagcttaaatgtacatcaaaggcattgtgctcagtgaaagatgcaagtaaaaaaaaaaaagagtacatgctgtatagttccattgacataaaactctggaaagtgaaaaacagtctatactgacagaaagcagatcattggttgcctgaggaggaggagtataggagaggtggagggaaaatgtacaaagtggcacaataaaaacttttggaatcatagatatattcactatcttgattgagtgatgatttcatgagtgcacgtgcgtgtgtcaaaaatgatcaatttatgcaactttaaatatgtgcagtttattgtatatatcaattatacctcagtacggctattaaaaagaaaccctctggctgcacaatgcagaactgattctaggaaagagtggagggaggatgaccatttacagtgctccaggtggaagagaacggtgccttctggaagtgaactaggttggcaacaacagagatgaaataaatgggcagatgtgtgagatacttaggaaataaaacccgatggtcaccattttccaaaggtcagctcatcctggctttccagagcaaagagctagggaagactttattaataaatccctcttgaagttgcagaggaagcttatagcagaaacttactctcaacctgactaatctgagagaacacctctggttccatttgattactaaaaaactgcaaagaacaggaggagaaagaagaagaaagctggtacaaacagtgaacttatataatattaatcaataattgtctcttgttcttaaaagcaatgggaagaaaatgagatttgagctggaagatcagagttcaaaatccaaataaagtatatggccctaatatgcttatagtagttaacctttcctgataatgatataattgttgacagcaccatctttaaaataaaataacatagtaatccttcagatttgtagaagatctttcctgtttacaagtttgttctatacacattatgtcttttaaatgacacactagccttctgagggtaacttatattggcaacagttttcagatgtggaaactgtgaagacaatgttggtgatgtggaagcaacataaactttggagtctttcagacccaggtttgaatgtcagactgctttttattcagagtaacttcagagcattatttctcaccttaattttttttcaggcctctttgtgtctatgtgtcctcttcactcctgtccattgtttcttcagtgatttttgccaccttccttcactgttagtgtgtagacacatagttctcctggctctgagagcctatgttaattccattctaccatcctgccacggcccactcaattcctattgagcaatgctagttgaaagttgtggtgggattaaatgttgcaatgagtattcaaatgaggttgaagtatctacgcattctacttacatatggtgaggtatattcaaggaagctgtagccattaaaatctcaggaaataatttttcacctcctcaggtgaaagggtcttcaggcctttgtgttctggaaggttcatttatagccatttcccaaatgacaatgcgattgatgagtctagagtctagctcaaatagcaatggactggaagactagtttaggttttactaatgtggaacatagaacaaattatgtccttgtttcagcctgttcatctgtgaaatagagcctatcatatccagtcttccttgcctttaggtttgagttaccttctttggtcaaggtaagtaaatgcctatgatgtttggctgtgcacaagataaagctacaacaaagctacaacccatcttttctctgtagaagactcaaaaagcaaaagagacccaggaaaatctcggaatgacttttggaacagagagcctccccagaatcagaagtcaaggaatttaaacatagggaaggcccaggtctctactgacataaaggaaagatgttttcttataggtttcacgtttacattttctctctcttgatcccattcccacttgcatctgccacctttacacagggcttatgggacctcctccacaaaagagcagttgcagtaacccacatcatcctctacgccctggctgtccatcaagaggcgaaaagcagccctatataggttctatccttggatagttccagttgtaaagtttaaaatatgcgaaggcaacttggaaaagcaagcggctgcatacaaagcaaacgtttacagagctctggacaaaattgagcgcctatgtgtacatggcaagtgtttttagtgtttgtgtgtttacctgcttgtctgggtgattttgcctttgagagtctggagagtagaagtactggttaaaggaacttccagacaggaagaaggcagagaagagggtagaaatgactctgattcttggggctgagggttcctagagcaaatggcacaatgccacgaggcccgatctatccctatgacggaatctaaggtttcagcaagtatctgctggcttggtcatggcttgctcctcagtttgtaggagactctcccactctcccatctgcgcgctcttatcagtcctgaaaagaacccctggcagccaggagcaggtattcctatcgtccttttcctccctccctcgccccaccctgttggttttttagattgggctttggaaccaaatttcctgagtgctggcctccaggaaatctggagccctggcgcctaaaccttggtttaggaaaccaggagctattcaggaagcaggggtcctccagggctagagctagcctctcctgccctcgcccacgctgcgccagcacttgtttctccaaagccactaggcaggcgttagcgcgcggtgaggggaggggagaaaaggaaaggggaggggagggaaaaggaggtgggaaggcaaggaggccggcccggtgggggcgggacccgactcgcaaactgttgcatttgctctccacctcccagcgccccctccgagatcccggggagccagcttgctgggagagcgggacggtccggagcaagcccacaggcagaggaggcgacagagggaaaaagggccgagctagccgctccagtgctgtacaggagccgaagggacgcaccacgccagccccagcccggctccagcgacagccaacgcctcttgcagcgcggcggcttcgaagccgccgcccggagctgccctttcctcttcggtgaagtttttaaaagctgctaaagactcggaggaagcaaggaaagtgcctggtaggactgacggctgcctttgtcctcctcctctccaccccgcctccccccaccctgccttccccccctcccccgtcttctctcccgcagctgcctcagtcggctactctcagccaacccccctcaccacccttctccccacccgcccccccgcccccgtcgcccagcgctgccagcccgagtttgcagagaggtaactccctttggctgcgagcgggcgagctagctgcacattgcaaagaaggctcttaggagccaggcgactggggagcggcttcagcactgcagccacgacccgcctggttaggctgcacgcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaacaagaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatg